(19) United States
(12) Patent Application Publication
(10) Pub. No.: US 2009/0054319 A1
(43) Pub. Date: Feb. 26, 2009

(54) Methods and Compositions for the Treatment of Hypertension and Gastrointestinal Disorders

(75) Inventors: John Jeffrey Talley, Somerville, MA (US); Mark G. Currie, Sterling, MA (US); Shannon Roberts, Cambridge, MA (US)

Correspondence Address: FISH & RICHARDSON PC, P.O. BOX 1022, MINNEAPOLIS, MN, 55440-1022, US

(73) Assignee: MICROBIA, INC., Cambridge, MA (US)

(21) Appl. No.: 11/908,667

(22) PCT Filed: Mar. 17, 2006
(86) PCT No.: PCT/US06/09696

§ 371 Date: May 15, 2008

Related U.S. Application Data
(60) Provisional application No. 60/662,946, filed on Mar. 17, 2005.
(60) Provisional application No. 60/701,258, filed on Jul. 21, 2005.

(51) Int. Cl. A61K 38/00; A61P 1/00

(52) U.S. Cl. 514/12; 514/14

(57) ABSTRACT

The use of guanylin potentiating agents for treating various disorders, including hypertension as well as IBS and other gastrointestinal disorders and conditions (e.g., gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, chronic constipation, and disorders and conditions associated with constipation is described.

This application is the National Stage of International Application No. PCT/US2006/009696, filed on Mar. 17, 2006, which claims the priority to U.S. provisional Application Ser. No. 60/662,946, filed on Mar. 17, 2005 and Ser. No. 60/701,258, filed on Jul. 21, 2005. The contents of all applications are hereby incorporated by reference in their entireties.

### BACKGROUND

Irritable bowel syndrome (IBS) is a common chronic disorder of the intestine that affects 20 to 60 million individuals in the United States alone (Lehman Brothers, Global Healthcare-Irritable Bowel Syndrome Industry Update, September 1999). IBS is the most common disorder diagnosed by gastroenterologists (28% of patients examined) and accounts for 12% of visits to primary care physicians (Camilleri 2001 Gastroenterology 120:652-668). In the United States, the economic impact of IBS is estimated at $25 billion annually, through direct costs of health care use and indirect costs of absenteeism from work (Talley 1995 Gastroenterology 109:1736-1741). Patients with IBS have three times more absenteeism from work and report a reduced quality of life. Sufferers may be unable or unwilling to attend social events, maintain employment, or travel even short distances (Drossman 1993 Dig Dis Sci 38:1569-1580). There is a tremendous unmet medical need in this population since few options exist to treat IBS.

Patients with IBS suffer from abdominal pain and a disturbed bowel pattern. Three subgroups of IBS patients have been defined based on the predominant bowel habit: constipation-predominant (c-IBS), diarrhea-predominant (d-IBS) or alternating between the two (a-IBS). Estimates of individuals who suffer from c-IBS range from 20-50% of the IBS patients with 30% frequently cited. In contrast to the other two subgroups that have a similar gender ratio, c-IBS is more common in women (ratio of 3:1) (Talley et al. 1995 Am J Epidemiol 142:76-83).

The definition of and diagnostic criteria for IBS have been formalized in the “Rome Criteria” (Drossman et al. 1999 Gut 45:Suppl II:1-81), which are well accepted in clinical practice. Briefly, the criteria specify that for at least 12 weeks (consecutive or non-consecutive) in the preceding 12 months of abdominal discomfort or pain at least two of the following three features must occur: (1) relieved with defecation, (2) onset associated with a change in frequency of stool, and (3) onset associated with a change in form (appearance) of stool. The Rome II criteria also state that the symptoms that cumulatively support the diagnosis of irritable bowel syndrome include: abnormal stool frequency (“abnormal” may be defined as greater than 3 bowel movements per day and less than 3 bowel movements per week), abnormal stool form (lumpy/hard or loose/watery stool), abnormal stool passage (straining, urgency, or feeling of incomplete evacuation), passage of mucus, and bloating or feeling of abdominal distension.

However, the complexity of symptoms has not been explained by anatomical abnormalities or metabolic changes. This has led to the classification of IBS as a functional GI disorder, which is diagnosed on the basis of the Rome criteria and limited evaluation to exclude organic disease (Ringel et al. 2001 Annu Rev Med 52: 319-338). IBS is considered to be a “biopsychosocial” disorder resulting from a combination of three interacting mechanisms: altered bowel motility, an increased sensitivity of the intestine or colon to pain stimuli (visceral sensitivity) and psychosocial factors (Camilleri 2001 Gastroenterology 120:652-668). Recently, there has been increasing evidence for a role of inflammation in the etiology of IBS. Reports indicate that subsets of IBS patients have small but significant increases in colonic inflammatory and mast cells, increased inducible nitric oxide (NO) and synthase (iNOS) and altered expression of inflammatory cytokines (reviewed by Talley 2000, Medscape Coverage of DDW Week).

Hypertension is a major risk factor for cardiovascular disease, the leading cause of death for both men and women in many industrialized countries. Almost one third of the adults in North America and 44% of the adults in Europe suffer from hypertension. The Seventh Report of the Joint National Committee on Prevention, Detection, Evaluation, and Treatment of High Blood Pressure (JNC 7) defines Stage I Hypertension as systolic blood pressure (SBP) of 140-159 mm Hg, or diastolic blood pressure (DBP) of 90-99 mmHg.

Blood pressure is a function of: the force/amount of blood the heart pumps, the diameter of arteries, and the volume of blood in the bloodstream. Generally speaking, blood pressure can be lowered by: (1) decreasing the blood volume, (2) increasing the diameter of arteries by decreasing vasoconstriction or increasing vasodilation, or (3) decreasing force of blood pumped by the heart.

The Renin-Angiotensin-Aldosterone Pathway (RAAS) plays a role in many aspects of blood pressure regulation. Many of the currently available blood pressure drugs affect the RAAS pathway. Most current therapies for hypertension fall into one of the following classes: (1) diuretics, (2) ACE inhibitors (ACEI), (3) Angiotensin Receptor Blockers (ARBs), (4) beta-blockers (BB), and (5) calcium channel blockers (CCBs).

About 63% of patients with hypertension require treatment with at least two antihypertensive drugs. Currently prescribed drug combinations include: ACEIs and CCBs; ACEIs and diuretics; ARBs and diuretics; BBs and diuretics; centrally acting drugs and diuretics; and two different diuretics. Sufficient reduction in hypertension can lead to reduced risk of stroke, reduce risk of myocardial infarction, reduced risk of heart failure and overall reduced risk of death.

Guanylin is an intestinal peptide that stimulates chloride secretion. In humans, guanylin is produced initially as a 115 amino acid protein referred to as preproguanylin. The mature protein, which is believed to be the active form, has 15 amino acids. Guanylin is inactivated by cleavage by the serine protease, chymotrypsin, which is present in the gastrointestinal tract, and by a chymotrypsin-like enzyme that is present in the kidney. Guanylin is an agonist of the transmembrane guanylate cyclase (GC-C) receptor. The GC-C receptor is present on the apical plasma membrane of enterocytes in intestinal tract and in other epithelia. Activation of the GC-C receptor by guanylin in the intestine increases cGMP levels. This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are fluid and electrolyte homeostasis, the regulation of epithelial cell proliferation and the induction of apoptosis (Shailubhai 2002 Curr Opin Drug Dis Devel 5:261-268). Other GC-C receptor agonists (GC-C agonists) include uroguanylin, renoguanylin, lymphoguanylin and the E. coli heat stable ST peptide.

### SUMMARY

Compositions and related methods for treating IBS and other gastrointestinal disorders and conditions (e.g., gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders) are described herein.

Also described herein are compositions and related methods for treating hypertension and/or reducing risk factors associated with hypertension (including: myocardial infarction, heart failure, and stroke).

The methods and compositions described herein relate to the administration of a chymotrypsin inhibitor, an inhibitor of a chymotrypsin-like enzyme, a prodrug of a chymotrypsin inhibitor, a prodrug of an inhibitor of a chymotrypsin-like enzyme, an antibody directed against chymotrypsin, and/or an antibody directed against a chymotrypsin-like enzyme.

Inhibitors of chymotrypsin and inhibitors of a chymotrypsin-like enzyme are useful in the methods and compositions described herein. Such inhibitors can inhibit the activity of chymotrypsin or a chymotrypsin-like enzyme, e.g., in the intestine, kidney or elsewhere in the body, and thereby interfere with the inactivation of guanylin by proteolytic cleavage. In this manner, the inhibitors are expected to potentiate the activity of guanylin present in the body whether the guanylin is produced by or administered to the patient.

The prodrugs of inhibitors can be metabolized to active inhibitors. In some instances the administered prodrug itself has some or considerable inhibitory activity. Some compounds that are inhibitors of a chymotrypsin-like enzyme will also have the ability to inhibit the activity of chymotrypsin. Because the inhibitors decrease the activity of chymotrypsin and/or chymotrypsin-like enzymes, they can interfere with inactivation of guanylin in the intestinal tract caused by chymotrypsin cleavage of guanylin, inactivation of guanylin in the kidney caused by a chymotrypsin-like enzyme cleavage of guanylin, and/or inactivation of guanylin caused by cleavage by chymotrypsin or a chymotrypsin-like enzyme elsewhere in the body. The inhibitors, whether administered directly or as a prodrug that is metabolized to an inhibitor, can potentiate the action of naturally-occurring guanylin or other GC-C receptor agonist (whether endogenous or administered), thereby increasing or regularizing intestinal motility, reducing hypertension, or increasing GC-C receptor activity. The prodrugs can also be used in combination therapy, for example in combination with a GC-C receptor agonist or some other treatment for a gastrointestinal disorder. In certain embodiments, the GC-C receptor agonist is guanylin or a biologically active variant or fragment thereof.

Antibodies directed against chymotrypsin or a chymotrypsin-like enzyme can be used in any of the methods and compositions described herein in which an inhibitor of chymotrypsin or a chymotrypsin-like enzyme or a prodrug of an inhibitor of chymotrypsin or a chymotrypsin-like enzyme can be used. The antibodies can inhibit the activity of chymotrypsin or a chymotrypsin-like enzyme and thereby interfere with the inactivation of guanylin by proteolytic cleavage.

Inhibitors of chymotrypsin, inhibitors of a chymotrypsin like enzyme, prodrugs of a chymotrypsin inhibitor, prodrugs of an inhibitor of a chymotrypsin-like enzyme, antibodies directed against chymotrypsin, and antibodies directed against a chymotrypsin-like enzyme are collectively referred to as “guanylin potentiating agents”.

The various compositions described herein can also be used for treating obesity, congestive heart failure and benign prostatic hyperplasia (BPH).

Without being bound by any particular theory, in the case of IBS and other gastrointestinal disorders the compositions described herein are useful because they can increase or regularize gastrointestinal motility, decrease inflammation, and/or decrease gastrointestinal pain or visceral pain by potentiating guanylin activity.

Without being bound by any particular theory, in the case of hypertension the compositions described herein are useful because they are expected to prevent proteolytic inactivation of guanylin in the kidney, resulting in higher renal levels of guanylin, which will act to increase electrolyte and fluid secretion into urine, resulting in lower blood volume and consequently lower blood pressure. In addition, increased renal guanylin levels might lead to increased plasma guanylin levels which may reduce release of aldosterone, a regulator of blood pressure.

Pharmaceutical compositions comprising certain compounds that are capable of reducing the activity of chymotrypsin or a chymotrypsin-like enzyme, particularly the ability of chymotrypsin or a chymotrypsin-like enzyme to inactivate guanylin by proteolytic cleavage are described herein. Also described herein are pharmaceutical compositions comprising a chymotrypsin inhibitor or a prodrug of an inhibitor of a chymotrypsin-like enzyme as well as combination compositions comprising a chymotrypsin inhibitor or an inhibitor of a chymotrypsin-like enzyme and a second therapeutic agent. Pharmaceutical compositions comprising a prodrug of a chymotrypsin inhibitor and a chymotrypsin inhibitor as well as combination compositions comprising a prodrug of a chymotrypsin inhibitor, a chymotrypsin inhibitor and an additional therapeutic agent (e.g, guanylin) are described as are pharmaceutical compositions comprising a prodrug of an inhibitor of a chymotrypsin-like enzymes and an inhibitor of a chymotrypsin-like enzyme. Also described are compositions comprising a prodrug of an inhibitor of a chymotrypsin-like enzyme, an inhibitor of a chymotrypsin-like enzyme and an additional therapeutic agent. In addition, a prodrug of a chymotrypsin inhibitor and a prodrug of an inhibitor of a chymotrypsin-like enzyme can be used in combination with or without an additional therapeutic agent. Also described are pharmaceutical compositions comprising an antibody directed against chymotrypsin or a chymotrypsin-like enzyme and, optionally, a second therapeutic agent, e.g., guanylin, a guanylin variant, or an analgesic agent.

Methods for treating other disorders such as congestive heart failure and benign prostatic hyperplasia by administering a guanylin potentiating agent are described. Such agents can be used in combination with natriuretic peptides (e.g., atrial natriuretic peptide, brain natriuretic peptide or C-type natriuretic peptide), a diuretic, or an inhibitor of angiotensin converting enzyme or an inhibitor of vasopressin.

Methods and compositions for increasing intestinal motility by administering a composition comprising a guanylin potentiating agent are described herein. Intestinal motility involves spontaneous coordinated distensions and contractions of the stomach, intestines, colon and rectum to move food through the gastrointestinal tract during the digestive process.

For the treatment of gastrointestinal disorders, the agent can be administered orally, by rectal suppository or parenterally.

In various embodiments, the patient is suffering from a gastrointestinal disorder; the patient is suffering from a disorder selected from the group consisting of: a gastrointestinal motility disorder, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, chronic constipation, hypertension, obesity, congestive heart failure, or benign prostatic hyperplasia.

In another aspect, the invention features a method for treating a patient suffering from constipation, the method comprising administering a composition comprising, consisting essentially of, or consisting of a guanylin potentiating agent and a pharmaceutically acceptable carrier. Clinically accepted criteria that define constipation include from the frequency of bowel movements, the consistency of feces and the ease of bowel movement. One common definition of constipation is less than three bowel movements per week. Other definitions include abnormally hard stools or defecation that requires excessive straining (Schiller 2001 Aliment Pharmacol Ther 15:749-763). Constipation may be idiopathic (functional constipation or slow transit constipation) or secondary to other causes including neurologic, metabolic or endocrine disorders. These disorders include diabetes mellitus, hypothyroidism, hyperthyroidism, hypocalcaemia, Multiple sclerosis, Parkinson's disease, spinal cord lesions, Neurofibromatosis, autonomic neuropathy, Chagas disease, Hirschsprung disease and cystic fibrosis. Constipation may also be the result of surgery or due to the use of drugs such as analgesics (like opioids), antihypertensives, anticonvulsants, antidepressants, antispasmodics and antipsychotics.

In various embodiments, the constipation is associated with use of a therapeutic agent; the constipation is associated with a neuropathic disorder; the constipation is post-surgical constipation (postoperative ileus); the constipation is associated with a gastrointestinal disorder; the constipation is idiopathic (functional constipation or slow transit constipation); the constipation is spinal chord injury induced; the constipation is thyroid disease related; the constipation is associated with neuropathic, metabolic or endocrine disorder (e.g., diabetes mellitus, hypothyroidism, hyperthyroidism, hypocalcaemia, Multiple Sclerosis, Parkinson's disease, spinal cord lesions, neurofibromatosis, autonomic neuropathy, Chagas disease, Hirschsprung disease or cystic fibrosis), surgery, the use of drugs such as analgesics (e.g., opioids), antihypertensives, anticonvulsants, antidepressants, antispasmodics and antipsychotics.

A method for treating a patient suffering from a gastrointestinal disorder is described. The method comprises administering a composition comprising, consisting essentially of, or consisting of a guanylin potentiating agent and a pharmaceutically acceptable carrier.

In various embodiments, the patient is suffering from a gastrointestinal disorder; the patient is suffering from a disorder selected from the group consisting of: a gastrointestinal motility disorder, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, chronic constipation, obesity, congestive heart failure, or benign prostatic hyperplasia.

A method for increasing gastrointestinal motility in a patient is described. The method comprises administering to the patient a composition comprising, consisting essentially of, or consisting of a guanylin potentiating agent and a pharmaceutically acceptable carrier.

A method for decreasing gastrointestinal pain or visceral pain in a patient is described. The method comprises administering to the patient a composition comprising, consisting essentially of, or consisting of a guanylin potentiating agent and a pharmaceutically acceptable carrier.

A method for treating hypertension is described. The method comprises: administering to the patient a pharmaceutical composition comprising, consisting essentially of, or consisting of a guanylin potentiating agent and a pharmaceutically acceptable carrier. The composition can be administered in combination with another agent for treatment of hypertension, for example, a diuretic, an ACE inhibitor, an angiotensin receptor blocker, a beta-blocker, and a calcium channel blocker.

A method for increasing the activity of an intestinal guanylate cyclase (GC-C) receptor in a patient is described. The method comprises administering to the patient a composition comprising, consisting essentially of, or consisting of a guanylin potentiating agent and a pharmaceutically acceptable carrier.

A method for increasing the level of cGMP in a patient is described. The method comprises administering to the patient a composition comprising, consisting essentially of, or consisting of a guanylin potentiating agent and a pharmaceutically acceptable carrier.

A method for treating a disorder ameliorated by increasing cGMP levels is described. The method comprises administering a composition comprising, consisting essentially of, or consisting of a guanylin potentiating agent and a pharmaceutically acceptable carrier.

A pharmaceutical composition comprising, consisting essentially of, or consisting of a guanylin potentiating agent and a pharmaceutically acceptable carrier is described. The composition can include a polymer that controls the release of the inhibitor. The composition can include a second agent, e.g., a GC-C receptor agonist such as guanylin or a biologically active variant or fragment thereof.

A method for treating obesity is described. The method comprises administering a pharmaceutical composition comprising, consisting essentially of, or consisting of a guanylin potentiating agent and a pharmaceutically acceptable carrier is described. The composition can be administered in combination with another agent for treatment of obesity.

A method for treating congestive heart failure is described. The method comprises: administering to the patient a pharmaceutical composition comprising, consisting essentially of, or consisting of a guanylin potentiating agent and a pharmaceutically acceptable carrier. The composition can be administered in combination with another agent for treatment of congestive heart failure, for example, a natriuretic peptide such as atrial natriuretic peptide, brain natriuretic peptide or C-type natriuretic peptide, a diuretic, or an inhibitor of angiotensin converting enzyme.

A method for treating benign prostatic hyperplasia is described. The method comprises: administering to the patient a pharmaceutical composition comprising, consisting essentially of, or consisting of a guanylin potentiating agent and a pharmaceutically acceptable carrier. The composition can be administered in combination with another agent for treatment of BPH, for example, a 5-alpha reductase inhibitor (e.g., finasteride) or an alpha adrenergic inhibitor (e.g., doxazosine).

A method for treating secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions is described. The method comprises: administering to the patient a pharmaceutical composition comprising, consisting essentially of, or consisting of a guanylin potentiating agent and a pharmaceutically acceptable carrier.

A guanylin potentiating agent can be administered in combination with guanylin (including mature or immature guanylin, e.g., guanylin having a pre-sequence, a pro-sequence or both a pre-sequence and a pro-sequence) or a biologically active fragment or variant thereof. A number of guanylin and variants thereof are depicted in FIGS. 3-5. Also useful are guanylin related polypeptides comprising, consisting of, or consisting essentially of the amino acid sequence: Xaa₁ Xaa₂ Xaa₃ Cys₄ Xaa₅ Xaa₆ Xaa₇ Xaa₈ Xaa₉ Xaa₁₀ Xaa₁₁ Cys₁₂ Xaa₁₃ Xaa₁₄ Xaa₁₅ Xaa₁₆ (SEQ ID NO:1) wherein:

Xaa₁ is Ser, Asn, Tyr, Ala, Gln, Pro, Lys, Gly, or Thr, or is missing;
Xaa₂ is His, Asp, Glu, Ala, Ser, Asn, Gly, or is missing;

### Xaa₃ is Thr, Asp, Ser, Glu, Pro, Val or Leu;

### Xaa₅ is Asp, Ile or Glu;

### Xaa₆ is Ile, Trp or Leu;

### Xaa₇ is Cys, Ser, or Tyr;

Xaa₈ is Ala, Val, Thr, Ile, Met or is missing;
Xaa₉ is a) any amino acid, b) Phe, Tyr, Asn, Trp, c) an amino acid other than Phe, Trp, or Tyr, d) non-aromatic amino acid or e) is missing;

### Xaa₁₀ is Ala, Val, Met, Thr or Ile;

### Xaa₁₁ is Ala or Val;

### Xaa₁₃ is Ala or Thr;

### Xaa₁₄ is Gly, Ala or Ser;

Xaa₁₅ is Cys, Tyr or is missing; and
Xaa₁₆ is: a) any amino acid; c) is missing or d) His or Leu or Ser.

As noted above, immature guanylin can include a pre-sequence and/or a prosequence. Immature guanylin is processed to yield the mature protein. Immature guanylin generally includes a so-called “pre sequence” followed by a “pro sequence” and then the mature polypeptide sequence. The pre sequence is important for secretion of the polypeptides. The pro sequence may be important for proper folding of the mature protein under at least some conditions. Various pre sequences and pro sequences are discussed in great detail below in the section describing guanylin and guanylin variants.

The publications and patents referenced herein are incorporated by reference in their entirety.

### FIGURES

FIG. 1 shows the results of a T84 cGMP assay to assess guanylin activity after a chymotrypsin digestion assay. The chymotrypsin digestion assay was performed in the presence or absence of chymostatin.

FIG. 2 shows the results of LC/MS analysis of the processing of guanylin in a chymotrypsin digestion assay. The chymotrypsin digestion assay was performed in the presence or absence of chymostatin.

FIG. 3 depicts the amino acid sequence of various deletion variants of human guanylin in which one, two, three or four amino acids are deleted. The deleted amino acids are between the first and fourth cysteines in human guanylin as well as amino terminal to the first cysteine in human guanylin.

FIG. 4 depicts the amino acid sequence of various insertion variants of human guanylin in which one, two, three or four amino acids are inserted. The inserted amino acids are between the first and fourth cysteines in human guanylin as well as amino terminal to the first cysteine in human guanylin and carboxy terminal to the fourth cysteine in human guanylin.

FIG. 5 depicts the amino acid sequence of various guanylins and variants thereof.

FIG. 6 is a table depicting the sequences of various guanylin polypeptides, including pre sequences, pro sequences, prepro sequences, mature sequences and combinations thereof, SEQ ID NOs 4734-4852 respectively (across each row).

### DETAILED DESCRIPTION

Guanylin binds to and activates the guanylate cyclase (GC-C) receptor, a key regulator of fluid and electrolyte balance in the intestine and kidney. When stimulated, this receptor, which is located on the apical membrane of the intestinal epithelial surface, causes an increase in intestinal epithelial cyclic GMP (cGMP). This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are fluid and electrolyte homeostasis, the regulation of epithelial cell proliferation and the induction of apoptosis (Shailubhai 2002 Curr Opin Drug Dis Devel 5:261-268).

In the human body an inactive form of chymotrypsin, chymotrypsinogen is produced in the pancreas. When this inactive enzyme reaches the small intestine it is converted to active chymotrypsin by the excision of two di-peptides. Active chymotrypsin will cleave peptides at the peptide bond on the carboxy-terminal side of Trp, Tyr or Phe and can cleave a peptide at the peptide bond on the amino terminal side of a Leu, Ile or Val (and, at elevated pH, His). Thus, chymotrypsin can cleave guanylin as shown by Greenberg et al. (J Investig Med 45:276-82, 1997). Chymotrypsin-mediated inactivation of guanylin can be prevented by chymostatin, a chymotrypsin inhibitor. Santos-Neto et al (2003 Pharm & Toxicol 92:114) observed that chymostatin was required in order to observe guanylin-induced intestinal fluid secretion in the suckling mouse model.

### Chymotrypsin Inhibitors

A wide variety of peptide and non-peptide chymotrypsin inhibitors are known and can be used in the methods described herein. For example:
1. tissue-factor-pathway inhibitor (TFPI) (Peterson et al 1996 Eur J Biochem 235:310-6; for examples see human (GENBANK® AAH15514 GI:15930156), mouse (GENBANK® AAH36146 GI:23271605), and dog (GENBANK® AAB32443 GI:833924));
2. α-2 antiplasmin (Potempa et al. 1988 Science 241: 699-700, GENBANK® Accession P08697, GI:112907, SEQ ID MALLWGLLVLSWSCLQGPCSVFSPVSAMEPLGRQLTSGPN
3. QEQVSPLTLLKLGNQEPGGQTALKSPPGVCSRDPTPEQTHRLARAMMAFTAD LFSLVAQTSTCPNLILSPLSVALALSHLALGAQNHTLQRLQQVLHAGSGPCLP HLLSRLCQDLGPGAFRLAARMYLQKGFPIKEDFLEQSEQLFGAKPVSLTGKQE DDLANINQWVKEATEGKIQEFLSGLPEDTVLLLLNAIHFQGFWRNKFDPSLTQ RDSFHLDEQFTVPVEMMQARTYPLRWFLLEQPEIQVAHFPFKNNMSFVVLVP THFEWNVSQVLANLSWDTLHPPLVWERPTKVRLPKLYLKHQMDLVATLSQL GLQELFQAPDLRGISEQSLVVSGVQHQSTLELSEVGVEAAAATSIAMSRMSLS SFSVNRPFLFFIFEDTTGLPLFVGSVRNPNPSAPRELKEQQDSPGNKDFLQSLK GFPRGDKLFGPDLKLVPPMEEDYPQFGSPK (SEQ ID NO:2);
4. members of the serpin α-1 antichymotrypsin family (Forsyth et al. 2003 Genomics 81: 336-45; for example see CAS Registry No. 141176-92-3; functional variants thereof are described in European patent application EP1415664 and in Plotnick et al. 2003 Biochemistry 33:29927 (for example the P2 (Leu-357) variant);
5. gelin (U.S. Pat. No. 5,397,694, partial sequence (aa 1-29) can be found at GENBANK® Accession AAB27871, GI:409493, (SEQ ID NO: 3) VDEKAEVTDGLCGDWTCSGAQVXQNDAAV), which has been proposed as a treatment for dermatitis as well a periodontitis and gingivitis;
6. hirustasin (Sollner et al. 1994 Eur J Biochem. 219: 937-43, GENBANK® Accession No P80302, GI:461516, (SEQ ID NO 4) QGNTCGGETCSAAQVCLKGKCVCNEVHCRIRCKYGLKKD ENGCEYPCSCAKASQ);
7. certain eglins including eglin C (GENBANK® Accession P01051, GI:124128, (SEQ ID NO. 5) TEFGSELKSFPEVVGKTVDQAREYFTLHYPQYDVYFLPEGSPVTLDLRYNRV RVFYNPGTNVVNHVPHVG) are peptide inhibitors of chymotrypsin. For other examples of eglins, see those disclosed in U.S. Pat. No. 5,180,667, U.S. Pat. No. 6,342,373, U.S. Pat. No. 4,636,489, Seemuller et al. 1981 Methods Enzymol. 804-816, Seemueller et al. 1986 Research Monographs in Cell and Tissue Physiology 337-59, Nick et al 1988 Adv in Experimental Medicine and Biology 240:83-8, and Schnebli et al 1986 Pulm. Emphysema Proteolysis (conference) CAN 107:228147 AN 1987:628147), which has been considered as a treatment for emphysema and for use as a non-steroidal anti-inflammatory agent;
8. inhibitors from Bombyx mori (see, e.g., JP 4013698 A2 and JP 04013697 A2; CA registry No. 142628-93-1, (SEQ ID NO. 6) DEPTTKPFCEQAFGDCGTPY and CA registry No. 142628-94-2, (SEQ ID NO. 7) DKPTTEPFIC EQRFGNCGTG);
9. the leech derived peptide thrombin inhibitor, hirudin (Zwilling 1968 Hoppe-Seyler's Zeitschrift fuer Physiologische Chemie 349:1787-8, CA Registry No. 8001-27-2, see for example, Genbank AAA01384 GI:269388, (SEQ ID NO. 8) ITYTDCTESGQNLCLCEGSNVCGKGNKClLGSQGKDNQCVTGEGTPKPQSHN QGDFEPIPEDAYDE). Hirudin variants are disclosed in the literature (for examples see those in U.S. Pat. No. 5,674,838, Great Britain patent application GB2242681 and those described in Wirsching et al 2003 Molecular Genetics and Metabolism 80:451-462);
10. a shorter hirudin variant, hirulog/BG 8967 (CA Registry No. 128270-60-0, (SEQ ID NO. 9); FPRPGGGGNGDFEEIPEEYL; Angiomax® (bivalirudin)) may also have chymotrypsin inhibition activity and may thus be useful in the present invention along with other peptides disclosed in PCT publication WO04076484 and U.S. Pat. Nos. 5,196,404; and
11. secretory leukocyte protease inhibitor (SLPI) (for examples see GENBANK® CAA28187 GI:758101 (human), GENBANK® NP_{—}445824.1 GI:16758102 (rat), and GENBANK® NP_{—}035544.1 GI:6755574 (mouse); also Farley et al 1997 Drugs and the Pharmaceutical Sciences 84:305-334.
12. α-1 anti-trypsin which can inhibit elastase as well as chymotrypsin and thus may be useful in the present invention (for examples see GENBANK® CAB06092 GI:2780174 (human) and GENBANK® NP_{—}001009663 GI:57527135 (rat)). This product has been sold to treate α-1 anti-trypsin deficiency (a genetic disorder) as Zemaira™ (Aventis Behring; FDA biologics license 2003 License #1281), Prolastin® (Bayer), and Aralast™ (Baxter).

In addition, a large number of peptide inhibitors of chymotrypsin are reviewed by Schoofs et al. (2002 Curr Pharm Des. 8: 483-91) and by Salier et al. (1996 Biochem J. 315:1-9). McBride et al. 1996 J Mol Biol. 259: 819-27 and McBride et al. 2000 J Pept Sci. 6:446-52 disclose chymotrypsin inhibitors derived from combinatorial peptide libraries including those represented by CAS registry Nos. 306762-66-3, 306762-67-4, 306762-68-5, 306762-69-6, 306762-70-9, 306762-71-0, 306762-72-1, 306762-73-2, 306762-74-3, 306762-75-4, 178330-92-2, 178330-93-3, 178330-94-4, and 178330-95-5.

In addition, various small molecule inhibitors of chymotrypsin have been described including:
1. the compound depicted below and described in EP 0071433 (CAS registry No. 81459-62-3)

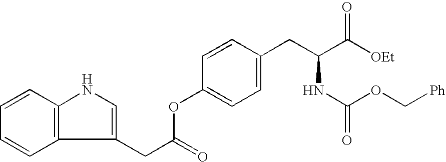

Other chymotrypsin inhibitors disclosed in EP 0071433 include those specifically identified as chymotrypsin inhibitors on pages 11-16 of the application and those identified by CA registry Nos. 81459-79-2, 81460-01-7, 85476-59-1, 85476-62-6 (also known as FK-401 CA Index name 1H-Indole-3-acetic acid, 5-methoxy-2-methyl-, 4-[[2-[4-[2-(4-morpholinyl)ethyl]-1-piperazinyl]ethoxy]carbonyl]phenyl ester, trihydrochloride), 85476-63-7, 85476-67-1, 85476-70-6, 85858-66-8, 85858-68-0, 85858-69-1, 85858-70-4, 85858-71-5, 85858-72-6, 85858-73-7, 85858-75-9, 85858-77-1, 85858-79-3, 85858-81-7, 85858-83-9, 85858-84-0, 85858-85-1, 85858-87-3, 85858-89-5, 85858-90-8, 85858-92-0, 85879-03-4, 85879-05-6, 85879-06-7, and 85879-08-9);
2. compounds with chymotrypsin inhibition activity described in JP 56092217 A2;
3. compounds with chymotrypsin inhibition activity described in U.S. Pat. No. 4,755,383 (including, 1-Naphthaleneacetic acid, 4-[[4-(1-methylethyl)-1-piperazinyl]carbonyl]phenyl ester (CA registry No. 90186-24-6), Acetic acid, 9H-fluoren-9-ylidene-, 4-[[4-(1-methylethyl)-1-piperazinyl]carbonyl]phenyl ester (CA registry No. 90185-93-6), 1-Naphthalenecarboxylic acid, 1,2,3,4-tetrahydro-4-[[4-(1-methylethyl)-1-piperazinyl]carbonyl]phenyl ester, monomethanesulfonate (CA registry No. 89703-10-6), 1-Naphthalenecarboxylic acid, 1,2,3,4-tetrahydro-, 4-[(4-cyclohexyl-1-piperazinyl)carbonyl]phenyl ester (CA registry No. 85858-74-8), and 1-isopropyl-4-[4-(1,2,3,4-tetrahydronaphthoyloxy) benzoyl]piperazine methanesulfonate).
4. chymotrypsin inhibitors described in U.S. Pat. Nos. 4,755,383 and 4,639,435 include those of Formula I:

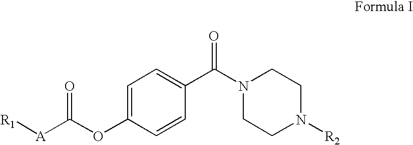

wherein A is a single bond, or an alkylene, vinylene, —O-alkylene or methine group; R¹ is a bicyclic carbocyclic residue which may partly be saturated and may optionally be substituted by at least one member of the class consisting of lower alkyl, lower alkoxy, oxo and nitro groups and halogen atoms; a fluorene residue which may optionally have an oxo group; a fluorenylidene group; an anthracene residue; a phenanthrene residue which may partly be saturated and may optionally be substituted by at least one lower alkyl group; a benzofuran or thianaphthene residue which may optionally be substituted by at least one member of the class consisting of lower alkyl and lower alkoxy groups; a benzopyran or benzazine residue which may partly be saturated and may optionally be substituted by at least one member of the class consisting of oxo and phenyl groups; a phthalimide residue; a benzodiazone residue; an isoxazole residue which may optionally be substituted by at least one member of the class consisting of lower alkyl and phenyl groups; an alkylenedioxybenzene residue; or a xanthene residue, and R² is a loweralkyl, cycloalkyl, cycloalkylalkyl or aralkyl group.
The methods of producing the compounds of the formula (I) and their chymotrypsin inhibitory activity characteristics have been described in Japanese Patent Application No. 109192/1982 and in Japanese Patent Application filed Apr. 28, 1983.
5. inhibitors described in EP 0128007 and U.S. Pat. No. 4,620,005 including: 1-isopropyl-4-[(4-(1,2,3,4-tetrahydro-1-naphthoyloxy)phenyl)carbonyloxymethyl carbonyl]piperazine; 1-isopropyl-4-[(4-(1,2,3,4-tetrahydro-1-naphthoyloxy)phenyl)ethyl carbonyl]piperazine; and 1-isopropyl-4-[(4-(1,2,3,4-tetrahydro-1-naphthoyloxy)phenyl)methylcarbonyl]piperazine;
6. inhibitors described U.S. Pat. No. 4,898,876 including inhibitors: 1-isopropyl-4-(4-(5,6,7,8-tetraphydronaphthalene-1-acetyloxy)benzoyl)piperazine hydrochloride; 1-isopropyl-4-(4-(9-fluorenylidene acetyloxy)benzoyl)piperazine; 1-isopropyl-4-(4-(thianaphthene-2-acetyloxy)benzoyl)piperazine methanesulfonate; 1-methyl-4-(4-(7-methoxyl-1,2,3,4-tetrahydro-1-naphtoyloxy) benxoyl)piperazine; methanesulfonate; and 1-methyl-4-(3-(1,2,3,4-tetrahydro-1-naphthoyloxy)benzoyl)piperazine hydrochloride);
7. YS3025 (CAS Registry No. 138320-33-9) disclosed by Rizzi et al. (1992 Biochem Int. 28:385-92);

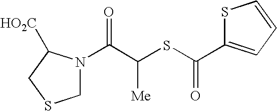

8. MR889 (CA registry No. 94149-41-4) disclosed by Luisetti et al. (1989 Biochem Biophys Res Commun. 165:568-73);

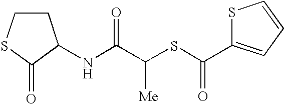

9. chymotrypsin inhibitors disclosed by Yokoo et al. (1987 Yakugaku Zasshi 107:732-7), many of which are of the phenyl ester type and which include those represented by CAS Registry Nos. 85858-76-0, 89703-10-6 (also known as FK-448 CA index name 1-Naphthalenecarboxylic acid, 1,2,3,4-tetrahydro-, 4-[[4-(1-methylethyl)-1 piperazinyl]carbonyl]phenyl ester, monomethanesulfonate), 90185-92-5, 90185-96-9, 90185-98-1, 90186-00-8, 90186-01-9, 90186-05-3, 90186-06-4, 90186-07-5, 90186-08-6, 90186-09-7, 90186-10-0, 90186-11-1, 90186-12-2, 90186-13-3, 90186-14-4, 90186-22-4, 90186-23-5, 90186-24-6, 90186-25-7, 90186-27-9, 90186-28-0, 90186-29-1, 90186-31-5, 90186-35-9, 90186-43-9, 90209-88-4, 90209-89-5, 90209-92-0, 90209-94-2, 90209-96-4, 90209-97-5, 90210-01-8, 90210-03-0, 90210-04-1, 90210-25-6, 90210-26-7, 90210-28-9, 90230-84-5, 90409-84-0, 95460-86-9, 95460-87-0, 95460-88-1, 95460-89-2, 95460-91-6, 114949-00-7, 114949-01-8, 114949-02-9, 114949-03-0, 114949-04-1, 114949-05-2, 114949-06-3, 114949-18-7, 114949-19-8, 114964-69-1, and 114964-70-4; and
10. the two chymotrypsin inhibitors shown below described by Boulanger (1986 Journal of Medicinal Chemistry 29:1483-7);

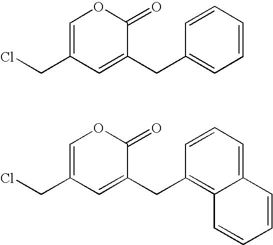

Additional chymotrypsin inhibitors include:
1. chymostatin (CAS registry No. 9076-44-2);

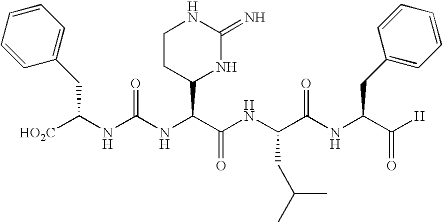

Chymostatin is often provided as a mixture of the three compounds depicted below

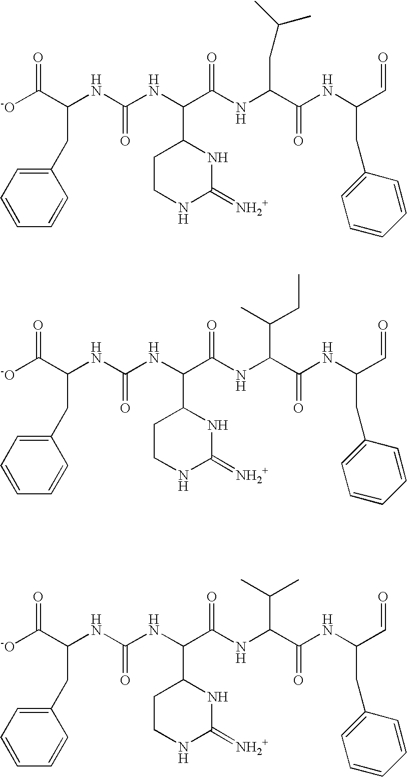

Thus, chymostatin can be considered to be N—(Na-Carbonyl-[S,S]-a-(2-Iminohexahydro-4-pyrimidyl)glycine)-X-Phe-al)-Phe where X is Leu, Val or Ile. In some cases the mixture is contains all three forms, but the form wherein X is Leu predominates.
Chymostatin variants with improved absorbtion can be useful. For example, the carboxyl group can be converted to a methyl ester, ethyl ester or another ester as shown below. The esters might be metabolized to the acid form.

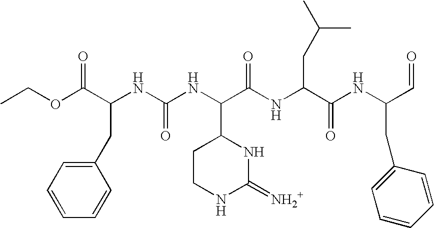

This esterification can be combined with modification of the pyrimidine ring and/or elimination of the backbone carbonyl group as shown in the structures below.

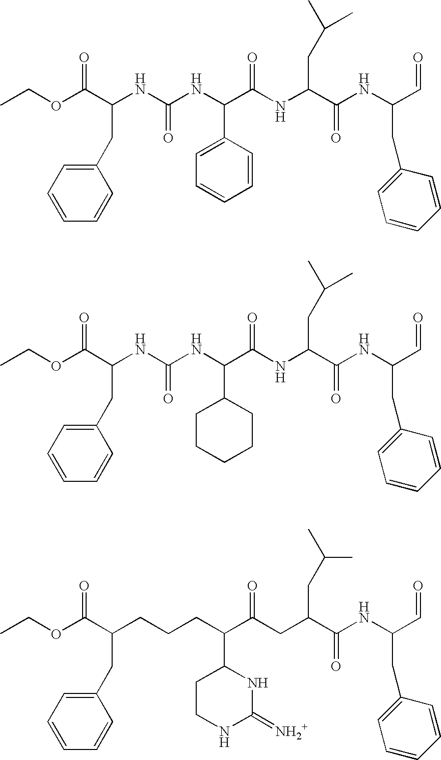

Further modification of chymostatin as shown below may also create variants that are more readily absorbed.

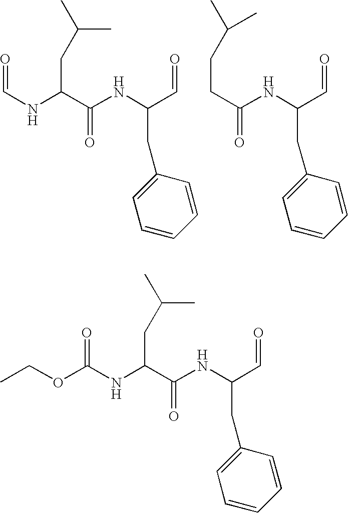

2. 4-(2-aminoethyl)-benzenesulfonylfluoride hydrochloride (Pefabloc; CAS Registry No. 30827-99-7);

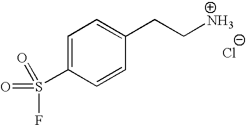

3. benzamidine (CA Registry No. 618-39-3);

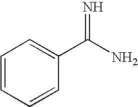

4. di-isopropyl phosphofluoridate (CAS Registry No. 55-91-4);

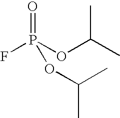

5. 6-aminocaproic acid (CAS Registry No. 60-32-2);

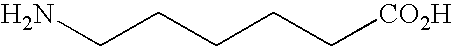

6. CAS Registry No. 88070-98-8;

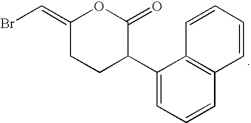

7. ecotin (a peptide inhibitor, CAS Registry No. 87928-05-0; ecotin variants with enhanced activity are described in PCT Publication Nos. WO0061634 and WO0061782);
8. PMSF;

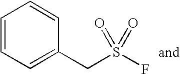

9. benzenesulfonamide (also known as N-tosyl-L-phenylalaninechloromethyl ketone (TPCK); CAS Registry No. 402-71-1).

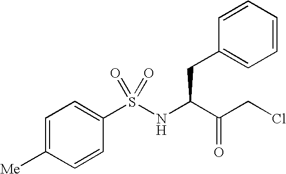

A number of chymotrypsin inhibitors are produced by plants, including: the peptide inhibitor CI2 (CA Registry No. 139466-47-0, GENBANK® Accession S18818, GI: 100574), and variants and homologs thereof including CI-2A (U.S. Pat. No. 5,167,483), CI-2A (WO 9205239), WCI-3 (Shibata et al. 1988 J Biochem (Tokyo) 104:537-43), WCI-2 (Habu et al. 1992 J Biochem (Tokyo) 111:249-58), and WCI-x (Habu et al., supra). Other plant-derived inhibitors have also been described (Bryant et al. 1976 Biochemistry 15:3418-24; Hass et al. 1982 Biochemistry 21:752-6; Birk 1985 Int J Pept Protein Res. 25:113-31; Pearce et al 1982 Archives of Biochemistry and Biophysics 213:456-62; Tamir et al 1996 Journal of Protein Chemistry 15: 219-29; Birk et al 1999 Khimiya beYisra'el 1:9-12; Polya 2003 Studies in Natural Products Chemistry 29: 567-641; Weder et al 2004 Journal of Agricultural and Food Chemistry 52:4219-4226; Teles et al 2004 Phytochemistry 65: 793-799; Tsoi et al 2004 Biological Chemistry 385:185-189 and Kollipara et al. 1992 Journal of Agricultural and Food Chemistry 40:2356-63). Hammond et al 1984 J. Biol. Chem. 259: 9883-9890 describe the Bowman-Birk protease inhibitor (BBI) in soybean (for example see GENBANK® AAO89509 GI:29691202). BBIs are reviewed in Bowman et al. 1993 Protease Inhib. Cancer Chemoprev. Agents 93-6. Synthetic peptides have been generated which mimic BBI inhibitors (McBride et al 2001 Current Medicinal Chemistry 8: 909-917 and McBride et al 2002 Biopolymers 66:79-92). Other chymotrypsin inhibitors are described in Bister et al 2004 Journal of Natural Products 67: 1755-1757; Szenthe et al 2004 Biochemistry 43:3376-3384; Zhou et al. 2004 Comparative Biochemistry and Physiology, Part B: Biochemistry & Molecular Biology 137B:219-224 and Mak et al. 2004 Biochimica et Biophysica Acta 1671:93-105. These chymotrypsin inhibitors and others are useful in the methods of the invention.

Additional small molecule chypotrypsin inhibitors include:
leupeptin (CA registry 55123-66-5)

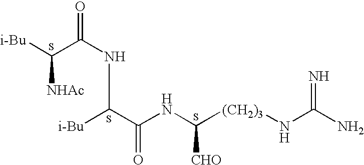

p-tosyl-L-arginine methyl ester (TAME) (CA registry 901-47-3)

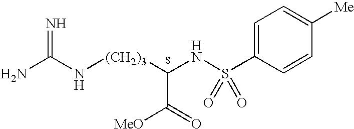

N-alpha-tosyl-L-lysine chloromethyl ketone (TLCK) (CA registry 4272-74-6)

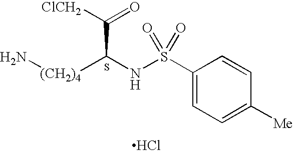

3,4-Dichloroisocoumarin (CA registry 51050-59-0)

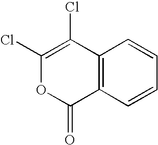

CA registry 221051-66-7 (Hayashi et al Peptide Science (1999) 35:249-252 and Iijima et al Journal of Medicinal Chemistry (1999), 42(2), 312-323).

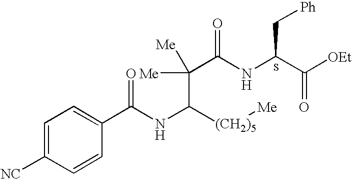

Additional useful peptide inhibitors of chymotrypsin include: urinistatin (bikuni) CA registry 80449-31-6; inhibitors disclosed in Ito et al. Biochemical and Biophysical Research Communications (1972), 49(2), 343-9 (e.g., Ac-Leu-Leu-phenylalinal; Ac-Leu-Leu-tyrosinal; and Ac-Leu-Leu-tryptophanal) and the inhibitors Imperiali and Abeles Biochemistry (1987), 26:4474-7 disclose extended binding inhibitors of chymotrypsin
Additional small molecule chymotrypsin inhibitors useful in the present invention include:

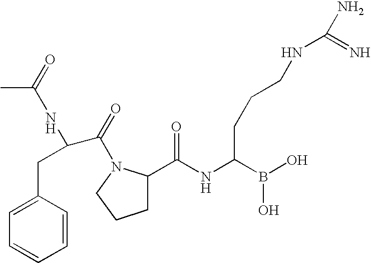

CA registry 130982-43-3 (DuP-714);

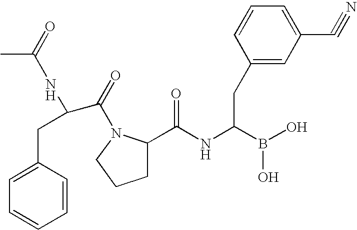

CA registry 197913-52-3 (disclosed in Biochemistry 36:13180-6 (1997));

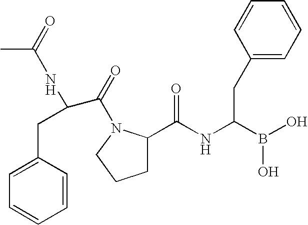

disclosed in Biochemistry 36:13180-6 (1997);

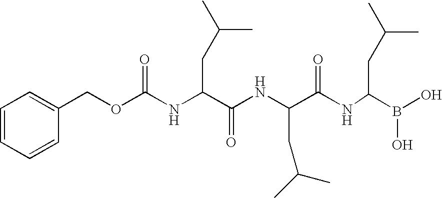

CA registry 179324-22-2 (MG-262 (Cbz-leu-leu-leu-B(OH)2));

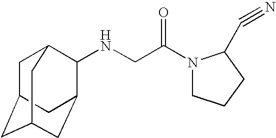

CA registry 274901-16-5 (LAF-237);

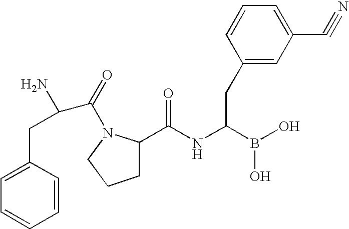

CA registry 792163-40-7 (disclosed in Pharm Res. 16:1786 1999);

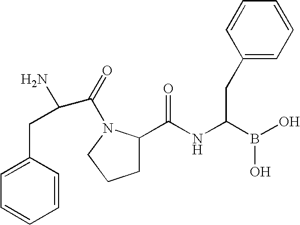

disclosed in Biochemistry 36:13180-6 (1997);

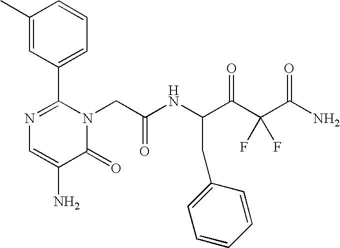

CA registry 339169-59-4;

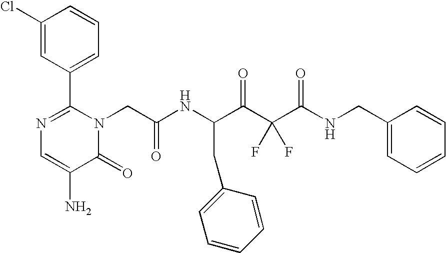

CA registry 243462-36-4;

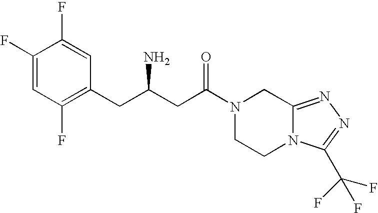

CA registry 654671-78-0 (sitagliptin/MK-0431 (Merck)); and

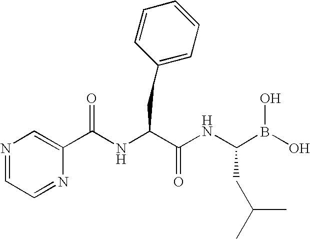

bortezomib/Velcade/PS341.

### Prodrugs of Chymotrypsin Inhibitors

Peptides that are inhibitors of chymotrypsin include those listed below. Prodrug forms of these peptides can be created by modifying the carboxy terminus of the peptide and/or one or more of the carboxylic acid side chains present in the peptide into, for example, an ester, carbonate, carbamate, or phosphate ester. Thus, the carboxylic acid portion of the side chains of aspartic acid and glutamic acid can be modified to an ester. Thus, the —CH₂C(O)OH side chain of aspartic acid can be modified to —CH₂C(O)OR and the —CH₂CH₂C(O)OH side chain of glutamic acid can be modified to —CH₂CH₂C(O)OR, wherein R is selected from a straight chain or branched C₁ to C₆ alkyl (C₁, C₂, C₃, C₄, C₅, C₆), alkenyl, alkynyl, or a C₆ aryl optionally independently substituted with one or more halogen (Cl, F, Br, I), —OH, —C(O)OH, or —NH₃. Alternatively R can be selected from:

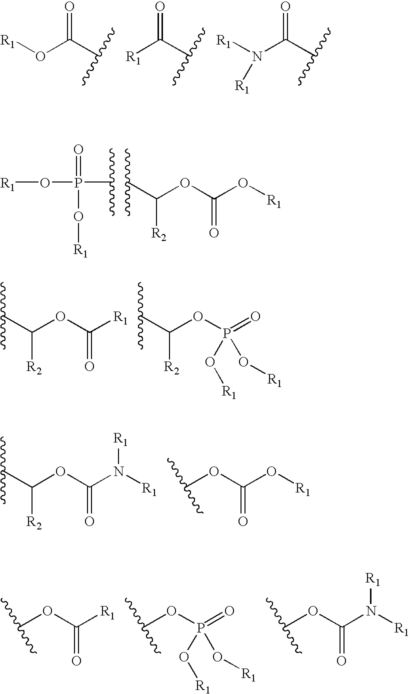

wherein R₁ is selected from H or a branched or straight chain C₁ to C₆ (e.g., C₁, C₂, C₃, C₄, C₅, C₆) alky alkenyl, alkynyl, aryl, cycloalkyl, or arylalkyl optionally independently substituted with one or more halogen —OH, —C(O)OH, or —NH₃. R₂ is selected from H and straight or branched chain C₁ to C₆ (C₁, C₂, C₃, C₄, C₅, C₆) alkyl.

In addition, symmetrical and non-symmetrical anhydride prodrug derivatives of the peptide chymotrypsin inhibitors can be created. Thus, two copies of the same peptide can be joined to form a symmetrical anhydride derivative. Such derivatives have the structure P—C(O)—O—C(O)—P where P represents the remainder of the peptide. The two copies of the peptide can be linked through terminal carboxyl groups or through internal carboxyl groups. Two different peptides can be joined to form a non-symmetrical anhydride prodrug derivative. Such derivatives have the structure P′—C(O)—O—C(O)—P where P and P′ represent the remainder of the two different peptides. The two peptides can be linked through terminal carboxyl groups or through internal carboxyl groups.

Prodrug forms of the peptides can also be created by modifying free hydroxyl groups of the peptide present in the peptide side chains into, for example, an ester, carbonate, carbamate, or phosphate ester. Thus, the —OH portion of the side chains of serine, threonine and tyrosine can be modified to an ester. Thus, the —CH₂OH side chain of serine can be modified to —CH₂C(O)OR, the —CH₂-phenyl-OH side chain of tyrosine can be modified to —CH₂-phenyl-C(O)OR and the —CH(OH)CH₃ side chain of threonine can be modified to —CH(C(O)OR)CH₃, wherein R is selected from a straight chain or branched C₁ to C₆ alkyl (e.g., C₁, C₂, C₃, C₄, C₅, C₆), alkenyl, alkynyl, or a C₆ aryl optionally independently substituted with one or more halogen (Cl, F, Br, I), —OH, —C(O)OH, —NH₃. Alternatively R can be selected from

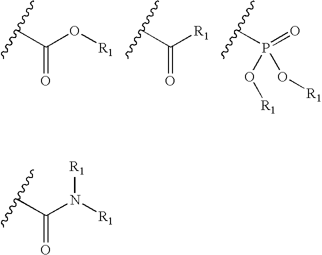

wherein R₁ is selected from H or a branched or straight chain C₁ to C₆ (C₁, C₂, C₃, C₄, C₅, C₆) alkyl, alkenyl, alkynyl, an aryl group, an cycloalkyl group, or an arylalkyl group optionally independently substituted with one or more halogen —OH, —C(O)OH, or —NH₃.

In some embodiments, the prodrug chymotrypsin inhibitor peptide molecules may comprise one or more modifications of —OH or —COOH groups to ester, carbamate, carbonate, phosphate or other derivatives described herein. For example, a prodrug chymotrypsin inhibitor peptide may comprise an ester at its carboxy terminus and an internal serine modified to a carbonate derivative. Thus, one, two or more of the —OH and —COOH groups in a peptide can be modified and the modifications can be different or identical. Examples of chymotrypsin inhibitors which are peptides that can be modified to create prodrug derivatives include but are not limited to:

1. tissue-factor-pathway inhibitor (TFPI) (Peterson et al. 1996 Eur J Biochem 235:310-6; for examples see human (GENBANK® AAH15514 GI:15930156), mouse (GENBANK® AAH36146 GI:23271605), and dog (GENBANK® AAB32443 GI:833924));

2. α-2 antiplasmin (Potempa et al. 1988 Science 241: 99-700, GENBANK® Accession P08697, GI:112907; (SEQ ID NO. 2); MALLWGLLVLSWSCLQGPCSVFSPVSAMEPLGRQLTSGPNQEQVSPLTLLKLGN QEPGGQTALKSPPGVCSRDPTPEQTHRLARAMMAFTADLFSLVAQTSTCPNLILS PLSVALALSHLALGAQNHTLQRLQQVLHAGSGPCLPHLLSRLCQDLGPGAFRLA ARMYLQKGFPIKEDFLEQSEQLFGAKPVSLTGKQEDDLANINQWVKEATEGKIQ EFLSGLPEDVLLLLNAIHFQGFWRNKFDPSLTQRDSFHLDEQFTVPVEMMQARTY PLRWFLLEQPEIQVAHFPFKNNMSFVVLVPTHFEWNVSQVLANLSWDTLHPPLV WERPTKVRLPKLYLKHQMDLVATLSQLGLQELFQAPDLRGISEQSLVVSGVQHQ STLELSEVGVEAAAATSIAMSRMSLSSFSVNRPFLFFIFEDTTGLPLFVGSVRNPNP SAPRELKEQQDSPGNKDFLQSLKGFPRGDKLFGPDLKLVPPMEEDYPQFGSPK);

3. members of the serpin α-1 antichymotrypsin family (Forsyth et al. 2003 Genomics 81: 336-45; for example see CAS Registry No. 141176-92-3; functional variants thereof are described in European patent application EP1415664 and in Plotnick et al. 2003 Biochemistry 33:29927 (for example the P2 (Leu-357) variant);

4. gelin (U.S. Pat. No. 5,397,694, partial sequence (aa 1-29) can be found at GENBANK® Accession AAB27871, GI:409493; (SEQ ID NO:3) VDEKAEVTDGLCGDWTCSGAQVXQNDAAV), which has been proposed as a treatment for dermatitis as well for periodontitis and gingivitis;

5. hirustasin (Sollner et al. 1994 Eur J Biochem. 219: 937-43, GENBANK® Accession No P80302, GI:461516: (SEQ ID NO:4); TQGNTCGGETCSAAQVCLKGKCVCNEVHCRIRCKYGLKKD ENGCEYPCSCAKASQ);

6. certain eglins, including eglin C (GENBANK® Accession P01051, GI:124128; (SEQ ID NO: 5) TEFGSELKSFPEVVGKTVDQAREYFTLHYPQYDVYFLPEGSPVTLDLRYNRVRVF YNPGTNVVNHVPHVG) are peptide inhibitors of chymotrypsin. For other examples of eglins, see those disclosed in U.S. Pat. No. 5,180,667, U.S. Pat. No. 6,342,373, U.S. Pat. No. 4,636,489, Seemuller et al. 1981 Methods Enzymol. 804-816, Seemueller et al. 1986 Research Monographs in Cell and Tissue Physiology 337-59, Nick et al 1988 Adv in Experimental Medicine and Biology 240:83-8, and Schnebli et al 1986 Pulm. Emphysema Proteolysis (conference) CAN 107:228147 AN 1987:628147), which has been considered as a treatment for emphysema and for use as a non-steroidal anti-inflammatory agent;

7. inhibitors from Bombyx mori (see, e.g., JP 4013698 A2 and JP 04013697 A2; CA registry No. 142628-93-1, (SEQ ID NO: 6) DEPTTKPFCEQAFGDCGTPY and CA registry No. 142628-94-2, (SEQ ID NO: 7) DKPTTEPFIC EQRFGNCGTG);

8. the leech derived peptide thrombin inhibitor, hirudin (Zwilling 1968 Hoppe-Seyler's Zeitschrift fuer Physiologische Chemie 349:1787-8, CA Registry No. 8001-27-2, see for example, GENBANK® Accession AAA01384 GI:269388, (SEQ ID NO: 8) ITYTDCTESGQNLCLCEGSNVCGKGNKClLGSQGKDNQCVTGEGTPKPQSHNQG DFEPIPEDAYDE). Hirudin variants are disclosed in the literature (for examples see those in U.S. Pat. No. 5,674,838, Great Britain patent application GB2242681 and those described in Wirsching et al 2003 Molecular Genetics and Metabolism 80:451-462);

9. a shorter hirudin variant, hirulog/BG 8967 (CA Registry No. 128270-60-0, (SEQ ID NO: 9); FPRPGGGGNGDFEEIPEEYL; Angiomax® (bivalirudin)) may also have chymotrypsin inhibition activity and may thus be useful in the present invention along with other peptides disclosed in PCT publication WO04076484 and U.S. Pat. No. 5,196,404;

secretory leukocyte protease inhibitor (SLPI) (for examples see GENBANK® CAA28187 GI:758101 (human), GENBANK® Accession NP_{—}445824.1 GI:16758102 (rat), and GENBANK® Accession NP_{—}035544.1 GI:6755574 (mouse); also Farley et al. 1997 Drugs and the Pharmaceutical Sciences 84:305-334;

10. α-1 anti-trypsin which can inhibit elastase as well as chymotrypsin and thus may be useful in the present invention (for examples see GENBANK® Accession CAB06092 GI:2780174 (human) and GENBANK® Accession NP_{—}001009663 GI:57527135 (rat)). This product has been sold to treate α-1 anti-trypsin deficiency (a genetic disorder) as Zemaira™ (Aventis Behring; FDA biologics license 2003 License #1281), Prolastin® (Bayer), and Aralast™ (Baxter); and

11. the inhibitor aprotinin or a derivative thereof (TRASYLOL®); CAS Registry No. 9087-70-1; MKMSRLCLSVALLVLLGTLAASTPGCDTSNQAKAQRPDFCLEPPYTGPCKARIIR YFYNAKAGLCQTFVYGGCRAKRNNFKSAEDCMRTCGGAIGPWENL; (SEQ ID NO. 10).

In addition, a large number of peptide inhibitors of chymotrypsin are reviewed by Schoofs et al. (2002 Curr Pharm Des. 8: 483-91) and by Salier et al. (1996 Biochem J. 315:1-9). McBride et al. 1996J Mol Biol. 259: 819-27 and McBride et al. 2000 J Pept Sci. 6:446-52 disclose chymotrypsin inhibitors derived from combinatorial peptide libraries including those represented by CAS registry Nos. 306762-66-3, 306762-67-4, 306762-68-5, 306762-69-6, 306762-70-9, 306762-71-0, 306762-72-1, 306762-73-2, 306762-74-3, 306762-75-4, 178330-92-2, 178330-93-3, 178330-94-4, and 178330-95-5.

In addition, various small molecule inhibitors of chymotrypsin are prodrugs of chymotrypsin inhibitors or can be converted into prodrugs. Examples include, but are not limited to:

compounds with chymotrypsin inhibition activity described in U.S. Pat. No. 4,755,383 which may act as prodrugs (including, 1-Naphthaleneacetic acid, 4-[[4-(1-methylethyl)-1-piperazinyl]carbonyl]phenyl ester (CA registry No. 90186-24-6), Acetic acid, 9H-fluoren-9-ylidene-, 4-[[4-(1-methylethyl)-1-piperazinyl]carbonyl]phenyl ester (CA registry No. 90185-93-6), 1-Naphthalenecarboxylic acid, 1,2,3,4-tetrahydro-4-[[4-(1-methylethyl)-1-piperazinyl]carbonyl]phenyl ester, monomethanesulfonate (CA registry No. 89703-10-6), 1-Naphthalenecarboxylic acid, 1,2,3,4-tetrahydro-, 4-[(4-cyclohexyl-1-piperazinyl)carbonyl]phenyl ester (CA registry No. 85858-74-8), and 1-isopropyl-4-[4-(1,2,3,4-tetrahydronaphthoyloxy) benzoyl]piperazine methanesulfonate);
chymotrypsin inhibitors described in U.S. Pat. Nos. 4,755,383 and 4,639,435 which may act as prodrugs including those of Formula I:

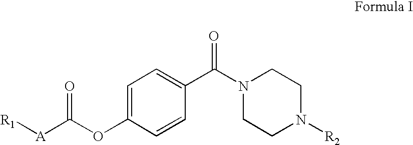

wherein A is a single bond, or an alkylene, vinylene, —O-alkylene or methine group; R¹ is a bicyclic carbocyclic residue which may partly be saturated and may optionally be substituted by at least one member of the class consisting of lower alkyl, lower alkoxy, oxo and nitro groups and halogen atoms; a fluorene residue which may optionally have an oxo group; a fluorenylidene group; an anthracene residue; a phenanthrene residue which may partly be saturated and may optionally be substituted by at least one lower alkyl group; a benzofuran or thianaphthene residue which may optionally be substituted by at least one member of the class consisting of lower alkyl and lower alkoxy groups; a benzopyran or benzazine residue which may partly be saturated and may optionally be substituted by at least one member of the class consisting of oxo and phenyl groups; a phthalimide residue; a benzodiazone residue; an isoxazole residue which may optionally be substituted by at least one member of the class consisting of lower alkyl and phenyl groups; an alkylenedioxybenzene residue; or a xanthene residue, and R² is a loweralkyl, cycloalkyl, cycloalkylalkyl or aralkyl group. The methods of producing the compounds of the formula (I) and their chymotrypsin inhibitory activity characteristics have been described in Japanese Patent Application No. 109192/1982 and in Japanese Patent Application filed Apr. 28, 1983;
   inhibitors described in EP 0128007 and U.S. Pat. No. 4,620,005 which may act as prodrugs including: 1-isopropyl-4-[(4-(1,2,3,4-tetrahydro-1-naphthoyloxy)phenyl)carbonyloxymethyl carbonyl]piperazine; 1-isopropyl-4-[(4-(1,2,3,4-tetrahydro-1-naphthoyloxy)phenyl)ethyl carbonyl]piperazine; and 1-isopropyl-4-[(4-(1,2,3,4-tetrahydro-1-naphthoyloxy)phenyl)methylcarbonyl]piperazine; inhibitors described U.S. Pat. No. 4,898,876 which may act as prodrugs including inhibitors: 1-isopropyl-4-(4-(5,6,7,8-tetraphydronaphthalene-1-acetyloxy)benzoyl)piperazine hydrochloride; 1-isopropyl-4-(4-(9-fluorenylidene acetyloxy)benzoyl)piperazine; 1-isopropyl-4-(4-(thianaphthene-2-acetyloxy)benzoyl)piperazine methanesulfonate; 1-methyl-4-(4-(7-methoxyl-1,2,3,4-tetrahydro-1-naphtoyloxy) benxoyl)piperazine; methanesulfonate; and 1-methyl-4-(3-(1,2,3,4-tetrahydro-1-naphthoyloxy)benzoyl)piperazine hydrochloride);
   Prodrug variants of YS3025 (CAS Registry No. 138320-33-9) disclosed by Rizzi et al. (1992Biochem Int. 28:385-92);

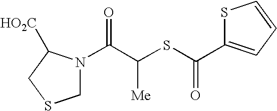

Suitable prodrugs of which include compounds having the structure:

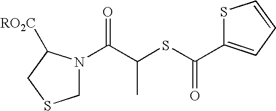

wherein R is selected from a straight chain or branched C₁ to C₆ alkyl (C₁, C₂, C₃, C₄, C₅, C₆), alkenyl, alkynyl, or a C₆ aryl optionally independently substituted with one or more halogen (Cl, F, Br, I), —OH, —C(O)OH, or —NH₃. Alternatively R can be selected from

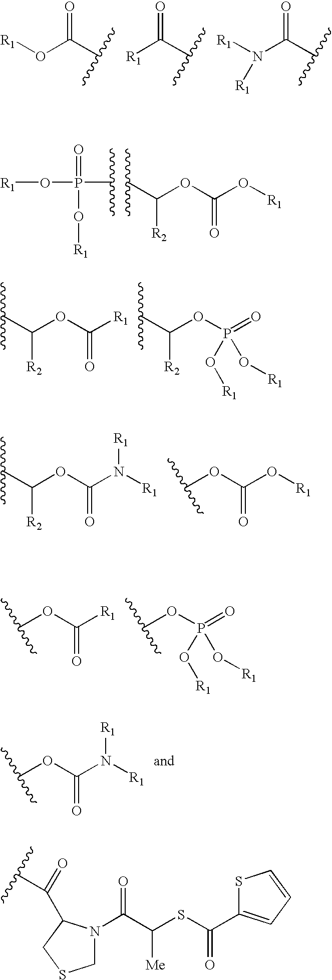

wherein R₁ is selected from H or a branched or straight chain C₁ to C₆ (C₁, C₂, C₃, C₄, C₅, C₆) alkyl, alkenyl, alkynyl, aryl, cycloalkyl, or arylalkyl optionally independently substituted with one or more halogen —OH, —C(O)OH, or —NH₃ and R₂ is selected from H, straight or branched chain C₁ to C₆ (C₁, C₂, C₃, C₄, C₅, C₆) alkyl. Thus, in the case where R is

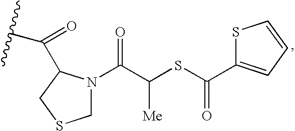

the resulting prodrug of YS3025 (CAS Registry No. 138320-33-9) is a symmetrical anhydride and has the structure:

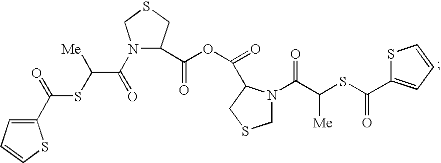

chymotrypsin inhibitors disclosed by Yokoo et al. (1987 Yakugaku Zasshi 107:732-7), many of which are of the phenyl ester type and may act as prodrugs, including those represented by CAS Registry Nos. 85858-76-0, 89703-10-6 (also known as FK-448 CA index name 1-Naphthalenecarboxylic acid, 1,2,3,4-tetrahydro-, 4-[[4-(1-methylethyl)-1-piperazinyl]carbonyl]phenyl ester, monomethanesulfonate), 90185-92-5, 90185-96-9, 90185-98-1, 90186-00-8, 90186-01-9, 90186-05-3, 90186-06-4, 90186-07-5, 90186-08-6, 90186-09-7, 90186-10-0, 90186-11-1, 90186-12-2, 90186-13-3, 90186-14-4, 90186-22-4, 90186-23-5, 90186-24-6, 90186-25-7, 90186-27-9, 90186-28-0, 90186-29-1, 90186-31-5, 90186-35-9, 90186-43-9, 90209-88-4, 90209-89-5, 90209-92-0, 90209-94-2, 90209-96-4, 90209-97-5, 90210-01-8, 90210-03-0, 90210-04-1, 90210-25-6, 90210-26-7, 90210-28-9, 90230-84-5, 90409-84-0, 95460-86-9, 95460-87-0, 95460-88-1, 95460-89-2, 95460-91-6, 114949-00-7, 114949-01-8, 114949-02-9, 114949-03-0, 114949-04-1, 114949-05-2, 114949-06-3, 114949-18-7, 114949-19-8, 114964-69-1, and 114964-70-4; and
Prodrugs of 6-aminocaproic acid (CAS Registry No. 60-32-2);

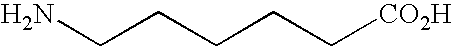

Suitable prodrugs of the compound include:

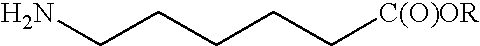

wherein R is selected from a straight chain or branched C₁ to C₆ alkyl (C₁, C₂, C₃, C₄, C₅, C₆), alkenyl, alkynyl, or a C₆ aryl optionally independently substituted with one or more halogen (Cl, F, Br, I), —OH, —C(O)OH, or —NH₃. Alternatively R can be selected from

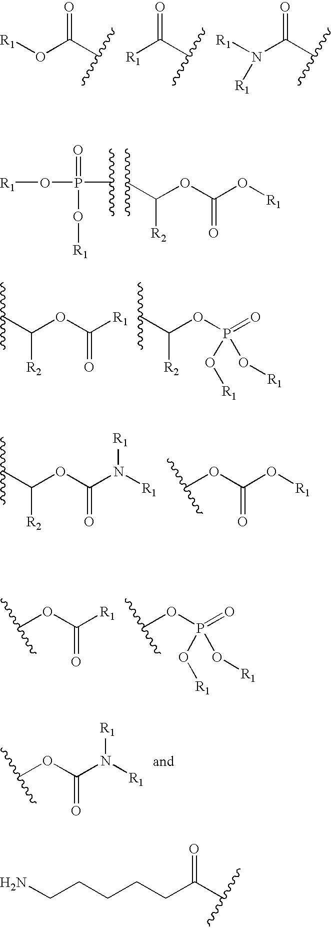

wherein R₁ is selected from H or a branched or straight chain C₁ to C₆ (C₁, C₂, C₃, C₄, C₅, C₆) alkyl, alkenyl, alkynyl, aryl, cycloalkyl, or arylalkyl optionally independently substituted with one or more halogen —OH, —C(O)OH, —NH₃ and R₂ is selected from H, straight or branched chain C₁ to C₆ (C₁, C₂, C₃, C₄, C₅, C₆) alkyl. For produrg forms of 6-aminocaproic acid (CAS Registry No. 60-32-2) where R is

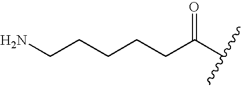

the resulting compound is a symmetrical anhydride and has the structure

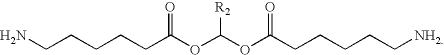

Other chymotrypsin inhibitors which might be modified to prodrugs by converting a —COOH or —OH group to an ester or other derivative including but not limited to those described herein include: chymotrypsin inhibitors that are produced by plants, including: the peptide inhibitor CI-2 (CA Registry No. 139466-47-0, GENBANK® Accession S18818, GI:100574), and variants and homologs thereof including CI-2A (U.S. Pat. No. 5,167,483), CI-2A (WO 9205239), WCI-3 (Shibata et al. 1988 J Biochem (Tokyo) 104:537-43), WCI-2 (Habu et al. 1992 J Biochem (Tokyo) 111:249-58), and WCI-x (Habu et al., supra). Other plant-derived inhibitors have also been described (Bryant et al. 1976 Biochemistry 15:3418-24; Hass et al. 1982 Biochemistry 21:752-6; Birk 1985 Int J Pept Protein Res. 25:113-31; Pearce et al 1982 Archives of Biochemistry and Biophysics 213:456-62; Tamir et al 1996 Journal of Protein Chemistry 15: 219-29; Birk et al 1999 Khimiya beYisra'el 1:9-12; Polya 2003 Studies in Natural Products Chemistry 29: 567-641; Weder et al 2004 Journal of Agricultural and Food Chemistry 52:4219-4226; Teles et al 2004 Phytochemistry 65: 793-799; Tsoi et al 2004 Biological Chemistry 385:185-189 and Kollipara et al. 1992 Journal of Agricultural and Food Chemistry 40:2356-63). Hammond et al 1984 J. Biol. Chem. 259: 9883-9890 describe the Bowman-Birk protease inhibitor (BBI) in soybean (for example see GENBANK® AAO89509 GI:29691202). BBIs are reviewed in Bowman et al. 1993 Protease Inhib. Cancer Chemoprev. Agents 93-6. Synthetic peptides have been generated which mimic BBI inhibitors (McBride et al 2001 Current Medicinal Chemistry 8: 909-917 and McBride et al 2002 Biopolymers 66:79-92). Other chymotrypsin inhibitors are described in Bister et al 2004 Journal of Natural Products 67: 1755-1757; Szenthe et al 2004 Biochemistry 43:3376-3384; Zhou et al. 2004 Comparative Biochemistry and Physiology, Part B: Biochemistry & Molecular Biology 137B:219-224 and Mak et al. 2004 Biochimica et Biophysica Acta 1671:93-105.

### Chymotrypsin-Like Enzymes

Inhibitors of a chymotrypsin-like enzyme and prodrugs thereof can be useful in any of the methods described herein. This is because certain inhibitors of chymotrypsin-like enzymes are also inhibitors of chymotrypsin and can interfere with the cleavage (and inactivation) of guanylin (or guanylin variants) caused by chymotrypsin. In other cases, inhibitors of a chymotrypsin-like enzyme (e.g., the chymotrypsin-like enzyme present in renal sources that is believed to cause cleavage of guanylin) are useful because they are inhibitors of a chymotrypsin-like enzyme that cleaves guanylin in the human body.

A chymotrypsin-like enzyme may exhibit one more similarities to chymotrypsin including but not limited to: (i) catalytic mechanism, (ii) substrate specificity, (iii) structure (i.e. how the enzyme is folded) or (iv) any additional property in which the enzyme resembles chymotrypsin. Chymotrypsin-like enzymes are described in the prior art and include: chymase, Ctrl/chymotrypsin A/CTRA-1 (Mouse: GENBANK® Accession No. AAL11034 GI:15963449; human: GENBANK® Accession No. NP_{—}001898 GI:4503137); the chymotrypsin-like enzyme described in Firth et al. 1996 J Periodontal Res. 31:345-54; clipsin (described in Nelson et al. 1990 J Biol Chem. 265:3836-43); and the chymotrypsin-like endopeptidases from opossum kidney cells and clonal osteogenic UMR-106 cells (described in Yamaguchi et al. 1988 Endocrinology 123:2812 and Yamaguchi et al. 1989 Biochim Biophys Acta. 1010:177 respectively). Other chymotrypsin-like enzymes are described by Arribas and Castano (1993 J Biol Chem. 268:21165-71). Bartuski et al 1998 Genomics 54:297 identify a murine ortholog of the human SCCA2 chymotrypsin-like serine proteinase. A number of virus encoded proteinases encode chymotrypsin-like enzymes. For examples see those described in Ziebuhr et al. 2000 Journal of General Virology 81:853-879, Dougherty and Semler 1993 Microbiological Reviews 57:781-822, Ryan and Flint 1997 Journal of General Virology 78:699-723, and Chang et al 1995 Biochem Biophys Res Commun. 213:475-83. Snijder et al. 1996 Journal of Biol Chem 271:4864 discuss the Arterivirus Nsp4 Protease as a prototype the 3C-like Serine Proteases family of chymotrypsin-like enzymes. The hepatitis C virus NS3-NS4A protease exhibits chymotrypsin like activity. (Perni 2000 Drug News Perspect. 13:69-77). Both cathepsin A (lysosomal) and cathepsin G have been reported to have chymotrypsin-like activity. The 20S proteasome also exhibits chymotrypsin-like activity.

### Inhibitors of Chymotrypsin-Like Enzymes

Inhibitors of chymotrypsin-like enzymes and prodrugs of inhibitors of chymotrypsin-like enzymes including, but not limited to, inhibitors of the 20S proteasome, inhibitors of cathepsin, inhibitors of chymase, and inhibitors of hepatitis C virus NS3-NS4A protease are useful in the pharmaceutical compositions and methods described herein.

Various inhibitors of chymotrypsin-like proteases (e.g. inhibitors of chymotrypsin-like enzymes) have been described. In some instances, the inhibitors are useful because they inhibit one or more chymotrypsin or chymotrypsin-like proteases. For example, the serine protease inhibitors disclosed in U.S. Pat. No. 6,262,069, U.S. Pat. No. 5,916,888, U.S. Pat. No. 5,900,400, U.S. Pat. No. 5,157,019, U.S. Pat. No. 4,829,052, U.S. Pat. No. 5,723,316, and U.S. Pat. No. 5,807,829 are useful as inhibitors of chymotrypsin, chymase or other chymotrypsin-like proteases. U.S. Pat. No. 5,314,815 discloses adducts of serine hydrolase with a phosphate or phosphonate which are useful as inhibitors of chymotrypsin and chymotrypsin-like enzymes (e.g., chymase and cathepsin G). Specifically disclosed species include: 4-nitrophenyl phenacyl methylphosphate; 4-nitrophenyl 4-nitrophenacyl methylphosphate; 4-nitrophenyl 4-methylphenacyl methylphosphate; 4-nitrophenyl 4-methoxyphenacyl methylphosphate; and 4-nitrophenyl 4-chlorophenacyl methylphosphate. US20030129720 discloses a plant serine protease inhibitor useful as an inhibitor of chymotrypsin or chymotrypsin-like proteases.

Inhibitors of the Chymotrypsin-Like Activity of the 20S Proteasome

Numerous inhibitors of the chymotrypsin-like activity of the 20S proteasome have been described. These include but are not limited to commercially available inhibitors such as:_N-Acetyl-L-leucyl-L-leucyl-L-norleucinal, Aclacinomycin A (Aclarubicin), (−)-Epigallocatechin gallate (EGCG), Epoxomicin, Lactacystin, clasto-Lactacystin β-lactone, N-Tosyl-Phe-chloromethylketone (TPCK), NIP-(Leu)₃-vinyl sulphone, Phepropeptin A, Phepropeptin B, Phepropeptin C, Phepropeptin A-D pack, Z-Ile-Glu(OBut)-Ala-Leu-H (PSI), Z-Leu-Leu-Leu-vinyl sulphone, Z-Leu-Leu-Nva-H (MG115), Z-Leu-Leu-Leu-H (MG132), Z-Leu-Leu-Leu-B(OH)₂ (MG262), and Z-Leu-Leu-Tyr-COCHO. Additional inhibitors of the chymotrypsin-like activity of the 20S proteasome include tannic acid (Tam et al. 2001 Cancer Epidemiology Biomarkers & Prevention 10: 1083-1088), 5-Methoxy-1-Indanone Dipeptide Benzamides (Lum et al 1998 Bioorganic & Medicinal Chemistry Letters 8:209-214), and peptide aldehydes (Fenteany and Schreiber 1996 Chem. Biol. 3:905).

Cathepsin Inhibitors

Cathepsin G is a chymotrypsin-like serine protease found in the azurophilic granules of polymorphonuclear leukocytes which functions to degrade proteins during inflammatory responses. Bania et al 1999 Eur. J. Biochem. 262:680-687 describe the primary structure and properties of a cathepsin G inhibitor from the larval hemolymph of Apis mellifera. US20010056180 discloses peptides which encode serine protease inhibitors including variants which have an improved ability to inhibit cathepsin G. U.S. Pat. No. 5,780,449 discloses cathepsin G inhibiting aptamers.

Inhibitors of the Hepatitis C Virus NS3-NS4A Chymotrypsin-Like Activity

Many inhibitors of Hepatitis C virus NS3-NS4A protease have been described including those disclosed in: Perni 2000 Drug News Perspect. 13:69-77, Bianchi and Pessi 2002 Biopolymers 66:101-14, Sperandio et al 2002 Bioorg Med Chem Lett. 12:3129-33 and US published patent applications US20040018986, US20030236242 US2003011975, US20030100768, US20040180815 (pyridazinonyl macrocyclic inhibitors), and US20030064962. US20040266668 describes quinoxalinyl macrocyclic inhibitors that inhibit the activity of NS3-NS4A.

Chymase Inhibitors

Chymase has a variety of functions, including degradation of extracellular matrix proteins, cleavage of angiotensin I to angiotensin II and activation of matrix proteases and cytokines. Inadequate control by endogenous chymase inhibitors can lead to degradation of healthy constituents of the extracellular matrix thereby contributing to inflammatory disorders. Chymase inhibitors and prodrugs thereof are useful in the pharmaceutical compositions and methods described herein. In certain embodiments, the chymase inhibitor inhibits human chymase activity. Human chymase inhibitors may inhibit human heart chymase, human mast cell chymase or human skin chymase.

Chymase inhibitors include but are not limited to: acetoamide derivatives (Japanese Unexamined Patent Publication No. 10-7661), acetoamide derivatives (WO99/41277), benzimidazole derivatives (US20040162311, US20040010004, US20040122042, WO00/03997, WO01/53272, and WO01/53291), cefam compounds (Japanese Unexamined Patent Publication No. 10-087493) acid anhydride derivatives (Japanese Unexamined Patent Publication No. 11-049739), heterocyclic amide compounds (WO 99/32459), hydantoin derivatives (Japanese Unexamined Patent Publication No. 9-31061), imidazolidine derivatives (WO 96/04248), isoxazole derivatives (Japanese Unexamined Patent Publication No. 11-1479), phosphonic compounds (US20030195172), phosphoric acid derivatives (Oleksyszyn et al. 1991 Biochemistry 30:485), pyrimidinone derivatives (Akahoshi 2003 Curr Pharm Des. 9:1191-9), quinazoline derivatives (US20020183338), quinazoline derivatives (WO 97/11941), thiazine derivatives (US20040097496), triazine derivatives (Japanese Unexamined Patent Publication No. 8-208654), and tripeptide compounds (WO 93/03625). Chymase inhibitors are also disclosed in patent publications US20040086537, WO 00/05204, WO 00/06594, WO 00/10982, WO 00/32587, WO 01/122261, WO 01/322214, WO 01/32621, WO 01/83471, WO 02/122595, WO 02/18378, WO 96/39373, WO 98/18794, WO 99/09977, and WO 99/45928. Additional examples of chymase inhibitors are disclosed in Japanese Unexamined Patent Publication Nos. 10-245384, 10-251239, 10-53579, 11-246437, 11-48739, 2000-95770, and 2001-97957. Other examples of chymase inhibitors include: SQN-5 (CA Registry No. 205944-60-1), MNEI (CA Registry No. 216503-90-1), NK3201 (Takai and Miyazaki 2003 Cardiovasc Drug Rev. 21:185-98); NK301 (Doggrell and Wanstall 2003 Expert Opin Investig Drugs. 12:1429-32); Methyllinderone (CA Registry No. 3984-73-4), and BCEAB (4-[1-[[bis-(4-methylphenyl)-methyl]-carbamoyl]-3-(2-ethoxybenzyl)-4-oxo-azetidine-2-yloyl]-benzoic acid. US20040058963 discloses indole derivatives as chymase inhibitors including but not limited to compounds 3-83 as defined in claim 4.

US20040018984 discloses peptidyl derivatives of aryl diesters of α-aminoalkylphosphonic acids. For example, Suc-Val-Pro-Phe^{p}(OPh)₂ and enantiomerically enriched preparations thereof are specifically disclosed.

US20030083315 discloses benzimidazole derivatives as chymase inhibitors. For example, the following species are disclosed: 4-((1-((1-naphthyl)methyl)-5-methoxybenzimidazole)-2-ylthio)butyric Acid; 4-((1-naphthyl)methyl)-5,6-difluorobenzimidazole)-2-ylthio)butyric Acid; 4-(1-((1-naphthyl)methyl)-5-cyanobenzimidazole-2-ylthio)butyric Acid; 4-(1-((1-naphthyl)methyl)-5-cyanobenzimidazole-2-yl)-3,3-dimethylbutyric Acid; 4-(1-((1-naphthyl)methyl)benzimidazole-2-ylthio)butyric Acid; Sodium 4-(1-((1-naphthyl)methyl)benzimidazole-2-ylthio)butyrate; 4-(1-((8-methyl-1-naphthyl)methyl)benzimidazole-2-ylthio)butyric Acid; 4-(1-((8-methyl-1-naphthyl)methyl)-5,6-dimethylbenzimidazole-2-ylthio)-butyric Acid; 5-(1-((1-naphthyl)methyl)benzimidazole-2-ylthio)-4-oxo-3,3-dimethylvaleric Acid; 4-(1-((1-naphthyl)methyl)-5-trifluoromethylbenzimidazole-2-ylthio)butyric Acid; 4-(1-((2,5-dimethylphenil)methyl)-5-methoxybenzimidazole-2-ylthio)butyric Acid; 4-(1-((2,5-dimethylphenyl)methyl)-5-cyanobenzimidazole-2-ylthio)butyric Acid; 4-(1-((1-naphthyl)methyl)-5-ethoxybenzimidazole-2-ylthio)butyric Acid; 3-(1-((1-naphthyl)methyl)-benzimidazole-2-ylthio)propanoic Acid; 5-(1-((1-naphthyl)methyl)-benzimidazole-2-ylthio)valeric Acid; 4-(1-((1-naphthyl)methyl)-5,6-dimethylbenzimidazole-2-ylthio)butyric Acid; 4(1-((1-naphthyl)methyl)-5,6-dimethylbenzimidazole-2-ylsulfonyl)butyric Acid; and 4-(1-((2,5-dimethylphenyl)methyl)-7-azabenzimidazole-2-ylthio)butyric Acid.

US20020187989 discloses quinazoline derivative as chymase inhibitors. For example, the following species are disclosed:
7-chloro-3-(3-hydroxybenzenesulfonyl)-2,4(1H,3H)-quinazolinedione;
3-(4-hydroxybenzenesulfonyl)-7-chloro-2,4(1H,3H)-quinazolinedione;
3-(3-carboxymethyl-benzenesulfonyl)-7-chloro-2,4(1H,3H)-quinazolinedione;
3-(4-carboxymethyl-benzenesulfonyl)-7-chloro-2,4(1H,3H)-quinazolinedione;
3-[(3-carboxymethylaminophenyl)sulfonyl]-7-chloro-2,4(1H,3H)-quinazolinedione;
(+)-2-{4-[(7-chloro-2,4(1H,3H)-quinazolin-3-yl)sulfonyl]phenyl}butyric acid;
2-{3-[(7-chloro-2,4(1H,3H)-quinazolinedion-3-yl)sulfonyl]phenylaminocarbonyl}propionic acid;
3-{3-[(7-chloro-2,4(1H,3H)-quinazolinedion-3-yl)sulfonyl]phenyl}acrylic acid;
4-[(7-chloro-2,4(1H,3H)-quinazolinedion-3-yl)sulfonyl]salicylic acid;
4-[(7-chloro-2,4(1H,3H)-quinazolinedion-3-yl)sulfonyl]salicylic acid monosodium salt;
4-[(7-chloro-2,4(1H,3H)-quinazolinedion-3-yl)sulfonyl]anthranilic acid;
4-[(7-chloro-2,4(1H,3H)-quinazolinedion-3-yl)sulfonyl]anthranilic acid monosodium salt;
4-[(2,4(1H,3H)-quinazolinedion-3-yl)sulfonyl]salicylic acid;
5-[(7-chloro-2,4(1H,3H)-quinazolinedion-3-yl)sulfonyl]salicylic acid;
4-[(7-chloro-2,4(1H,3H)-quinazolinedion-3-yl)sulfonyl]-1-hydroxy-nanhthalene-2-carboxylic acid;
5-[(7-chloro-2,4(1H,3H)-quinazolinedion-3-yl)sulfonyl]anthranilic acid;
4-[(7-methoxy-2,4(1H,3H)-quinazolinedion-3-yl)sulfonyl]anthranilic acid;
(+)-7-[(7-chloro-2,4(1H,3H)-quinazolinedion-3-yl)sulfonyl]-2-oxo-1H,3H-quinoline-3-carboxylic acid;
(+)-6-[(7-chloro-2,4(1H,3H)-quinazolinedion-3-yl)sulfonyl]-3-oxo-1,4-benzoxazine-2-carboxylic acid;
4-[(7-hydroxy-2,4(1H,3H)-quinazolinedion-3-yl)sulfonyl]anthranilic acid;
4-[(7-chloro-2,4(1H,3H-quinazolinedion-3-yl)sulfonyl]-2-N-propionylanthranilic acid;
4-[(6-chloro-2,4(1H,3H)-quinazolinedion-3-yl)sulfonyl]anthranilic acid; 4-[(7-chloro-2,4(1H,3H)-quinazolinedion-3-yl)sulfonyl]-2-N-methanesulfonylanthranilic acid;
3-(3-acetamide-4-methoxybenzenesulfonyl)-7-chloro-2,4(1H,3H)-quinazolinedione;
3-(3-amino-4-methoxybenzenesulfonyl)-7-chloro-2,4(1H,3H)-quinazolinedione;
7-chloro-3-(4-methoxy-3-methylsulfonylaminobenzenesulfonyl)-2,4(1H,3H)-quinazolinedione;
3-(2-aminobenzenesulfonyl)-7-chloro-2,4(1H,3H)-quinazolinedione;
7-chloro-3-(2-methylsulfonylaminobenzenesulfonyl)-2,4(1H,3H-quinazolinedione;
3-(4-aminobenzenesulfonyl)-7-chloro-2,4(1H,3H)-quinazolinedione;
3-(3-amino-4-chlorobenzenesulfonyl)-7-chloro-2,4(1H,3H)-quinazolinedione;
3-(4-amino-3,5-dichlorobenzenesulfonyl)-7-chloro-2,4(1H,3H)-quinazolinedione;
3-(3-amino-4-methylbenzenesulfonyl)-7-chloro-2 4(in 3H)-quinazolinedione; and
3-(3-aminobenzenesulfonyl)-7-chloro-2,4(1H,3H)-quinazolinedione;

US200201833393 discloses quinazoline derivative as chymase inhibitors. For example, (3-aminobenzenesulfonyl)-7-chloro-2,4-(1H,3H)quinazolinedione Methanesulfonic Acid Salt and 7-chloro-3-[4-(pyrazol-3-yl)benzenesulfonyl]-2,4(1H,3H)-quinazolinedione Hydrochloride are disclosed.

US20030229126 discloses N-substituted benzothiophenesulfonamide derivatives as chymase inhibitors. For example, the following species are disclosed:
5-chloro-N-(4-hydroxymethyl-2-methanesulfonylphenyl)-3-methylbenzo[b]thiophene-2-sulfonamide;
5-fluoro-N-[4-(4-hydroxymethyloxazol-2-y-l)-2-methanesulfonylphenyl]-3-methylbenzo[b]thiophene-2-sulfonamide;
5-fluoro-N-[4-(4-hydroxymethylthiazol-2-yl)-2-methanesulfonylphenyl]-3-m-ethylbenzo[b]thiophene-2-sulfonamide;
2-[4-(5-chloro-3-methylbenzo[b]thiophene-2-sulfonylamino)-3-methanesulfonylphenyl]oxazole-4-carboxylic acid;
2-[4-(5-fluoro-3-methylbenzo[b]thiophene-2-sulfonylamino)-3-methanesulfonylphenyl]oxazole-4-carboxylic acid;
disodium 2-[4-(5-chloro-3-methylbenzo[-b]thiophene-2-sulfonylamino)-3-methanesulfonylphenyl]oxazole-4-carboxylate;
disodium 2-[4-(5-fluoro-3-methylbenzo[b]thiophene-2-sulfonylamino)-3-methanesulfonylphenyl]oxazole-4-carboxylate;
2-[4-(5-fluoro-3-methylbenzo[b]thiophene-2-sulfonylamino)-3-methanesulfonylphenyl]thiazole-4-carboxylic acid;
methyl 4-(5-chloro-3-methylbenzo[b]thiophene-2-sulfonylamino)-3-methanesulfonylbenzoate;
sodium methyl 4-(5-chloro-3-methylbenzo[b]thiophene-2-sulfonylamino)-3-methanesulfonylbenzoate;
isopropyl 4-(5-chloro-3-methylbenzo[b]thiophene-2-sulfonylamino)-3-methanesulfonylbenzoate;
N-(4-acetyl-2-methanesulfonylphenyl)-5-chloro-3-methylbenzo[b]thiophene-2-sulfonamide; N-(4-benzoyl-2-methanesulfonylphenyl)-5-chloro-3-methylbenzo[b]thiophene-2-sulfonamide;
ethyl 4-(5-chloro-3-methylbenzo[b]thiophene-2-sulfonylamino-)-3-methanesulfonylbenzoate;
tert-butyl 4-(5-chloro-3-methylbenzo[b]thioph-ene-2-sulfonylamino)-3-methanesulfonylbenzoate;
methyl 4-(5-chloro-3-methylbenzo[b]thiophene-2-sulfonylamino)-3-ethanesulfonylbenzoate;
methyl 4-(5-chloro-3-methylbenzo[b]thiophene-2-sulfonylamino)-5-methanesulfonyl-2-methylbenzoate;
dimethyl 4-(5-chloro-3-methylbenzo[b]thiop-hene-2-sulfonylamino)isophthalate,
methyl 4-(5-chloro-3-methylbenzo[b]thio-phene-2-sulfonylamino)-3-methoxybenzoate;
methyl 4-(5-chloro-3-methylbenzo-[b]thiophene-2-sulfonylamino)-3-nitrobenzoate;
ethyl 4-(5-chloro-3-methylbenzo[b]thiophene-2-sulfonylamino) benzoate;
5-chloro-N-(2,4-dimethanesulfonylphenyl)-3-methylbenzo[b]thiophene-2-sulfonamide;
N-(4-acetyl-2-nitrophenyl)-5-chloro-3-methylbenzo[b]thiophene-2-sulfonamide;
N-(4-benzoylphenyl)-5-chloro-3-methylbenzo-[b]thiophene-2-sulfonamide;
5-chloro-N-(2-methanesulfonylphenyl)-3-methylbenzo[b]thiophene-2-sulfonamide;
methyl 4-(5-fluoro-3-methylbenzo[b]thiophe-ne-2-sulfonylamino)-3-methanesulfonylbenzoate;
methyl 4-(5-methyl-3-methylbenzo[b]thiophene-2-sulfonylamino)-3-methanesulfonylbenzoate;
N-(4-acetyl-2-methanesulfonylphenyl)-5-fluoro-3-methylbenzo[b]thiophene-2-sulfonamide;
methyl 4-(3-methylbenzo[b]thiophene-2-sulfonylamino)-3-methanesulfonylbenzoate;
methyl 2-[4-(5-chloro-3-methylbenzo[b]thiophene-2-sulfonylamino)-3-methanesulfonylphenyl]oxazole-4-carboxylate;
methyl 2-[4-(5-fluoro-3-methylbenzo[b]thiophene-2-sulfonylamino)-3-methanesulfonylphenyl]oxazole-4-carboxylate;
5-fluoro-N-(2-methanesulfonyl-4-oxazol-5-ylphenyl)-3-methylbenzo[b]thiophene-2-sulfonamide;
methyl 2-[4-(5-fluoro-3-methylbenzo[b]thiophene-2-sulfonylamino)-3-methanesulfonylphenyl]thiazole-4-carboxylate;

N-[4-(4-chloromethylthiazol-2-yl)-2-methanesulfonylphenyl]-5-fluoro-3-methylbenzo[b]thiophene-2-sulfonamide;
5-fluoro-N-[2-methanesulfonyl-4-(4-methylthiazol-2-yl)phenyl]-3-methylbenzo[b]thiophene-2-sulfonamide;
5-fluoro-N-[2-methanesulfonyl-4-(2-methylthi-azol-4-yl)phenyl]-3-methylbenzo[b]thiophene-2-sulfonamide;
5-fluoro-N-[2-methanesulfonyl-4-(5-methylthiazol-2-yl)phenyl]-3-methylbenzo[b]thiophene-2-sulfonamide;
5-fluoro-N-[2-methanesulfonyl-4-(5-methoxy-4-methylthiazol-2-yl)phenyl]-3-methylbenzo[b]thiophene-2-sulfonamide;
5-fluoro-N-[2-methanesulfonyl-4-(4,5-dimethylthiazol-2-yl)phenyl]-3-methyl-lbenzo[b]thiophene-2-sulfonamide;
N-[4-(4-chloromethyloxazol-2-yl)-2-methanesulfonylphenyl]-5-fluoro-3-meth-ylbenzo[b]thiophene-2-sulfonamide;
5-fluoro-N-[2-methanesulfonyl-4-(4-methyloxazol-2-yl)phenyl]-3-methylbenzo[b]thiophene-2-sulfonamide;
5-fluoro-N-[2-methanesulfonyl-4-(5-methoxy-4-methyloxazol-2-yl)phenyl]-3-methylbenzo[b]thiophene-2-sulfonamide;
5-fluoro-N-[2-methanesulfonyl-4-(5-ethoxy-4-methyloxazol-2-yl)phenyl]-3-methylbenzo[b]thiophene-2-sulfonamide;
5-fluoro-N-[2-methanesulfonyl-4-(4,5-dimethyloxazol-2-yl)phenyl]-3-methyl-lbenzo[b]thiophene-2-sulfonamide;
5-fluoro-N-[2-methanesulfonyl-4-(5-methyl-loxazol-2-yl)phenyl]-3-methylbenzo[b]thiophene-2-sulfonamide; and
5-fluoro-N-[2-methanesulfonyl-4-((E)-2-methanesulfinyl-2-methylsulfanylvinyl)phenyl]-3-methylbenzo[b]thiophene-2-sulfonamide.

US20040102384 discloses pyrrolidine derivatives including the following species:
N-[(1S)-2-((2S)-2-{N-[(1S)-1-(benzo[b]thiophen-3-ylmethyl)-3,3,3-trifluoro-2-oxopropyl]carbamoyl}pyrrolidinyl)-1-(methylethyl)-2-oxoethyl](3,5-dimethylisoxazol-4-yl)carboxamide; N-[(1S)-2-((2S)-2-{N-[(1S)-3,3,3-trifluoro-1-1-(2-naphthylmethyl)-2-oxopropyl]carbamoyl}pyrrolidinyl)-1-(methylethyl)-2-oxoethyl](3,5-dimethylisoxazol-4-yl)carboxamide; N-[(1S)-2-((2S)-2-{N-[(1S)-1-(benzo[b]thiophen-3-ylmethyl)-3,3,3-trifluoro-2-oxopropyl]carbamoyl}pyrrolidinyl)-1-ethyl-2-oxoethyl](3,5-dimethylisoxazol-4-yl)carboxamide; N-[1-((1R)-1-methylpropyl)(1S)-2-((2S)-2-{N-[(1S)-1-(benzo[b]thiophen-3-ylmethyl)-3,3,3-trifluoro-2-oxopropyl]carbamoyl}pyrrolidinyl)-2-oxoethyl](3,5-dimethylisoxazol-4-yl)carboxamide; N-[(1S)-2-((2S)-2-{N-[(1S)-1-(benzo[b]thiophen-3-ylmethyl)-3,3,3-trifluor-o-2-oxopropyl]carbamoyl}pyrrolidinyl)-1-methyl-2-oxoethyl](3,5-dimethyliso-xazol-4-yl)carboxamide; and N-[(1S)-2-((2S)-2-{N-[(1S)-1-(benzo[b]thiophen-1-3-ylmethyl)-3,3,3-trifluoro-2-oxopropyl]carbamoyl}pyrrolidinyl)-1-(methyl-ethyl)-2-oxoethyl](4-methyl(1,2,3-thiadiazol-5-yl))carboxamide.

US20040082544 discloses phosphonic acid compounds which are chymase inhibitors including:
[2-[3-[[[1-[(6-Hydroxy-2-naphthalenyl)carbonyl]-4-piperidinyl]methylamino]carbonyl]-2-naphthalenyl]-1-(1-naphthalenyl)-2-oxoethyl]-phosphonic acid;
[2-[3-[[Methyl(4-phenylcyclohexyl)-amino]carbonyl]-2-naphthalenyl]-1-(1-naphthalenyl)-2-oxoethyl]-phosphonic acid;
[2-[3-[[Methyl[1-(2-naphthalenylcarbonyl)-4-piperidinyl]amino]carbonyl]-2-naphthalenyl]-1-(1-naphthalenyl)-2-oxoethyl]-phosphonic acid;
[2-[3-[[[1-[(6-Methoxy-2-naphthalenyl)carbonyl]-3-pyrrolidinyl]methylamino]carbonyl]-2-naphthalenyl]-1-(1-naphthalenyl)-2-oxoethyl]-phosphonic acid;
[2-[3-[[[1-[(6-Bromo-2-naphthalenyl)carbonyl]-4-piperidinyl]methylamino]carbonyl]-2-naphthalenyl]-1-(1-naphthalenyl)-2-oxoethyl]-phosphonic acid;
[2-[3-[[[1-[(2E)-3-(4-Fluorophenyl)-1-oxo-2-propenyl]-3-pyrrolidinyl]methylamino]carbonyl]-2-naphthalenyl]-1-(1-naphthalenyl)-2-oxo ethyl]-phosphonic acid;
[2-[3-[[Methyl[1-[(2E)-1-oxo-3-phenyl-2-propenyl]-4-piperidinyl]amino]carbonyl]-2-naphthalenyl]-1-(1-naphthalenyl)-2-oxoethyl]-phosphonic acid;
[2-[3-[[Methyl[1-[(2E)-3-(4-methylphenyl)-1-oxo-2-propenyl]-4-piperidinyl]amino]carbonyl]-2-naphthalenyl]-1-(1-naphthalenyl)-2-oxoethyl]-phosphonic acid;
[2-[3-[[Methyl[1-[(2E)-1-oxo-3-[4-(trifluoromethyl)phenyl-]-2-propenyl]-4-piperidinyl]amino]carbonyl]-2-naphthalenyl]-1-(1-naphthalenyl)-2-oxoethyl]-phosphonic acid;
[2-[3-[[[1-[(2E)-3-[4-(Dimethylamino)phenyl]-1-oxo-2-propenyl]-4-piperidinyl]methylamino]carbonyl]-2-naphthalenyl]-1-(1-naphthalenyl)-2-oxoethyl]-phosphonic acid;
[2-[3-[[(1-Benzoyl-4-piperidinyl)methylamino]carbonyl]-2-naphthalenyl]-1-(1-naphthalenyl)-2-oxoethyl-1]-phosphonic acid;
[2-[3-[(Cyclohexylmethylamino)carbonyl]-2-naphthalenyl-]-1-(1-naphthalenyl)-2-oxoethyl]-phosphonic acid;
[2-[3-[[Methyl[1-[1-oxo-3-[4-(trifluoromethyl)phenyl]propyl]-4-piperidinyl]amino]carbonyl]-2-naphthalenyl]-1-(1-naphthalenyl)-2-oxoethyl]-phosphonic acid;
[2-[3-[[Methyl[1-(2-methyl-1-oxopropyl)-4-piperidinyl]amino]carbonyl]-2-naphthalenyl]-1-(1-naphthalenyl)-2-oxoethyl]-phosphonic acid;
[2-[3-[(Cyclopentylmethylamino)carbonyl]-2-naphthalenyl]-1-(1-naphthaleny-1)-2-oxoethyl]-phosphonic acid;
[2-[3-[[[4-(1,1-Dimethylethyl)cyclohexyl]methylamino]carbonyl]-2-naphthalenyl]-1-(1-naphthalenyl)-2-oxoethyl]-phosphonic acid;
[2-[3-[[(1-Acetyl-4-piperidinyl)methylamino]carbonyl]-2-naphthalenyl]-1-(1-naphthalenyl)-2-oxoethyl]-phosphonic acid;
[2-[3-[[Methyl(4-methylcyclohexyl)amino]carbonyl]-2-naphthalenyl]-1-(1-naphthalenyl)-2-oxoethyl]-phosphonic acid;
[2-[1-[[Methyl(tricyclo[3.3.1.1.sup.3,7]dec-1-ylmethyl)amino]carbonyl]-2-naphthalenyl]-1-(1-naphthalenyl)-2-oxoethyl]-phosphonic acid;
[2-[3-[[Methyl(4-phenyl-3-cyclohexenlyl)amino]carbonyl]-2-naphthalenyl]-1-(1-naphthalenyl)-2-oxoethyl]-phosphonic acid;
[1-(1-Naphthalenyl)-2-[3-[[[1-(2-naphthalenylcarbonyl)-4-piperidinyl]amino]carbonyl]-2-naphthalenyl]-2-oxoethyl]-phosphonic acid;
[1-(1-Naphthalenyl)-2-oxo-2-[3-[(4-phenyl-1-piperidinyl)carbonyl]-2-naphthalenyl]ethyl]-phosphonic acid;
[1-(1-Naphthalenyl)-2-oxo-2-[3-[(4-oxo-1-phenyl-1,3,8-triazaspiro[4.5]dec-8-yl)carbonyl]-2-naphthalenyl]ethyl]-phosphonic acid;
[2-[3-[[4-(4-Methoxyphenyl)-1-piperidinyl]carbonyl]-2-naphthalenyl]-1-(1-naphthalenyl)-2-oxoethyl]-phosphonic acid;
[2-[3-[[4-(3-Methoxyphenyl)-1-piperidinyl]carbonyl]-2-naphthalenyl]-1-(1-naphthalenyl)-2-oxoethyl]-phosphonic acid;
[2-[3-[[4-(2-Benzothiazolyl)-1-piperidinyl]carbonyl]-2-naphthalenyl]-1-(1-naphthalenyl)-2-oxoethyl]-phosphonic acid;
[1-(1-Naphthalenyl)-2-oxo-2-[3-[(3-phenyl-1-pyrrolidinyl)carbonyl]-2-naphthalenyl]ethyl]-phosphonic acid;
[1-(1-Naphthalenyl)-2-oxo-2-[3-[[3-(2-phenylethyl)-1-pyrrolidinyl]carbonyl]-2-naphthalenyl]ethyl]-phosphonic acid;
[2-[3-[[Methyl [1-(2-naphthalenylcarbonyl)-4-piperidinyl]amino]carbonyl]-2-naphthalenyl]-2-oxo-1-phenylethyl]-phosphonic acid;
[2-[2-[[Methyl [1-(2-naphthalenylcarbonyl)-4-piperidinyl]amino]carbonyl]phenyl]-1-(1-naphthalenyl)-2-oxoethyl]-phosphonic acid;
Methyl[2-[3-[[methyl[1-(2-naphthalenylcarbonyl)-4-piperidinyl]amino]carbonyl]-2-naphthalenyl]-1-(1-naphthalenyl)-2-oxoethyl]-phosphinic acid;
[1-(5-Chlorobenzo[b]thien-3-yl)-2-[3-[[methyl[1-(2-naphthalenylcarbonyl)-4-piperidinyl]amino]carbonyl]-2-naphthalenyl]-2-oxoethyl]-phosphonic acid; and
[2-[3-[[Methyl [1-(2-naphthalenylcarbonyl)-4-piperidinyl]amino]carbonyl]-2-naphthalenyl]-1-(1-naphthalenyl)-2-oxoethyl]-phosphonic acid methyl ester.

U.S. Pat. No. 5,306,824 discloses chymase inhibitors including: 7-biotinylamino-4-chloro-3-(2-phenylethoxy)isocoumarin and 7-(6-biotinylaminocaproyl)amino-4-chloro-3-(2-phenylethoxy)isocoumarin.

WO93/25574 discloses chymase inhibitor including: N-[(1,1-dimethylethoxy)carbonyl]-L-alanyl-N-[2,3-dioxo-3-methoxy-1-(phenylmethyl)propyl]-L-prolinamide; N-[(1,1-dimethylethoxy)carbonyl]-L-valyl-N-[2,3-dioxo-3-methoxy-1-(phenylmethyl)propyl]-L-prolinamide; N-[4-[N-methylamino]piperidine-1-carbonyl]-L-valyl-N-[3,3,3-trifluo-ro-2-oxo-1(S)-(phenylmethyl)propyl]-L-prolinamide hydrochloride; N-[4-[N-methylamino]piperidine-1-carbonyl]-L-valyl-N-[3,3,3-trifluo-ro-2-oxo-1(S)-(phenylmethyl)propyl]-L-prolinamide hydrochloride; N-[4-[N-methylamino]piperidine-1-carbonyl]-L-valyl-N-[2,3-dioxo-3-(-(1-methyl)ethoxy)-1(S)(phenylmethyl)propyl]-L-prolinamide hydrochloride; N-[4-[N-methylamino]piperidine-1-carbonyl-L-valyl-N-[2,3-dioxo-3-((−1-methyl)ethoxy)-1-(S)(phenylmethyl)propyl]-L-prolinamide hydrochloride; N-(4-oxopiperidine-1-carbonyl)-L-phenylalanyl-N-[2,3-dioxo-3-((1-me-thyl)ethoy)-1-(S)-(phenyl-methyl)propyl]-L-prolinamide; and N-[4-[N-methylamino]piperidine-1-carbonyl]-L-phenylalanyl-N-[3,3,3-trifluoro-2-oxo-1(S)-(phenyl-methyl)propyl]-L-prolinamide.

WO96/04248 discloses imidazoline derivatives including: 3-(3-Methoxycarbonylbenzensulfonyl)-1-phenyl-imidazolidine-2,4-dione; 3-(4-Chlorobenzenesulfonyl)-1-phenyl-imidazolidine-2,4-dione; 3-(3,4 Dimethylbenzenesulfonyl)-1-(3,4-dimethylphenyl)-imidazolidine-2,4-dione; 3-(4-Allyloxycarbonylbenzenesulfonyl)-1-(3,4-dimethylphenyl)-imidazolidine-2,4-dione; and 1-(3,4-Dichlorophenyl)-3-(2-naphthyl-sulfonyl)imidazolidine-2,4-dione.

Japanese Unexamined Patent Publication No. 9-31061 discloses hydantoin derivatives including: (5R)-5-Benzyl-3-(4-chlorobenzenesulfonyl)-imidazolidine-2,4-dione and (5R)-5-benzyl-3-(3,4-dimethylbenzenesulfonyl)-imidazolidine-2,4-dione.

WO97/11941 discloses quinazoline derivatives including: [7-Chloro-3-(4-chlorobenzenesulfonyl)-2,4(1H, 3H)-quinazolinedion-1-yl]acetanilide; [7-chloro-3-(4-chlorobenzenesulfonyl)-2,4(1H, 3H)-quinazolinedion-1-yl]acetic acid 4-hydroxyanilide; [7-chloro-3-(4-chlorobenzenesulfonyl)-2,4(1H, 3H)-quinazolinedion-1-yl]acetic acid 3-pyridinamide; [7-chloro-3-(4-chlorobenzenesulfonyl)-2,4(1H, 3H)-quinazolinedion-1-yl]acetic acid 4-pyridinamide; [7-chloro-3-(4-chlorobenzenesulfonyl)-2,4(1H, 3H)-quinazolinedion-1-yl]acetic acid 2-pyridinamide; 3-{[7-chloro-3-(4-chlorobenzenesulfonyl)-2,4(1H,3H)-quinazolinedion-1-yl]acetylamino}-N-ethylpyridinium iodide; [7-chloro-3-(4-chlorobenzenesulfonyl)-2,4(1H, 3H)-quinazolinedion-1-yl]acetic acid 5-indolamide; 3-{[7-chloro-3-(4-chlorobenzenesulfonyl)-2,4 (1H,3H)-quinazolinedion-1-yl]acetylamino}-(N-t-butoxycarbonylmethylpyridinium)bromide; 3-{[7-chloro-3-(4-chlorobenzenesulfonyl)-2,4(1H,3H)-quinazolinedion-1-yl]acetylamino)-N-carboxymethylpyridinium bromide; 3-[7-chloro-3-(4-chlorobenzenesulfonyl)-2,4(1H,3H)-quinazolinedion-1-yl]acetylaminophenoxy-phosphoric acid; 7-chloro-3-(4-chlorobenzenesulfonyl)-1-(3-pyridylmethyl)-2,4(1H,3H)-quinazolinedione; and [7-chloro-3-(4-chlorobenzenesulfonyl)-2,4-(1H, 3H)-quinazolinedion-1-yl]acetic acid 4-chloroanilide.

EP0713876 discloses triazine derivatives including: 5-(4-Chlorobenzylsulfinyl)-8-hydroxyimidazo-[1,2-d][1,2,4]triazine; 8-(4-chlorobenzyloxy)-5-(4-chlorobenzylsulfinyl)imidazo[1,2-d][1,2,-4]triazine; 8-(4-methylbenzyloxy)-5-(4-methylbenzylsulfinyl)imidazo[1,2-d][1,2,-4]triazine; 8-(3-chlorobenzyloxy)-5-(3-chlorobenzylsulfinyl)imidazo[1,2-d][1,2,-4]triazine; 1-(4-chlorobenzyloxy)-4-(4 chlorobenzylsulfinyl)-8-methylimidazo[1,-5-d][1,2,4]triazine; 2-(4-chlorobenzyl)-4-(4-chlorobenzylsulfinyl)-8-methylimidazo[1,5-d-][1,2,4]triazin-1(2H)-one; 5-(4-chlorobenzylsulfinyl)-8-ethoxycarbonyl-methyloxyimidazo[1,2-d]-[1,2,4]triazine; 4-(4-chlorobenzylsulfinyl)-1-(ethoxycarbonylmethyloxy)-8-methylimid-azo[1,5-d][1,2,4]triazine; and 2-ethoxycarbonylmethyl-4-(4-chlorobenzylsulfinyl)-8-methylimidazo[1-,5-d][1,2,4]triazin-1(2H)-one.

Japanese Unexamined Patent Publication No. 10-87567 discloses phenol derivatives including:
1-[4-(3-Indolylacetoxy)benzoyl]-4-piperidine-carboxylic acid;
(S)-2-[4-(3-indolylacetoxy)benzoyl]-1,2,3,4-tetrahydroisoquinoline-3-carboxylic acid;
(S)-1-[4-(3-indolylacetoxy)benzoyl]-pyrrolidine-2-carboxylic acid; and
(S)-1-[4-(3-(2-methyl)indolylacetoxy)benzoyl]-pyrrolidine-2-carboxylic acid.

WO96/33974 discloses heterocyclic amide derivatives including 2-[5-Amino-2-(3,5-diaminophenyl)-6-oxo-1,6-dihydro-1-pyrimidyl]-N-(1-benzyl-3,3,3-trifluoro-2-oxopropyl)acetamide and 2-(5-hydroxysuccinylamino-6-oxo-2-phenyl-1,6-dihydro-1-pyrimidyl)-N-(1-benzyl-3,3,3-trifluoro-2-oxopropyl)acetamide.

Japanese Unexamined Patent Publication No. 9-124691 discloses chymase inhibitors including:
N-[4(S)—[N—[N-(tert-butyloxycarbonyl)-L-valyl-L-prolyl]amino]-2,2-d-ifluoro-3-oxo-5-phenylvaleryl]glycine;
3-[N-[4(S)—[N—[N-(tert-butyloxycarbonyl)-L-glutamyl-L-prolyl]amino]-2,2-difluoro-3-oxo-5-phenylvaleryl]amino]benzoic acid; and
3-[N-[4(S)—[N—[N-(phenylsulfonyl)-L-glutamyl-L-prolyl]amino]-2,2-di-fluoro-3-oxo-5-phenylvaleryl]amino]benzoic acid.

Japanese Unexamined Patent Publication No. 10-7661 discloses heterocyclic amide derivatives including 2-[1,6-Dihydro-5-hydroxysuccinylamino-2-(3-methylphenyl)-6-oxo-1 pyrimidinyl]-N-[1-benzyl-3,3-difluoro-2-oxo-3-[N-(carboxymethyl)carbamoyl]propyl]acetamide and 2-[1,6-dihydro-5-hydroxysuccinylamino-2-(3-methylphenyl)-6-oxo-1-py-rimidinyl]-N-[1-benzyl-3,3-difluoro-2-oxo-3-[N-(3-carboxyphenyl)carbamoyl]-propyl]acetamide.

Japanese Unexamined Patent Publication No. 10-53579 discloses chymase inhibitors including: 2-[5-[(Benzyloxycarbonyl)amino]-2-(4-fluorophenyl)-1,6-dihydro-6-ox-o-1-pyrimidinyl]-N-[2-(4,4,4-trifluoro-3-oxo-1-phenyl)butyl]acetamide; 2-[5-amino-2-(4-fluorophenyl)-1,6-dihydro-6-oxo-1-pyrimidinyl]-N-[2-(4,4,4-trifluoro-3-oxo-1-phenyl)butyl]acetamide; 2-[5-amino-2-phenyl-1,6-dihydro-6-oxo-1-pyrimidinyl]-N-[2-(4,4,4-tr-ifluoro-3-oxo-1-phenyl)butyl]acetamide; 2-(5-amino-1,6-dihydro-6-oxo-2-phenyl-1-pyrimidinyl)-N-[2-(3-methox-ylcarbonyl-3-oxo-1-phenyl)butyl]acetamide; and 2-(3-amino-2,3-dihydro-2-oxo-5-phenyl-1H-1,4-benzodiazepin-1-yl)-N-[2-(4,4,4-trifluoro-3-oxo-1-phenyl)butyl]acetamide.

WO 98/09949 discloses acetamide derivatives useful as chymase inhibitors including:

2-(5-Amino-6-oxo-2-phenyl-1,6-dihydropyrimidin-1-yl)-N-(2,3-dioxo-1-phenylmethyl-6-(2-pyridyloxy)}hexylacetamide.2HCl; 2-{6-oxo-2-phenyl-5-(4-pyridylmethyloxycarbonylamino)-1,6-dihydropyrimidin-1-yl}-N-(2,3-dioxo-1-phenylmethyl-6-(2-pyridyloxy)}hexylacetamide; 2-(5-formylamino-6-oxo-2-phenyl-1,6-dihydropyrimidin-1-yl)-N-{2,3-d-ioxo-1-phenylmethyl-6-(2-pyridyloxy)}hexylacetamide; and 2-(5-benzylaminosulfonylamino-6-oxo-2-phenyl-1,6-dihydropyrimidin-1-yl)-N-{2,3-dioxo-1-phenylmethyl-6-(2-pyridyloxy)}hexylacetamide.

WO00/005204 discloses monocyclic β-lactams as chymase inhibitors.

WO98/18794 discloses heterocyclic amide derivatives that are chymase inhibitors.

### Anti-Chymotrypsin and Anti-Chymotrypsin-Like Enzyme Antibodies

As discussed above, antibodies directed against chymotrypsin or a chymotrypsin-like enzyme can be used in any of the methods described herein. Useful antibodies inhibit the ability of the enzyme to cleave guanylin, for example, by binding to the active site of the enzyme or by interfering with the interaction between the enzyme and guanylin.

There a number of commercially available sources of anti-chymotrypsin antibodies including: Research Diagnostics Inc. (Flanders, N.J.; e.g., catalog nos. RDI-CHYMOTabm and RDI-CHYMOT-AG), Biodesign International (Saco, Me.; e.g., catalog nos. H55601M, H55136M), Chemicon International, Inc (Temecula, Calif.; e.g., catalog no. MAB1476), Fitzgerald Industries International (Concord, Mass. e.g. catalog no. 20-CR79), QED Biosciences (San Diego, Calif.; catalog nos. 13601, 13602, 13603, and 13604), and US Biological (Swampscott, Mass.; e.g., catalog nos. C5070, C5070-01, C5070-02, and C5070-07).

Chymase and cathepsin are examples of chymotrypsin-like enzymes. There a number of commercially available sources of anti-chymase antibodies including: Abcam (Cambridge, UK; e.g., catalog no. ab2377), Calbiochem (San Diego, Calif.; e.g., catalog no. 444904), Chemicon International, Inc (Temecula, Calif.; e.g., catalog nos. MAB1254B and MAB1254), Lab Vision (Fremont, Calif.; e.g., catalog no. MS-1217), Novus Biologicals (Littleton, Colo.; e.g., catalog no. ab2377), Serotec, Inc. (Raleigh, N.C.; e.g., catalog no. MCA1930), and US Biological (Swampscott, Mass.; e.g. catalog no. M2414). There a number of commercially available sources of anti-cathepsin antibodies including: Abcam (Cambridge, UK; e.g. catalog nos. ab15988 and ab8816), BD Biosciences Pharmingen (San Diego, Calif.; e.g. catalog no. 554248), Biodesign International (Saco, Me.; e.g. catalog nos. K50806R and K90050C), Biovision (Mountain View, Calif.; e.g. catalog no. 3370-200), Calbiochem (San Diego, Calif.; e.g. catalog no. 219358), and Chemicon International, Inc (Temecula, Calif.; e.g. catalog no. MAB1054).

The antibodies can be a whole antibody (e.g., IgM, IgG, IgA, IgD, or IgE) molecule that is generated by any one of a variety of methods that are known in the art. The antibody can be made in or derived from any of a variety of species, e.g., humans, non-human primates (e.g., monkeys, baboons, or chimpanzees), rabbits, rats, and mice. The antibody can be a purified or a recombinant antibody. Also useful are antibody fragments and chimeric antibodies and humanized antibodies derived from non-human (e.g., mouse, rat, gerbil, or hamster) antibodies. Also useful are antibody fragments that bind antigen, e.g., Fab, F(ab′)₂, Fv, and single chain Fv (scFv) fragments. An scFv fragment is a single polypeptide chain that includes both the heavy and light chain variable regions of the antibody from which the scFv is derived. In addition, diabodies (Poljak (1994) Structure 2(12):1121-1123; Hudson et al. (1999) J. Immunol. Methods 23(1-2):177-189) and intrabodies (Huston et al. (2001) Hum. Antibodies 10(3-4):127-142; Wheeler et al. (2003) Mol. Ther. 8(3):355-366; Stocks (2004) Drug Discov. Today 9(22): 960-966) can be used.

Antibody fragments that contain the binding domain of the molecule can be generated by known techniques. For example: F(ab′)₂ fragments can be produced by pepsin digestion of antibody molecules; and Fab fragments can be generated by reducing the disulfide bridges of F(ab′)₂ fragments or by treating antibody molecules with papain and a reducing agent. See, e.g., National Institutes of Health, 1 Current Protocols In Immunology, Coligan et al., ed. 2.8, 2.10 (Wiley Interscience, 1991). scFv fragments can be produced, for example, as described in U.S. Pat. No. 4,642,334.

Chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Human antibodies can be produced in transgenic mice (Jakovits 1999 Exp Opin Invest Drugs 7:607; Green 199 J. Immunol Methods 231:11) and can be obtained commercially from Medarex, Inc. (Princeton, N.J.) or Abgenix, Inc. (Fremont, Calif.). Human antibodies can also be obtained using phage display libraries (Cambridge Antibody Technology; Cambridge, UK)

Prodrugs of Inhibitors of Chymotrypsin-Like Enzymes

Prodrug forms of any of the inhibitors of a chymotrypsin-like enzyme described herein that are acids can be created by replacing the acidic proton with R_wherein R is selected from a straight chain or branched C₁ to C₆ alkyl (C₁, C₂, C₃, C₄, C₅, C₆), alkenyl, alkynyl, or a C₆ aryl optionally independently substituted with one or more halogen (Cl, F, Br, I), —OH, —C(O)OH, or —NH₃. In cases where there are two or more acidic protons (e.g., certain phosphoric acids), one or more can be replaced. Alternatively R can be selected from

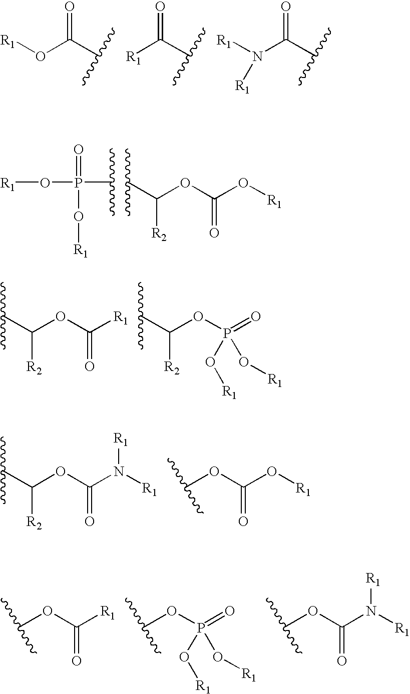

wherein R₁ is selected from H or a branched or straight chain C₁ to C₆ (C₁, C₂, C₃, C₄, C₅, C₆) alkyl, alkenyl, alkynyl, aryl, cycloalkyl, or arylalkyl optionally independently substituted with one or more halogen —OH, —C(O)OH, or —NH₃. R₂ is selected from H, straight or branched chain C₁ to C₆ (C₁, C₂, C₃, C₄, C₅, C₆) alkyl.

Among the inhibitors of chymotrypsin-like enzymes that are acids and might be converted to prodrugs in this manner are:
(4-[1-[[bis-(4-methylphenyl)-methyl]-carbamoyl]-3-(2-ethoxybenzyl)-4-oxo-azetidine-2-yloyl]-benzoic acid;
4-((1-((1-naphthyl)methyl)-5-methoxybenzimidazole)-2-ylthio)butyric acid;
4-((1-naphthyl)methyl)-5,6-difluorobenzimidazole)-2-ylthio)butyric acid;
4-(1-((1-naphthyl)methyl)-5-cyanobenzimidazole-2-ylthio)butyric acid;
4-(1-((1-naphthyl)methyl)-5-cyanobenzimidazole-2-yl)-3,3-dimethylbutyric acid;
4-(1-((1-naphthyl)methyl)benzimidazole-2-ylthio)butyric acid;
Sodium 4-(1-((1-naphthyl)methyl)benzimidazole-2-ylthio)butyrate;
4-(1-((8-methyl-1-naphthyl)methyl)benzimidazole-2-ylthio)butyric acid;
4-(1-((8-methyl-1-naphthyl)methyl)-5,6-dimethylbenzimidazole-2-ylthio)-butyric acid;
5-(1-((1-naphthyl)methyl)benzimidazole-2-ylthio)-4-oxo-3,3-dimethylvaleric acid;
4-(1-((1-naphthyl)methyl)-5-trifluoromethylbenzimidazole-2-ylthio)butyric acid;
4-(1-((2,5-dimethylphenyl)methyl)-5-methoxybenzimidazole-2-ylthio)butyric acid;
4-(1-((2,5-dimethylphenyl)methyl)-5-cyanobenzimidazole-2-ylthio)butyric acid;
4-(1-((1-naphthyl)methyl)-5-ethoxybenzimidazole-2-ylthio)butyric acid;
3-(1-((1-naphthyl)methyl)-benzimidazole-2-ylthio)propanoic acid; 5-(1-((1-naphthyl)methyl)-benzimidazole-2-ylthio)valeric acid;
4-(1-((1-naphthyl)methyl)-5,6-dimethylbenzimidazole-2-ylthio)butyric acid;
4(1-((1-naphthyl)methyl)-5,6-dimethylbenzimidazole-2-ylsulfonyl)butyric acid;
4-(1-((2,5-dimethylphenyl)methyl)-7-azabenzimidazole-2-ylthio)butyric acid;
(+)-2-{4-[(7-chloro-2,4(1H,3H)-quinazolin-3-yl)sulfonyl]phenyl}butyric acid;
2-{3-[(7-chloro-2,4(1H,3H)-quinazolinedion-3-yl)sulfonyl]phenylaminocarbonyl}propionic acid;
3-{3-[(7-chloro-2,4(1H,3H)-quinazolinedion-3-yl)sulfonyl]phenyl}acrylic acid;
4-[(7-chloro-2,4(1H,3H)-quinazolinedion-3-yl)sulfonyl]salicylic acid;
4-[(7-chloro-2,4(1H,3H)-quinazolinedion-3-yl)sulfonyl]salicylic acid monosodium salt;
4-[(7-chloro-2,4(1H,3H)-quinazolinedion-3-yl)sulfonyl]anthranilic acid;
4-[(7-chloro-2,4(1H,3H)-quinazolinedion-3-yl)sulfonyl]anthranilic acid monosodium salt;
4-[(2,4(1H,3H)-quinazolinedion-3-yl)sulfonyl]salicylic acid;
5-[(7-chloro-2,4(1H,3H)-quinazolinedion-3-yl)sulfonyl]salicylic acid;
4-[(7-chloro-2,4(1H,3H)-quinazolinedion-3-yl)sulfonyl]-1-hydroxy-nanhthalene-2-carboxylic acid;
5-[(7-chloro-2,4(1H,3H)-quinazolinedion-3-yl)sulfonyl]anthranilic acid;
4-[(7-methoxy-2,4(1H,3H)-quinazolinedion-3-yl)sulfonyl]anthranilic acid;
(+)-7-[(7-chloro-2,4(1H,3H)-quinazolinedion-3-yl)sulfonyl]-2-oxo-1H,3H-quinoline-3-carboxylic acid;
(+)-6-[(7-chloro-2,4(1H,3H)-quinazolinedion-3-yl)sulfonyl]-3-oxo-1,4-benzoxazine-2-carboxylic acid;
4-[(7-hydroxy-2,4(1H,3H)-quinazolinedion-3-yl)sulfonyl]anthranilic acid;
4-[(7-chloro-2,4(1H,3H-quinazolinedion-3-yl)sulfonyl]-2-N-propionylanthranilic acid;
4-[(6-chloro-2,4(1H,3H)-quinazolinedion-3-yl)sulfonyl]anthranilic acid;
4-[(7-chloro-2,4(1H,3H)-quinazolinedion-3-yl)sulfonyl]-2-N-methanesulfonylanthranilic acid;
2-[4-(5-chloro-3-methylbenzo[b]thiophene-2-sulfonylamino)-3-methanesulfonylphenyl]oxazole-4-carboxylic acid;
2-[4-(5-fluoro-3-methylbenzo[b]thiophene-2-sulfonylamino)-3-methanesulfon-ylphenyl]oxazole-4-carboxylic acid;
disodium 2-[4-(5-chloro-3-methylbenzo[-b]thiophene-2-sulfonylamino)-3-methanesulfonylphenyl]oxazole-4-carboxylate;
disodium 2-[4-(5-fluoro-3-methylbenzo[b]thiophene-2-sulfonylamino)-3-met-hanesulfonylphenyl]oxazole-4-carboxylate;
2-[4-(5-fluoro-3-methylbenzo[b]thiophene-2-sulfonylamino)-3-methanesulfonylphenyl]thiazole-4-carboxylic acid;
[2-[3-[[[1-[(6-Hydroxy-2-naphthalenyl)carbonyl]-4-piperidinyl]methylamino]carbonyl]-2-naphthalenyl]-1-(1-naphthalenyl)-2-oxoethyl]-phosphonic acid;
[2-[3-[[Methyl(4-phenylcyclohexyl)-amino]carbonyl]-2-naphthalenyl]-1-(1-naphthalenyl)-2-oxoethyl]-phosphonic acid;
[2-[3-[[Methyl[1-(2-naphthalenylcarbonyl)-4-piperidinyl]amino]carbonyl]-2-naphthalenyl]-1-(1-naphthalenyl)-2-oxoethyl]-phosphonic acid;
[2-[3-[[[1-[(6-Methoxy-2-naphthalenyl)carbonyl]-3-pyrrolidinyl]methylamino]carbonyl]-2-naphthalenyl]-1-(1-naphthalenyl)-2-oxoethyl]-phosphonic acid;
[2-[3-[[[1-[(6-Bromo-2-naphthalenyl)carbonyl]-4-piperidinyl]methylamino]carbonyl]-2-naphthalenyl]-1-(1-naphthalenyl)-2-oxoethyl]-phosphonic acid;
[2-[3-[[[1-[(2E)-3-(4-Fluorophenyl)-1-oxo-2-propenyl]-3-pyrrolidinyl]methylamino]carbonyl]-2-naphthalenyl]-1-(1-naphthalenyl)-2-oxoethyl]-phosphonic acid;
[2-[3-[[Methyl[1-[(2E)-1-oxo-3-phenyl-2-propenyl]-4-piperidinyl]amino]carbonyl]-2-naphthalenyl]-1-(1-naphthalenyl)-2-oxoethyl]-phosphonic acid;
[2-[3-[[Methyl[1-[(2E)-3-(4-methylphenyl)-1-oxo-2-propenyl]-4-piperidinyl]amino]carbonyl]-2-naphthalenyl]-1-(1-naphthalenyl)-2-oxoethyl]-phosphonic acid;
[2-[3-[[Methyl[1-[(2E)-1-oxo-3-[4-(trifluoromethyl)phenyl-]-2-propenyl]-4-piperidinyl]amino]carbonyl]-2-naphthalenyl]-1-(1-naphthalenyl)-2-oxoethyl]-phosphonic acid;
[2-[3-[[[1-[(2E)-3-[4-(Dimethylamino)phenyl]-1-oxo-2-propenyl]-4-piperidinyl]methylamino]carbonyl]-2-naphthalenyl]-1-(1-naphthalenyl)-2-oxoethyl]-phosphonic acid;
[2-[3-[[(1-Benzoyl-4-piperidinyl)methylamino]carbonyl]-2-naphthalenyl]-1-(1-naphthalenyl)-2-oxoethyl-1]-phosphonic acid;
[2-[3-[(Cyclohexylmethylamino)carbonyl]-2-naphthalenyl-]-1-(1-naphthalenyl)-2-oxoethyl]-phosphonic acid;
[2-[3-[[Methyl[1-[1-oxo-3-[4-(trifluoromethyl)phenyl]propyl]-4-piperidinyl]amino]carbonyl]-2-naphthalenyl]-1-(1-naphthalenyl)-2-oxoethyl]-phosphonic acid;
[2-[3-[[Methyl[1-(2-methyl-1-oxopropyl)-4-piperidinyl]amino]carbonyl]-2-naphthalenyl]-1-(1-naphthalenyl)-2-oxoethyl]-phosphonic acid;
[2-[3-[(Cyclopentylmethylamino)carbonyl]-2-naphthalenyl]-1-(1-naphthaleny-1)-2-oxoethyl]-phosphonic acid;
[2-[3-[[[4-(1,1-Dimethylethyl)cyclohexyl]methylamino]carbonyl]-2-naphthalenyl]-1-(1-naphthalenyl)-2-oxoethyl]-phosphonic acid;
[2-[3-[[(1-Acetyl-4-piperidinyl)methylamino]carbonyl]-2-naphthalenyl]-1-(1-naphthalenyl)-2-oxoethyl]-phosphonic acid;
[2-[3-[[Methyl(4-methylcyclohexyl)amino]carbonyl]-2-naphthalenyl]-1-(1-naphthalenyl)-2-oxoethyl]-phosphonic acid;
[2-[1-[[Methyl(tricyclo[3.3.1.1.s-up.3,7]dec-1-ylmethyl)amino]carbonyl]-2-naphthalenyl]-1-(1-naphthalenyl)-2-oxoethyl]-phosphonic acid;
[2-[3-[[Methyl(4-phenyl-3-cyclohexen 1 yl)amino]carbonyl]-2-naphthalenyl]-1-(1-naphthalenyl)-2-oxoethyl]-phosphonic acid;
[1-(1-Naphthalenyl)-2-[3-[[[1-(2-naphthalenylcarbonyl)-4-piperidinyl]amino]carbonyl]-2-naphthalenyl]-2-oxoethyl]-phosphonic acid;
[1-(1-Naphthalenyl)-2-oxo-2-[3-[(4-phenyl-1-piperidinyl)carbonyl]-2-naphthalenyl]ethyl]-phosphonic acid;
[1-(1-Naphthalenyl)-2-oxo-2-[3-[(4-oxo-1-phenyl-1,3,8-triazaspiro[4.5]dec-8-yl)carbonyl]-2-naphthalenyl]ethyl]-phosphonic acid;
[2-[3-[[4-(4-Methoxyphenyl)-1-piperidinyl]carbonyl]-2-naphthalenyl]-1-(1-naphthalenyl)-2-oxoethyl]-phosphonic acid;
[2-[3-[[4-(3-Methoxyphenyl)-1-piperidinyl]carbonyl]-2-naphthalenyl]-1-(1-naphthalenyl)-2-oxoethyl]-phosphonic acid;
[2-[3-[[4-(2-Benzothiazolyl)-1-piperidinyl]carbonyl]-2-naphthalenyl]-1-(1-naphthalenyl)-2-oxoethyl]-phosphonic acid;
[1-(1-Naphthalenyl)-2-oxo-2-[3-[(3-phenyl-1-pyrrolidinyl)carbonyl]-2-naphthalenyl]ethyl]-phosphonic acid;
[1-(1-Naphthalenyl)-2-oxo-2-[3-[[3-(2-phenylethyl)-1-pyrrolidinyl]carbonyl]-2-naphthalenyl]ethyl]-phosphonic acid;
[2-[3-[[Methyl [1-(2-naphthalenylcarbonyl)-4-piperidinyl]amino]carbonyl]-2-naphthalenyl]-2-oxo-1-phenylethyl]-phosphonic acid;
[2-[2-[[Methyl [1-(2-naphthalenylcarbonyl)-4-piperidinyl]amino]carbonyl]phenyl]-1-(1-naphthalenyl)-2-oxoethyl]-phosphonic acid;
Methyl[2-[3-[[methyl[1-(2-naphthalenylcarbonyl)-4-piperidinyl]amino]carbonyl]-2-naphthalenyl]-1-(1-naphthalenyl)-2-oxoethyl]-phosphinic acid;
[1-(5-Chlorobenzo[b]thien-3-yl)-2-[3-[[methyl[1-(2-naphthalenylcarbonyl)-4-piperidinyl]amino]carbonyl]-2-naphthalenyl]-2-oxoethyl]-phosphonic acid;
1-[4-(3-Indolylacetoxy)benzoyl]-4-piperidine-carboxylic acid;
(S)-2-[4-(3-indolylacetoxy)benzoyl]-1,2,3,4-tetrahydroisoquinoline-3-carboxylic acid;
(S)-1-[4-(3-indolylacetoxy)benzoyl]-pyrrolidine-2-carboxylic acid;
(S)-1-[4-(3-(2-methyl)indolylacetoxy)benzoyl]-pyrrolidine-2-carboxylic acid.
N-[4(S)—[N—[N-(tert-butyloxycarbonyl)-L-valyl-L-prolyl]amino]-2,2-d-ifluoro-3-oxo-5-phenylvaleryl]glycine;
3-[N-[4(S)—[N—[N-(tert-butyloxycarbonyl)-L-glutamyl-L-prolyl]amino]-2,2-difluoro-3-oxo-5-phenylvaleryl]amino]benzoic acid; and
3-[N-[4(S)—[N—[N-(phenylsulfonyl)-L-glutamyl-L-prolyl]amino]-2,2-di-fluoro-3-oxo-5-phenylvaleryl]amino]benzoic acid.

In certain cases an inhibitor of a chymotrypsin-like enzyme having a carboxylic acid group can be converted into a symmetrical anhydride having the structure Z-C(O)—O—C(O)-Z where Z represents the remainder of the inhibitor. Two different inhibitors can be converted into a non-symmetrical anhydride having the structure Z′-C(O)—O—C(O)-Z where Z′ and Z represent the remainder of the two different inhibitors.

Prodrug forms of any of the inhibitors of a chymotrypsin-like enzyme described herein that are alcohols can be created by replacing the H of the —OH group with R wherein R is selected from a straight chain or branched C₁ to C₆ alkyl (C₁, C₂, C₃, C₄, C₅, C₆), alkenyl, alkynyl, or a C₆ aryl optionally independently substituted with one or more halogen (Cl, F, Br, I), —OH, —C(O)OH, —NH₃. Alternatively R can be selected from

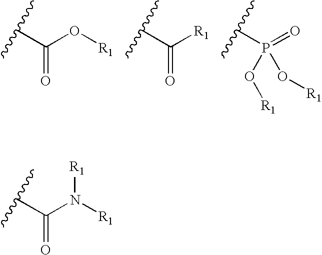

wherein R₁ is selected from H or a branched or straight chain C₁ to C₆ (C₁, C₂, C₃, C₄, C₅, C₆) alkyl, alkenyl, alkynyl, aryl, cycloalkyl, or arylalkyl optionally independently substituted with one or more halogen —OH, —C(O)OH, or —NH₃.

Among the inhibitors of chymotrypsin-like enzymes that are alcohols and might be converted to prodrugs in this manner are: 7-chloro-3-(3-hydroxybenzenesulfonyl)-2,4(1H,3H)-quinazolinedione;
3-(4-hydroxybenzenesulfonyl)-7-chloro-2,4(1H,3H)-quinazolinedione;
3-(3-carboxymethyl-benzenesulfonyl)-7-chloro-2,4(1H,3H)-quinazolinedione;
3-(4-carboxymethyl-benzenesulfonyl)-7-chloro-2,4(1H,3H)-quinazolinedione;
3-[(3-carboxymethylaminophenyl)sulfonyl]-7-chloro-2,4(1H,3H)-quinazolinedione;
5-chloro-N-(4-hydroxymethyl-2-methanesulfonylphenyl)-3-methylbenzo[b]thiophene-2-sulfonamide;
5-fluoro-N-[4-(4-hydroxymethyloxazol-2-y-l)-2-methanesulfonylphenyl]-3-methylbenzo[b]thiophene-2-sulfonamide;
5-fluoro-N-[4-(4-hydroxymethylthiazol-2-yl)-2-methanesulfonylphenyl]-3-m-ethylbenzo[b]thiophene-2-sulfonamide; and
[2-[3-[[[1-[(6-Hydroxy-2-naphthalenyl)carbonyl]-4-piperidinyl]methylamino]carbonyl]-2-naphthalenyl]-1-(1-naphthalenyl)-2-oxoethyl]-phosphonic acid.

Prodrug forms of inhibitors of chymotrypsin-like enzymes that are peptides can be generenated in the manner described above for peptide inhibitors of chymotrypsin.

### Chymotrypsin Activity Assays

Any standard chymotrypsin activity assay can be used to assess known chymotrypsin inhibitors and compounds which may inhibit chymotrypsin.

For example chymotrypsin activity can be measured using N-Glutaryl-Lphenylalanine p-nitroanilide (Sigma-Aldrich, Inc; Catalog No. 49738) as a substrate and cc-Chymotrypsin from bovine pancreas (EC 3 2 1. 1; Sigma-Aldrich; Catalog No. C4129) in an assay described by Kakade et al. (Cereal Chemistry 51:376 (1974)). In this assay, chymotrypsin hydrolyzes the substrate N-Glutaryl-L-phenylalanine-p-nitroanilide present in excess. The release of p-nitroanilide, a yellow dye, is measured spectrophotometrically.

An additional chymotrypsin assay is described by Kourteva et al. (Analytical Biochemistry 162:345-9, 1987). This assay is rapid and particularly useful for assessing higher molecular weight inhibitors. Briefly, a test compound is spotted onto an agar film which contains TLCK-chymotrypsin. Enzyme inhibition is visualized as colorless zones on a pink background after the films are stained with a chromogenic substrate N-acetyl-DL-phenylalanine-β-naphthyl ester. A variation on this assay can be use to assess trypsin activity and this can be useful for assessing the selectivity of an inhibitor.

### Guanylin Degradation Assay

Since guanylin is susceptible to digestion by chymotrypsin, chymotrypsin activity can be assayed based on guanylin cleavage. Guanylin (Sigma, G-116) or a guanylin variant were resuspended in 5 ml of 100 mM Tris-HCl, 2 mM CaCl₂, pH 7.8 at 30° C. for a final concentration of 0.01 mg/ml. From this stock, six (6) 500 μl aliquots were prepared in 2 ml Eppendorf tubes and labeled “Control”, “T0”, “T15”, “T30”, “T60”, and “T180”, with “T_” representing timepoints, in minutes. 5 μl of a 10 mM chymostatin (Sigma, C7268) stock (5 mg of chymostatin resuspended in 824 μl of DMSO) was added to the “Control” samples and all samples were incubated for 5 minutes at 30° C. Chymostatin inhibits chymotrypsin activity and this sample served as a negative control. Next, 20 μl of a chymotrypsin (Sigma, C6423) enzyme solution (0.01 mg/ml bovine chymotrypsin enzyme in 1 mM HCl, 2 mM Calcium Chloride) were added to the samples and mixed by inversion. Samples are incubated at 30° C. The “T0” samples were collected at time=0 minutes by adding 5 μl of a 10 mM chymostatin stock and subsequently stored at −80° C. All other timepoint samples are taken in a similar manner, with the “Control” samples collected parallel to the time=180 minutes samples. Determination of sensitivity to digestion by chymotrypsin was determined by LCMS analysis and by in vitro activity in the T84 cGMP assays described below.

For LC/MS analysis, samples were injected (10 μL) onto a reverse phase HPLC column (Waters Atlantis dC₁₈ 1.0×150 mm, 3 um particle size, 186001283) at 20° C., and were eluted with a reverse phase gradient (Mobile Phase A: 5 mM NH₄OAc in dH₂O, 0.1% formic acid, Mobile Phase B: 5 mM NH₄OAc in 80% methanol and 20% dH₂O, 0.1% formic acid; Initial condition of 5% B, ramping to 95% B over 35 minutes, and holding for 3 minutes, then returning to initial conditions over the next 7 minutes, all at a flow rate of 0.07 mL/min.). At 45 minutes, the gradient was at initial conditions of 5% B and held for 15 minutes. Guanylin samples were detected by quadrapole-time of flight mass spectrometry in TOF scan mode (cone voltage=30 V; collision=4 eV). Chymotrypsin sensitivity was determined by the loss of the initial mass species and the formation of the product mass species, with respect to time. Instrument response was converted into percentage units by comparison of the response of the initial mass versus the product mass, with “T0” representing total response of initial mass for all samples.

### Guanylins and Guanylin Variants

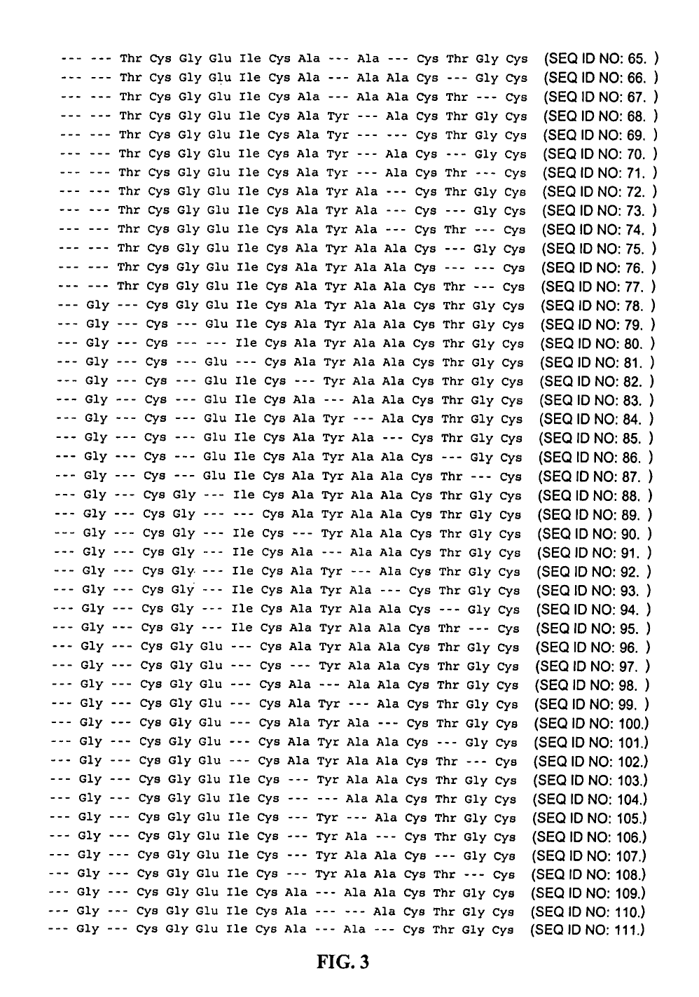

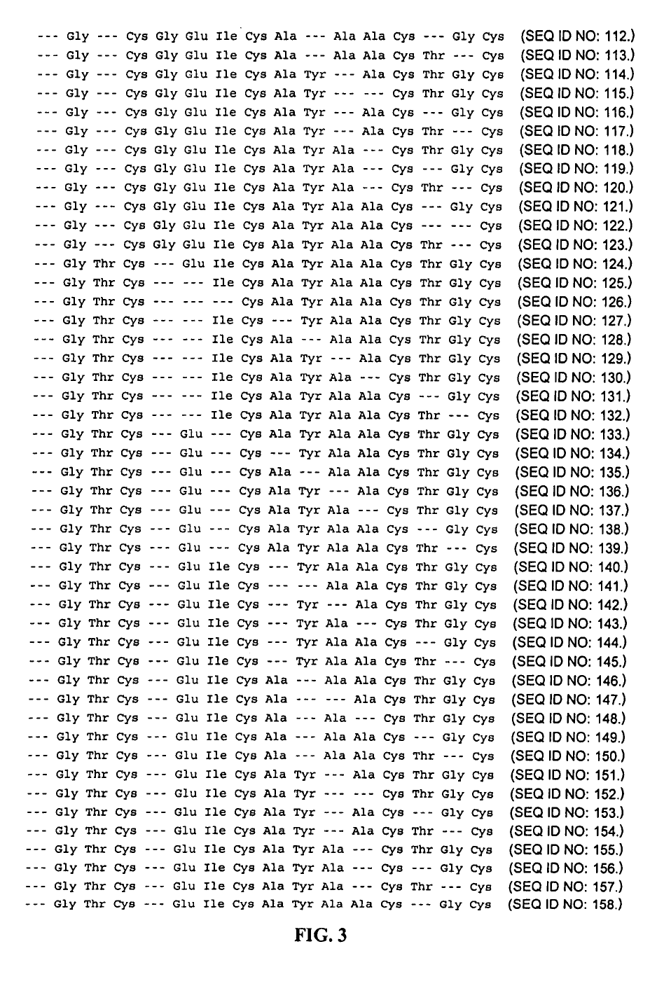

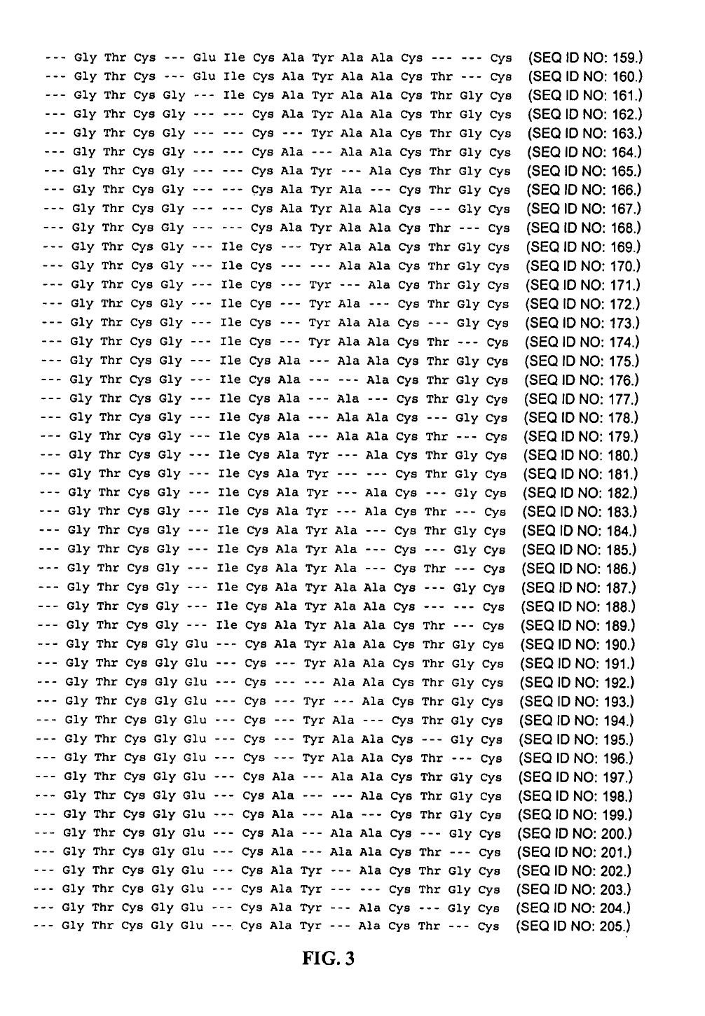

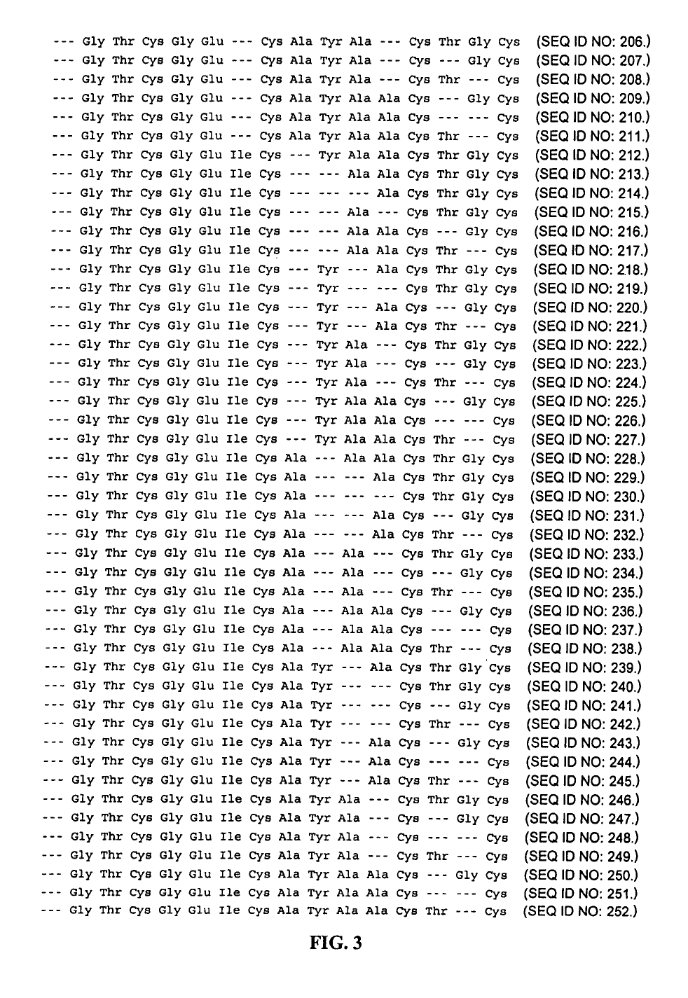

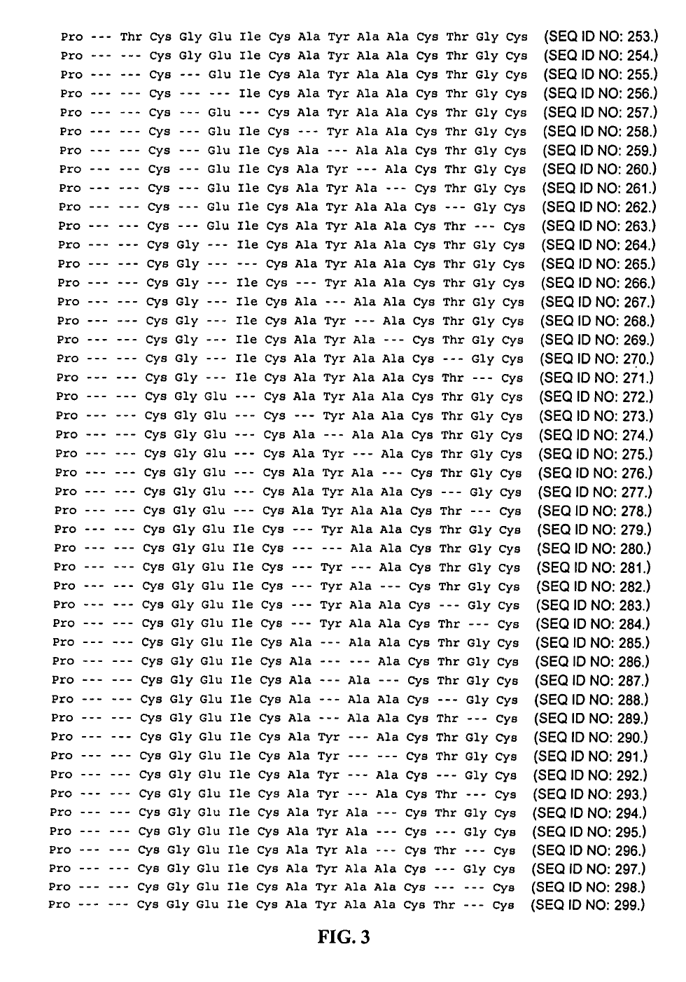

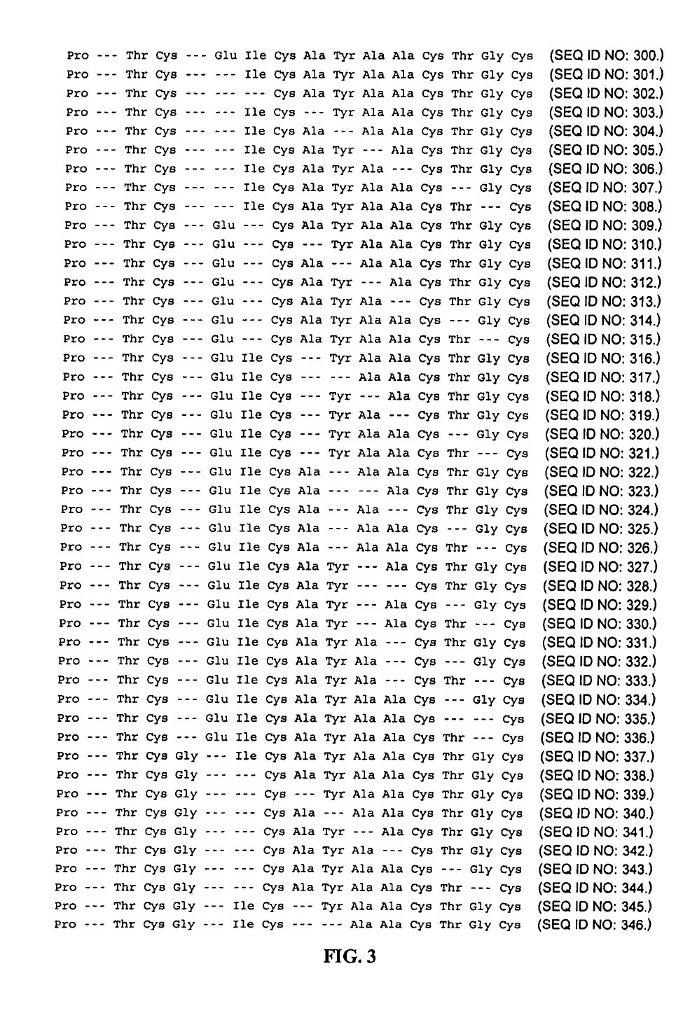

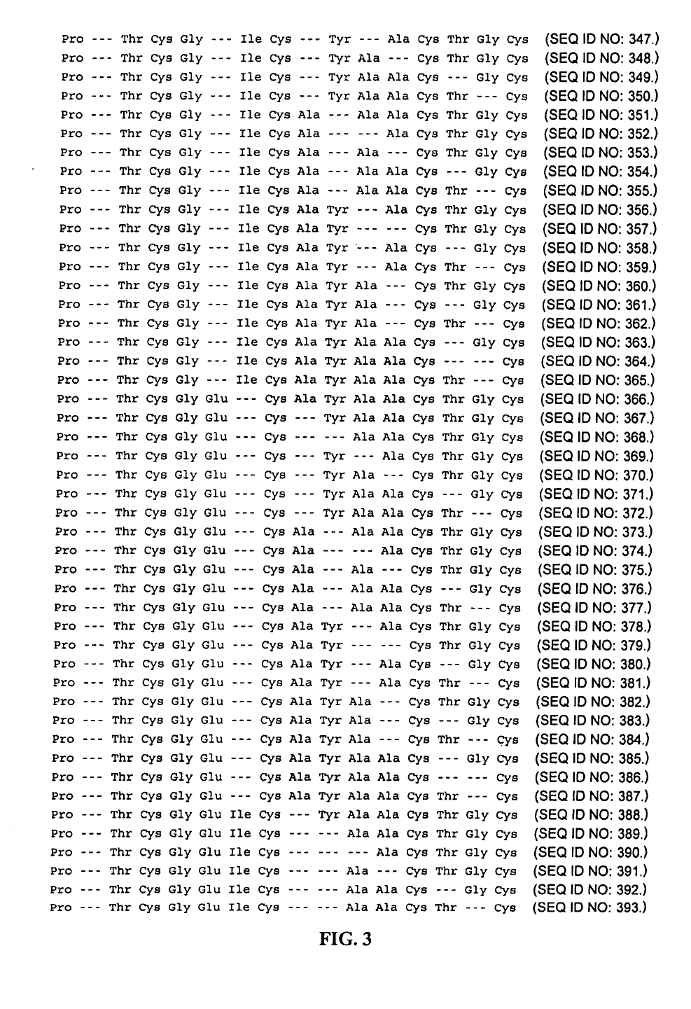

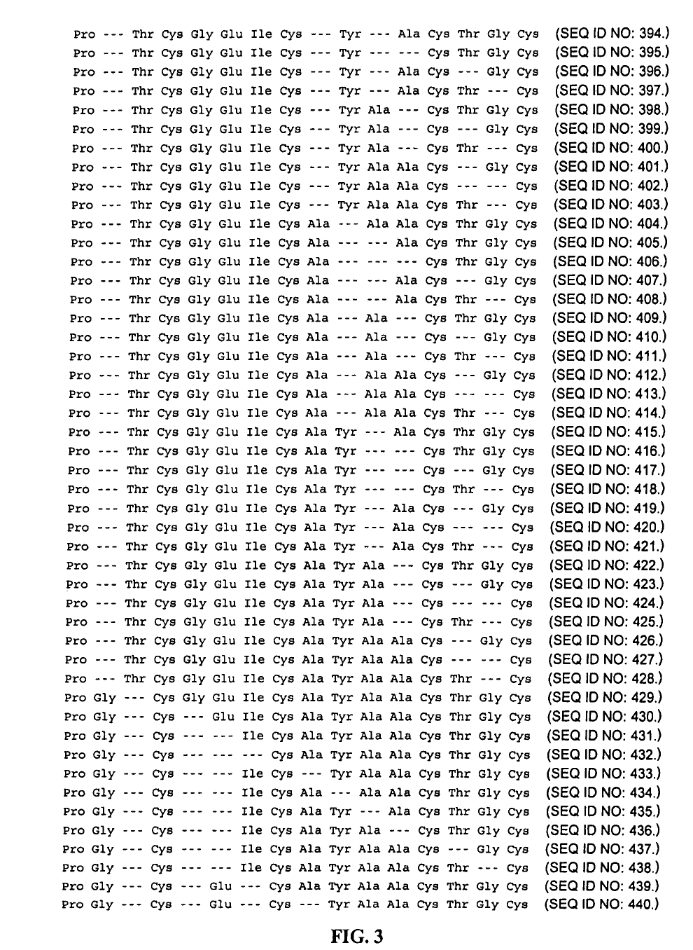

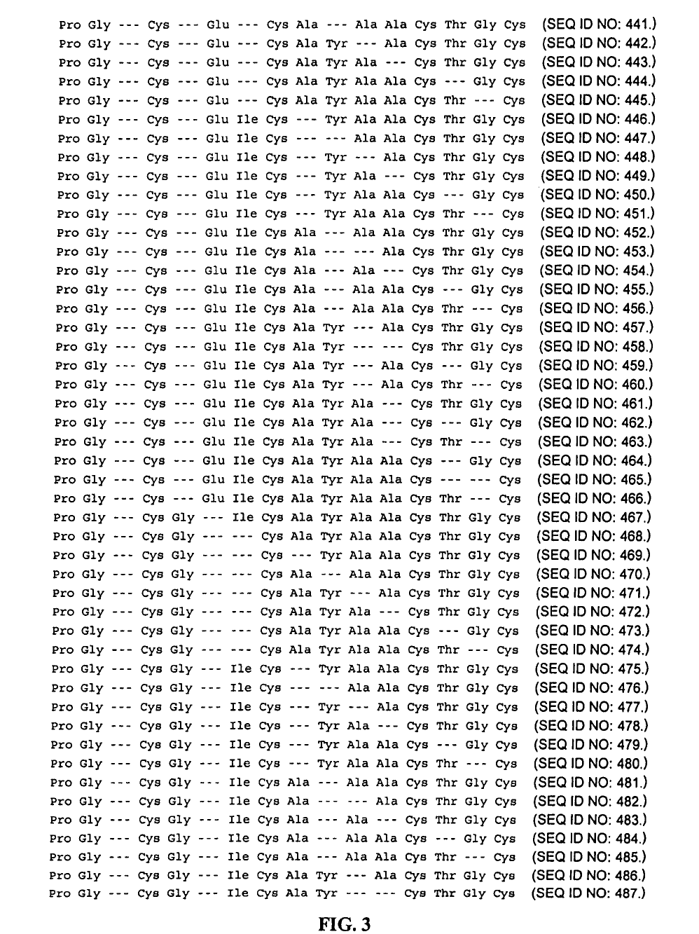

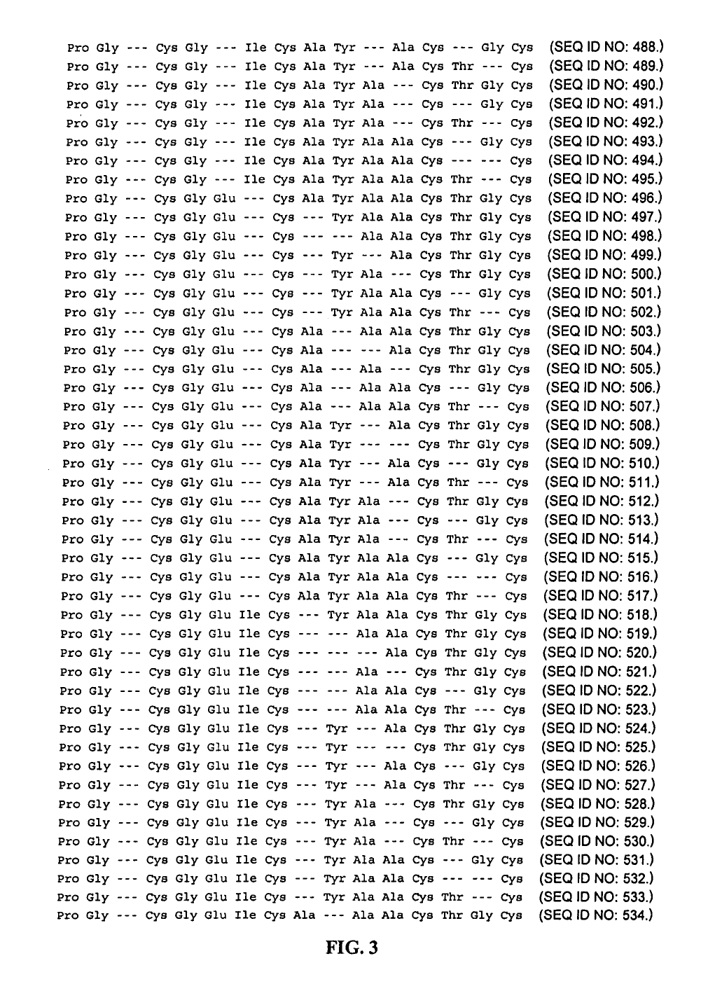

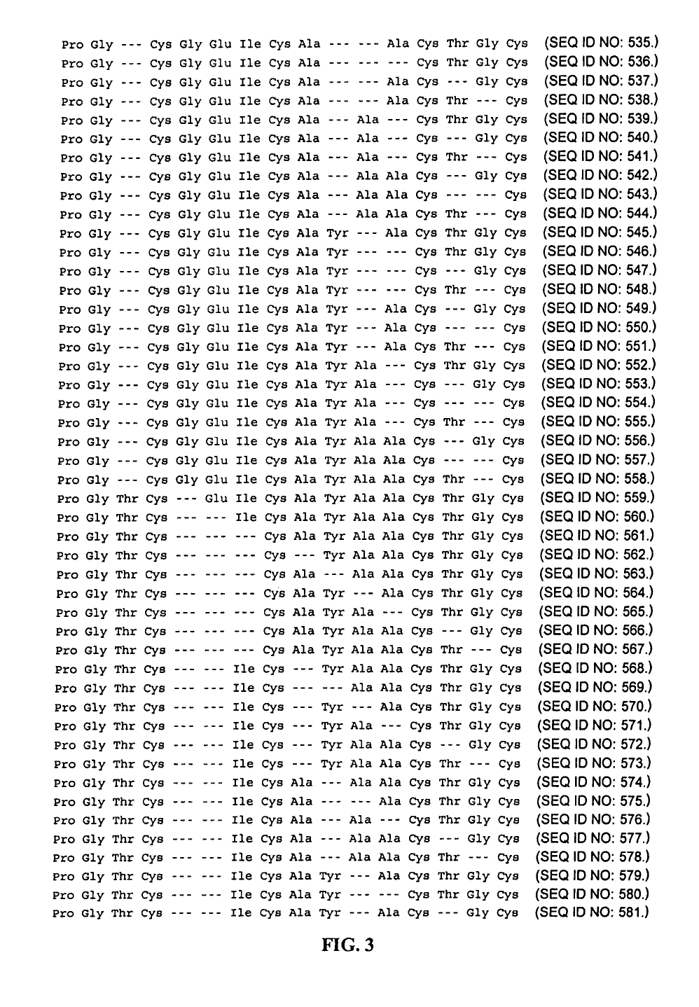

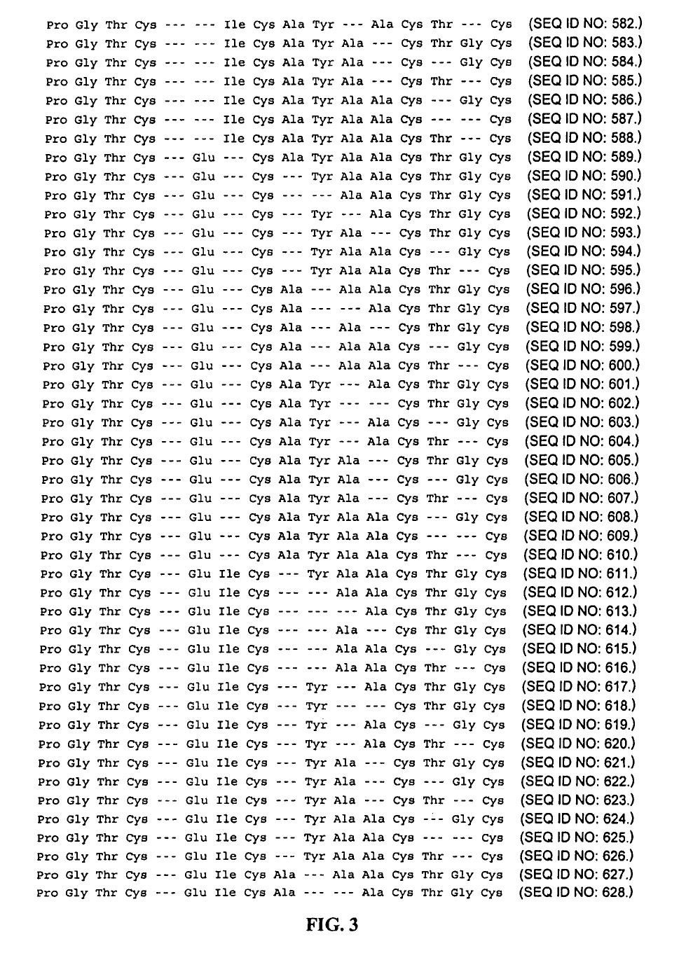

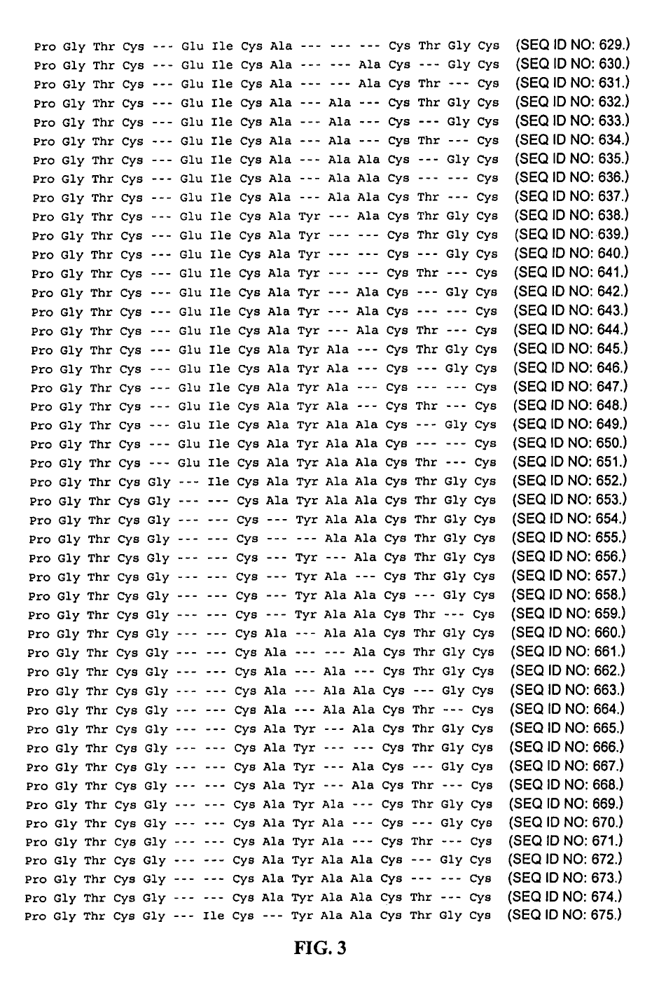

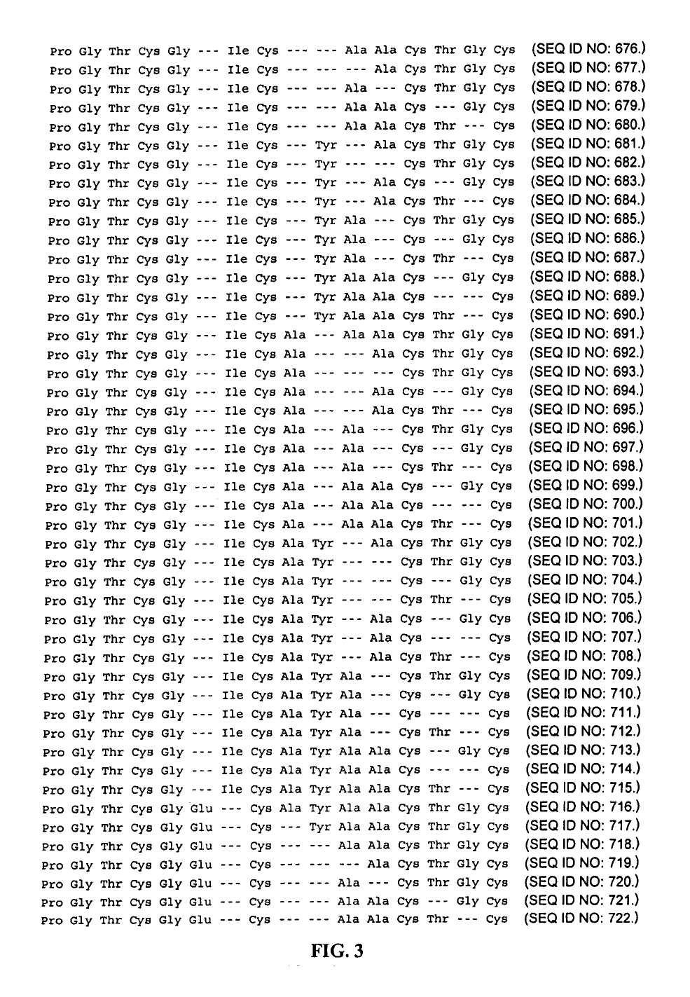

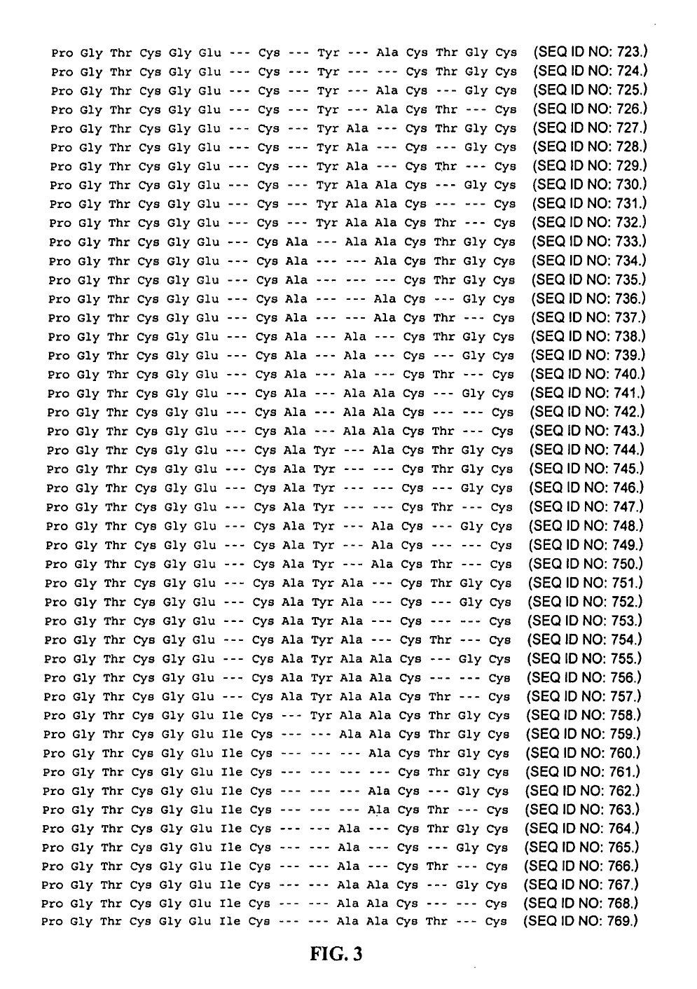

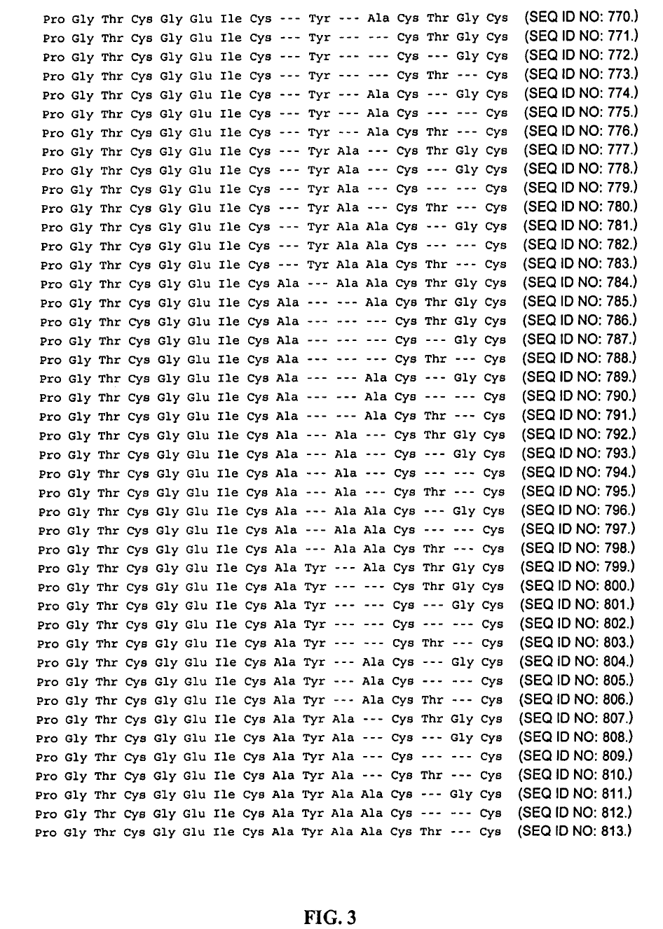

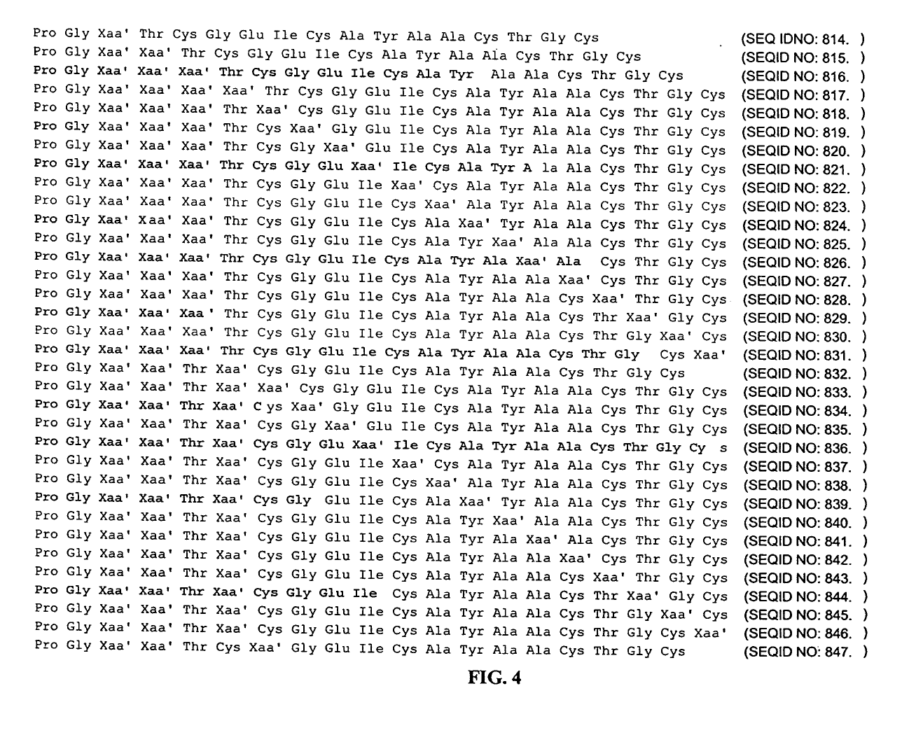

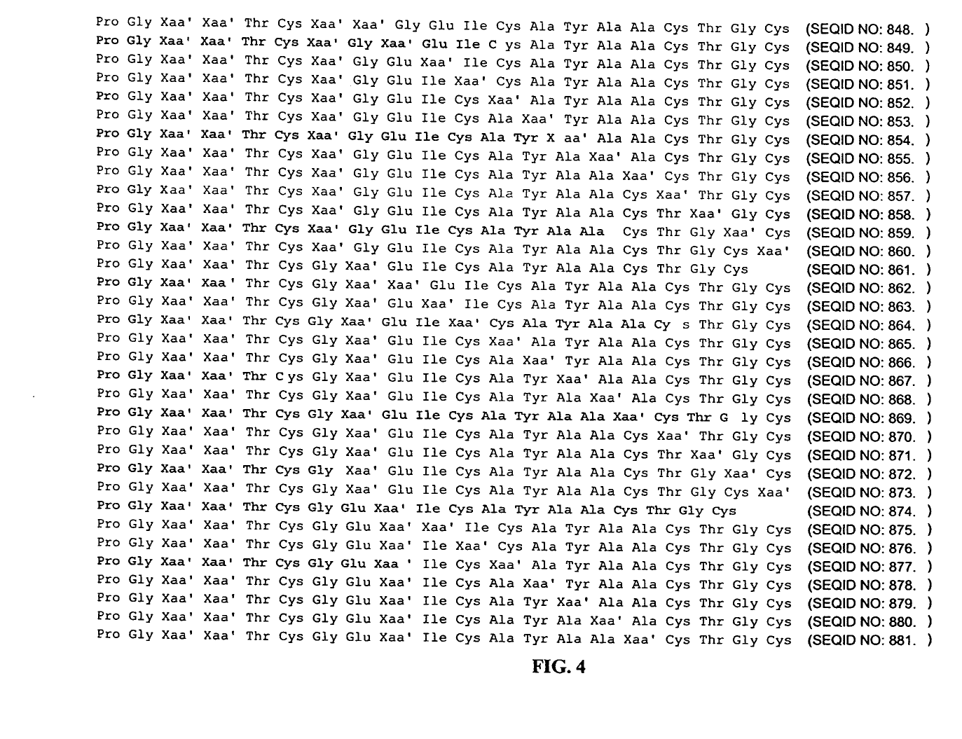

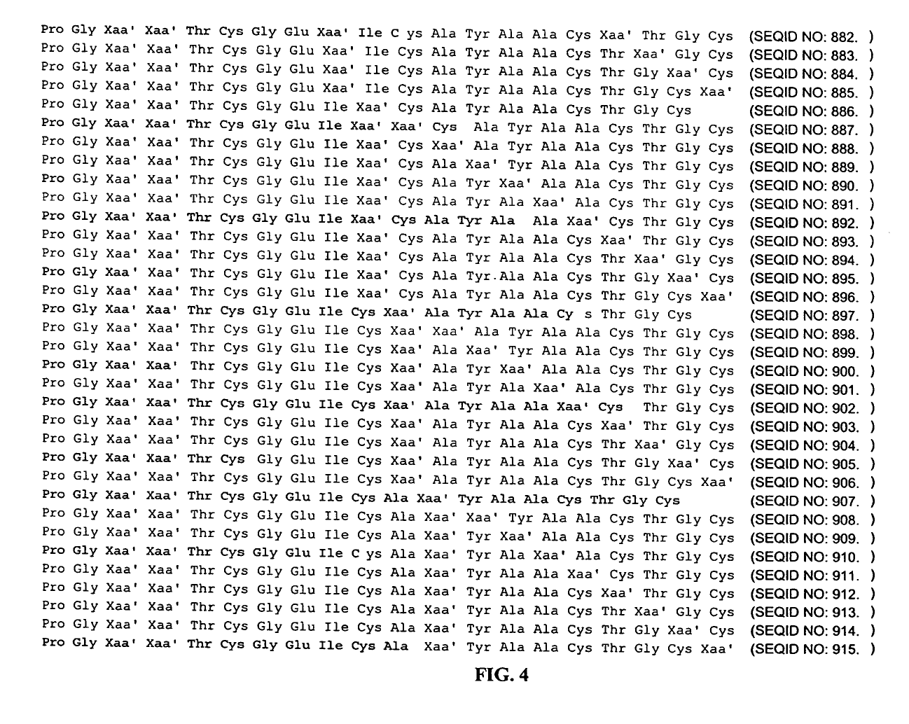

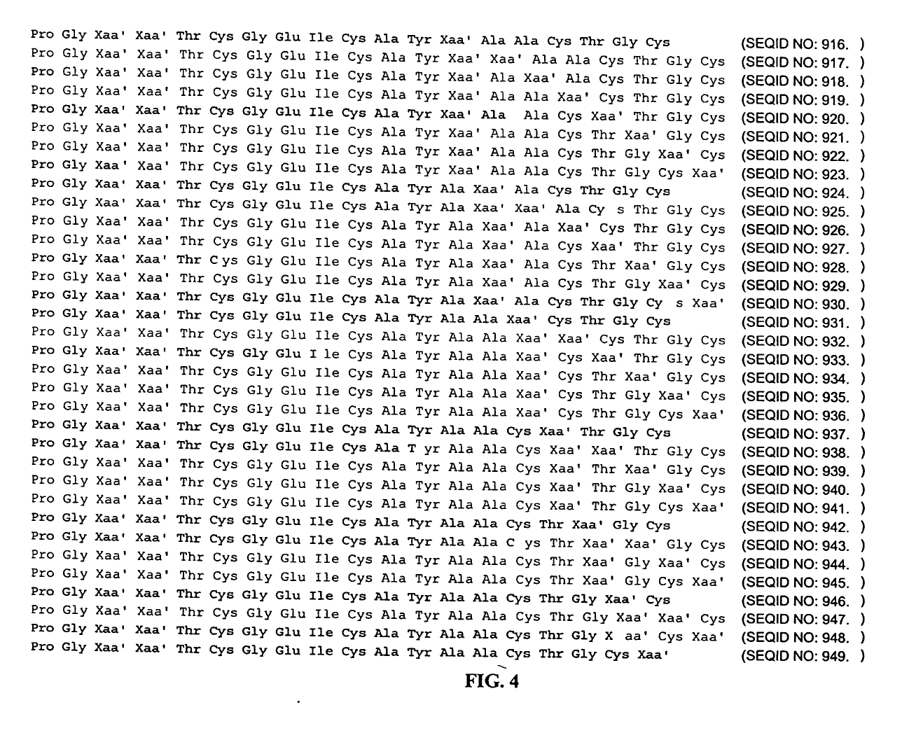

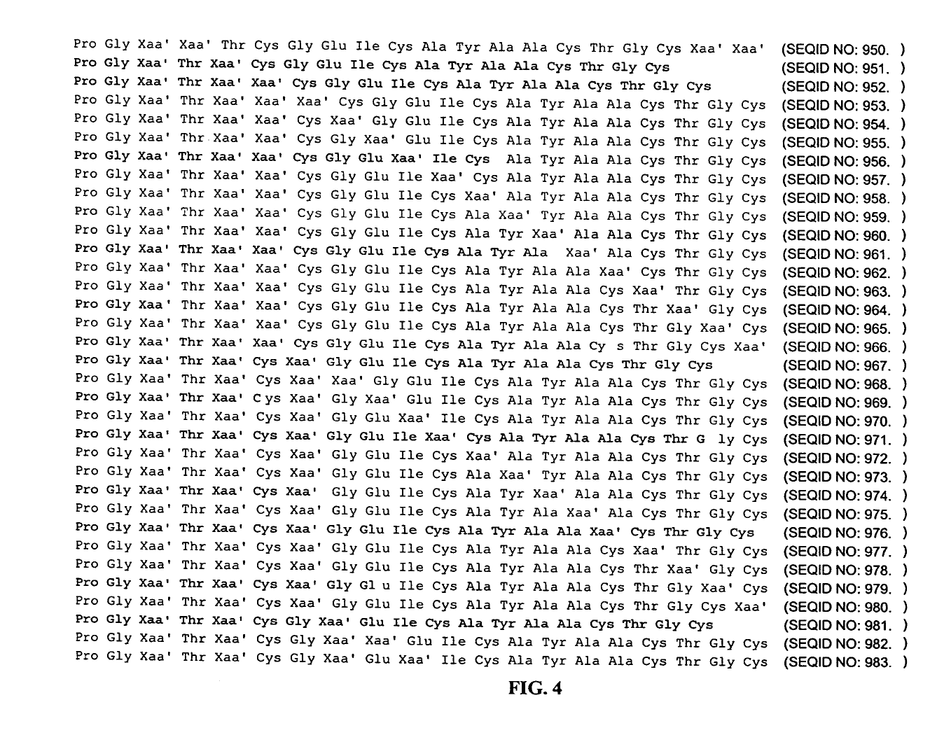

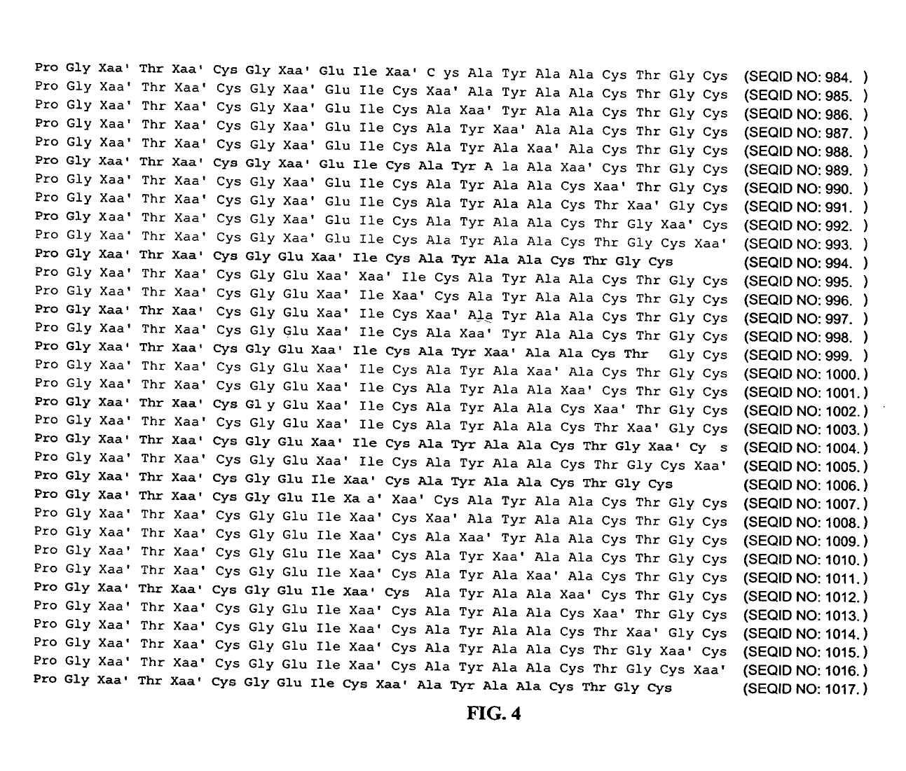

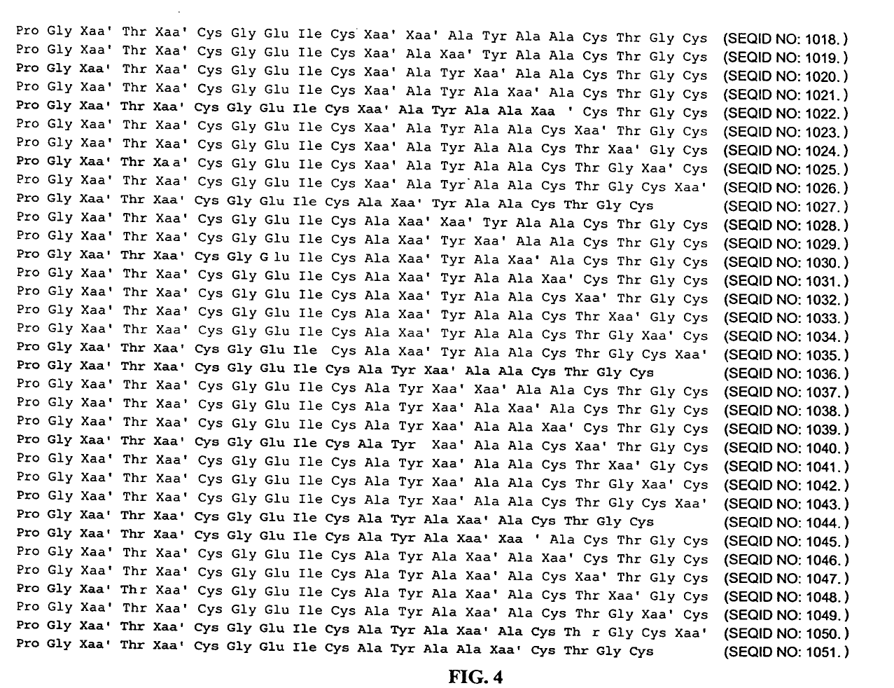

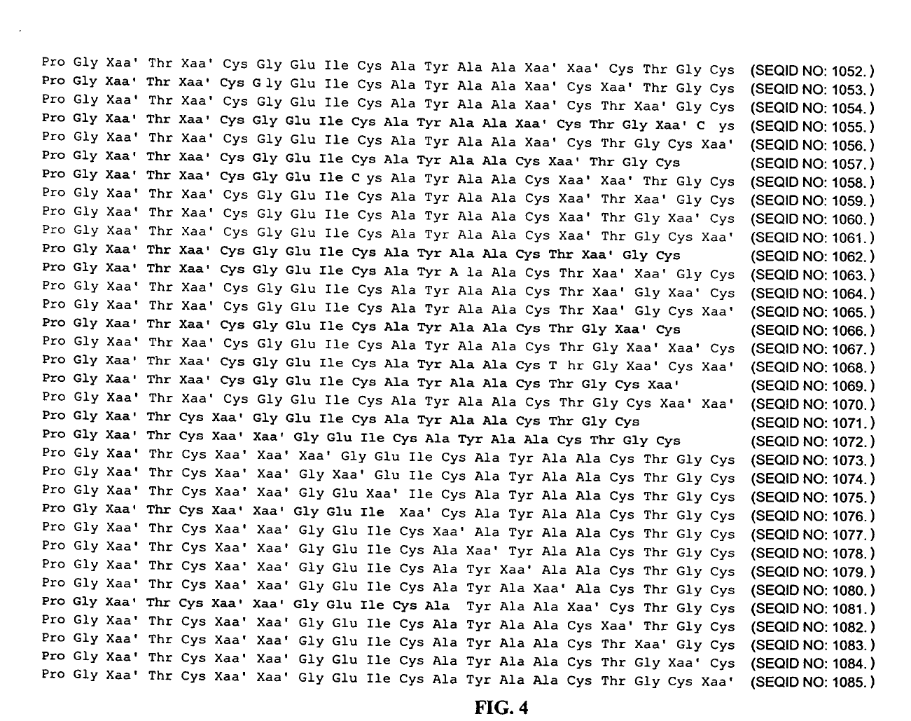

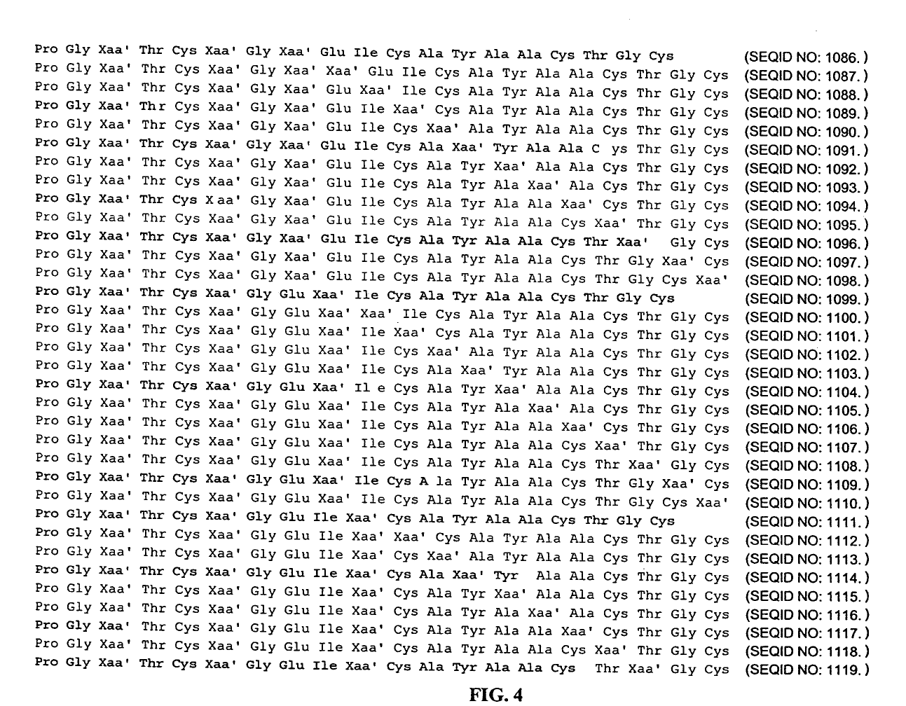

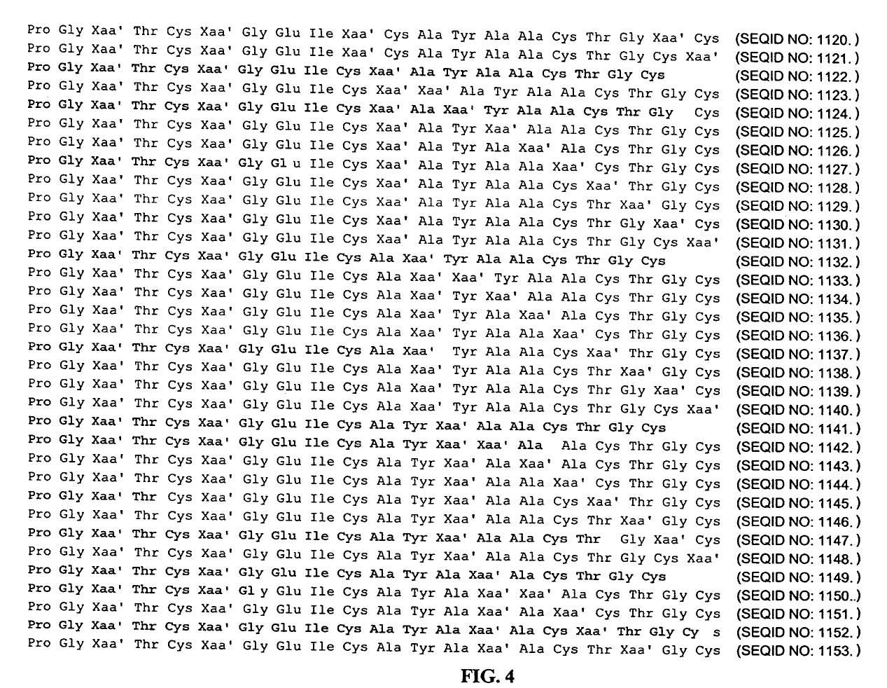

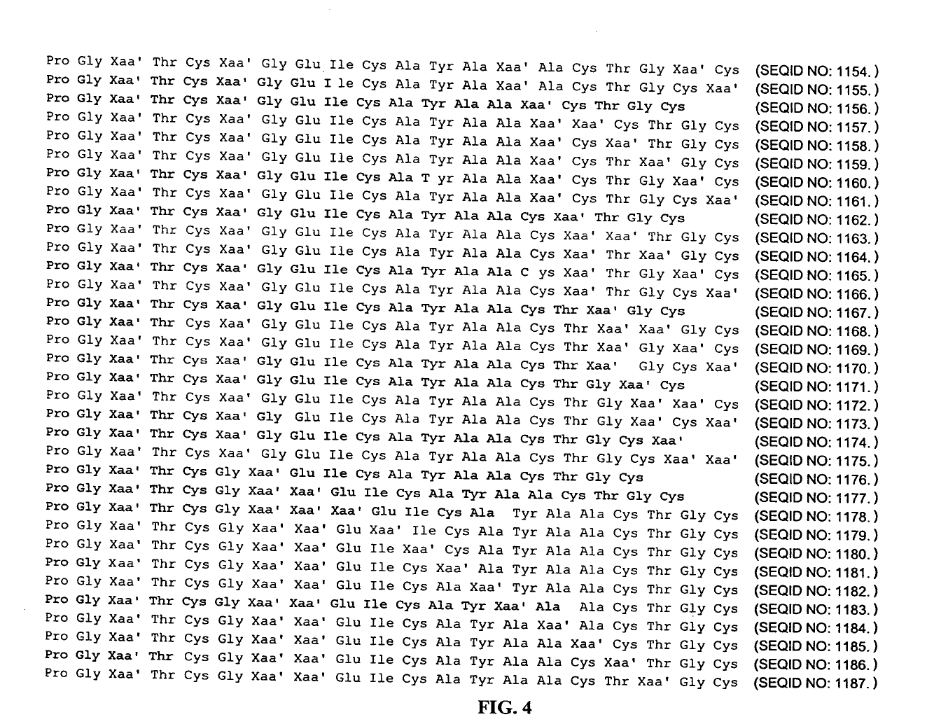

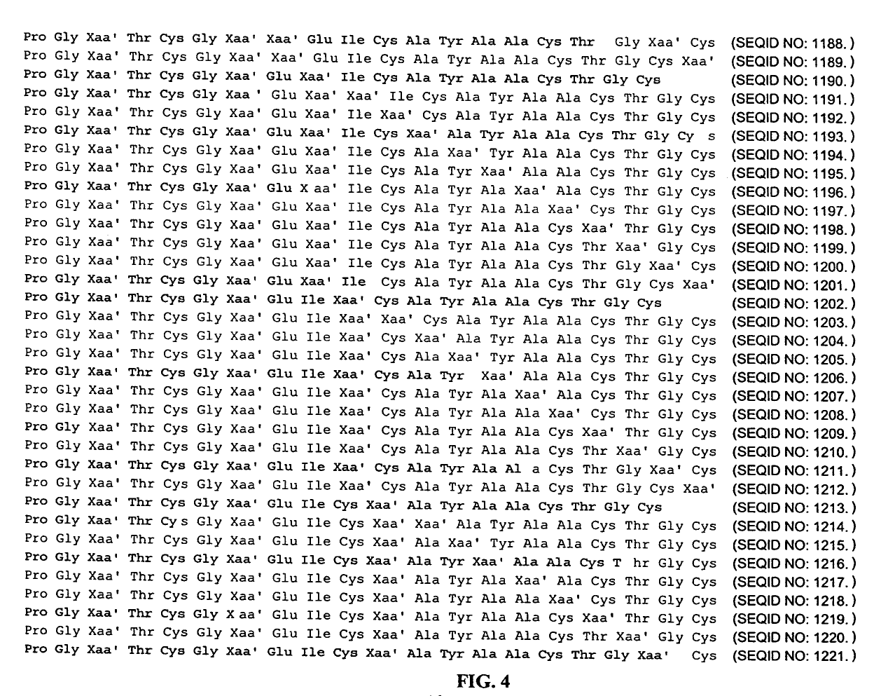

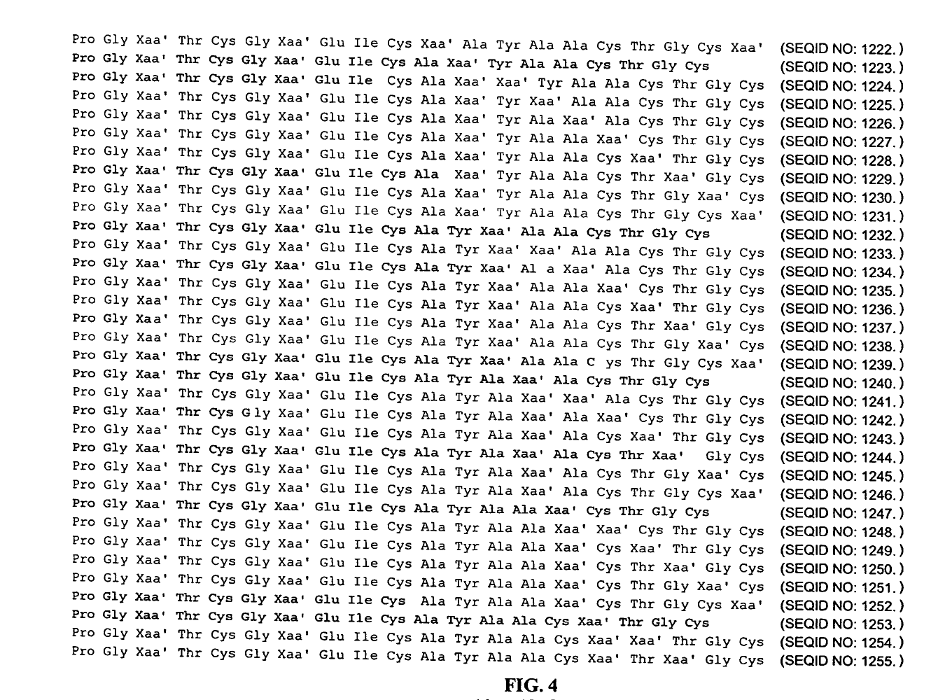

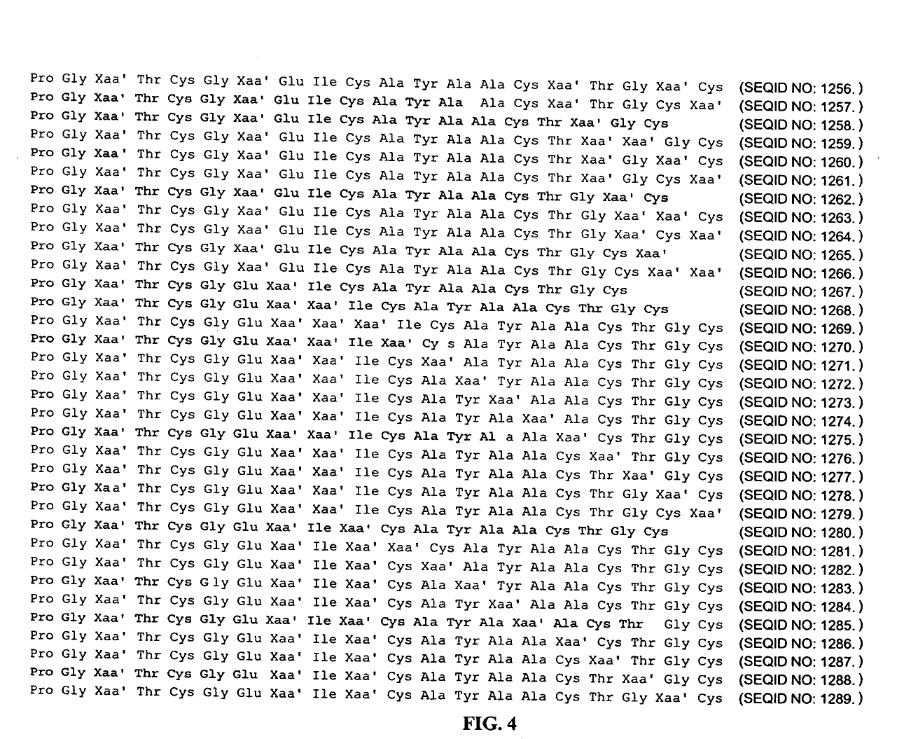

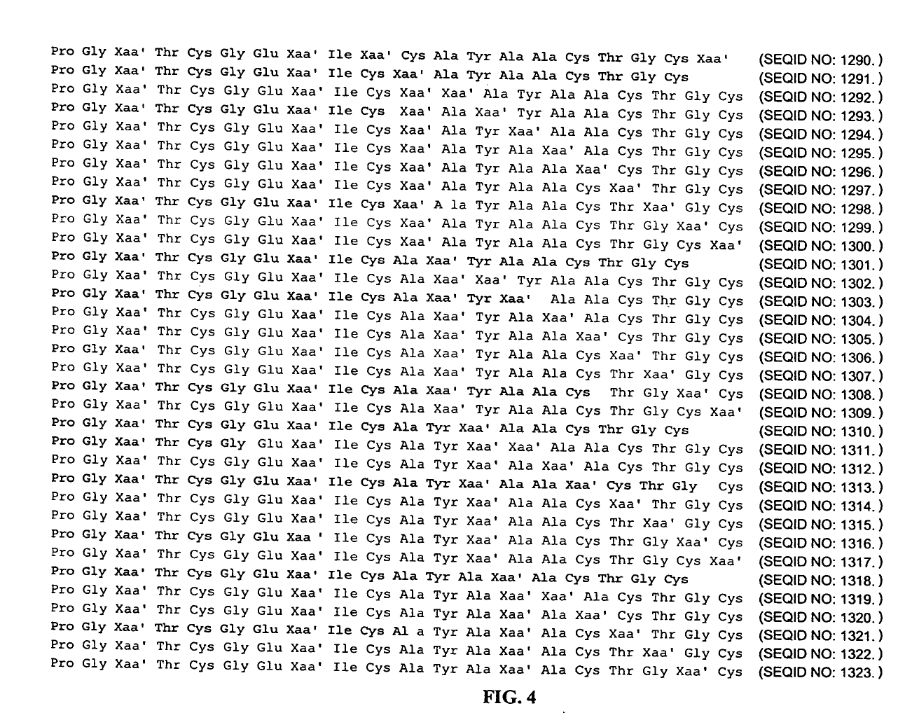

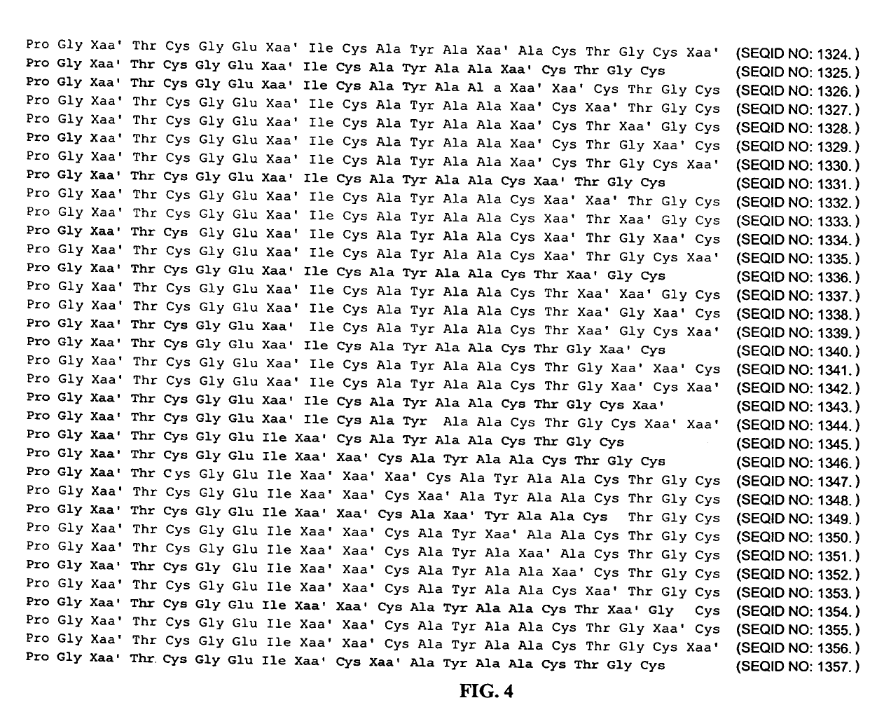

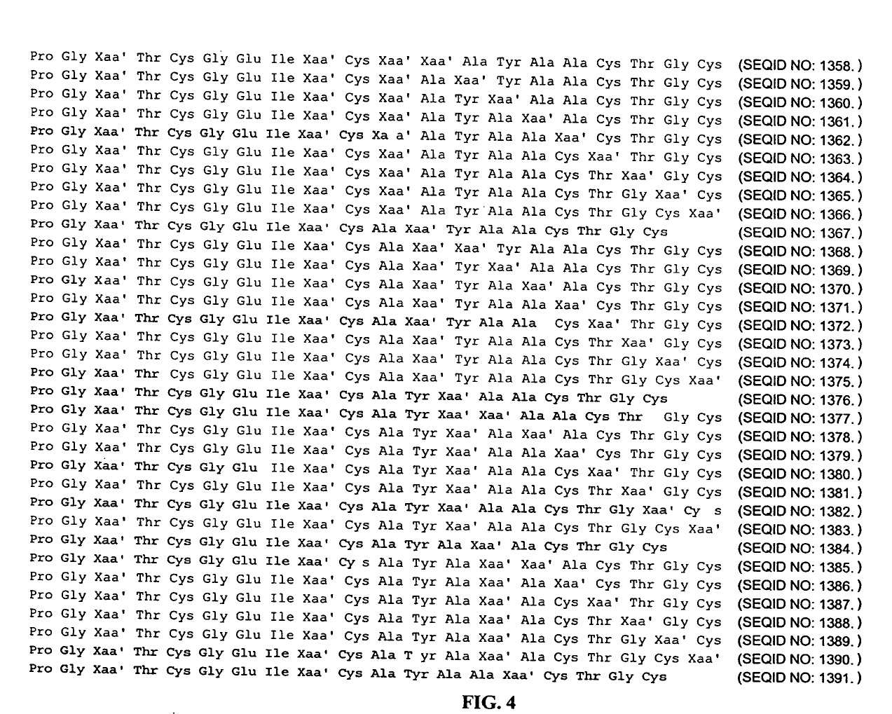

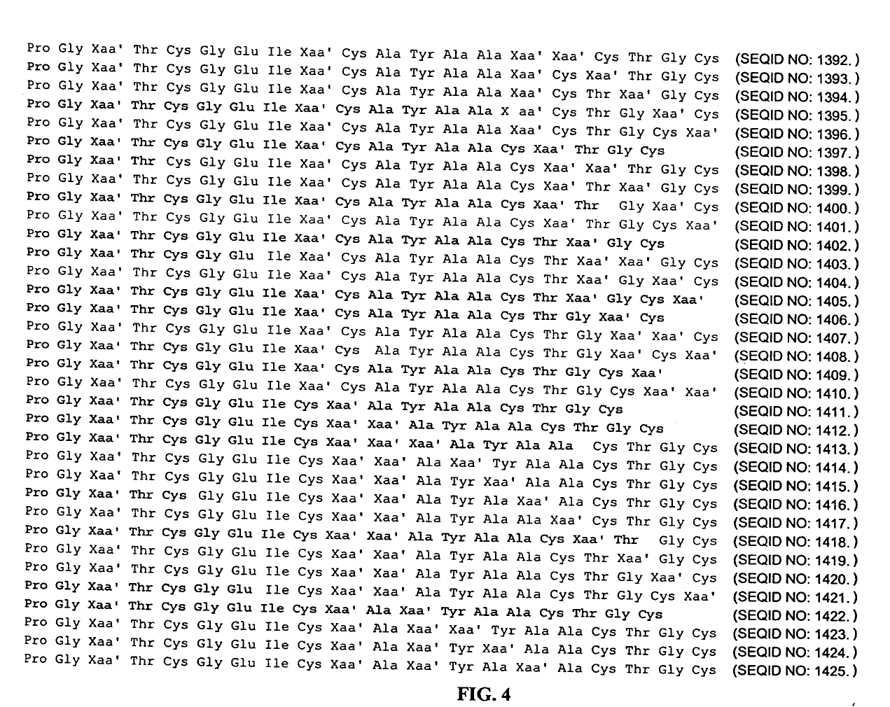

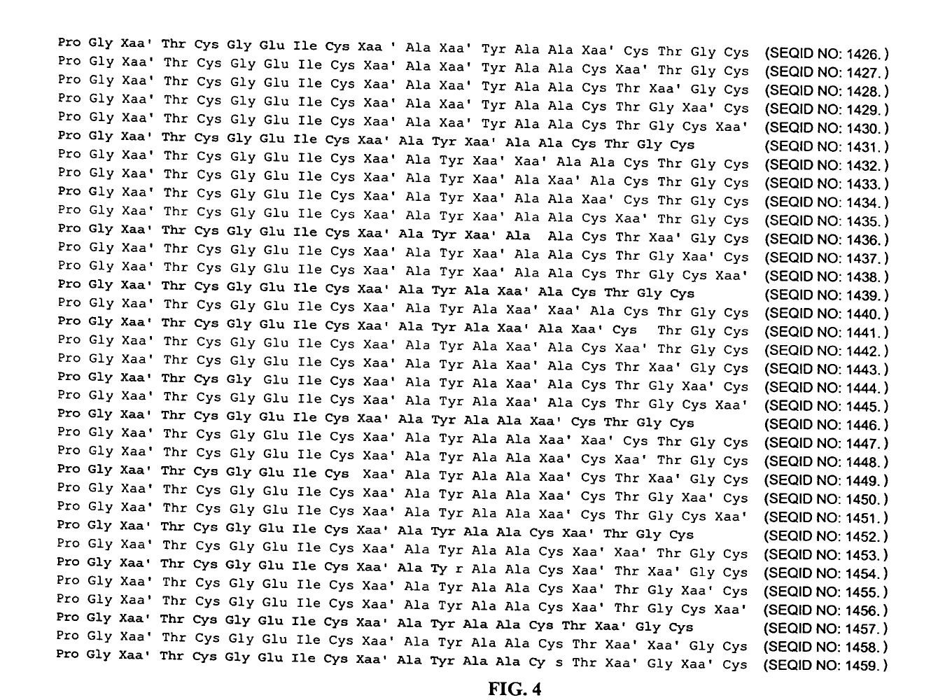

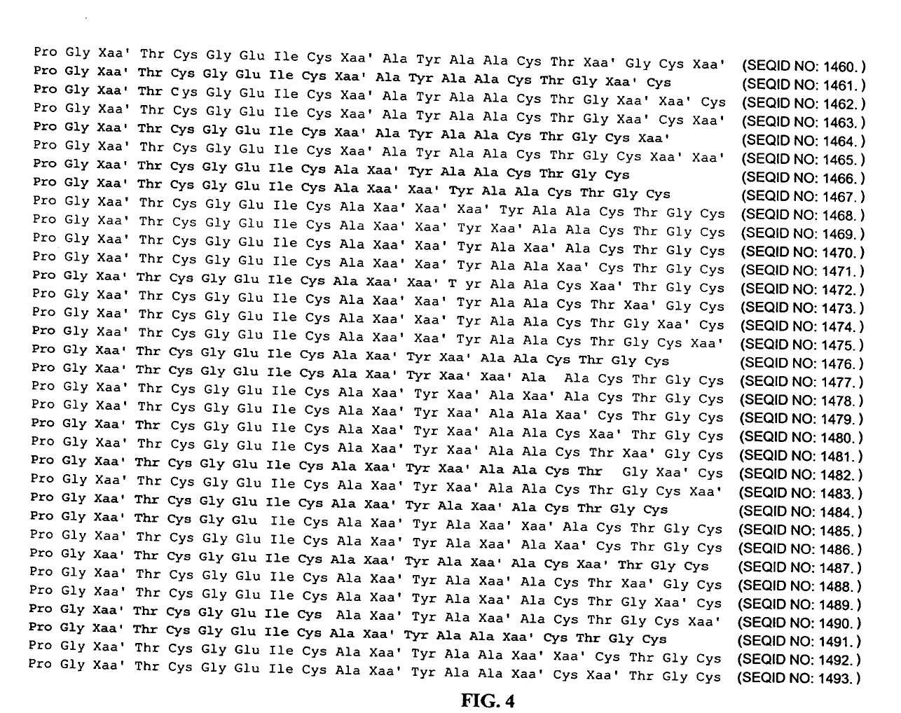

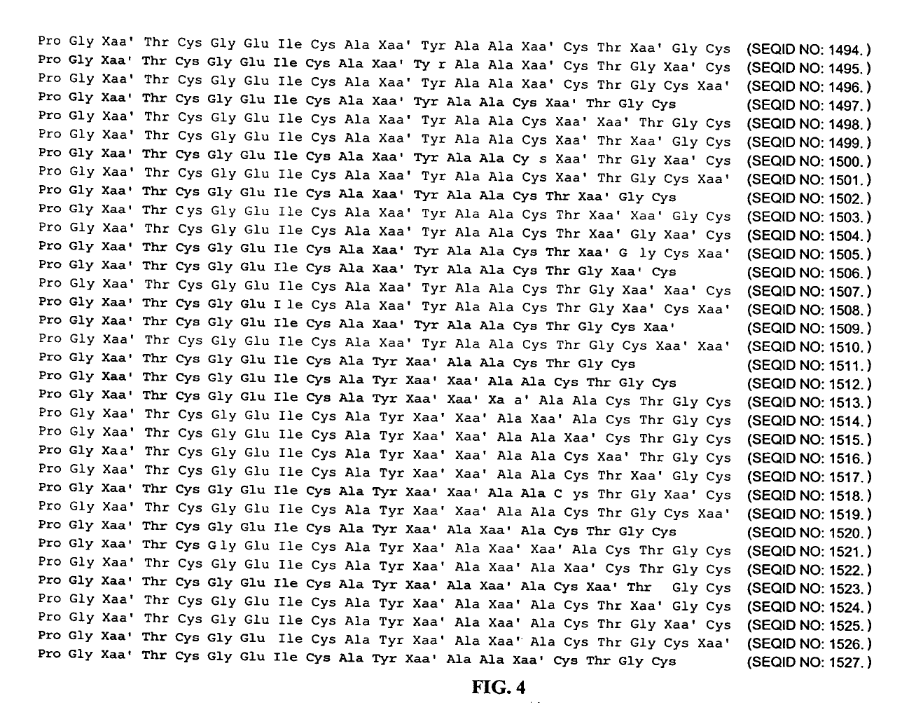

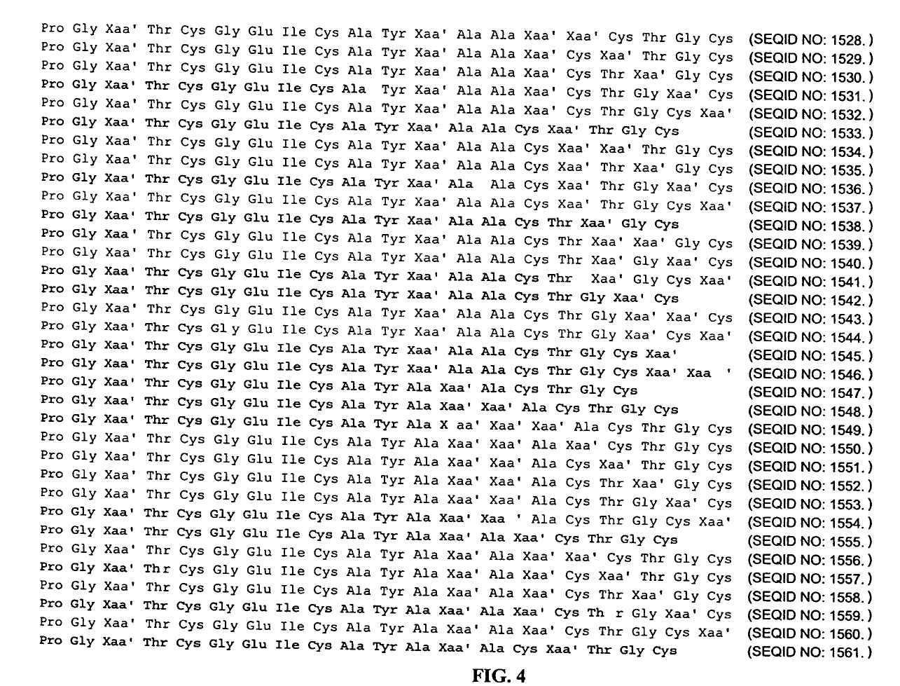

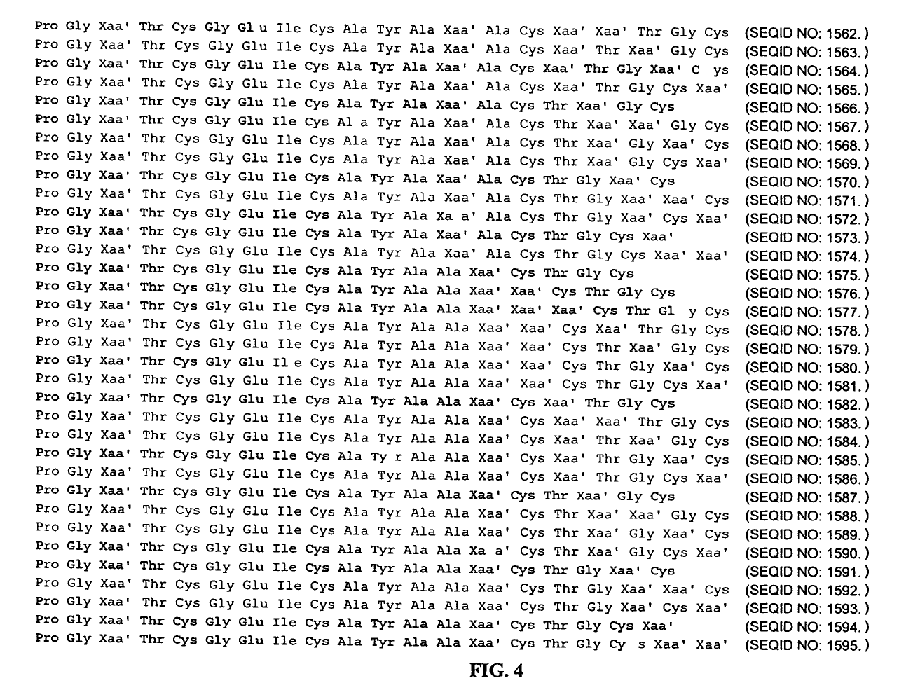

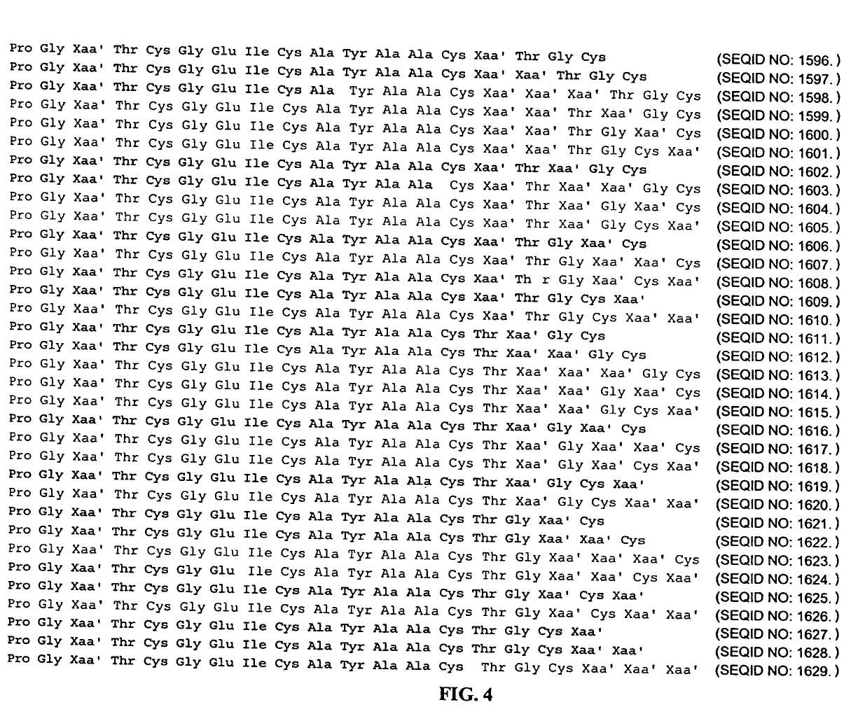

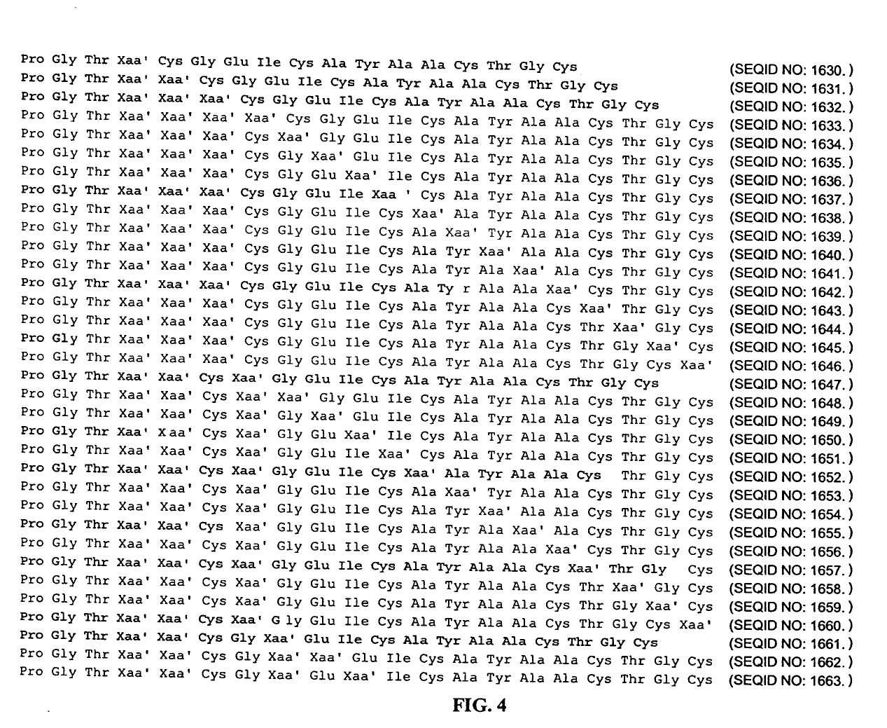

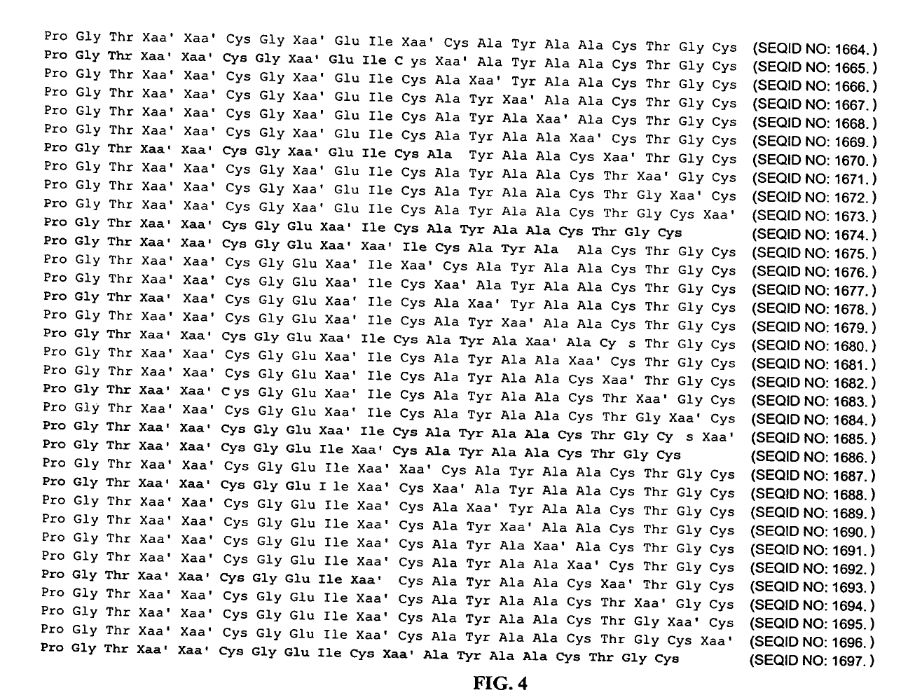

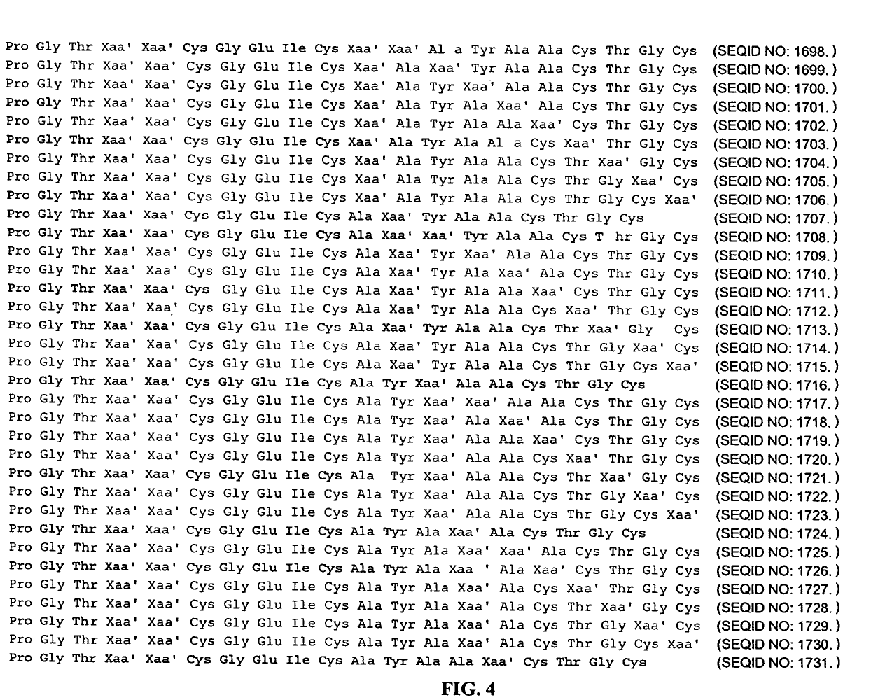

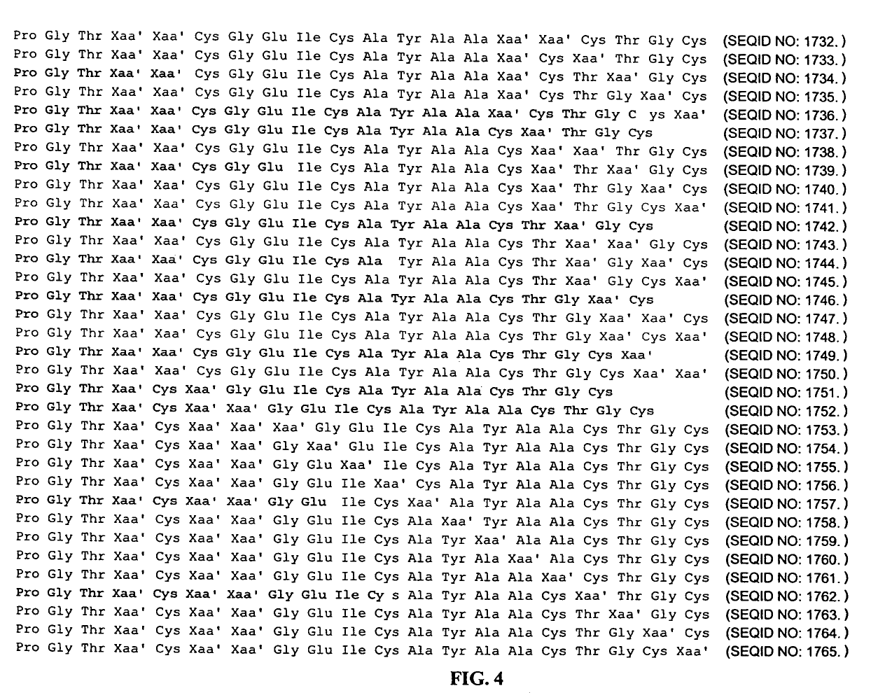

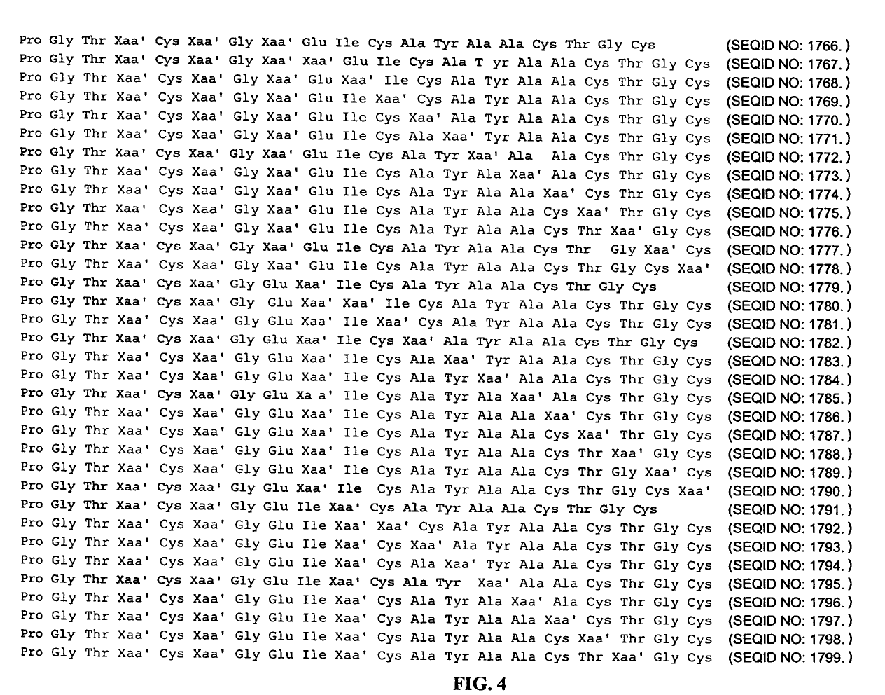

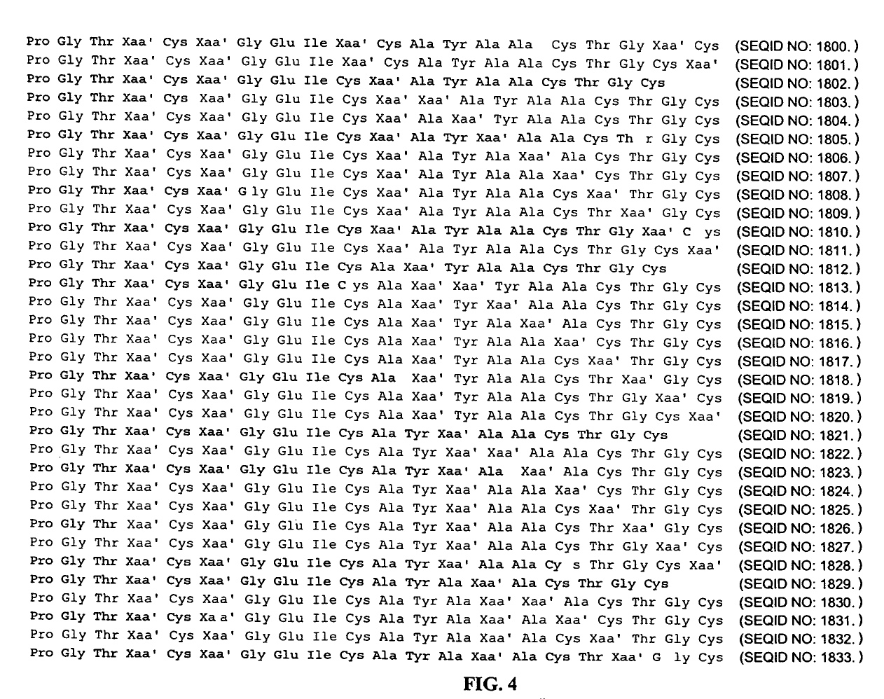

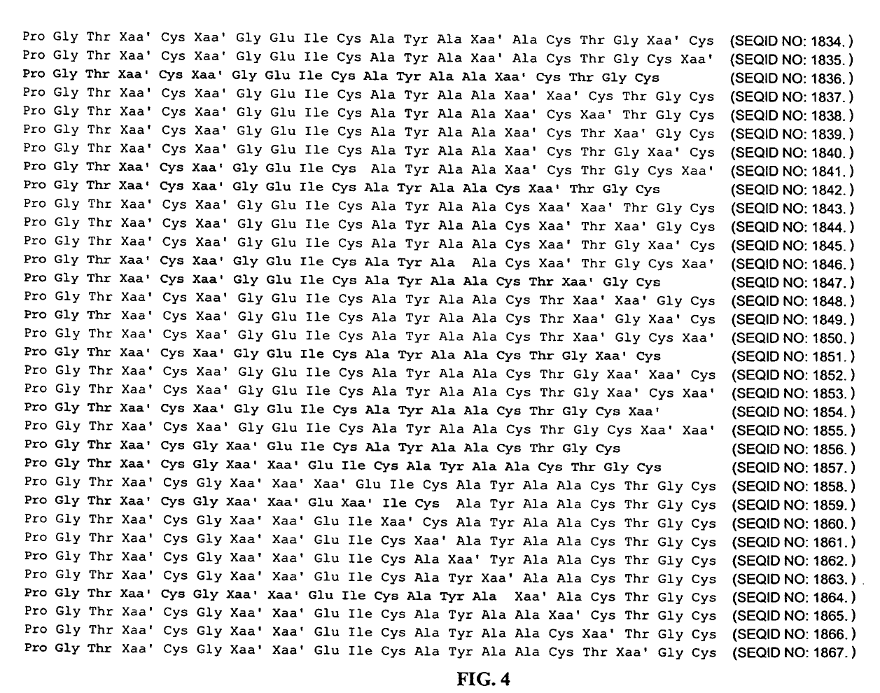

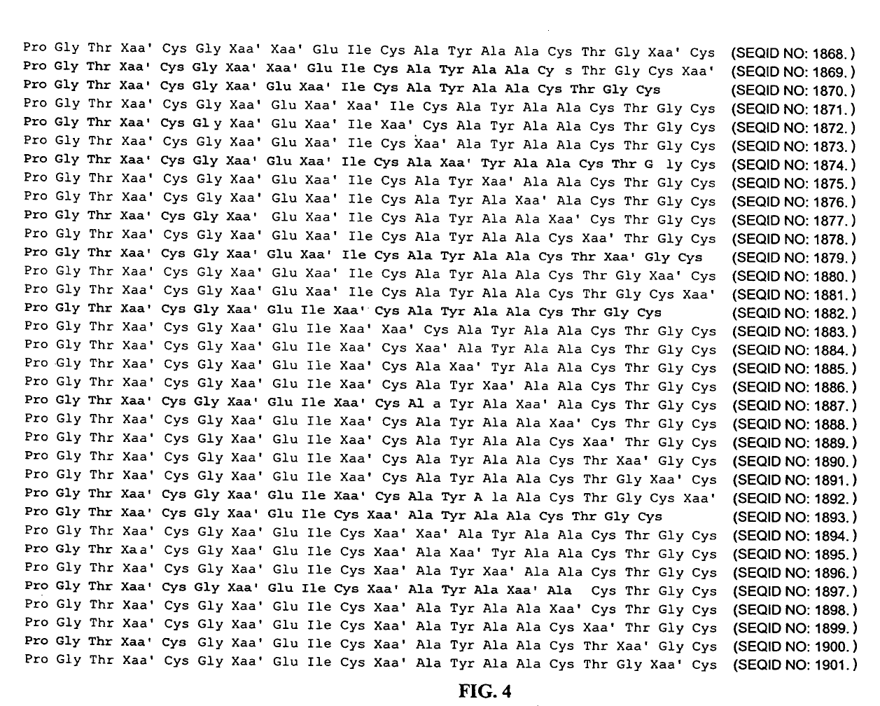

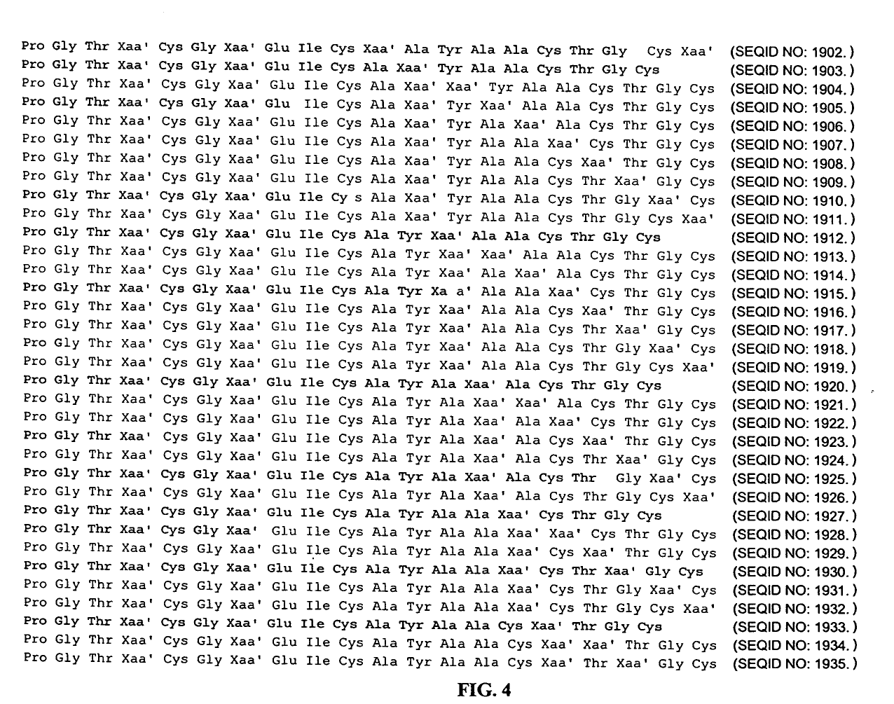

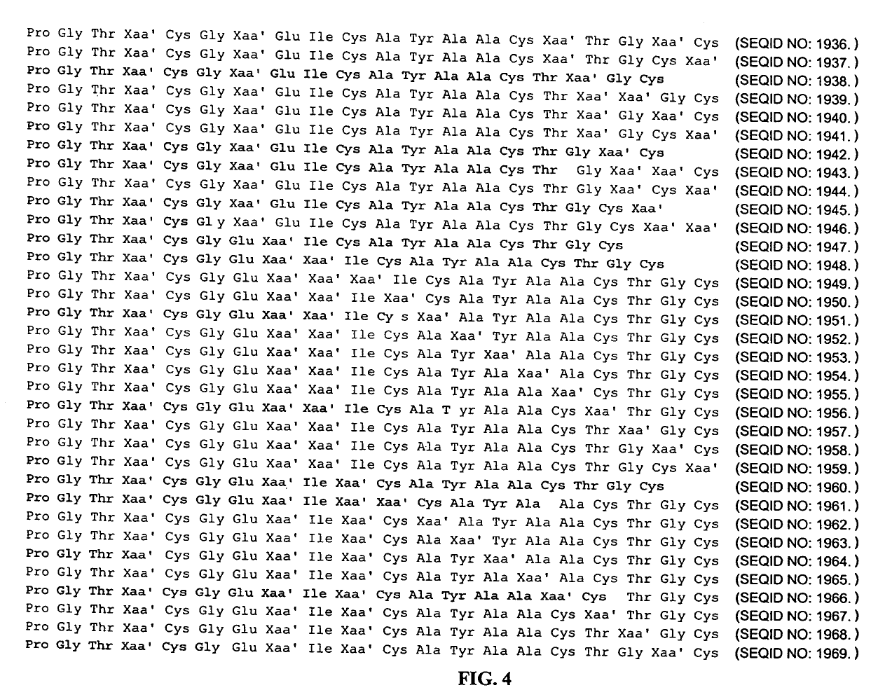

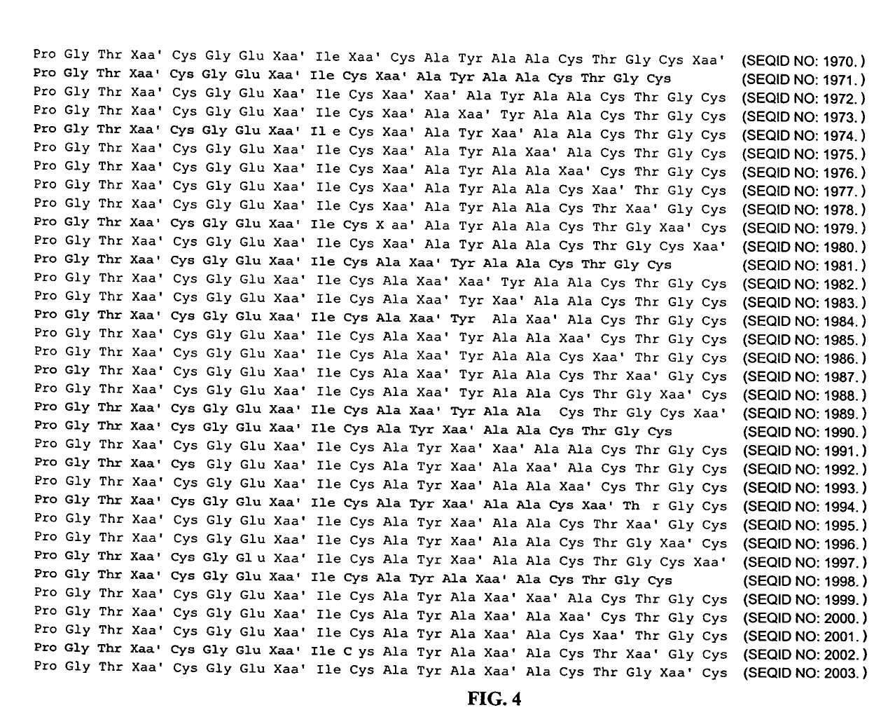

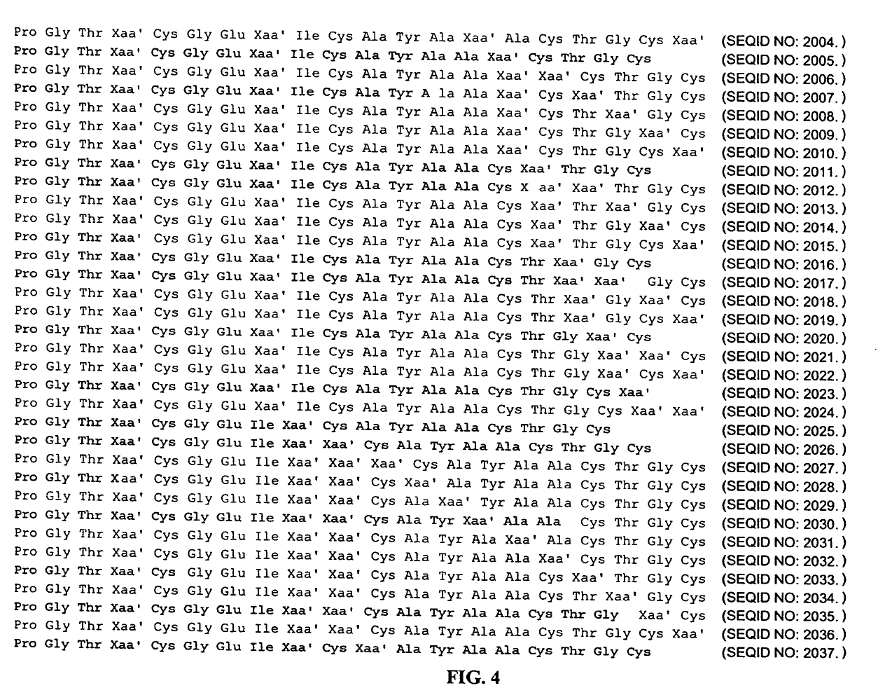

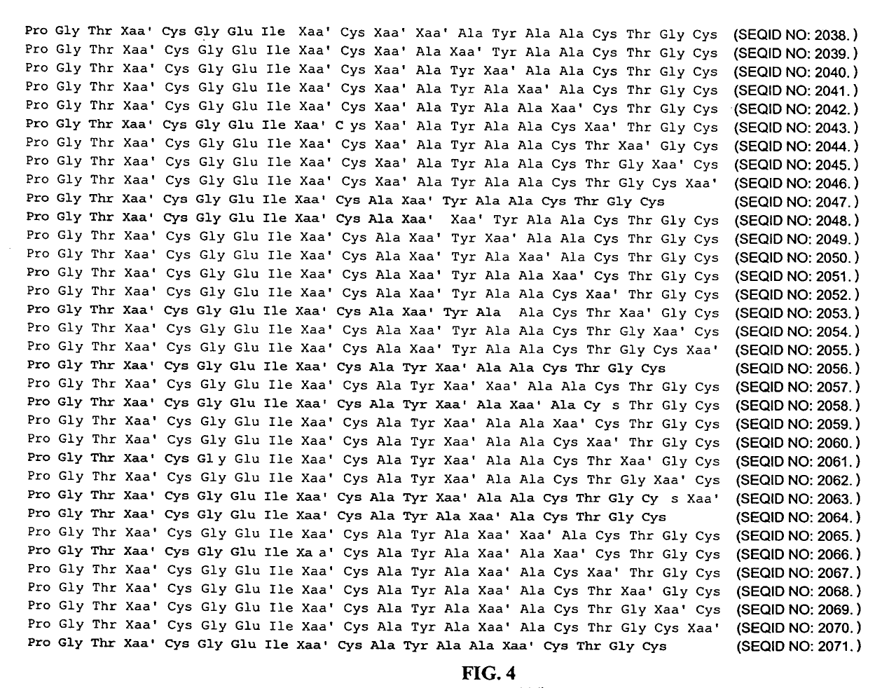

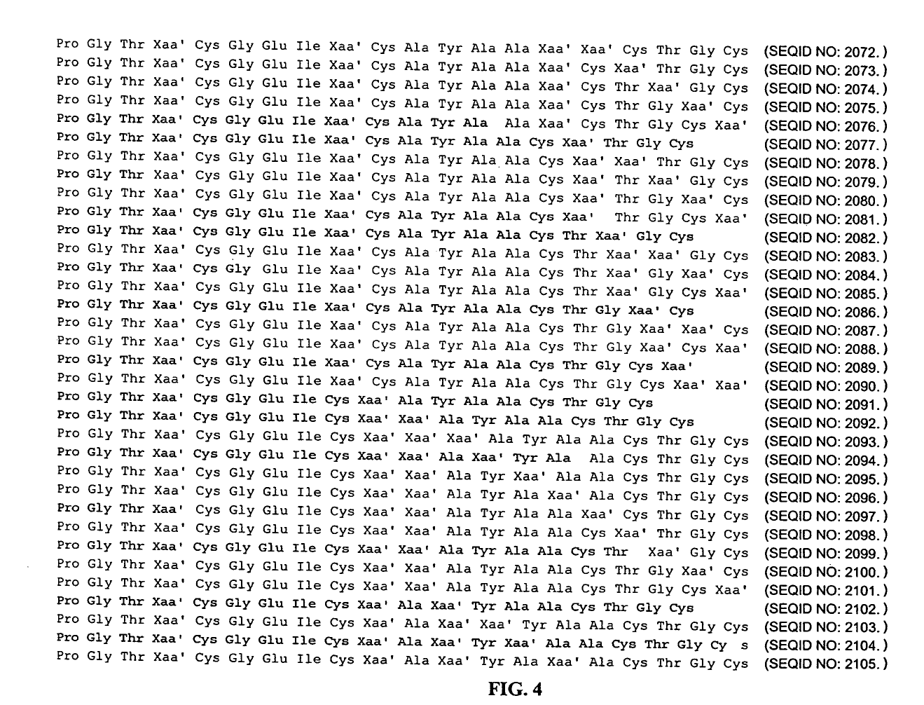

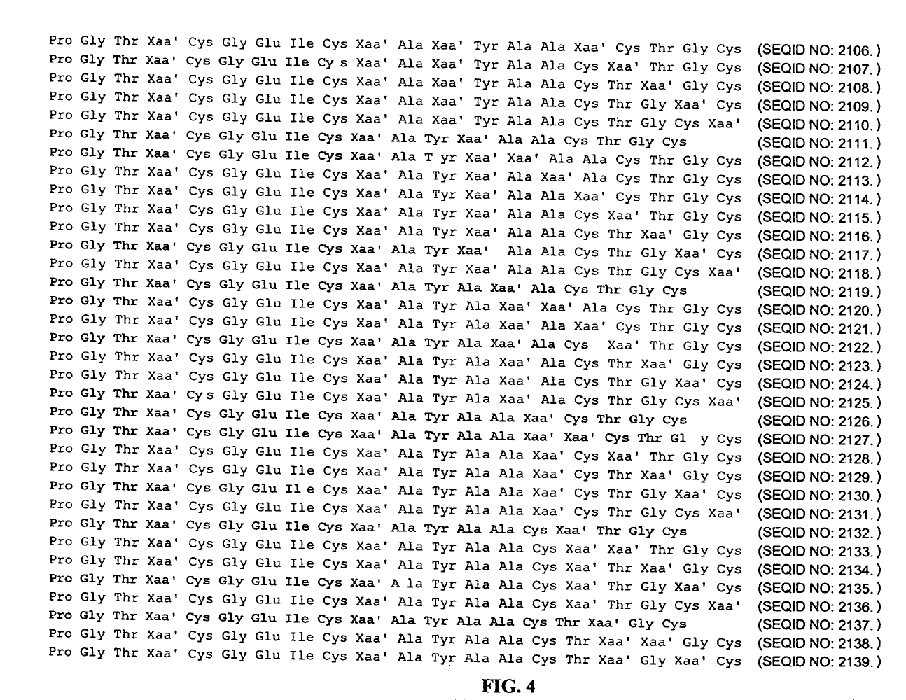

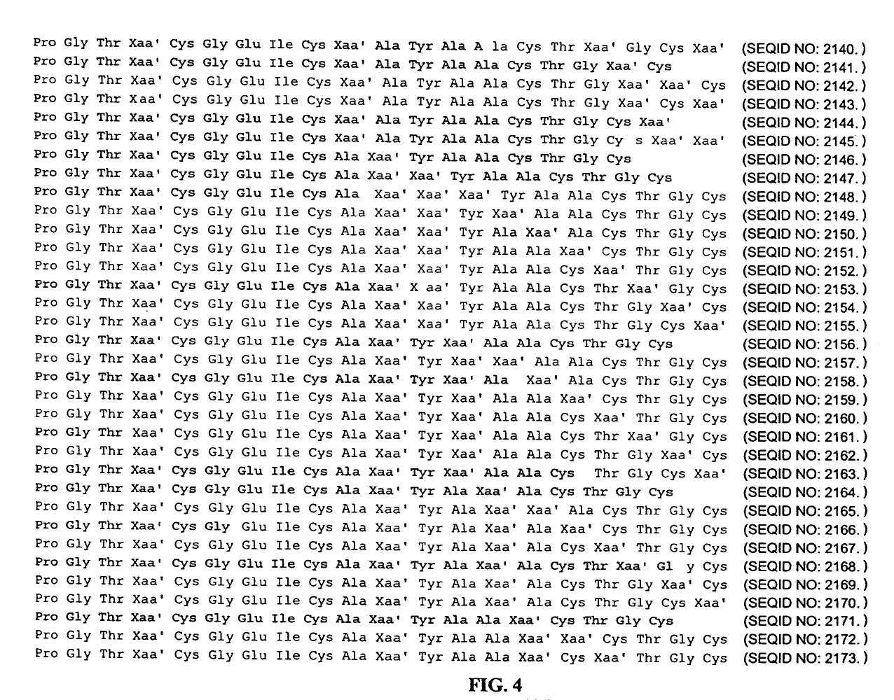

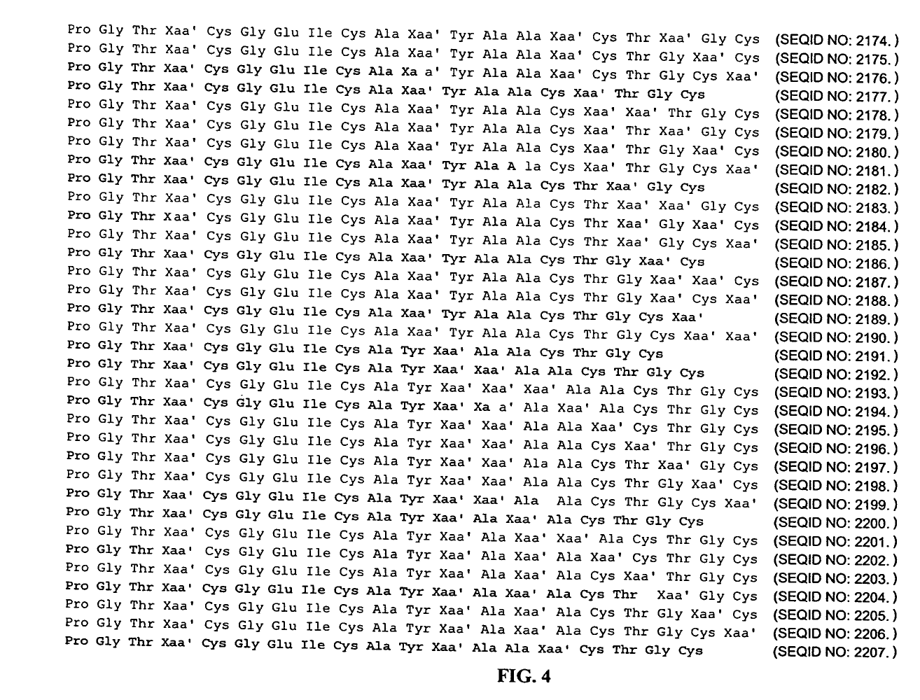

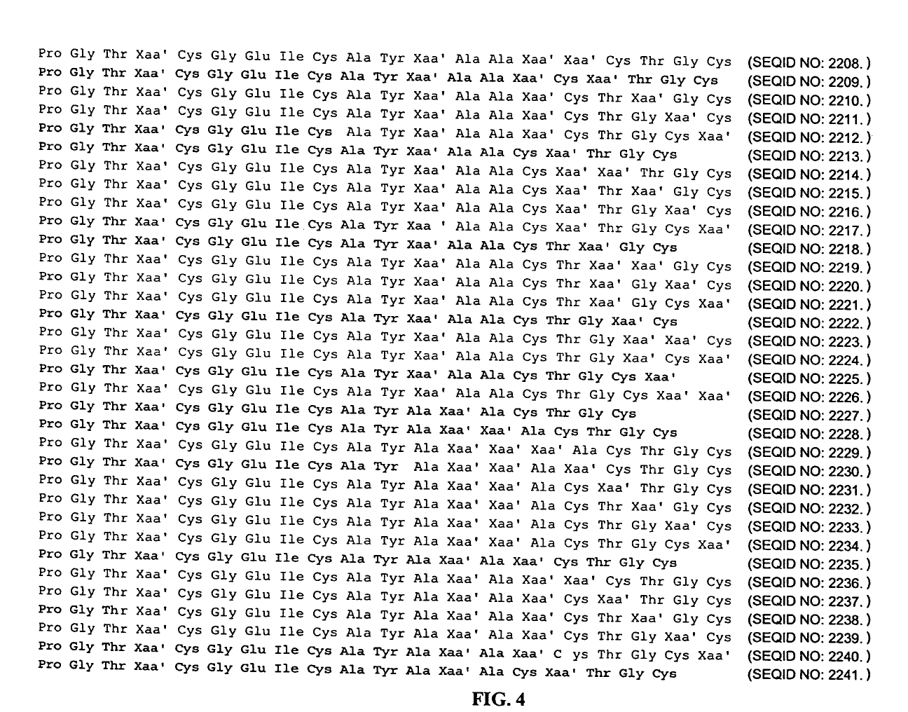

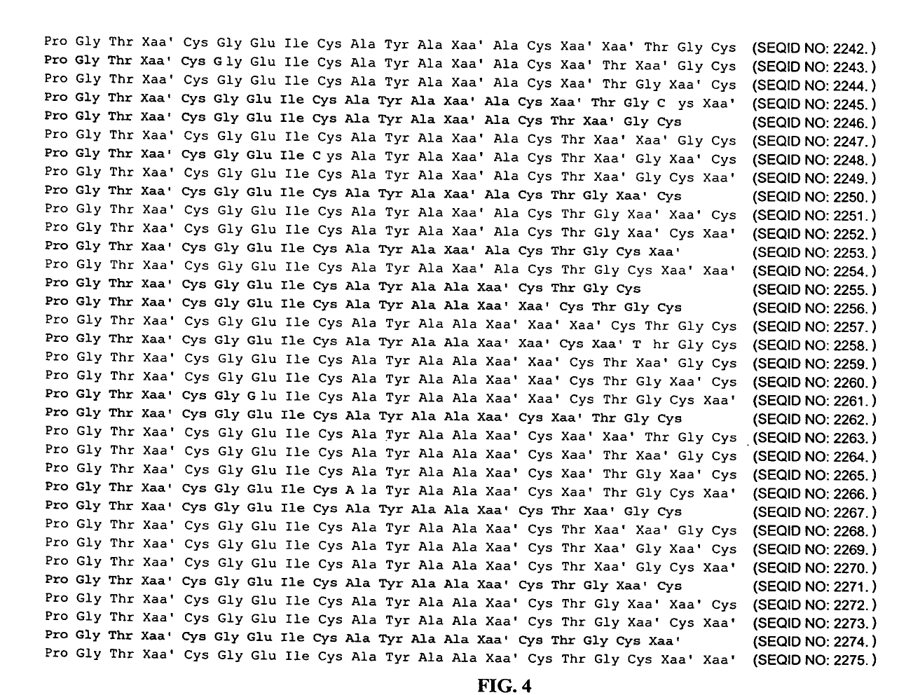

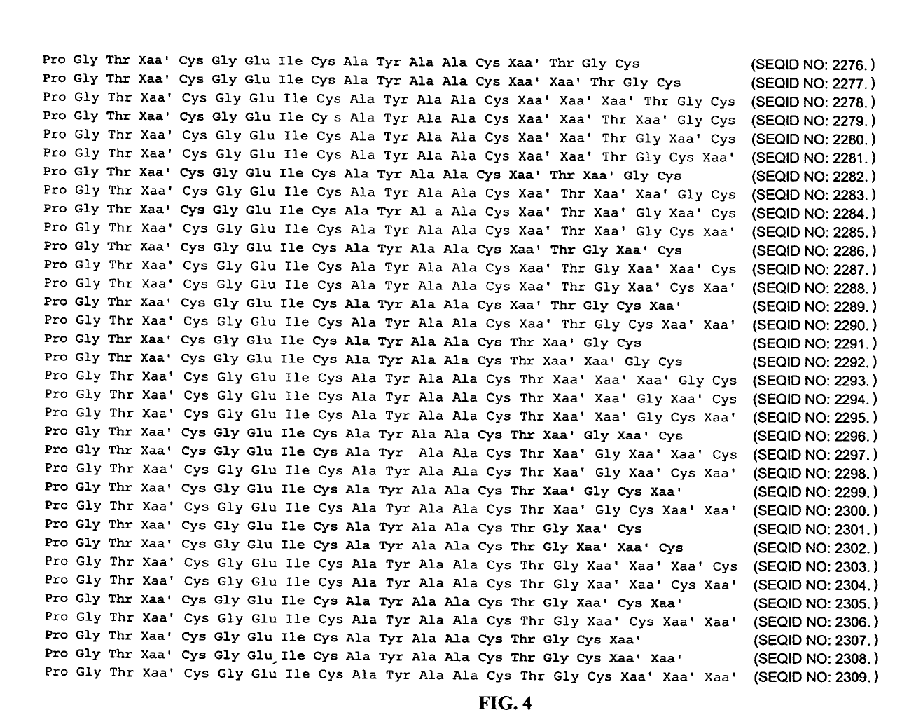

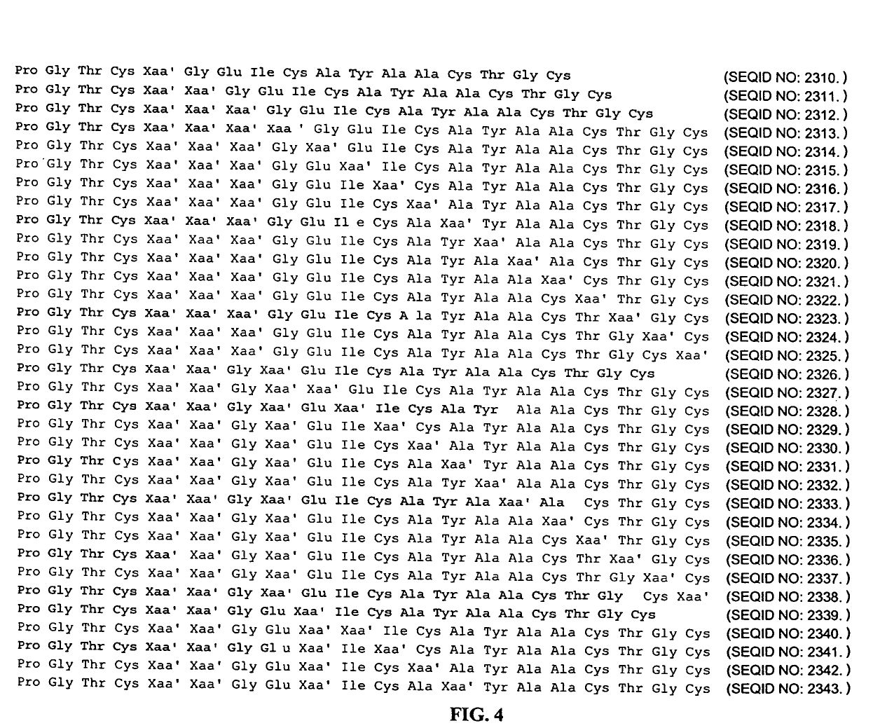

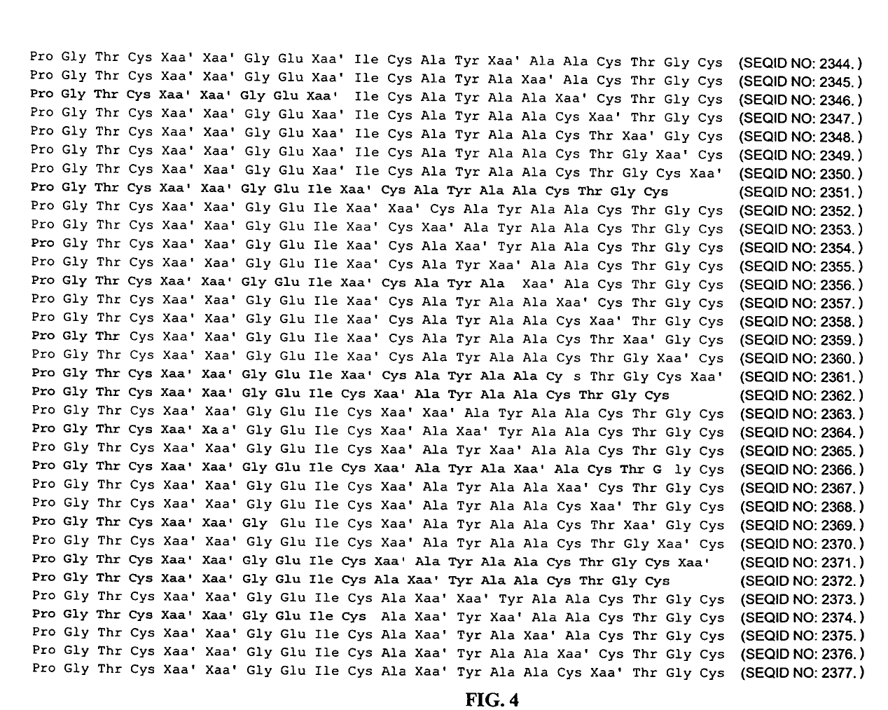

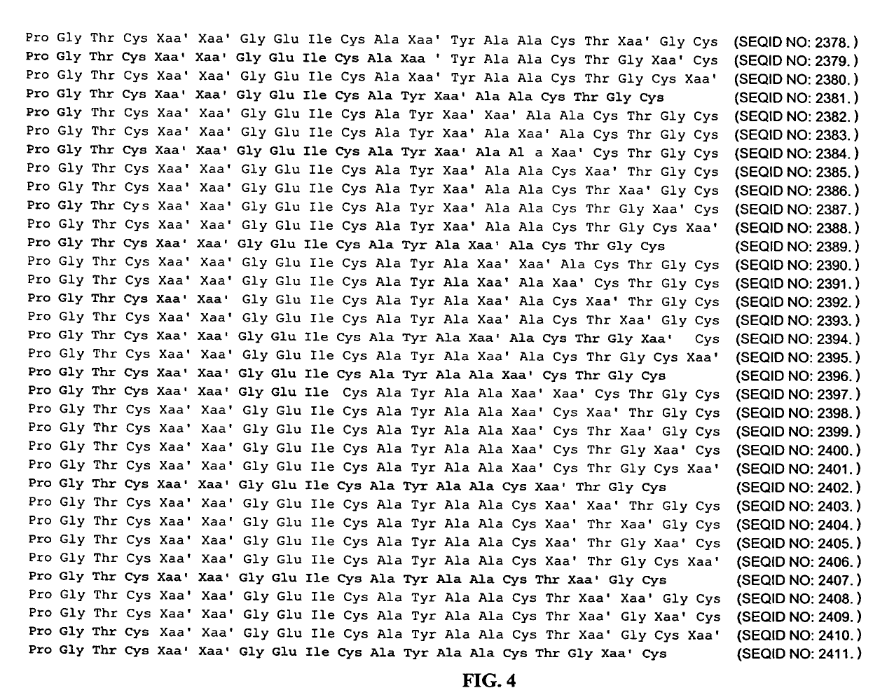

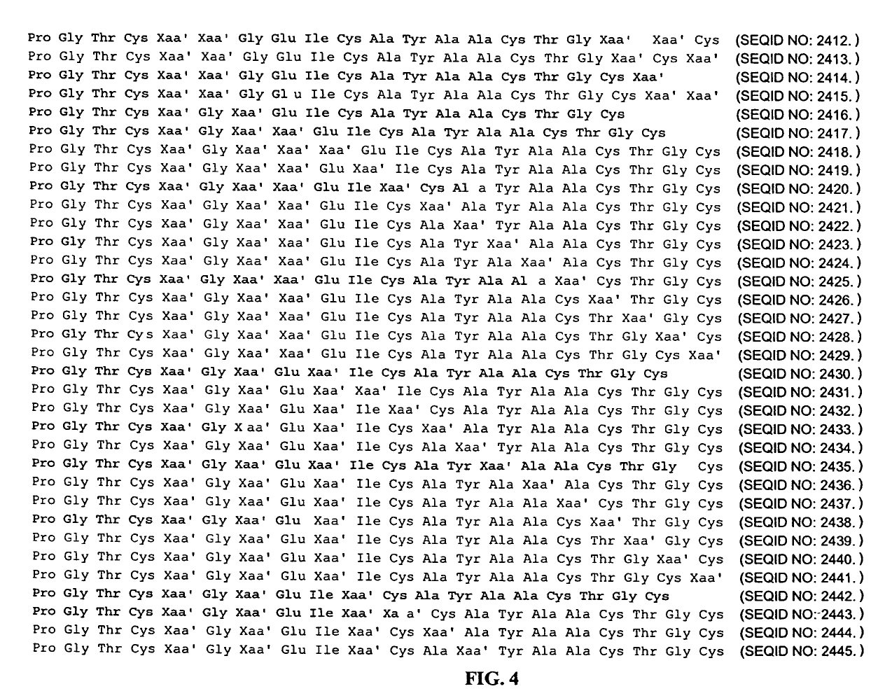

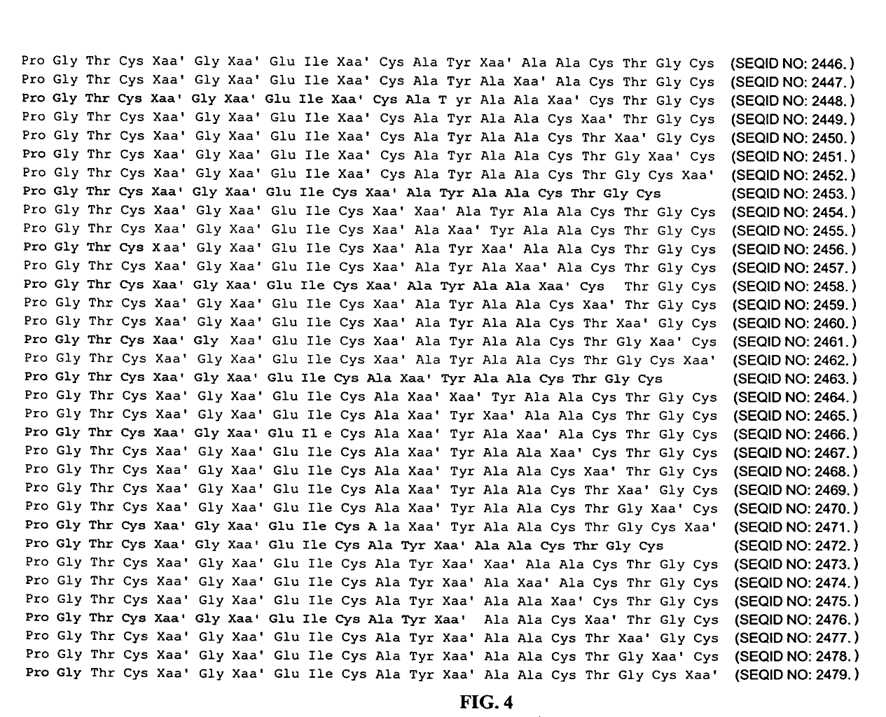

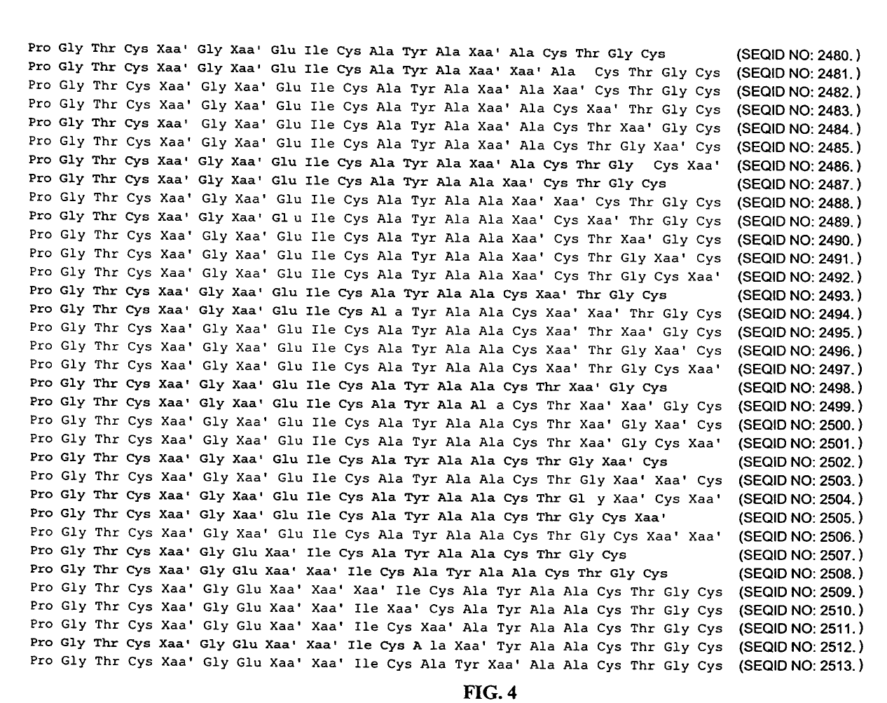

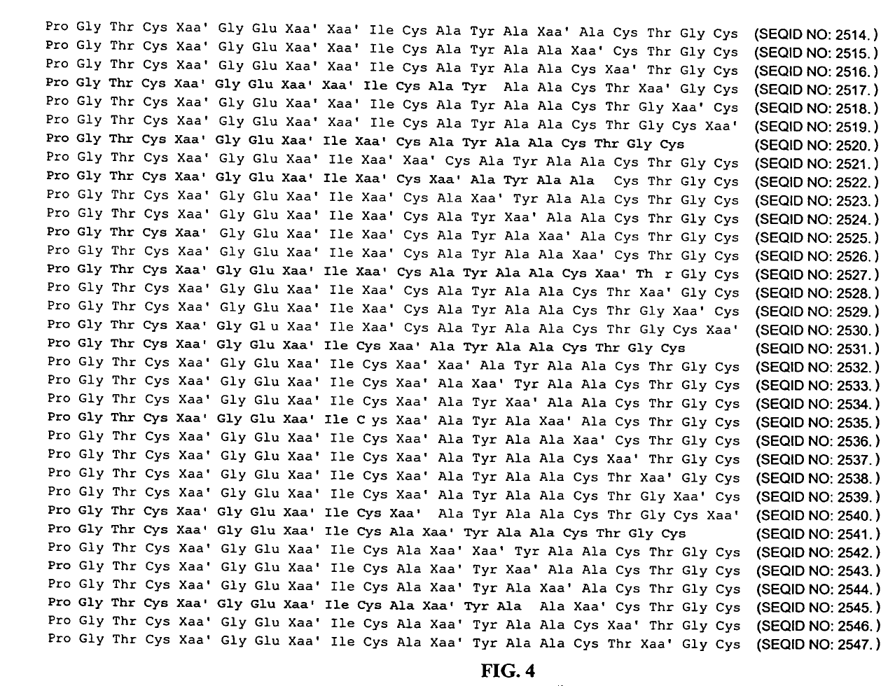

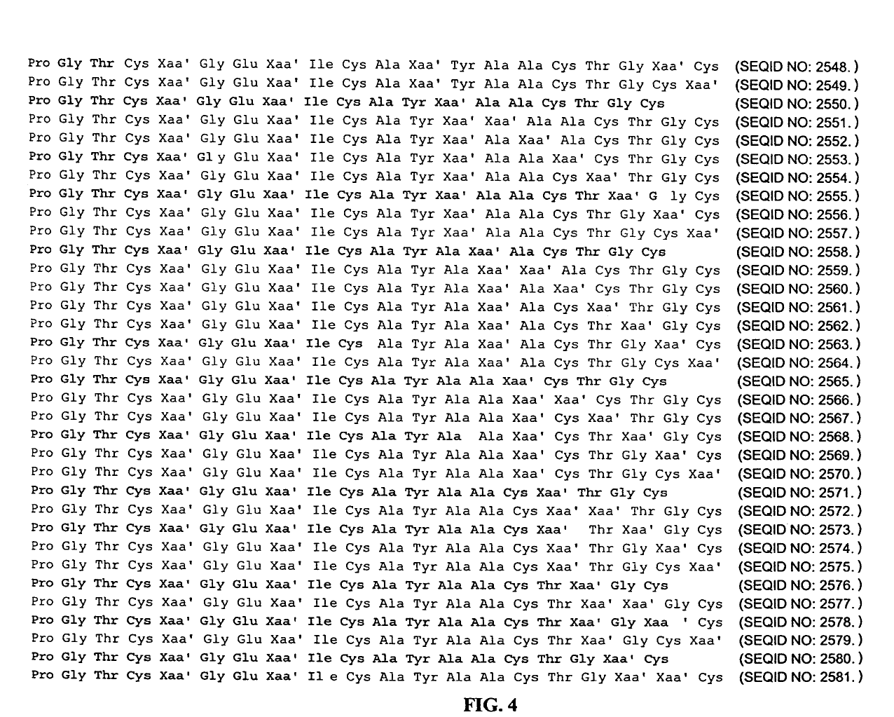

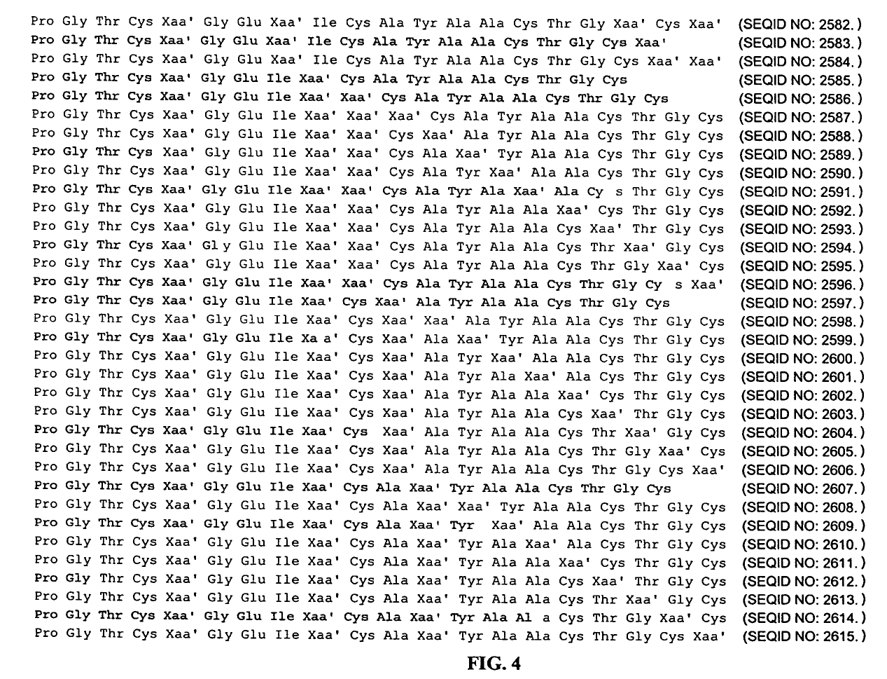

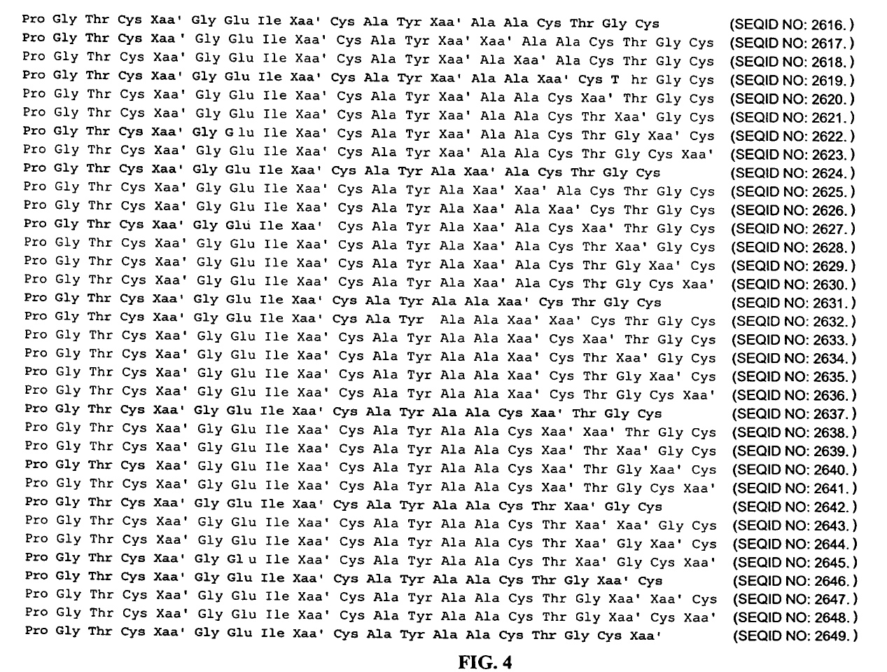

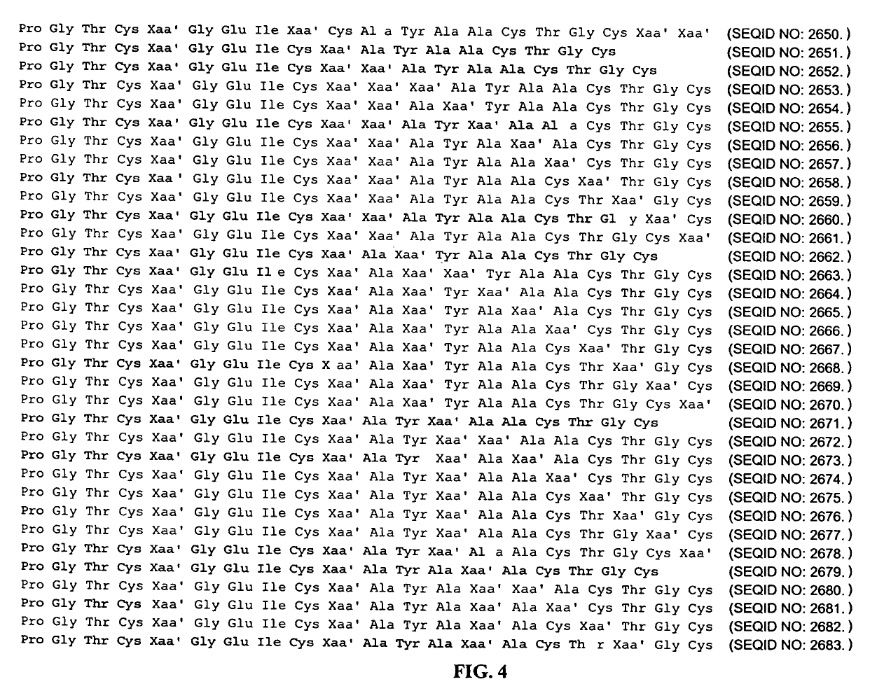

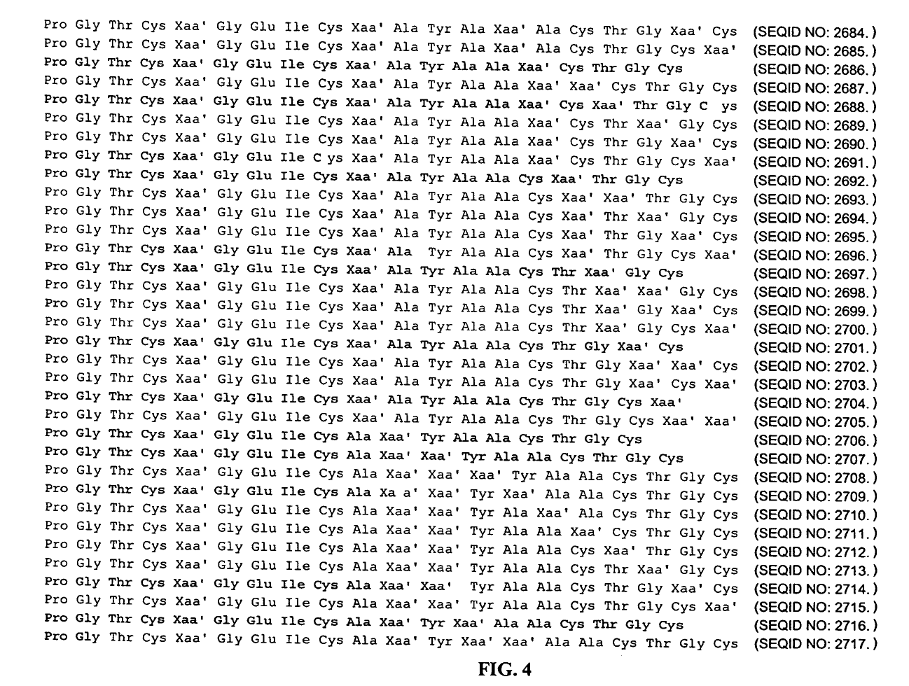

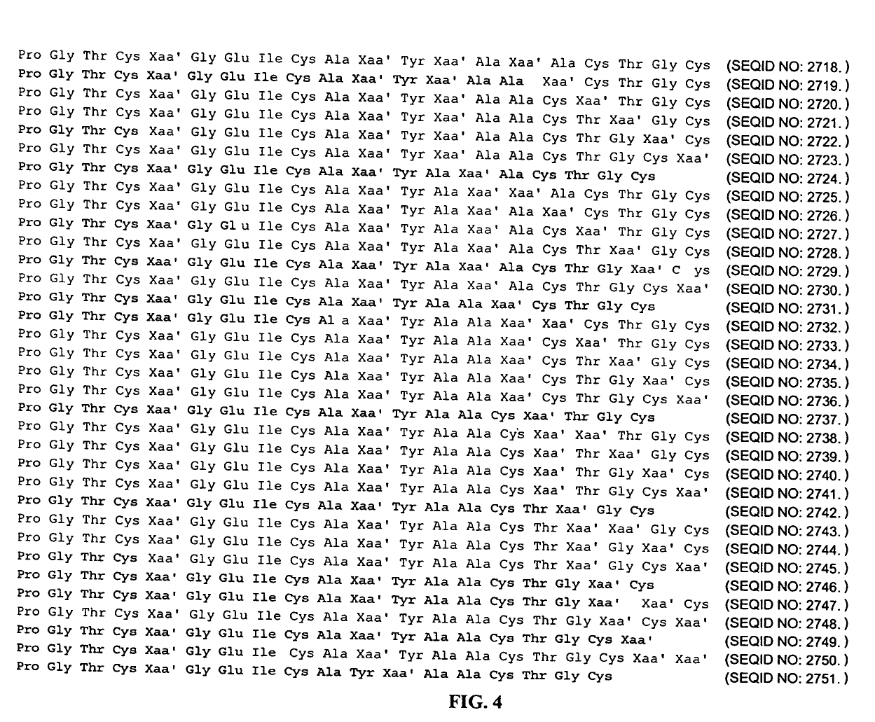

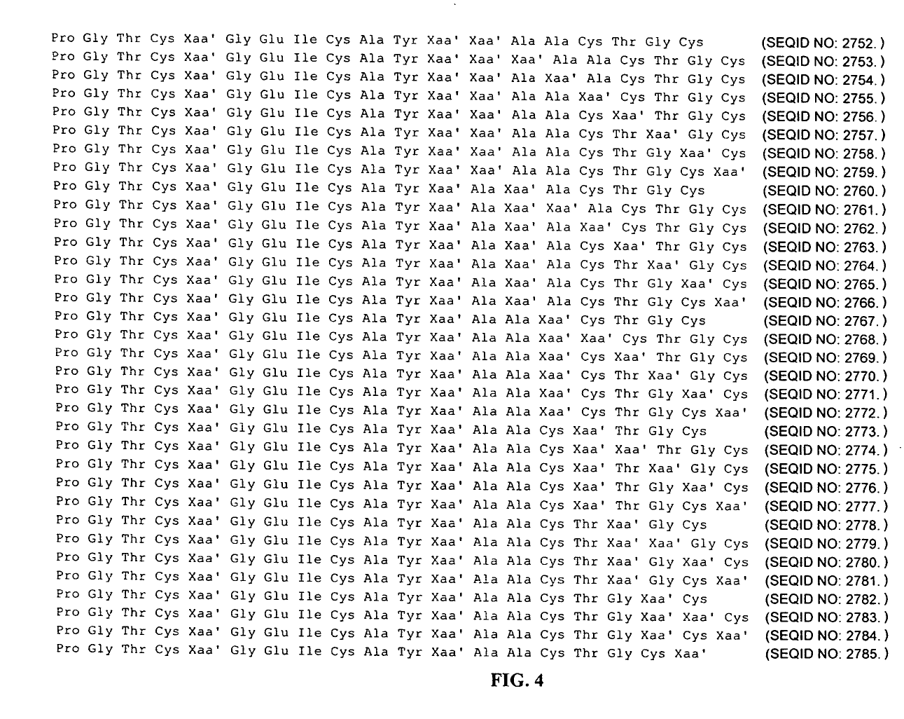

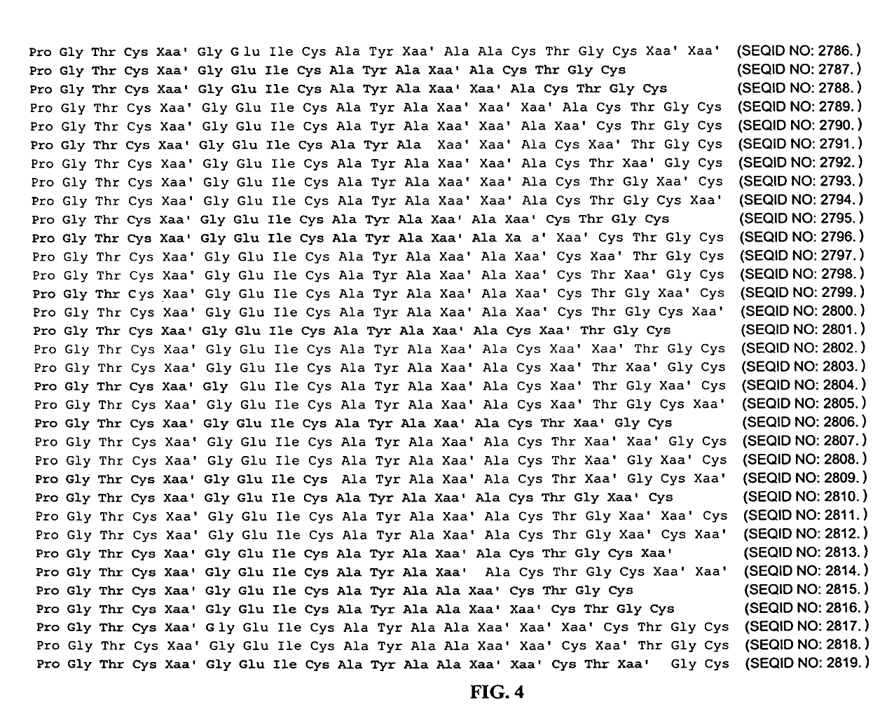

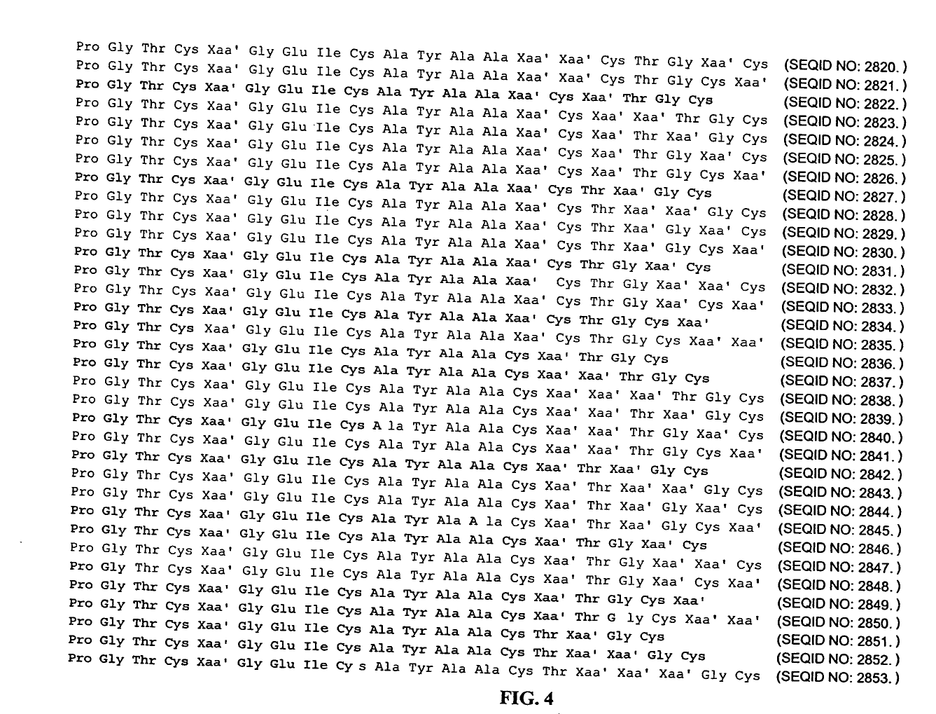

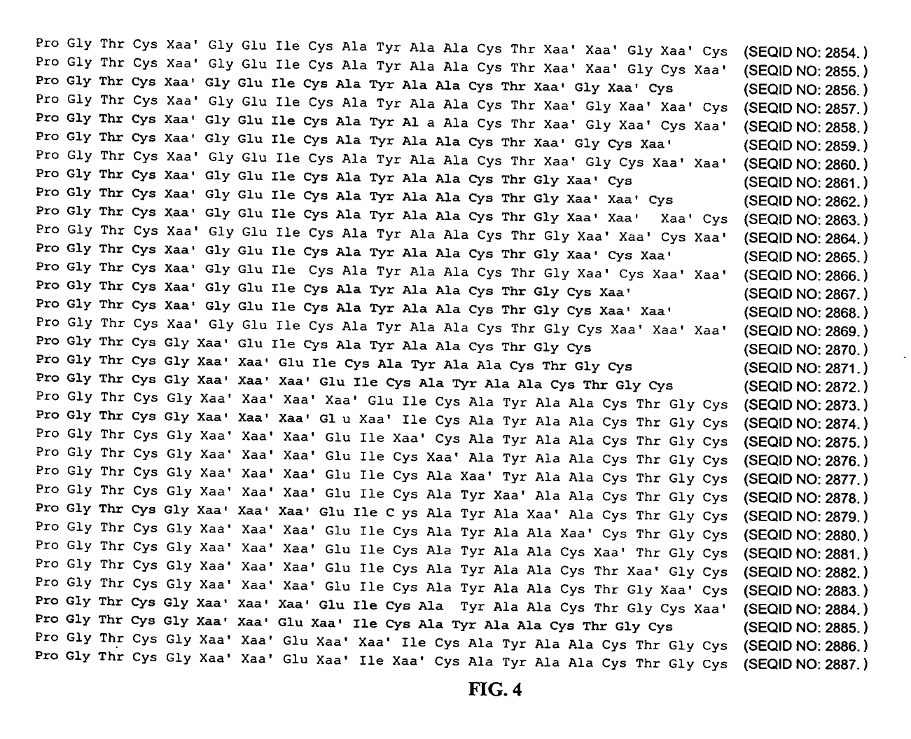

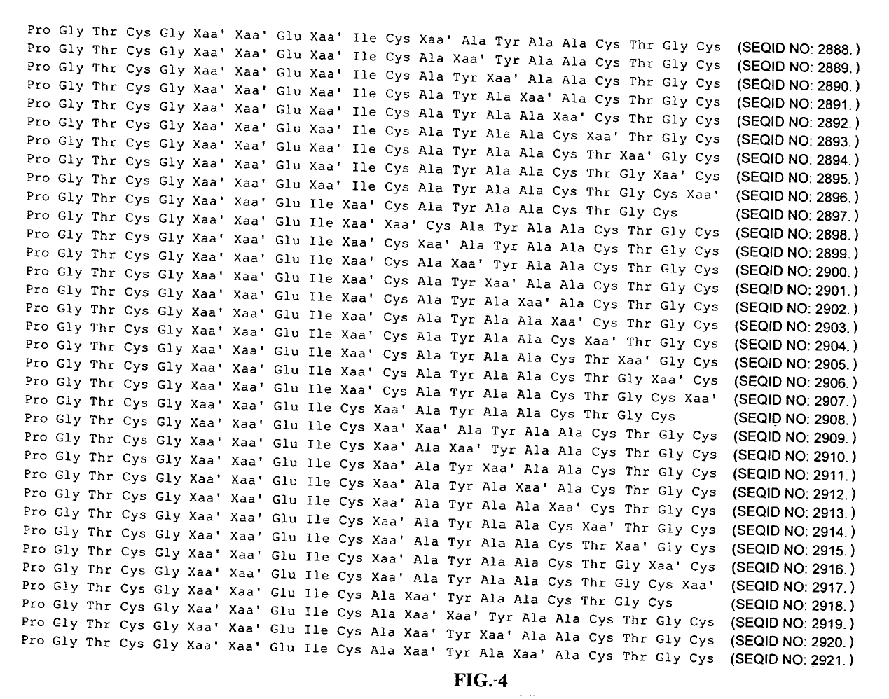

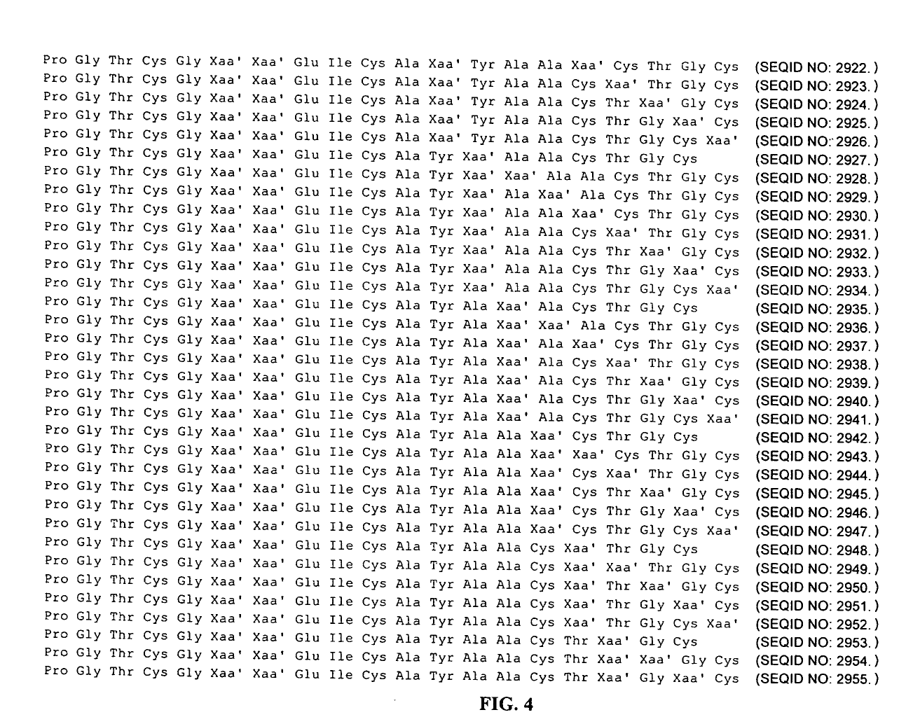

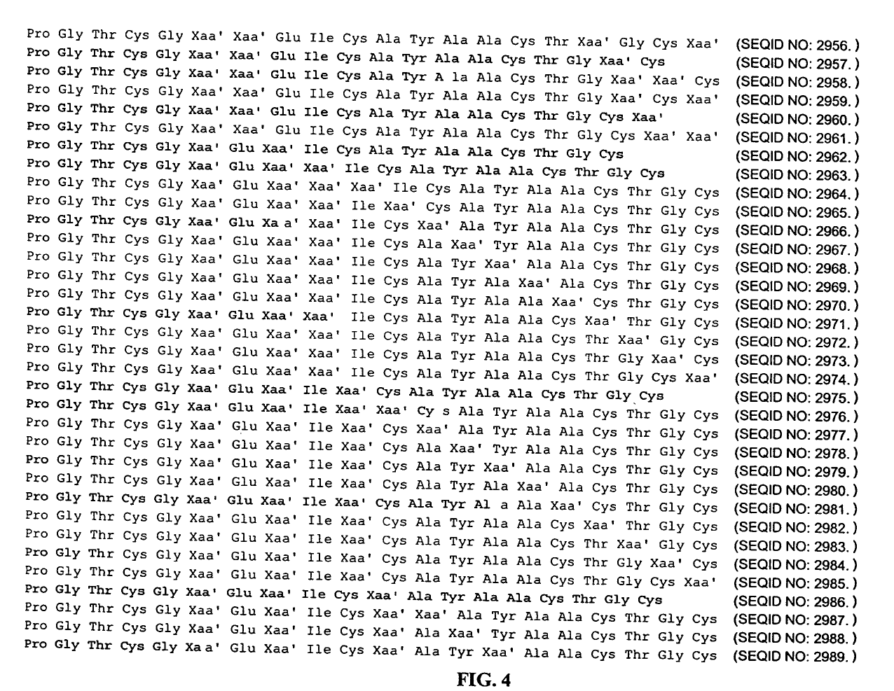

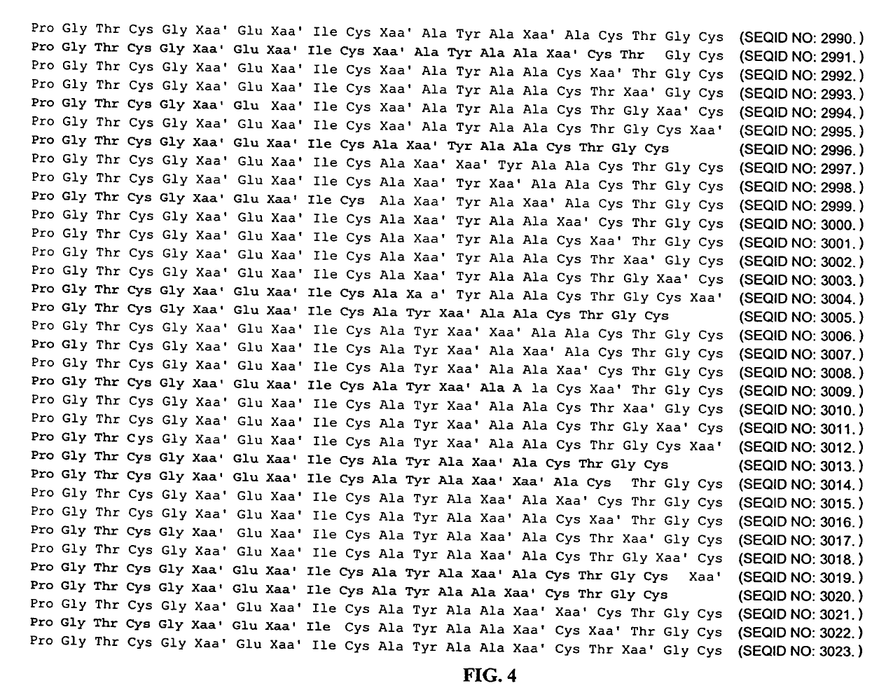

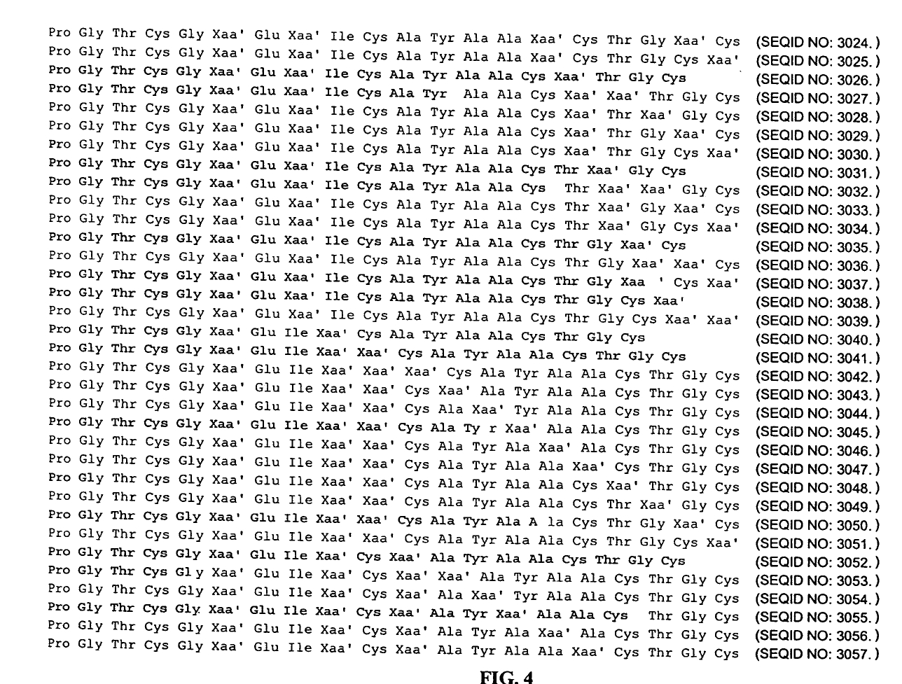

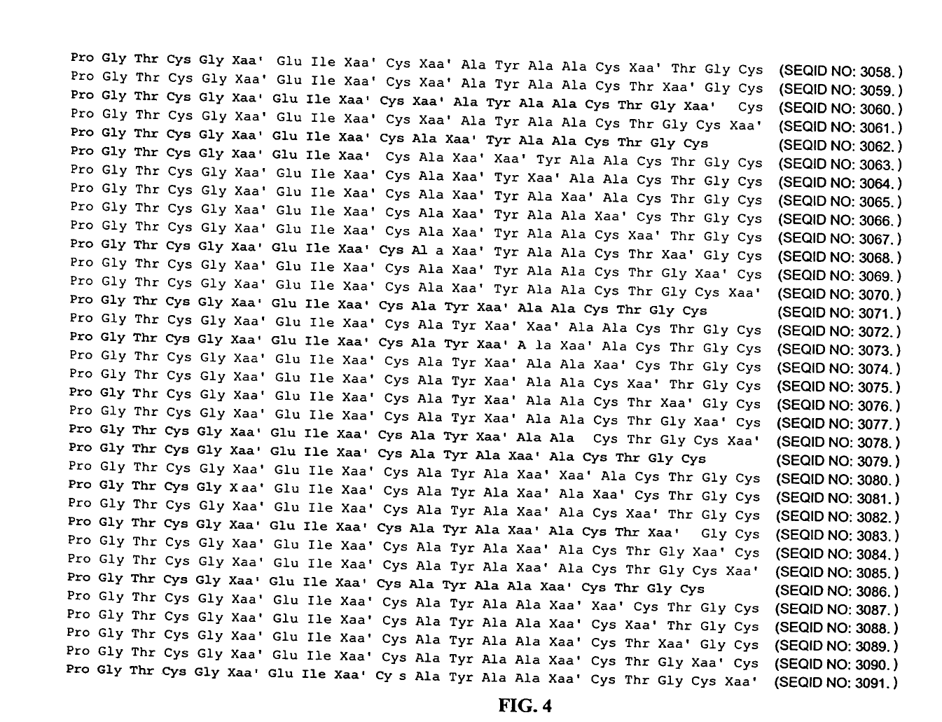

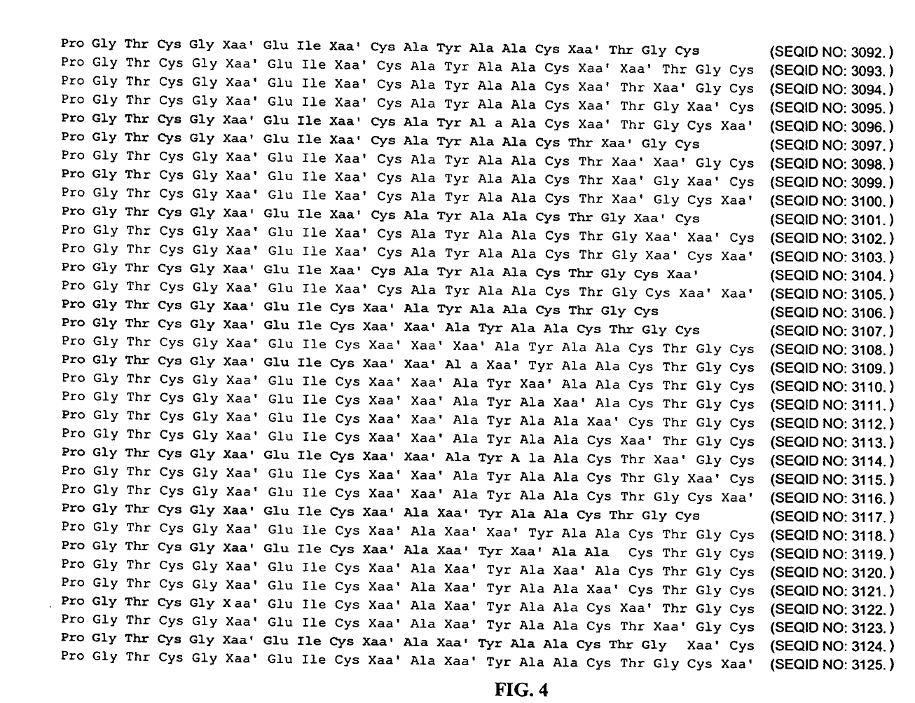

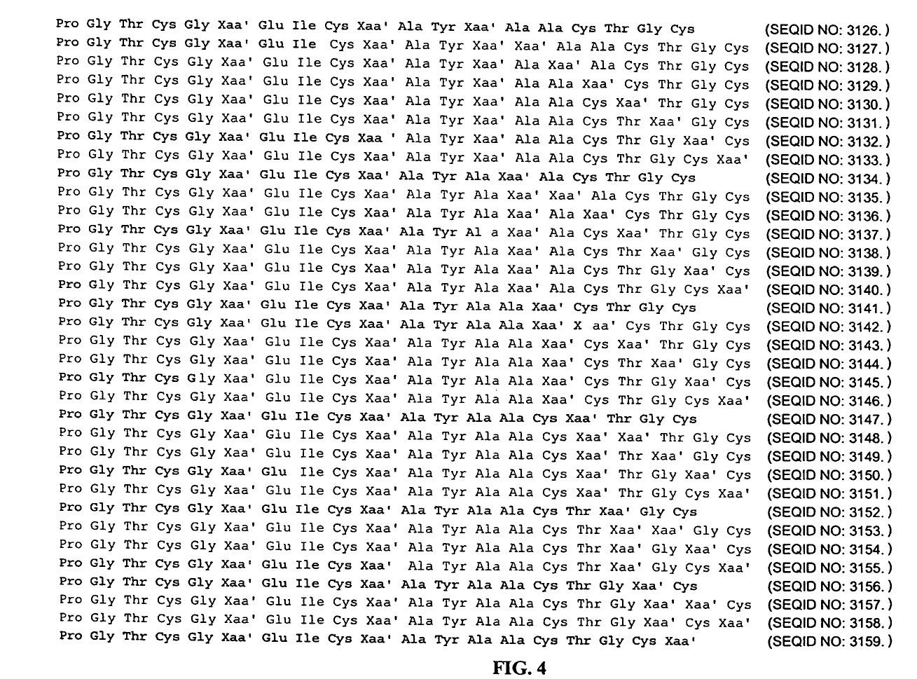

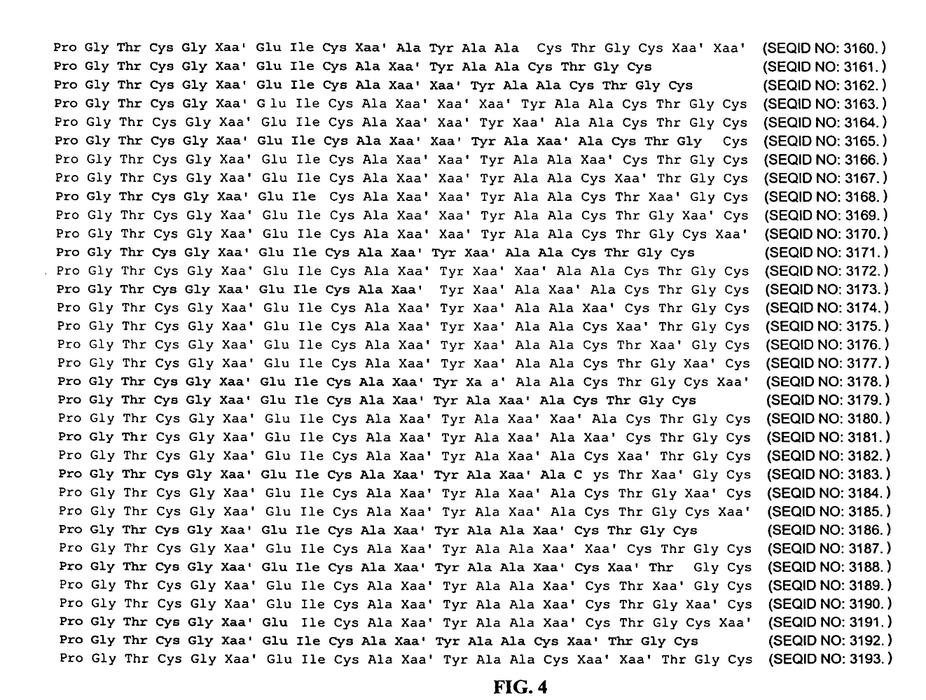

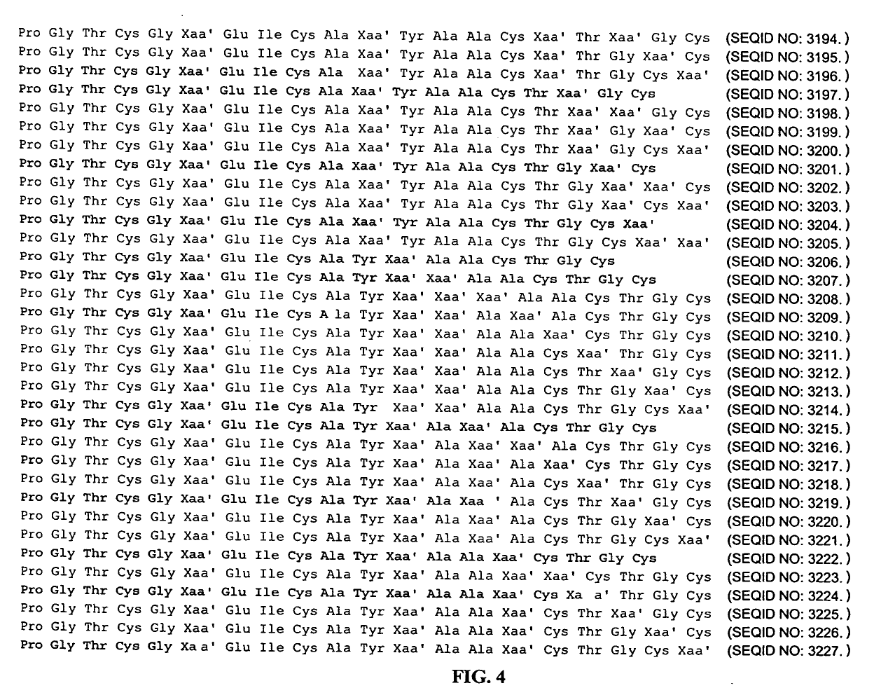

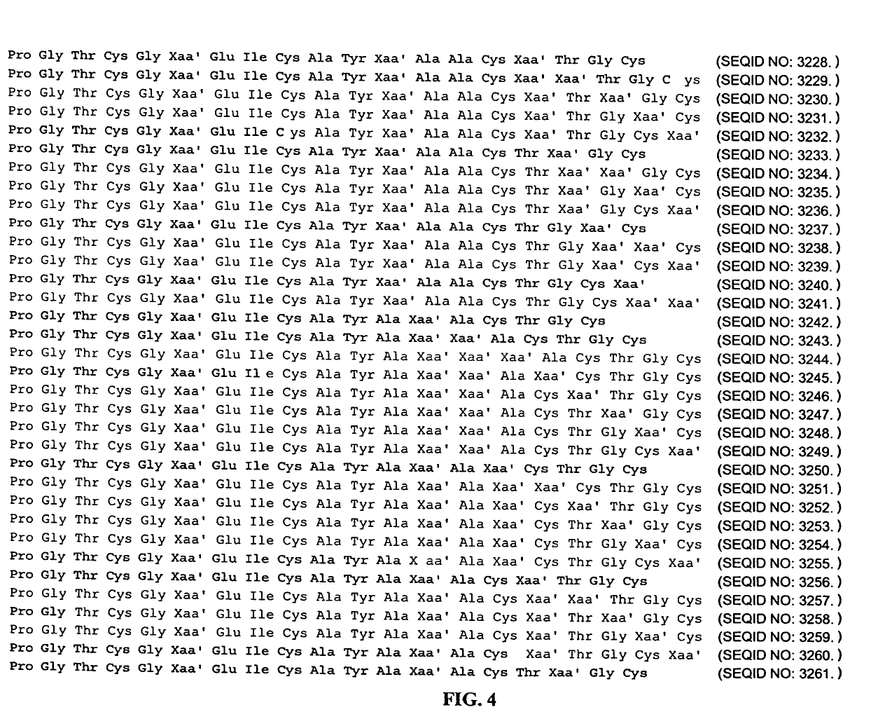

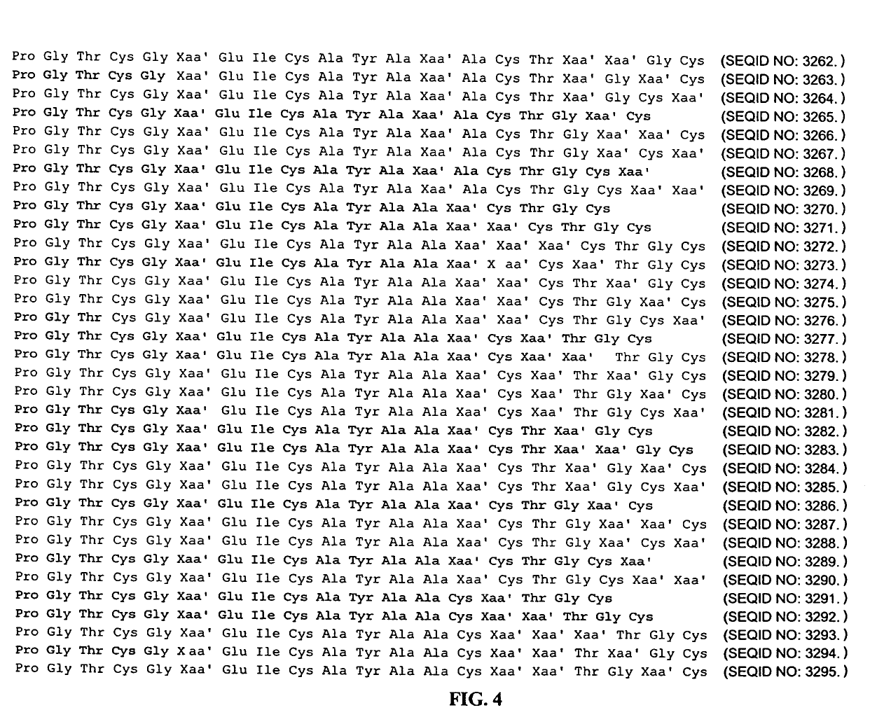

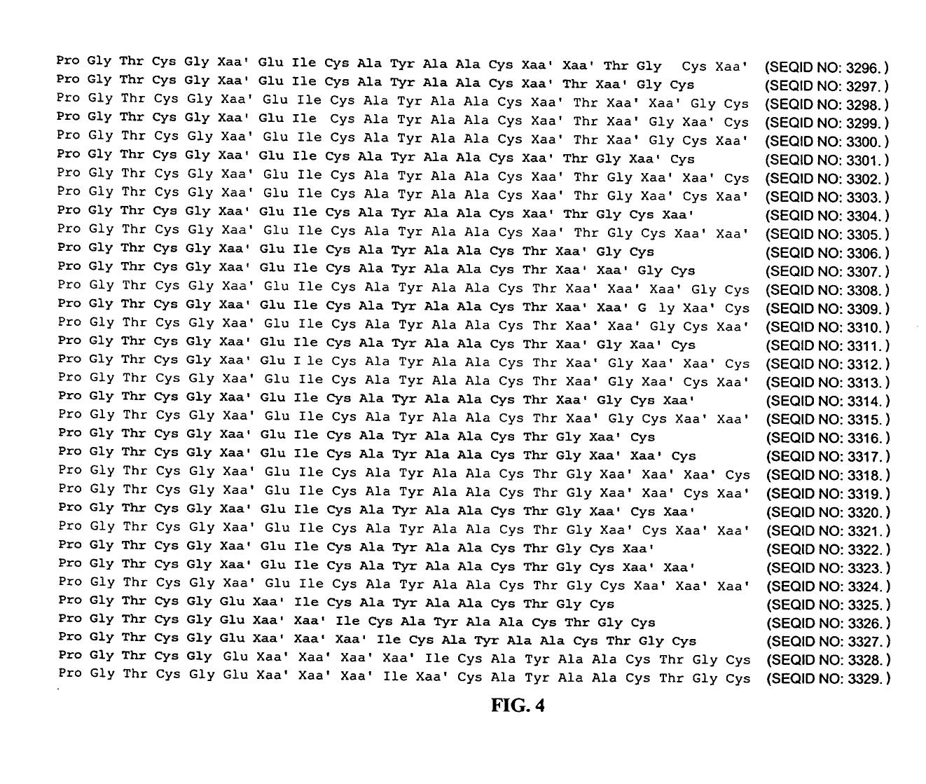

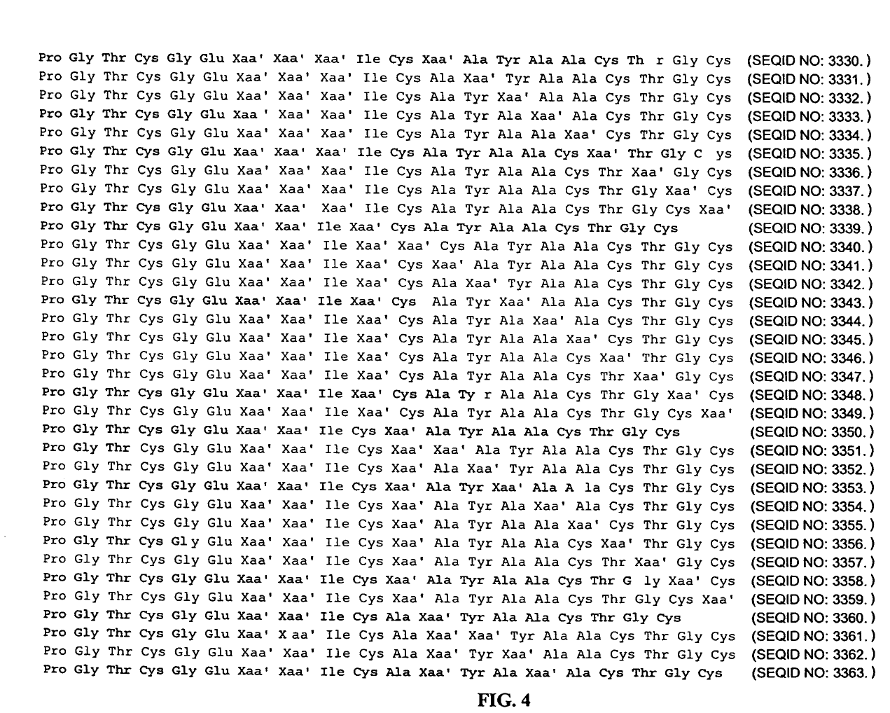

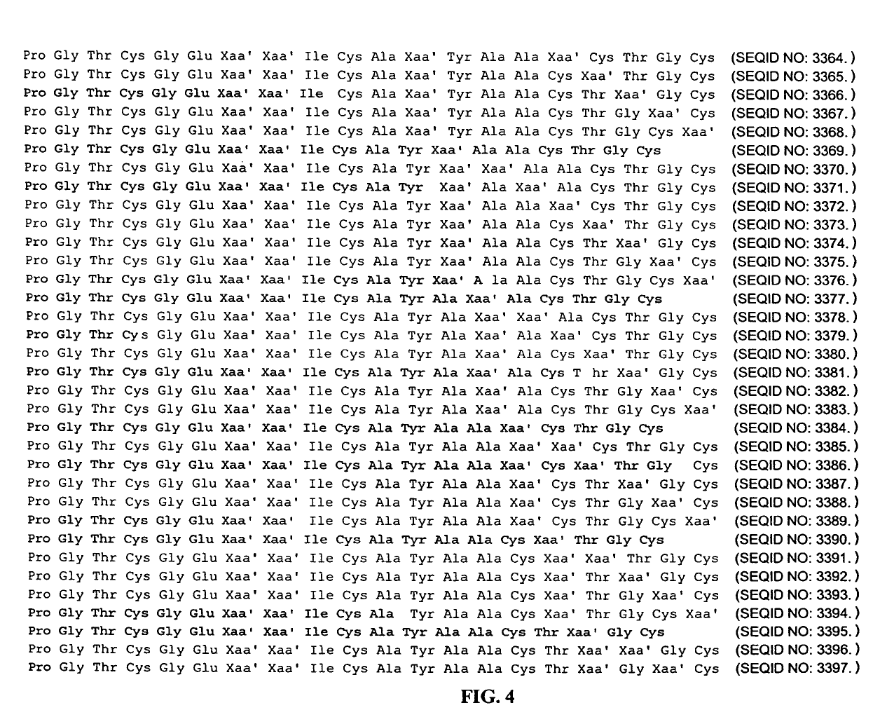

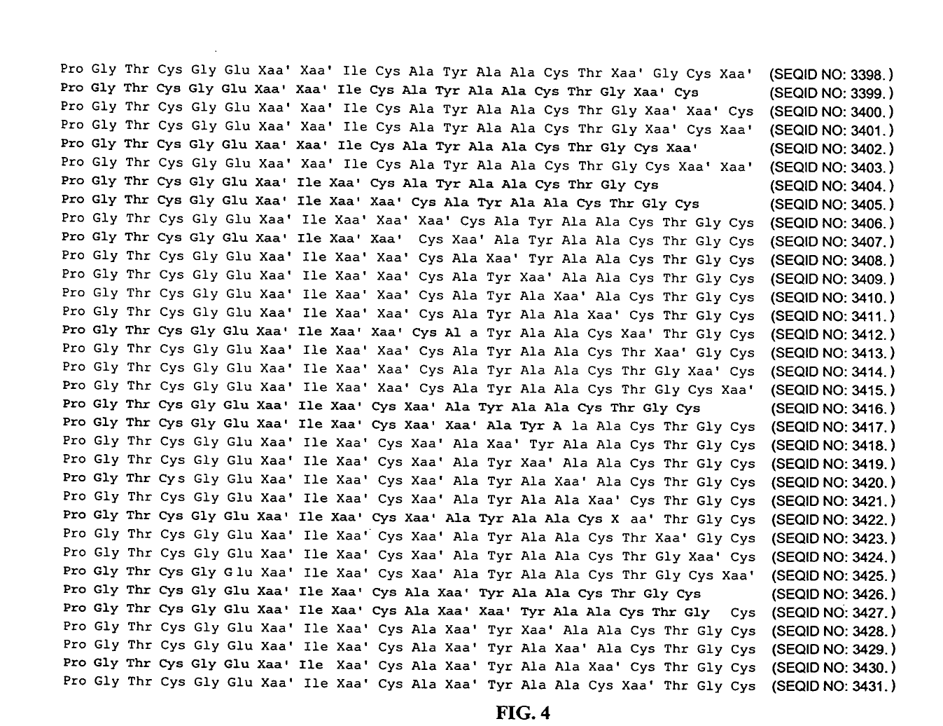

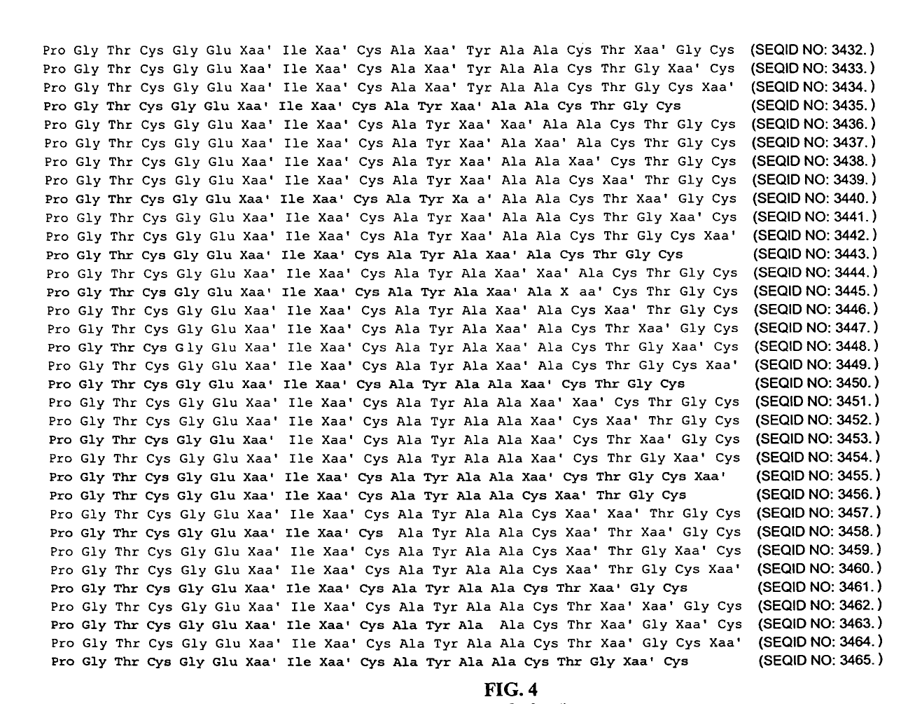

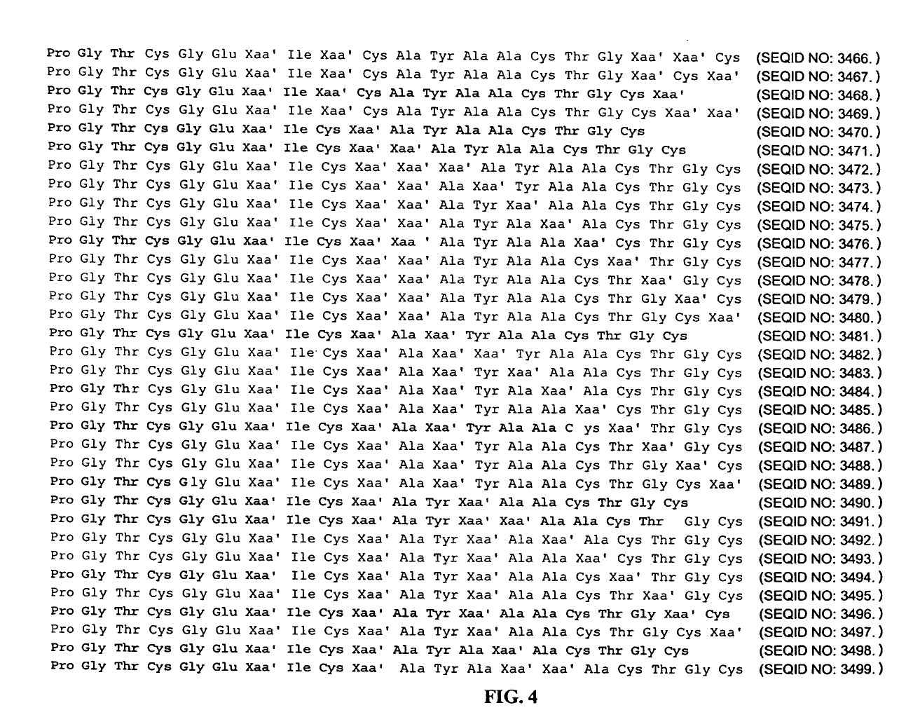

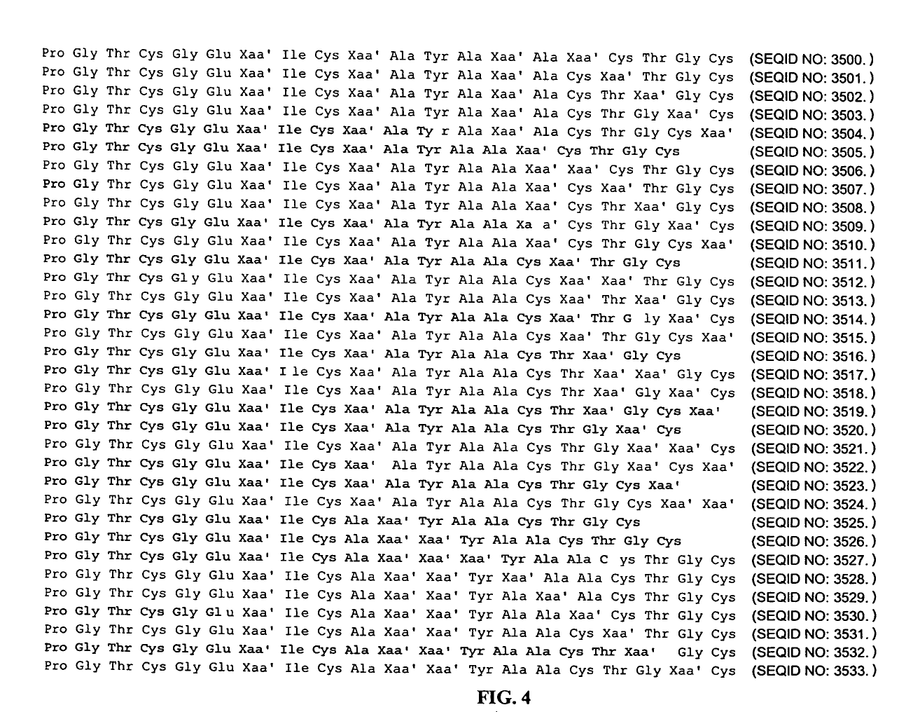

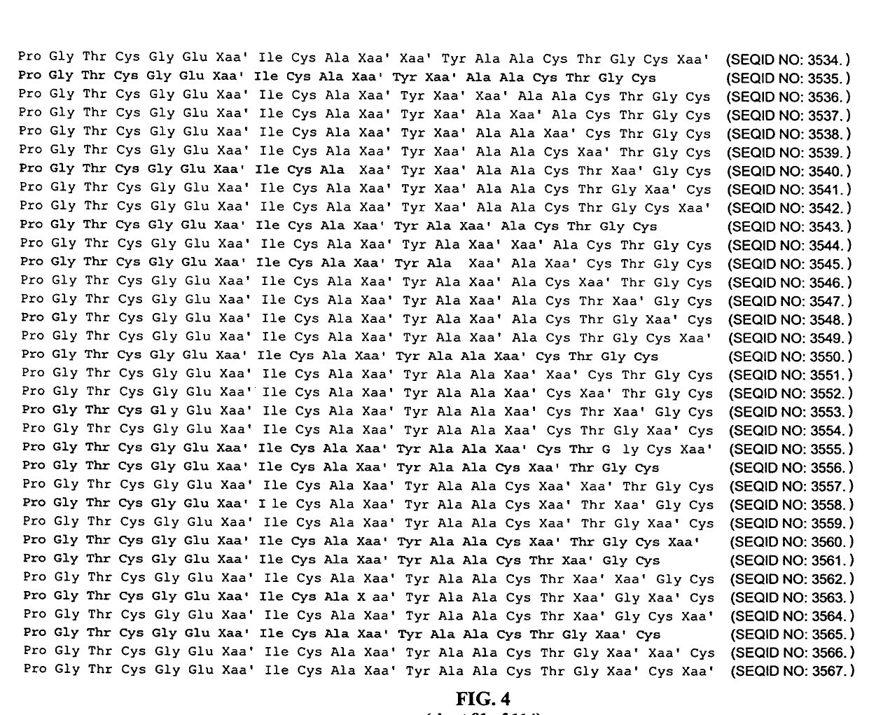

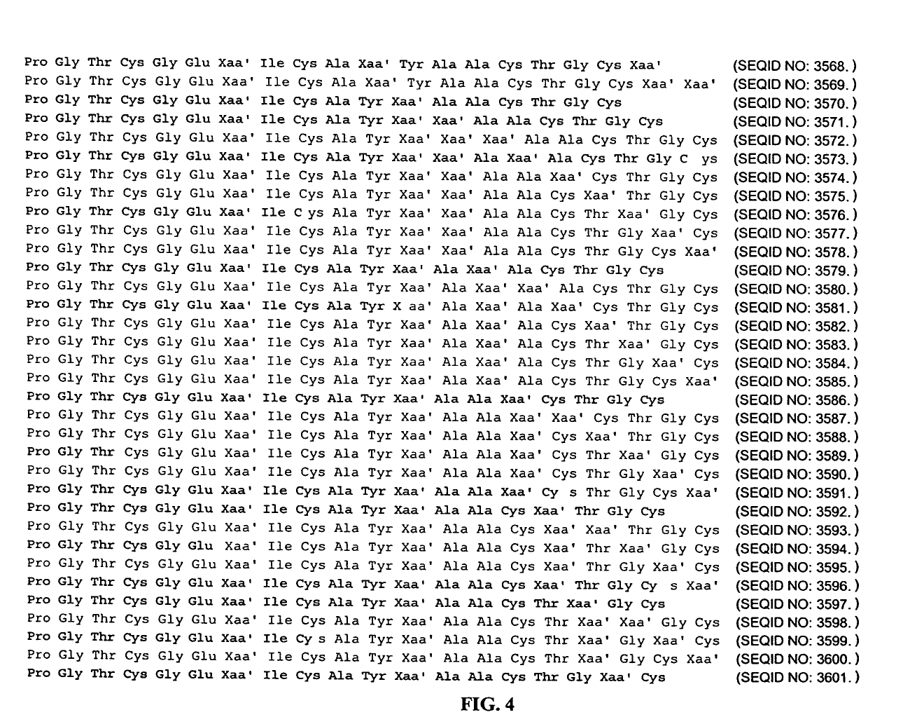

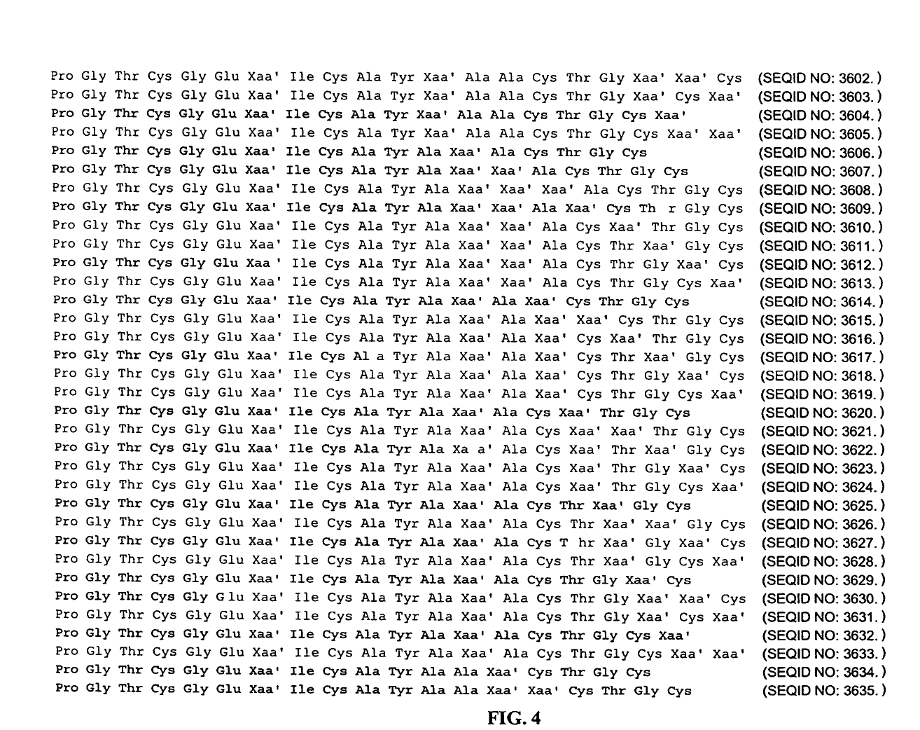

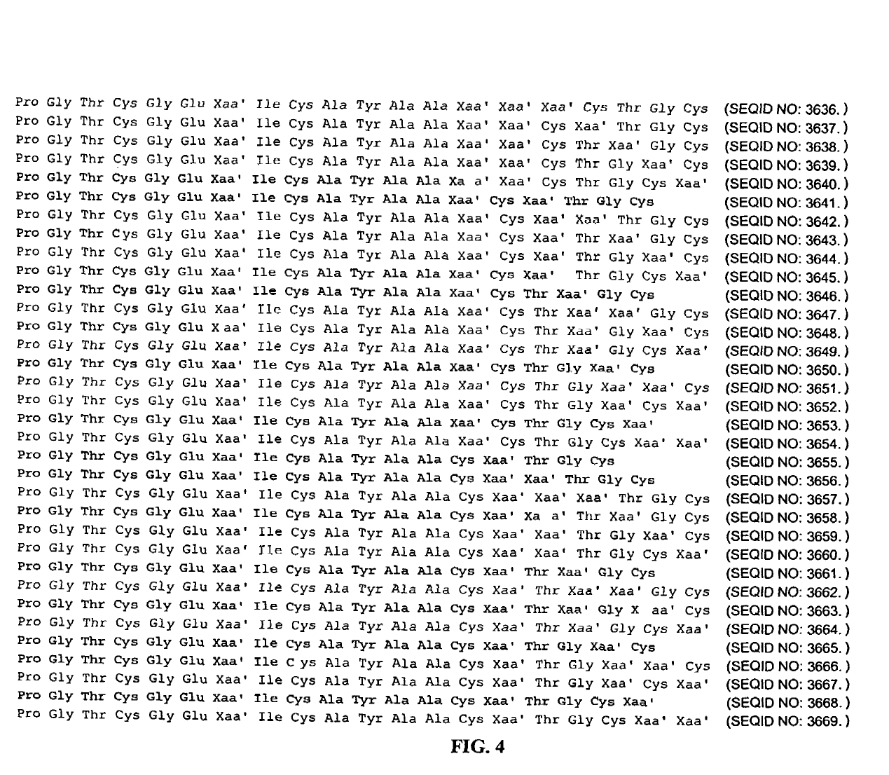

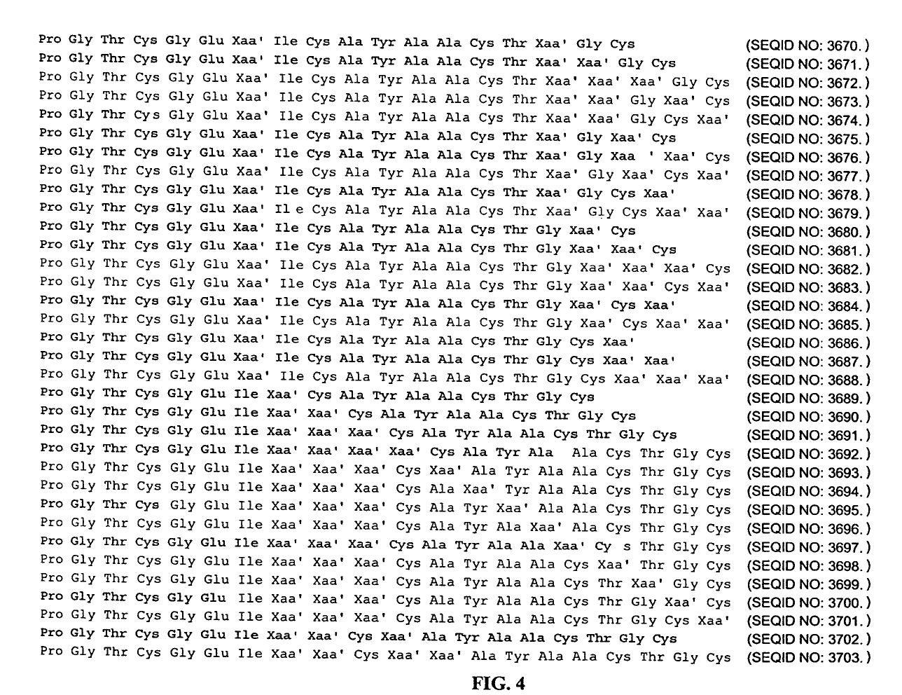

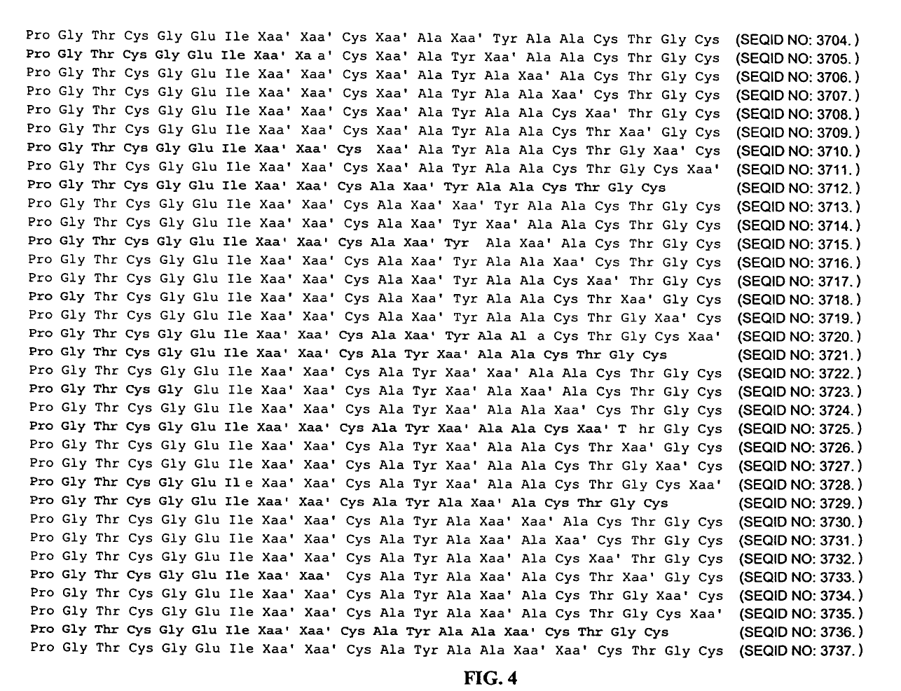

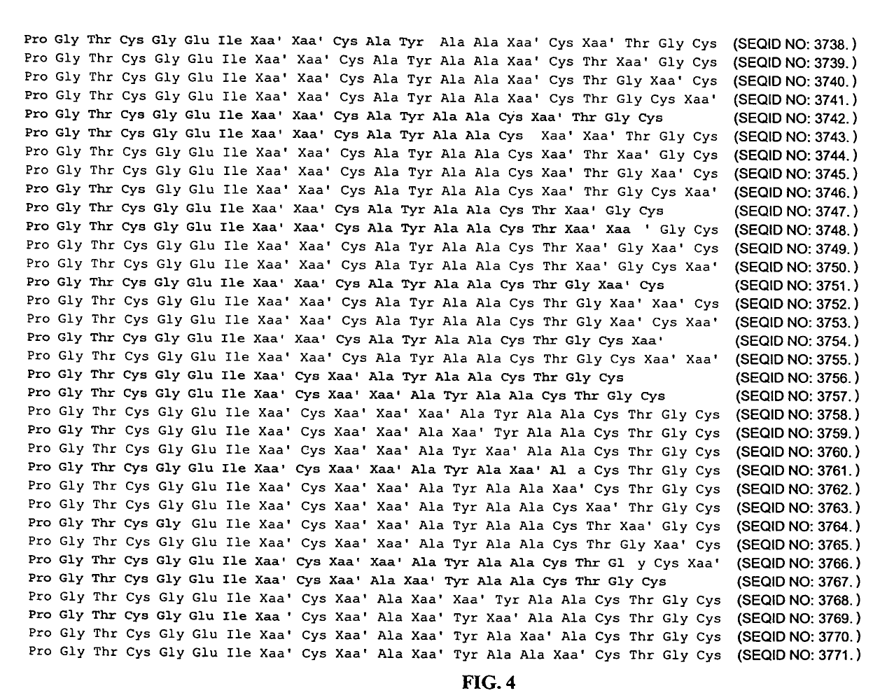

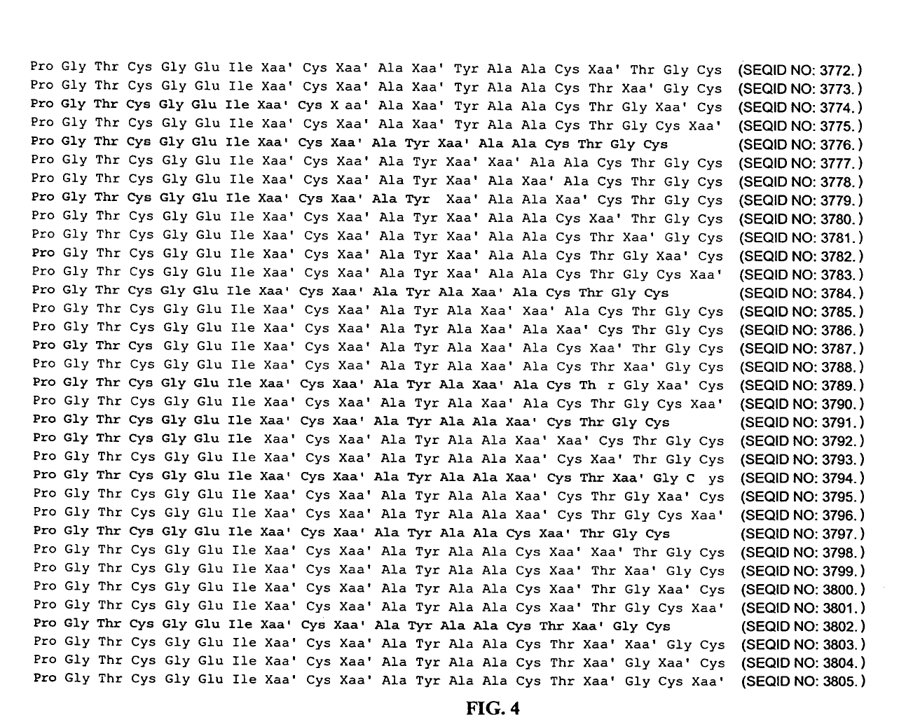

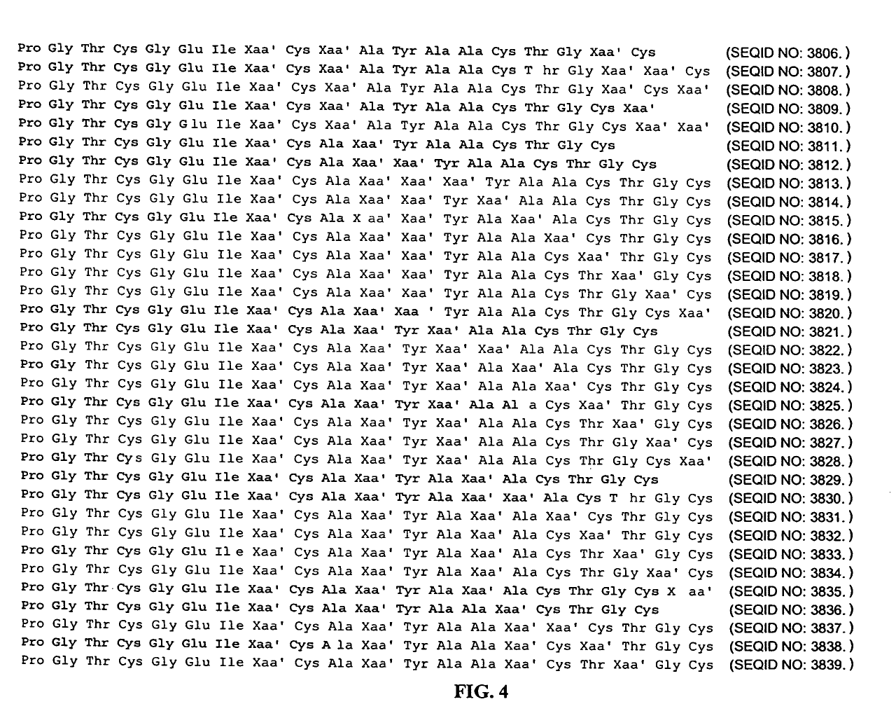

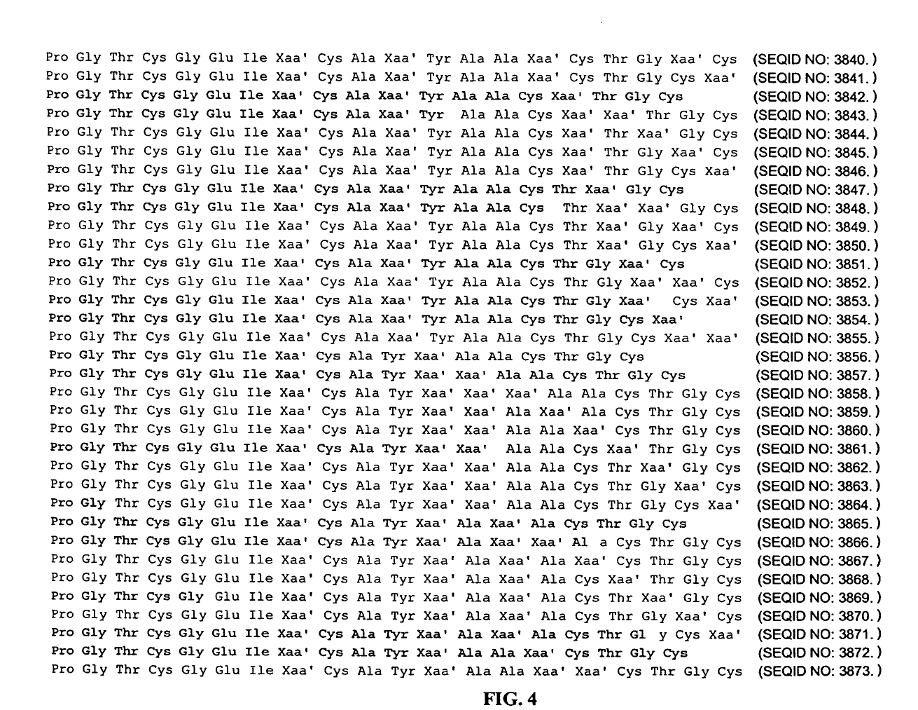

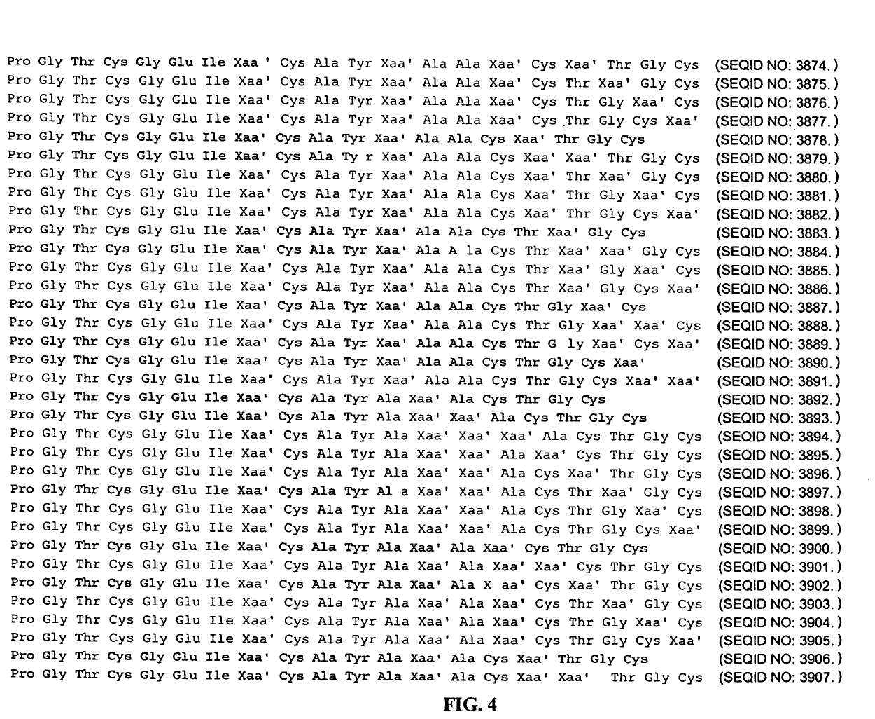

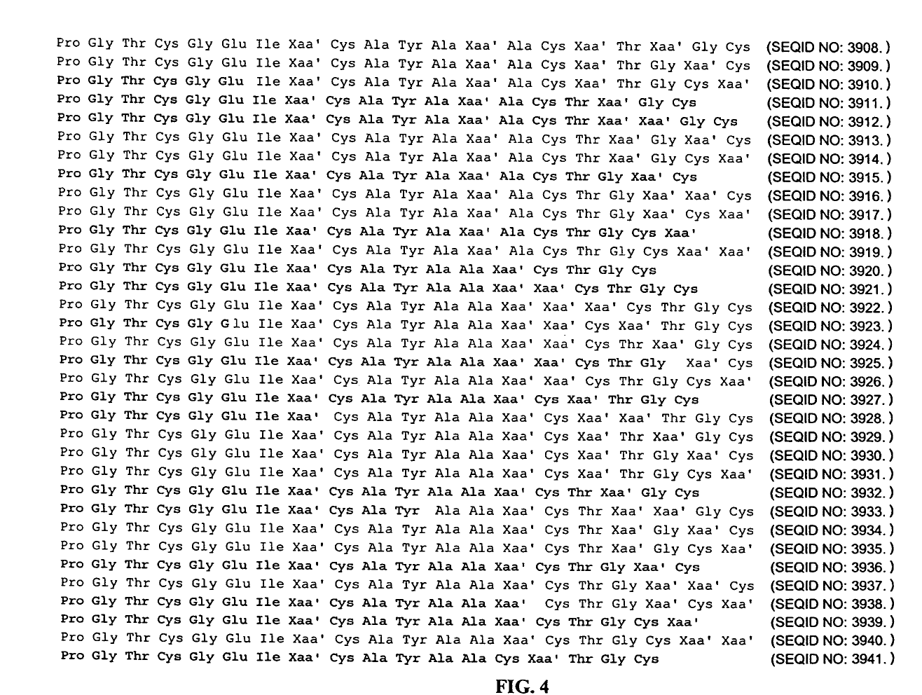

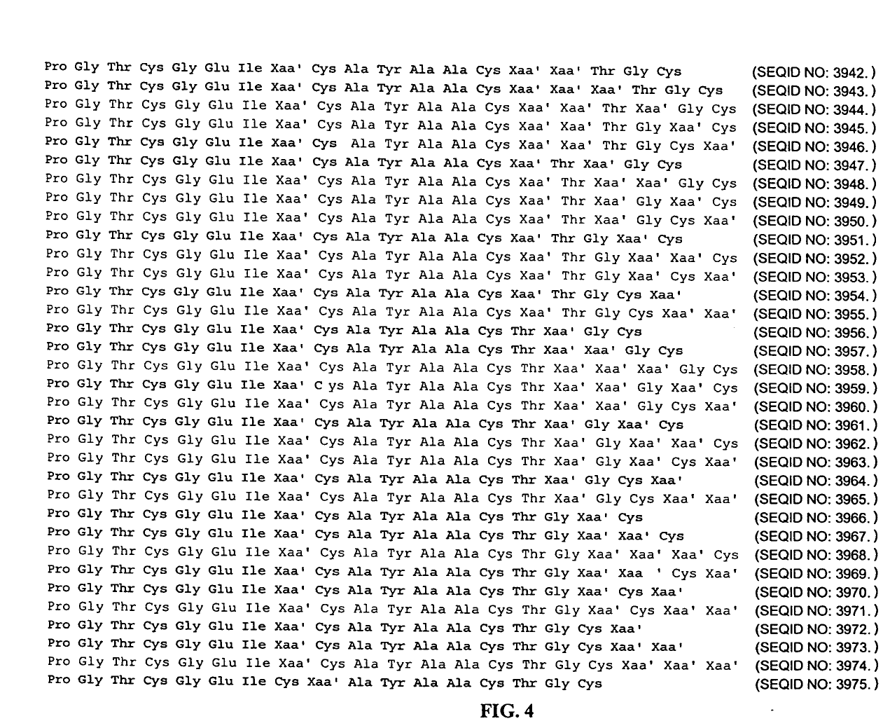

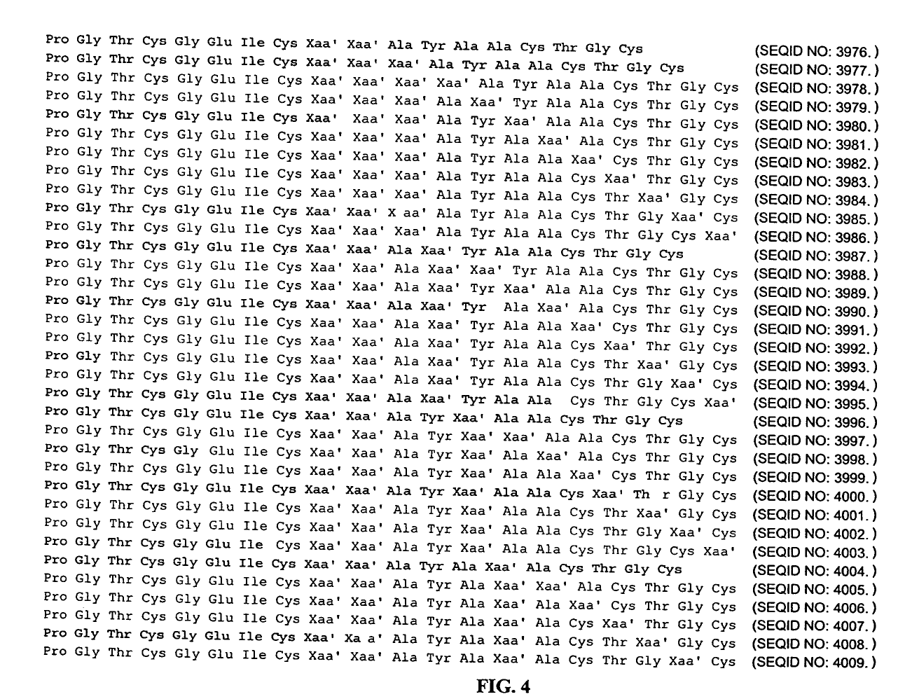

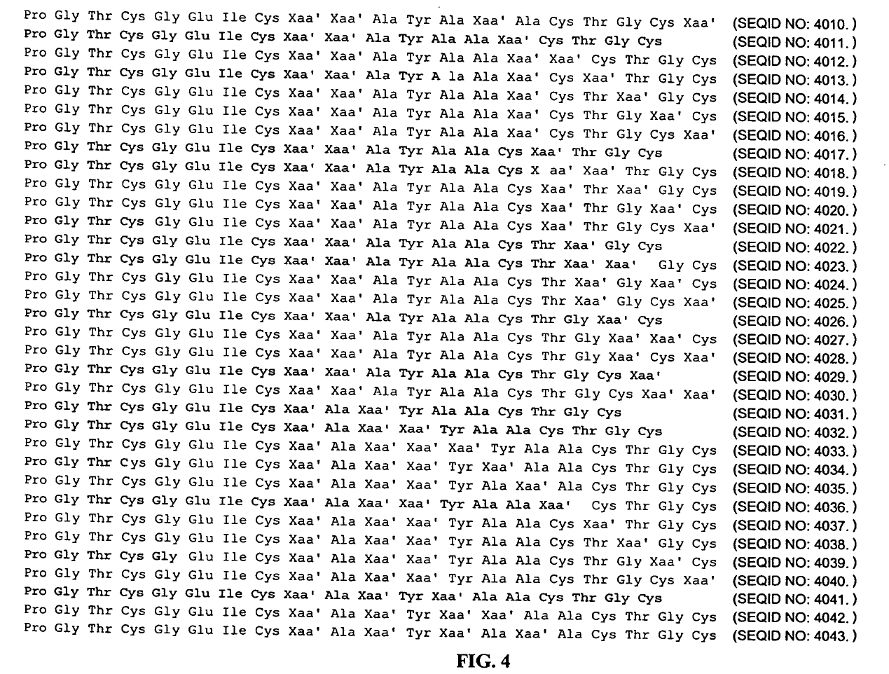

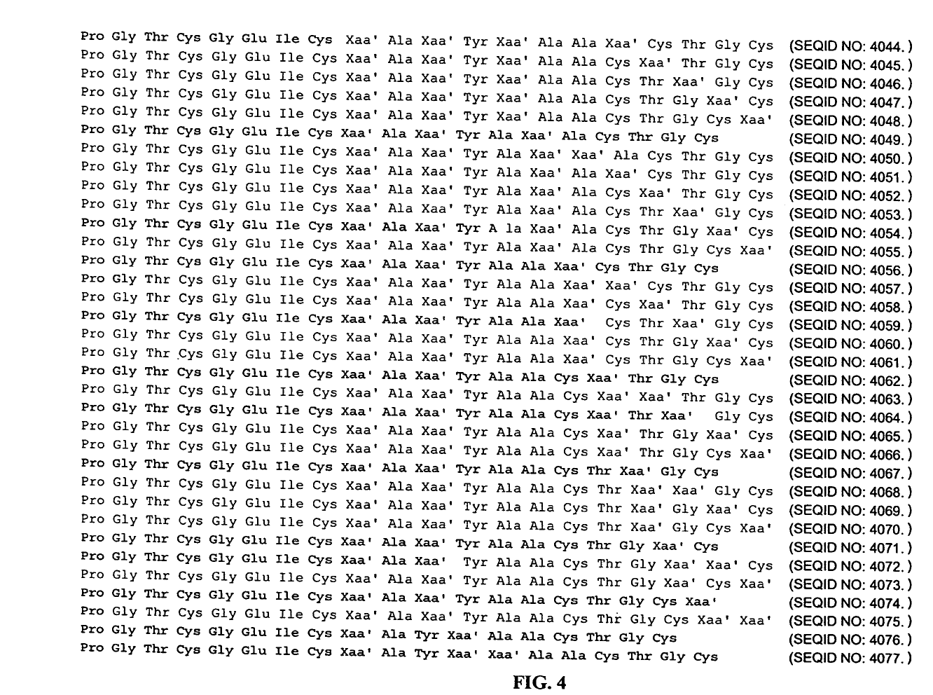

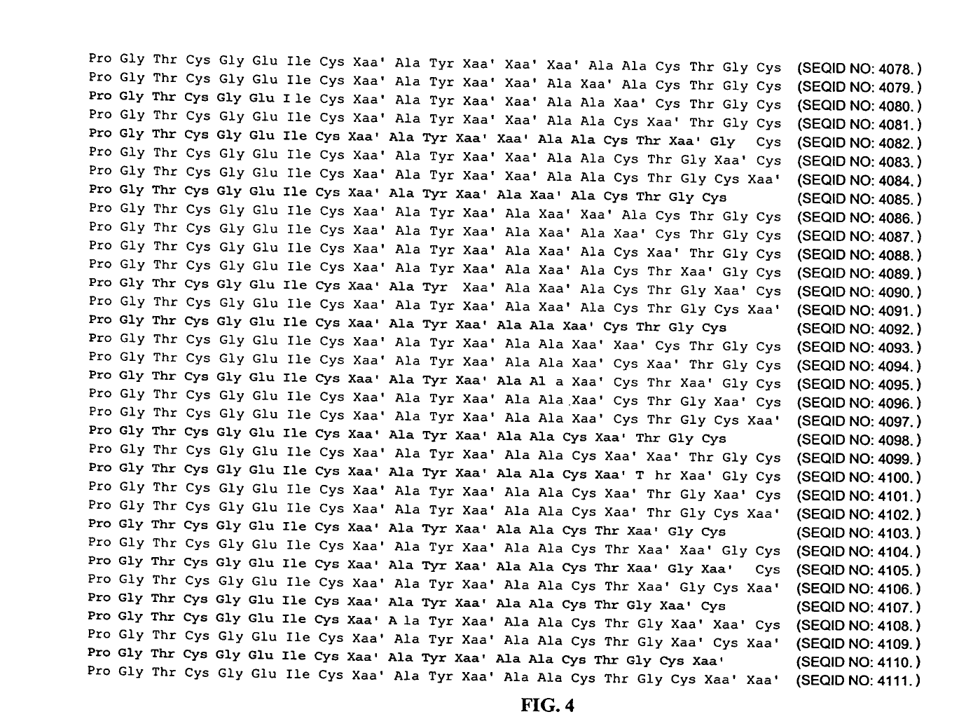

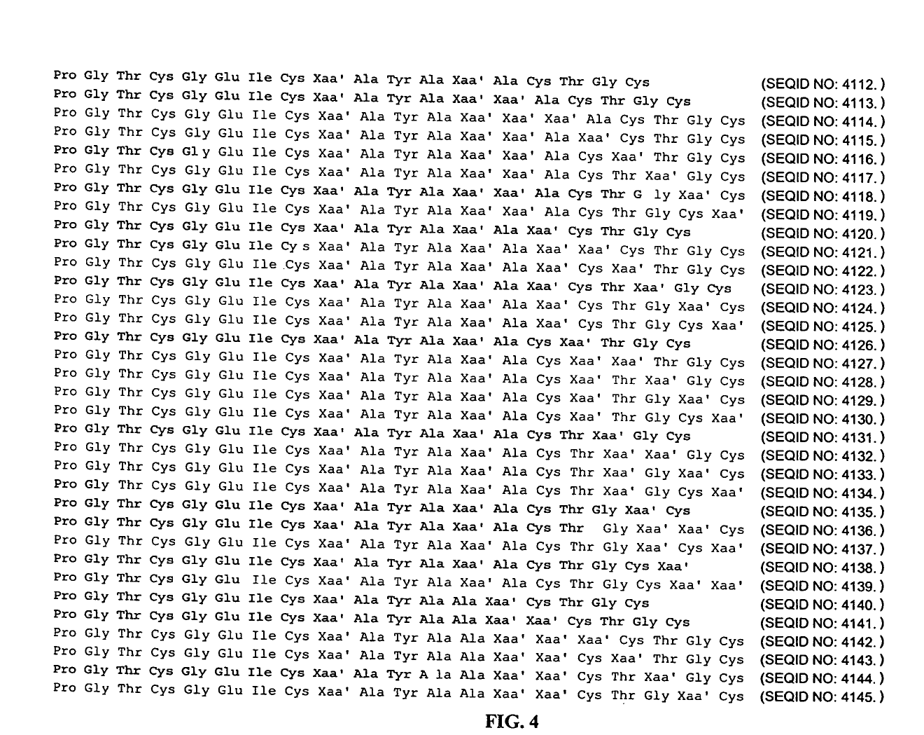

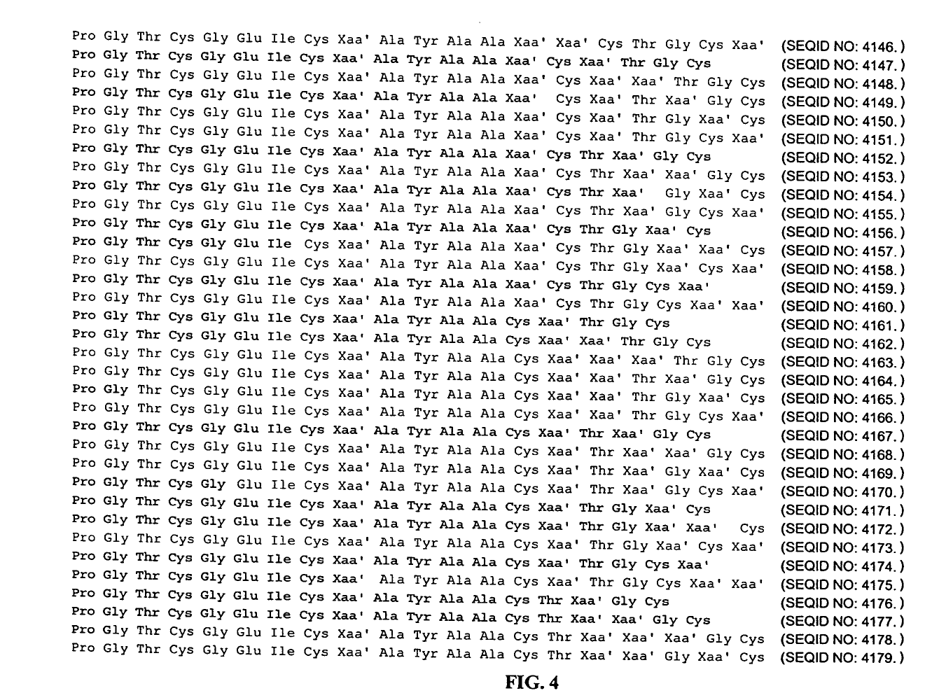

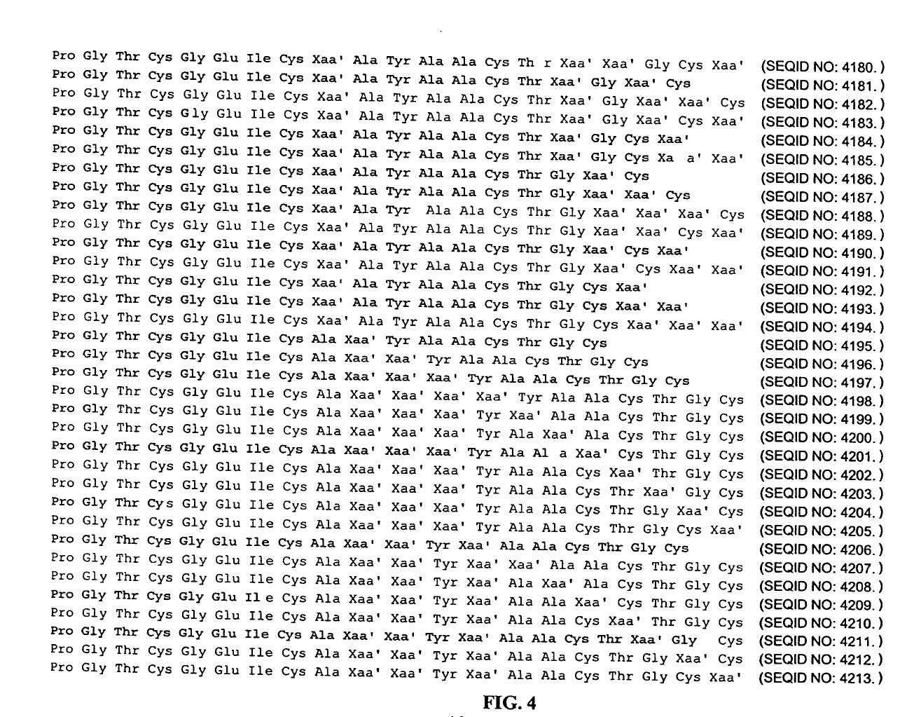

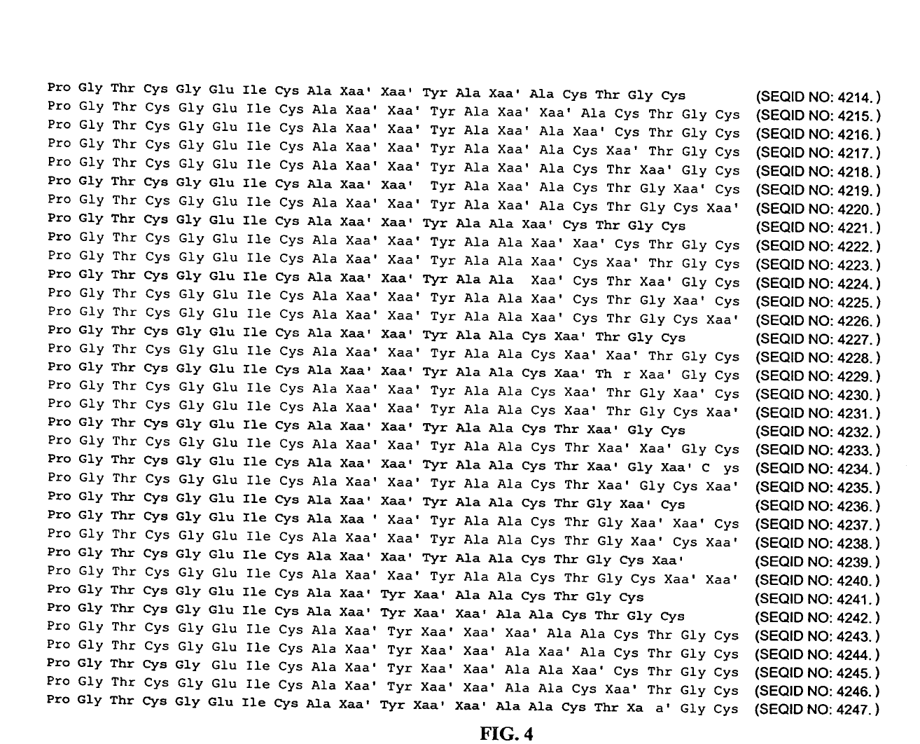

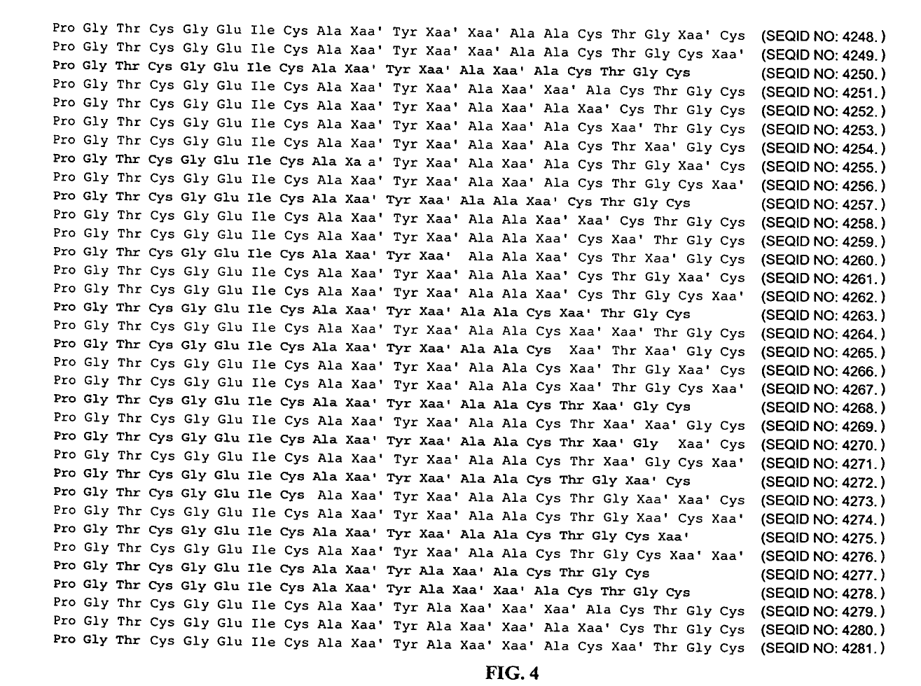

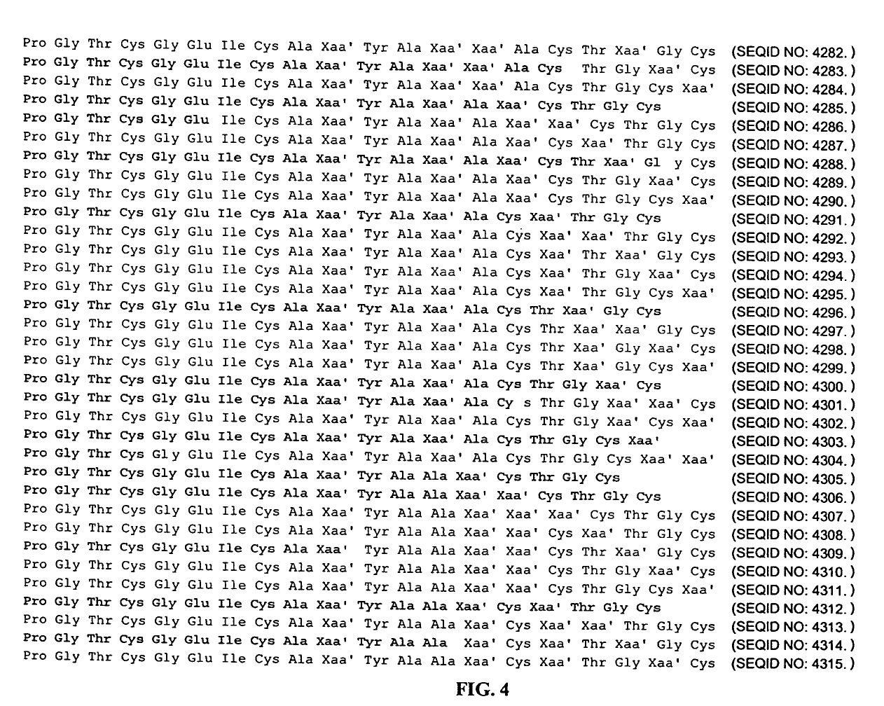

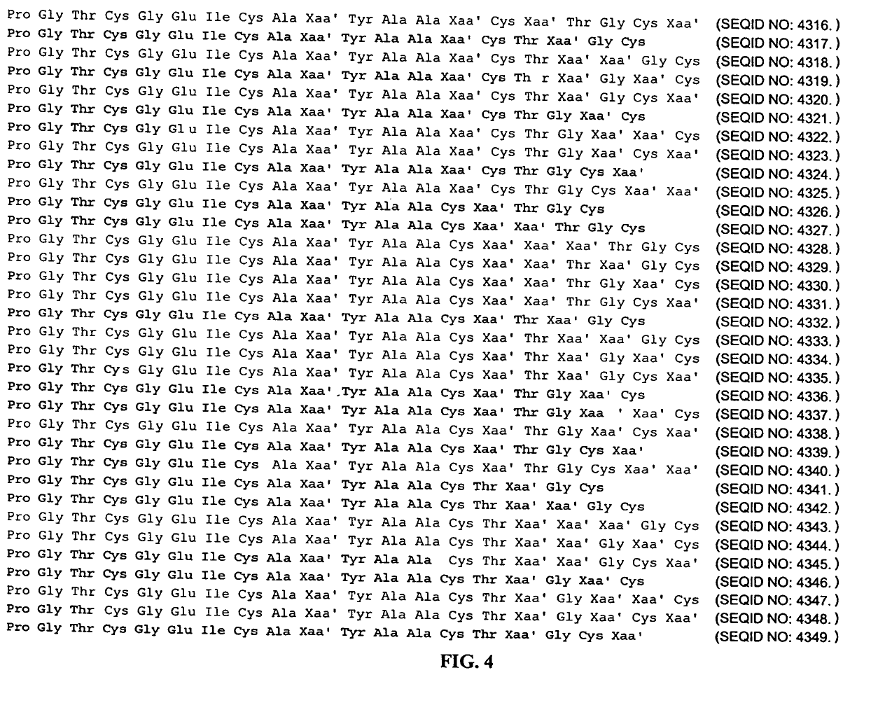

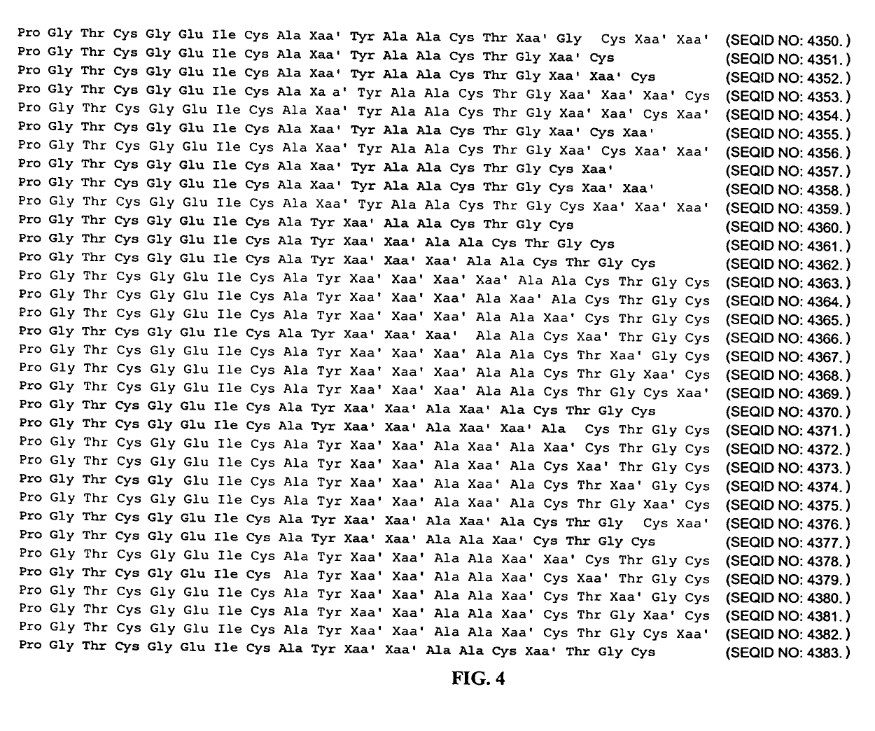

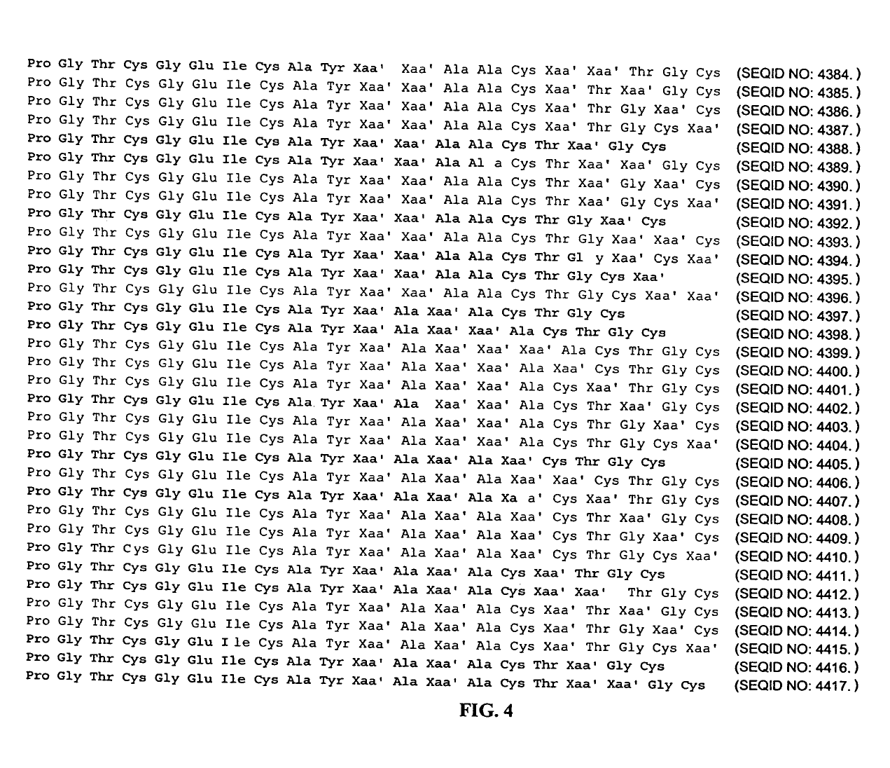

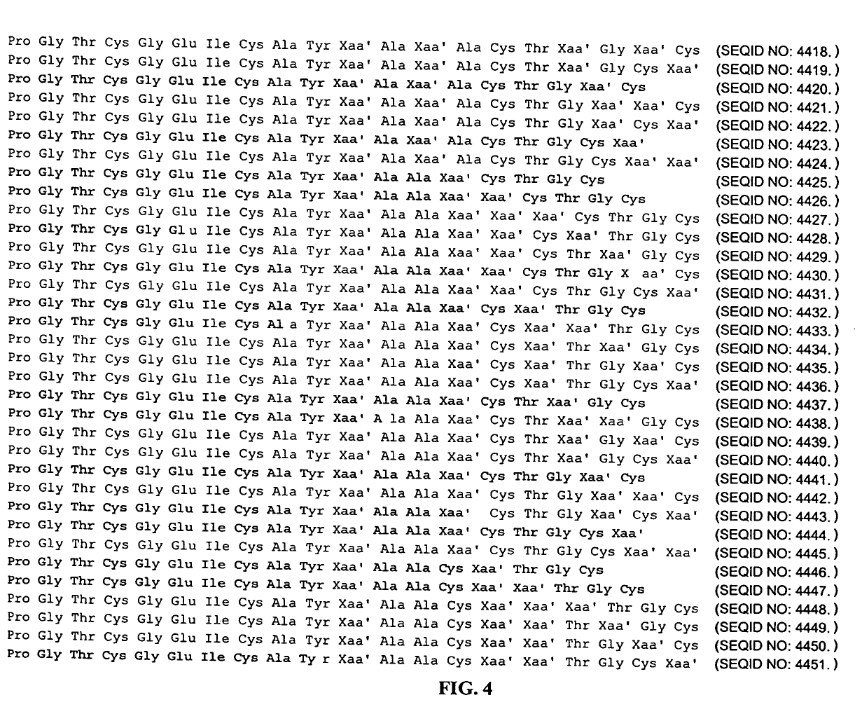

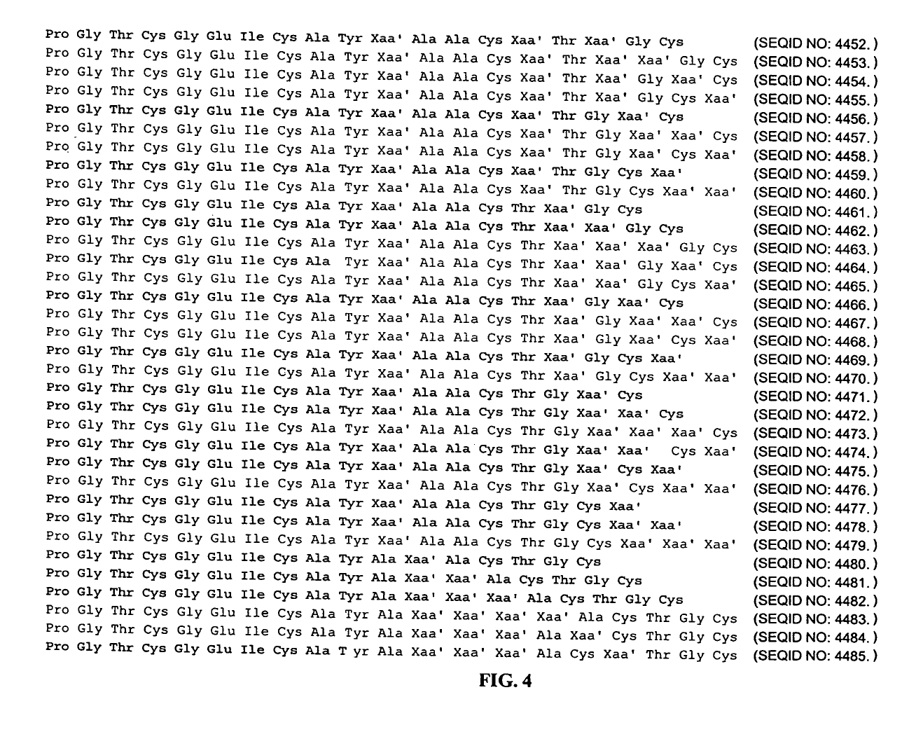

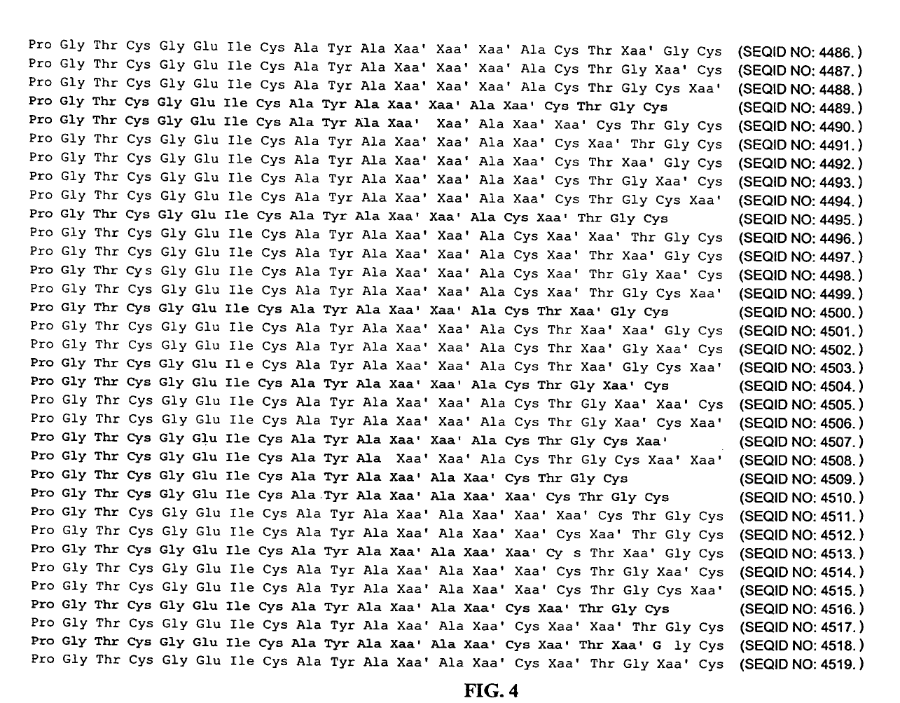

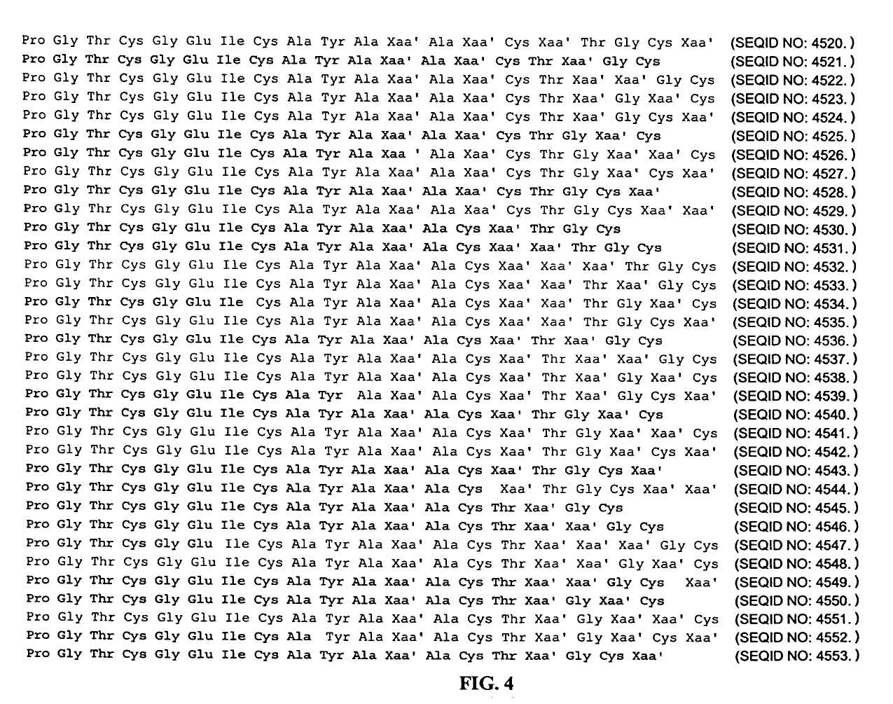

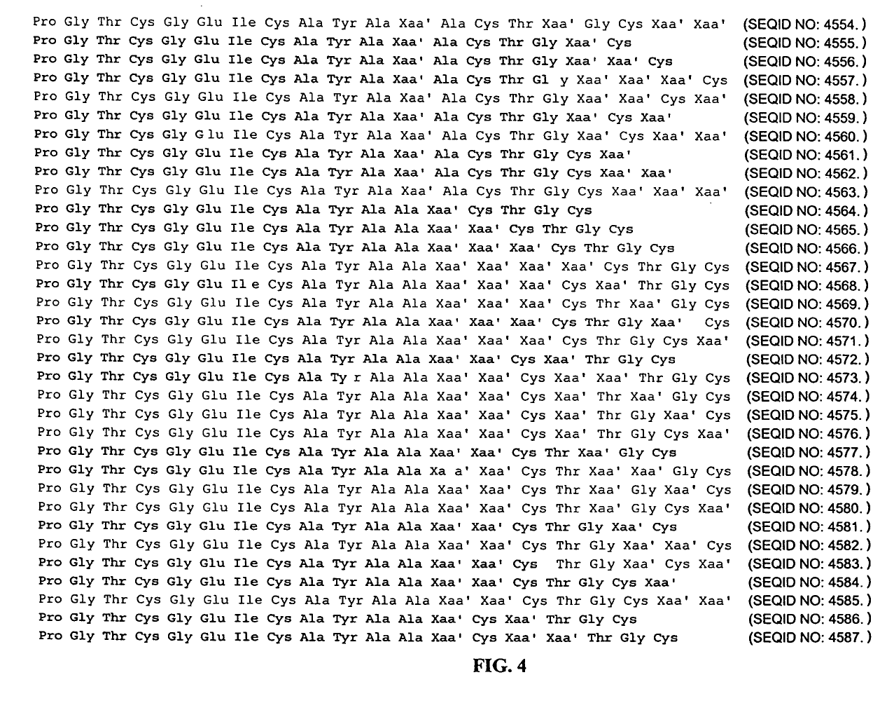

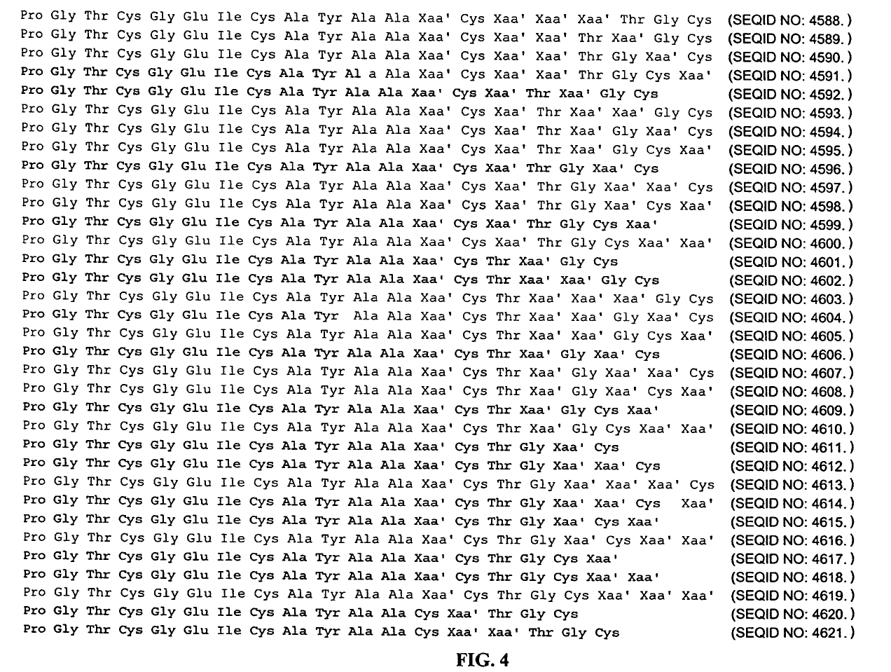

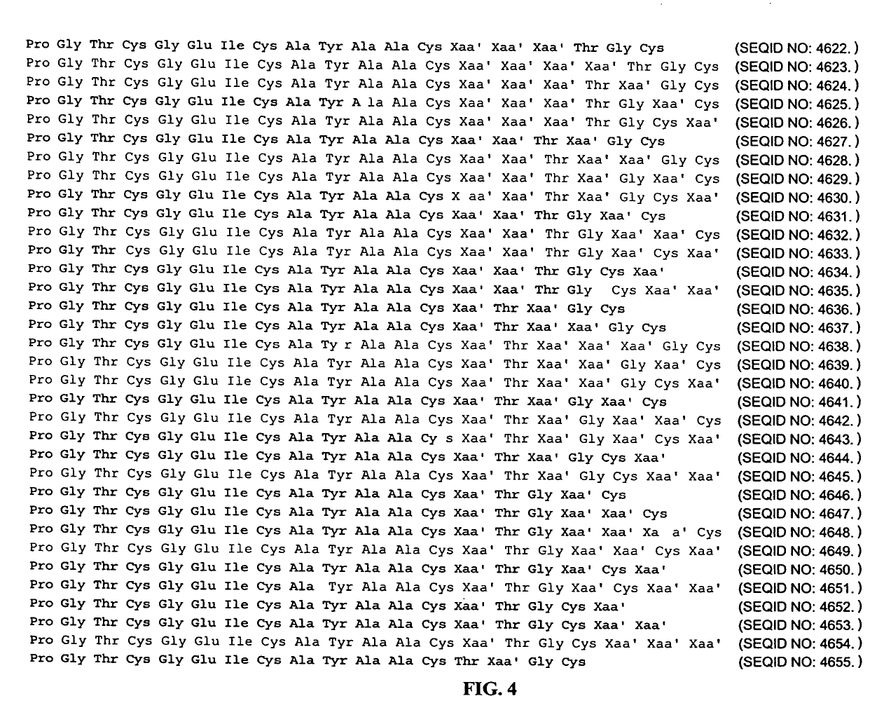

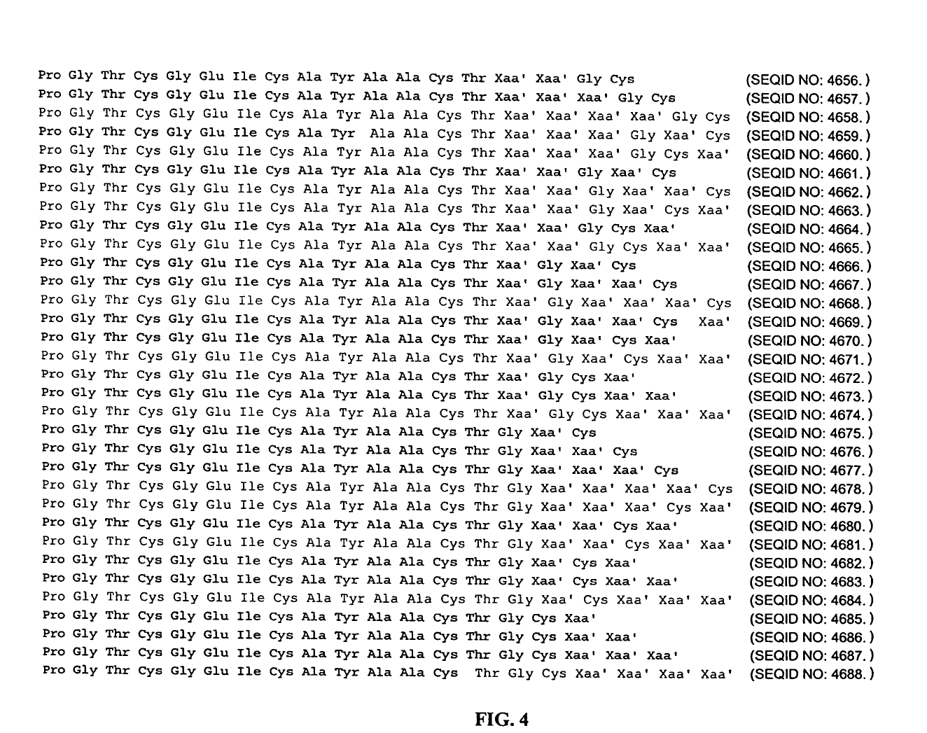

When it is desirable to potentiate the activity of guanylin by the administration of a chymotrypsin inhibitor or prodrug derivative thereof or some other guanylin potentiating agent, it may also be desirable to administer guanylin or a biologically active variant or fragment thereof. A human guanylin is depicted in FIG. 5 along with various other guanylins. A number of guanylin variants are depicted in FIGS. 3, 4 and 6.

As noted above, immature guanylin can include a pre-sequence and/or a prosequence. Immature guanylin is processed to yield the mature protein. Immature guanylin generally includes a so-called “pre sequence” followed by a “pro sequence” and then the mature polypeptide sequence. The pre sequence is important for secretion of the polypeptides. The pro sequence may be important for proper folding of the mature protein under at least some conditions. Various pre sequences and pro sequences are discussed in great detail below in the section describing guanylin and guanylin variants.

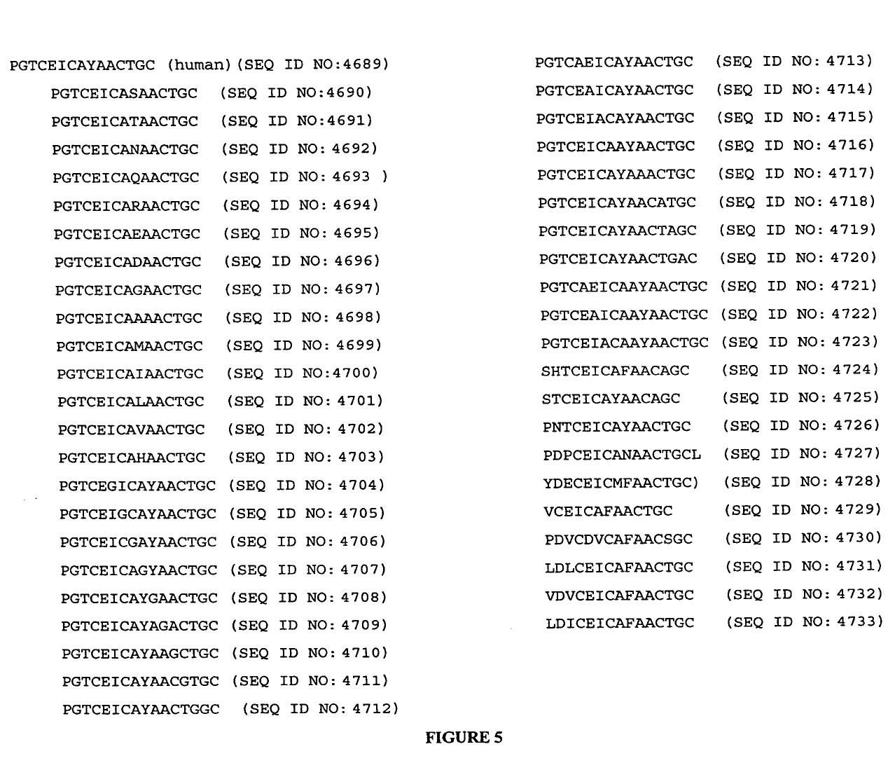

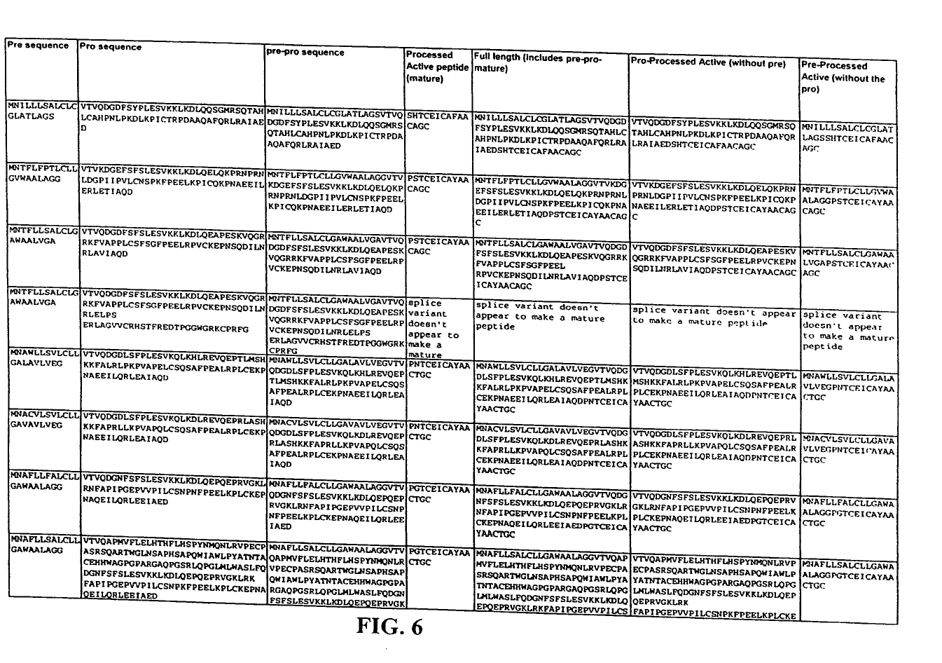

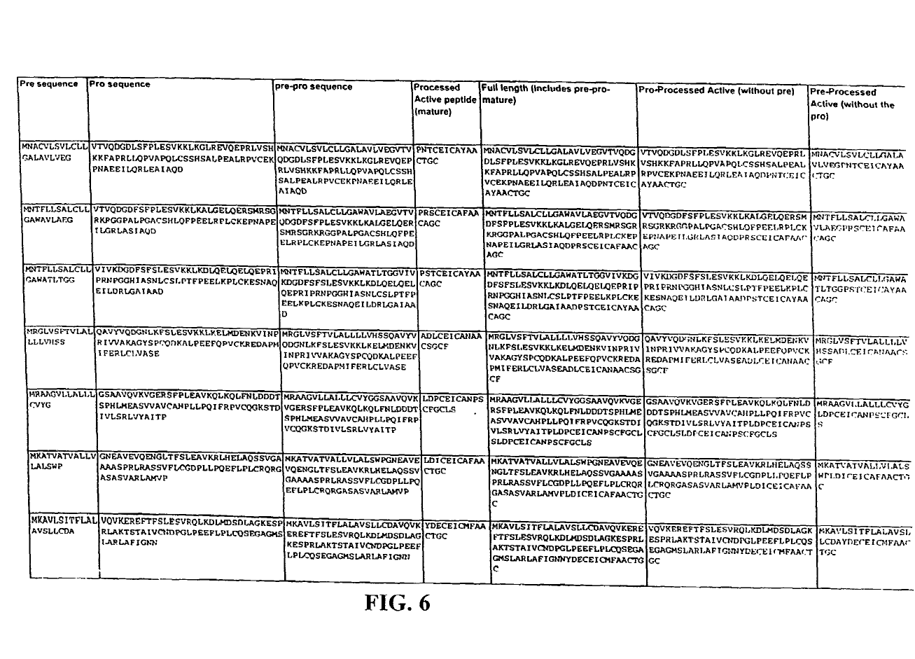

Mature guanylin has disulfide bonds between the first and third cysteines and between the second and fourth cysteines. The pro sequence of a guanylin is thought to be important for proper disulfide bond formation. Moreover, guanylin is thought to exist as an A-isomer and a B-isomer. The A- and B-isomers have the same disulfide bond connectivity but differ in three-dimensional conformation. It is thought that only the A-isomer has biological activity (see Lauber 2005 Protein and Peptide Letters 12:153). Pro sequences might be important for formation of the active A-isomer. In addition, such sequences can protect the mature polypeptide from premature degradation in the body or stabilize a particular isomer of the polypeptide. In some cases, such sequences may influence oligomerization. Accordingly, in some embodiments, a guanylin or gaunylin variant is produced and/or administered in a form that includes a pro sequence, a pre sequence or both a pre sequence and a pro sequence (a “prepro sequence”) at the amino terminus. Thus, useful polypeptides can include a pre sequence, a pro sequence or a prepro sequence preceding (amino-terminal to) a GC-C receptor agonist polypeptide described herein. FIG. 6 depicts the pre sequence (SEQ ID NOs: ______-______), pro sequence (SEQ ID NOs: ______-______), prepro sequence (SEQ ID NOs: ______-______), and mature sequence for a number guanylin polypeptides as well as various combinations thereof (e.g., a polypeptide consisting of a pre sequence and a mature polypeptide).

One or more of a pre sequence, a pro sequence and a prepro sequence can be present at the amino terminus of a GC-C receptor agonist polypeptide described herein. Thus, described herein are polypeptides comprising, consisting of or consisting essentially of (from amino terminus to carboxy terminus) one or more of: a pre sequence (SEQ ID NOs: ______-______; pre sequences; FIG. 6) and a pro sequence (SEQ ID NOs: ______-______; pro sequences; FIG. 6) followed by a GC-C receptor agonist polypeptide described herein, e.g., mature human guanylin. Among the useful GC-C receptor polypeptides that can modified by the addition of pre, pro, and/or prepro sequences are those depicted in the FIGS. 3-6 as well as those depicted below:

| | |
|---|---|
| PGTCEICASAACTGC | (SEQ ID NO: 4690) |
| PGTCEICATAACTGC | (SEQ ID NO: 4691) |
| PGTCEICANAACTGC | (SEQ ID NO: 4692) |
| PGTCEICAQAACTGC | (SEQ ID NO: 4693) |
| PGTCEICARAACTGC | (SEQ ID NO: 4694) |
| PGTCEICAEAACTGC | (SEQ ID NO: 4695) |
| PGTCEICADAACTGC | (SEQ ID NO: 4696) |
| PGTCEICAGAACTGC | (SEQ ID NO: 4697) |
| PGTCEICAAAACTGC | (SEQ ID NO: 4698) |
| PGTCEICAMAACTGC | (SEQ ID NO: 4699) |
| PGTCEICAIAACTGC | (SEQ ID NO: 4700) |
| PGTCEICALAACTGC | (SEQ ID NO: 4701) |
| PGTCEICAVAACTGC | (SEQ ID NO: 4702) |
| PGTCEICAHAACTGC | (SEQ ID NO: 4703) |
| PGTCEGICAYAACTGC | (SEQ ID NO: 4704) |
| PGTCEIGCAYAACTGC | (SEQ ID NO: 4705) |
| PGTCEICGAYAACTGC | (SEQ ID NO: 4706) |
| PGTCEICAGYAACTGC | (SEQ ID NO: 4707) |
| PGTCEICAYGAACTGC | (SEQ ID NO: 4708) |
| PGTCEICAYAGACTGC | (SEQ ID NO: 4709) |
| PGTCEICAYAAGCTGC | (SEQ ID NO: 4710) |
| PGTCEICAYAACGTGC | (SEQ ID NO: 4711) |
| PGTCEICAYAACTGGC | (SEQ ID NO: 4712) |
| PGTCAEICAYAACTGC | (SEQ ID NO: 4713) |
| PGTCEAICAYAACTGC | (SEQ ID NO: 4714) |
| PGTCEIACAYAACTGC | (SEQ ID NO: 4715) |
| PGTCEICAAYAACTGC | (SEQ ID NO: 4716) |
| PGTCEICAYAAACTGC | (SEQ ID NO: 4717) |
| PGTCEICAYAACATGC | (SEQ ID NO: 4718) |
| PGTCEICAYAACTAGC | (SEQ ID NO: 4719) |
| PGTCEICAYAACTGAC | (SEQ ID NO: 4720) |
| PGTCAEICAAYAACTGC | (SEQ ID NO: 4721) |
| PGTCEAICAAYAACTGC | (SEQ ID NO: 4722) |
| and | |
| PGTCEIACAAYAACTGC | (SEQ ID NO: 4723) |

In some cases it may be desirable to have a polypeptide that includes a pre sequence from a first guanylin polypeptide and a pro sequence from a second guanylin polypeptide. In other cases, the pre sequence and the pro sequence are from the same guanylin polypeptide.

Useful polypeptides can include a naturally-occurring or variant guanylin polypeptide in its mature form, as a prepro polypeptide (includes, from amino terminus to carboxy terminus, pre sequence, pro sequence and mature polypeptide), as a propolypeptide (includes, from amino terminus to carboxy terminus, pro sequence and mature polypeptide) or as a prepolypeptide (includes, from amino terminus to carboxy terminus, pre sequence and mature polypeptide). FIG. 6 depicts these various guanylin polypeptides. In some cases a polypeptide will be produced, e.g., recombinantly, with a pre sequence and/or a pro sequence. In certain cases the pre sequence and/or pro sequence is removed prior to administration of the polypeptide to a patient. In other cases the prepropolypeptide, propolypeptide or the prepolypeptide is administered to the patient. The pre sequence and/or the pro sequence may stabilize the polypeptide or an active isomer thereof, facilitate efficient folding of the polypeptide or protect the polypeptide from degradation in the patient's body. Thus, pre sequences, pro sequences and/or prepro sequences that do not significantly interfere with GC-C receptor agonist activity can be beneficial. In some cases the pre sequence and/or the pro sequence are removed by physiological processes after administration.

In some cases useful polypeptides will include only a portion (e.g., 20, 15, 12, 11, 10, 9, 8, 6, 5, 4 or fewer) of the amino acids of a pre sequence (SEQ ID NOs: ______-______), pro sequence (SEQ ID NOs: ______-______), prepro sequence (SEQ ID NOs: ______-______).

As can be seen in FIG. 6, pro sequences include Cys residues that may form a disulfide bond. For example, many pro sequences include two Cys residues separated by 12 amino acids. These Cys residues may form a disulfide bond. These Cys residues can be replaced by homocysteine, penicillamine, 3-mercaptoproline (Kolodziej et al. 1996 Int J Pept Protein Res 48:274); β, β-dimethylcysteine (Hunt et al. 1993 Int J Pept Protein Res 42:249) or diaminopropionic acid (Smith et al. 1978 J Med Chem 21:117) to form alternative internal cross-links at the positions of the normal disulfide bonds.

Activation of the Intestinal GC-C Receptor by Guanylin (T84 cGMP Assay)

The ability of guanylin to activate the intestinal GC-C receptor was assessed in an assay employing the T84 human colon carcinoma cell line (American Type Culture Collection (Bethesda, Md.). For the assays cells were grown to confluency in 24-well culture plates with a 1:1 mixture of Ham's F12 medium and Dulbecco's modified Eagle's medium (DMEM), supplemented with 5% fetal calf serum and were used at between passages 54 and 60.

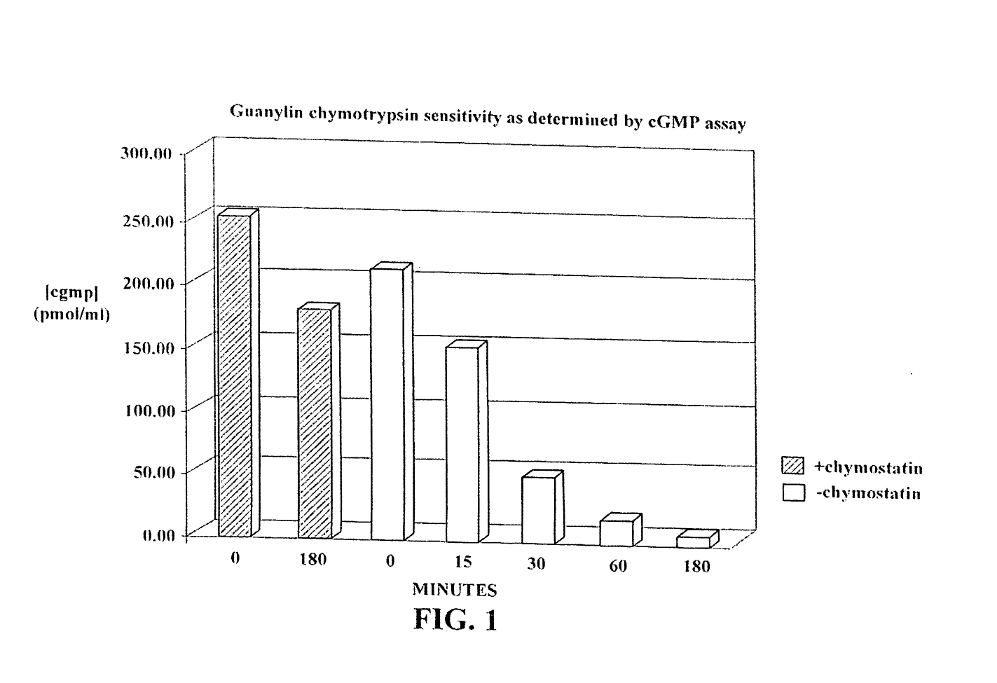

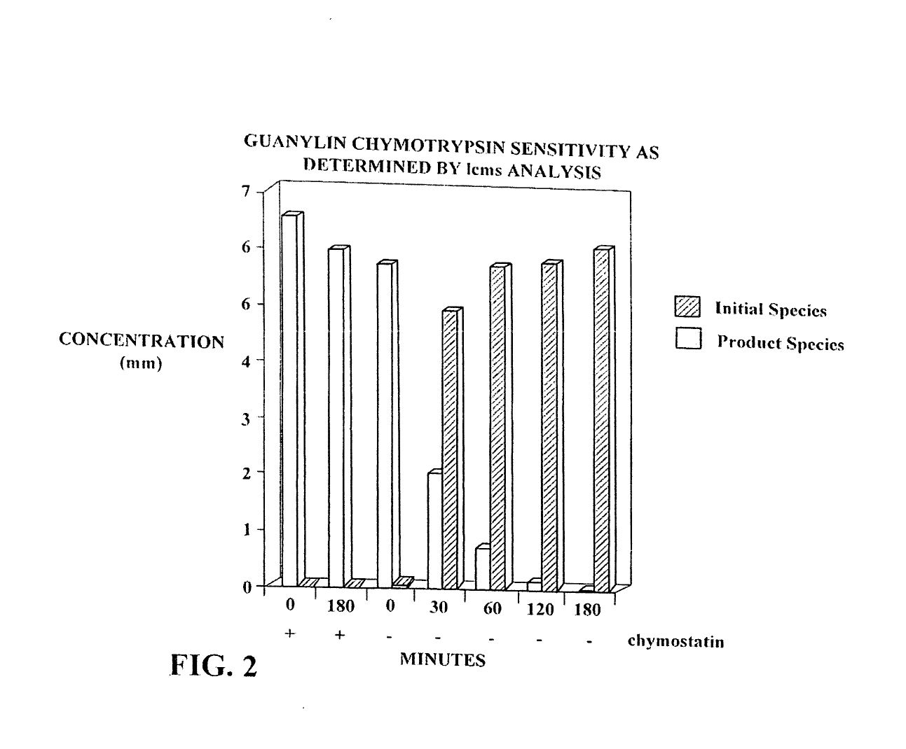

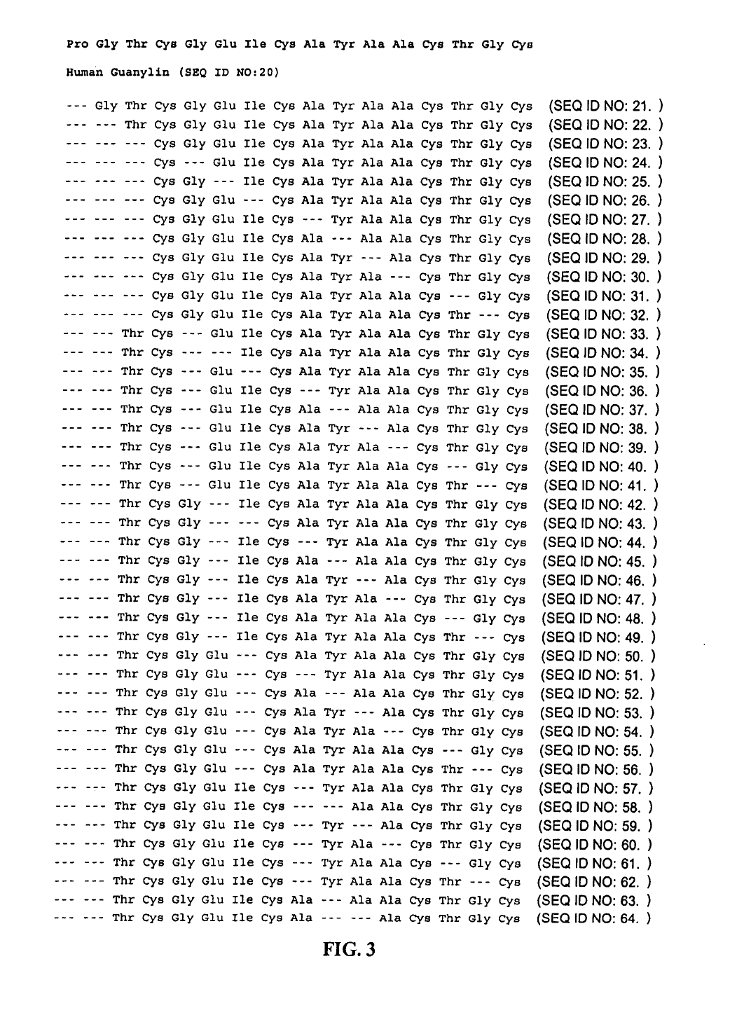

Briefly, monolayers of T84 cells in 24-well plates were washed twice with 1 ml/well DMEM, then incubated at 37° C. for 10 min with 0.45 ml DMEM containing 1 mM isobutylmethylxanthine (IBMX), a cyclic nucleotide phosphodiesterase inhibitor. Test peptides (50 μl) were then added and incubated for 30 minutes at 37° C. The media was aspirated and the reaction was then terminated by the addition of ice cold 0.5 ml of 0.1N HCl. The samples were held on ice for 20 minutes and then evaporated to dryness using a heat gun or vacuum centrifugation. The dried samples were resuspended in 0.5 ml of phosphate buffer provided in the Cayman Chemical Cyclic GMP EIA kit (Cayman Chemical, Ann Arbor, Mich.). Cyclic GMP was measured by EIA according to procedures outlined in the Cayman Chemical Cyclic GMP EIA kit. FIG. 1 shows that guanylin stimulated cGMP activity decreases over time in the presence of chymotrypsin. This activity was retained when the chymotrypsin inhibitor, chymostatin was present. FIG. 2 shows the results of LC/MS analysis of the processing of guanylin by chymotrypsin in the guanylin degradation assay.

### Intestinal GC-C Receptor Binding Assay

The ability of peptides and other agents to bind to the intestinal GC-C receptor can be tested as follows. Cells of the T84 human colon carcinoma cell line (American Type Culture Collection (Bethesda, Md.) are grown to confluence in 24-well culture plates with a 1:1 mixture of Ham's F12 medium and Dulbecco's modified Eagle's medium (DMEM), supplemented with 5% fetal calf serum. Cells used in the assay are typically between passages 54-60. Briefly, T84 cell monolayers in 24-well plates are washed twice with 1 ml of binding buffer (DMEM containing 0.05% bovine serum albumin and 25 mM HEPES, pH 7.2), then incubated for 30 min at 37° C. in the presence of mature radioactively labeled E. coli ST peptide and the test material at various concentrations. The cells are then washed four times with 1 ml of DMEM and solubilized with 0.5 ml/well 1N NaOH. The level of radioactivity in the solubilized material is then determined using standard methods.

In some cases, intestinal epithelial cell preparations may be used instead of T84 cells to assess receptor binding.

### Murine Gastrointestinal Transit (GIT) Assay

In order to determine if anagent has an effect on intestinal motility, the agent can be tested in the murine gastrointestinal transit (GIT) assay (Moon et al. Infection and Immunity 25:127, 1979). This assay can also be used to determine the effect of an agent of the invention on intestinal motility. In this assay, charcoal, which can be readily visualized in the gastrointestinal tract is administered to mice after the administration of a test compound. The distance traveled by the charcoal is measured and expressed as a percentage of the total length of the colon.

Mice are fasted with free access to water for 12 to 16 hours before the treatment with peptide or control buffer. A test agent is orally administered in buffer (20 mM Tris pH 7.5) seven minutes before being given an oral dose of 5% Activated Carbon (Aldrich 242276-250G). Control mice are administered buffer only before being given a dose of Activated Carbon. After 15 minutes, the mice are sacrificed and their intestines from the stomach to the cecum are dissected. The total length of the intestine as well as the distance traveled from the stomach to the charcoal front is measured for each animal and the results are expressed as the percent of the total length of the intestine traveled by the charcoal front. Results are reported as the average of 10 mice±standard deviation. A comparison of the distance traveled by the charcoal between the mice treated with the agent versus the mice treated with vehicle alone is performed using a Student's t test and a statistically significant difference is considered for P<0.05. Positive controls for this assay may include Zelnorm®, a drug approved for IBS that is an agonist for the serotonin receptor 5HT4.

### Suckling Mouse Model of Intestinal Secretion (SuMi Assay)

Guanylin potentiating agentscan be tested for their ability to increase intestinal secretion using a suckling mouse model of intestinal secretion. In this model a test compound is administered to suckling mice that are between seven and nine days old. After the mice are sacrificed, the gastrointestinal tract from the stomach to the cecum is dissected (“guts”). The remains (“carcass”) as well as the guts are weighed and the ratio of guts to carcass weight is calculated. If the ratio is above 0.09, one can conclude that the test compound increases intestinal secretion. Controls for this assay may include Zelnorm®

### Phenylbenzoquinone-Induced Writhing Model

The PBQ-induced writhing model can be used to assess whether administration of a an agent reduces pain. This model is described by Siegmund et al. (1957 Proc. Soc. Exp. Bio. Med. 95:729-731). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5^{th} to the 10^{th} minute after PBQ injection, and can also be counted between the 35^{th} and 40^{th} minute and between the 60^{th} and 65^{th} minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

Effect on Bowel Habits

Guanylin potentiating agent of the invention can be administered to mammals (e.g. humans) to determine the effect on bowel habits (including Bristol Stool Form Scale score, stool frequency (number of stools per week), ease of passage and stool weight). A guanylin potentiating agent of the invention is administered in a single dose or multiple doses (for example, once daily over a consecutive 7 day period) and alterations in bowel habit are evaluated (for each collected bowel movement), for example, prior to dose, during dosage (for multiple dosing), and postdose.

The Bristol Stool Form Scale is: 1: Separate hard lumps, like nuts

2: Sausage-shaped but lumpy

3: Like a sausage or snake but with cracks on its surface

4: Like a sausage or snake, smooth and soft

5: Soft blobs with clear-cut edges

6: Fluffy pieces with ragged edges, a mushy stool

7: Watery, no solid pieces

The scale used to determine ease of passage is:

1. Manual disimpaction

2 Enema needed

3. Straining needed

4. Normal

5. Urgent without pain

6. Urgent with pain

7. Incontinent

Rat Model of Postoperative Ileus.

Female CD rats are used to test the effect of guanylin potentiating agents of the invention on delayed transit induced by abdominal surgery and manual manipulation of the small intestine. Groups of at least nine rats undergo abdominal surgery under isoflurane anesthesia. Surgery consists of laparotomy and 5 minutes of gentle manual intestinal massage. Following recovery from anesthesia, rats are dosed orally with either guanylin potentiating agent (for example, 10 μg/kg) of the invention or vehicle (20 mM Tris) in a volume of 300 μl. 1 hour after dosing, intestinal transit rate is measured. Animals are again dosed with 300 μl of the test article followed immediately by 500 μl of a charcoal meal (10% charcoal, 10% gum arabic in water). To calculate the distance of the small intestine traveled by the charcoal front, after 20 minutes, the total length of the intestine as well as the distance traveled from the stomach to the charcoal front are measured for each animal.

Effect on cGMP Levels and Secretion in Ligated Loops Rodent Models

The effect of guanylin potentiating agents of the invention on cGMP levels and secretion are studied by injecting guanylin potentiating agents of the invention directly into an isolated loop in either wild-type or GC-C KO mice. This is done by surgically ligating a loop in the small intestine of the mouse. The methodology for ligated loop formation is similar to that described in London et al. 1997 Am J Physiol p. G93-105. The loop is roughly centered and is a length of 1-3 cm. The loops are injected with 100 μl of either pontentiating agent or vehicle (20 mM Tris, pH 7.5 or Krebs Ringer, 10 mM Glucose, HEPES buffer (KRGH)). Following a recovery time of 90 minutes the loops are excised. Weights are recorded for each loop before and after removal of the fluid contained therein. The length of each loop is also recorded. A weight to length ratio (W/L) for each loop is calculated to determine the effects of the guanylin potentiating agent of the invention on secretion.

To determine the effect of the guanylin potentiating agent of the invention on cGMP activity, fluid from the loop is collected in ice-cold trichloracetic acid (TCA) and stored at −80° C. for use in an assay to measure cGMP levels in the fluid. Intestinal fluid samples are TCA extracted, and cyclic GMP is measured by EIA according to procedures outlined in the Cayman Chemical Cyclic GMP EIA kit (Cayman Chemical, Ann Arbor, Mich.) to determine cyclic GMP levels in the intestinal fluid of the mouse in the presence of either guanylin potentiating agent of the invention or vehicle.

The effects of guanylin potentiating agents of the invention on cGMP levels and secretion in ligated loops in female CD rats can also be determined using protocols similar to those described above. In the case of the rat, however four loops of intestine are surgically ligated. The first three loops are distributed equally in the small intestine and the fourth loop is located in colon. Loops are 1 to 3 centimeters, and are injected with 200 μL of either peptide/agonist of the invention (5 μg) or vehicle (Krebs Ringer, 10 mM glucose, HEPES buffer (KRGH)).

Effect on Diuresis and Natriuresis

The effect of guanylin potentiating agents of the invention on diuresis and natriuresis can be determined using methodology similar to that described in WO06/001931 (examples 6 (p. 42) and 8 (p. 45)). Briefly, the guanylin potentiating agent of the invention (180-pmol) is infused for 60 min into a group of 5 anesthetized rats. Given an estimated rat plasma volume of 10 mL, the infusion rate is approximately 3 pmol/mL/min. Blood pressure, urine production, and sodium excretion are monitored for approximately 40 minutes prior to the infusion, during the infusion, and for approximately 50 minutes after the infusion to measure the effect of the peptide/GC-C agonist on diuresis and natriuresis. For comparison, a control group of five rats is infused with regular saline. Urine and sodium excretion can be assessed. Dose response can also be determined. Guanylin potentiating agent of the invention is infused intravenously into rats over 60 minutes. Urine is collected at 30 minute intervals up to 180 minutes after termination of guanylin potentiating agent infusion, and urine volume, sodium excretion, and potassium excretion are determined for each collection interval. Blood pressure is monitored continuously. For each dose a dose-response relationship for urine volume, sodium and potassium excretion can be determined. Plasma concentration of the peptide/GC-agonist is also determined before and after iv infusion.

### Colonic Hyperalgesia Animal Models

Hypersensitivity to colorectal distension is a common feature in patients with IBS and may be responsible for the major symptom of pain. Both inflammatory and non-inflammatory animal models of visceral hyperalgesia to distension have been developed to investigate the effect of compounds on visceral pain in IBS and can be used to assess the impact of a compound such as a chymotrypsin inhibitor or other agent administered alone with guanylin or biologically active variant or fragment thereof.

I. Trinitrobenzenesulphonic Acid (TNBS)-Induced Rectal Allodynia Model

Male Wistar rats (220-250 g) are premedicated with 0.5 mg/kg of acepromazine injected intraperitoneally (IP) and anesthetized by intramuscular administration of 100 mg/kg of ketamine. Pairs of nichrome wire electrodes (60 cm in length and 80 μm in diameter) are implanted in the striated muscle of the abdomen, 2 cm laterally from the white line. The free ends of electrodes are exteriorized on the back of the neck and protected by a plastic tube attached to the skin. Electromyographic (EMG) recordings are started 5 days after surgery. Electrical activity of abdominal striated muscle is recorded with an electroencephalograph machine (Mini VIII, Alvar, Paris, France) using a short time constant (0.03 sec.) to remove low-frequency signals (<3 Hz).

Ten days post surgical implantation, trinitrobenzenesulphonic acid (TNBS) is administered to induce rectal inflammation. TNBS (80 mg kg⁻¹ in 0.3 ml 50% ethanol) is administered intrarectally through a silicone rubber catheter introduced at 3 cm from the anus under light diethyl-ether anesthesia, as described (Morteau et al. 1994 Dig Dis Sci 39:1239). Following TNBS administration, rats are placed in plastic tunnels where they are severely limited in mobility for several days before colorectal distension (CRD). Experimental compound is administered one hour before CRD which is performed by insertion into the rectum, at 1 cm of the anus, a 4 cm long balloon made from a latex condom (Gue et al, 1997 Neurogastroenterol. Motil. 9:271). The balloon is fixed on a rigid catheter taken from an embolectomy probe (Fogarty). The catheter attached balloon is fixed at the base of the tail. The balloon, connected to a barostat is inflated progressively by step of 15 mmHg, from 0 to 60 mmHg, each step of inflation lasting 5 min. Evaluation of rectal sensitivity, as measured by EMG, is performed before (1-2 days) and 3 days following rectal instillation of TNBS.

The number of spike bursts that corresponds to abdominal contractions is determined per 5 min periods. Statistical analysis of the number of abdominal contractions and evaluation of the dose-effects relationships is performed by a one way analysis of variance (ANOVA) followed by a post-hoc (Student or Dunnett tests) and regression analysis for ED50 if appropriate.

II. Stress-Induced Hyperalgesia Model

Male Wistar Rats (200-250 g) are surgically implanted with nichrome wire electrodes as in the TNBS model. Ten days post surgical implantation, partial restraint stress (PRS), is performed as described by Williams et al. for two hours (Williams et al. 1988 Gastroenterology 64:611). Briefly, under light anaesthesia with ethyl-ether, the foreshoulders, upper forelimbs and thoracic trunk are wrapped in a confining harness of paper tape to restrict, but not prevent body movements. Control sham-stress animals are anaesthetized but not wrapped. Thirty minutes before the end of the PRS session, the animals are administered test-compound or vehicle. Thirty minutes to one hour after PRS completion, the CRD distension procedure is performed as described above for the TNBS model with barostat at pressures of 15, 30, 45 and 60 mm Hg. Statistical analysis on the number of bursts is determined and analyzed as in the TNBS model above.

### TNBS Colitis in Rats

This model is described in, for example, Morris et al. (Gastroenterology 96(3):795-803, 1989). Briefly, to induce chronic colonic inflammation in rats, a rubber catheter is inserted rectally into the colon such that the tip is 8 cm proximal to the anus. Next, 2,4,6-Trinitrobenzenesulfonic acid (TNBS 5-30 mg) dissolved in 50% ethanol is instilled into the lumen of the colon through the rubber catheter. Rats are euthanized at various times (24 hours and 1-8 weeks) following rectal TNBS administration and the colon tissue is examined for damage, inflammation and ulceration. Colon weight and colonic myeloperoxidase (MPO) activity are also assessed. Complete protocol details for an alternative model can be found in Dohi et al. (Gastroenterology 119:724-733, 2000). Briefly, mice (C57BL/6; 40 μg/g and Balb/c 36 μg/g) are given a solution of TNBS dissolved in a mixture of phosphate-buffered saline and then mixed with an equal volume of ethanol for a final concentration of 2% TNBS in 50% ethanol. On days 0 and 7, the TNBS enema is administered to mice anesthetized with ketamine and xylazine via a glass microsyringe equipped with a gastric intubation needle. Tissues and cells are assessed 3 days later (day 10).

### Oxazolone Colitis in Mice

This model is described in, for example, Kojima et al. (J. Pharmacol. Sci. 96:307-313, 2004). Briefly, a metal catheter is inserted 4 cm into the lumen of the colon via the anus in the anesthetized mouse. Oxazolone solution (0.15 mL/mouse) is administered into the colon through the catheter. Colonic tissues from mice on days 0 (before colitis induction), 1, 2, 4 and 7 are collected and examined for evidence of colitis and myeloperoxidase (MPO) activity.

### Colitis Models

In general any model of colitis can be used, in particular, mouse or rat models in which a chemical, hapten or antigen is used to induce colitis. Other colitis models are described Elson et al. (Gastroenterology 109:1344-1367, 1995) and Kim et al. (Scand. J. Gastroenterol 27:529-537, 1992).

### Oral Antigen-Induced Gastrointestinal Allergy in Mice

Complete protocol details for one model can be found in, for example, Hogan et al. (Nat Immunol. 2(4):353-60, 2001). Briefly, mice are sensitized by intraperitoneal injection with ovalbumin (50 μg) in alum (1 mg) in 0.9% sterile saline on day 0. On days 12 and 15, mice are orally administered with encapsulated ovalbumin or placebo enteric-coated beads (20 mg) followed by oral administration of acidified water (300 μl, pH 2.0). In some experiments, mice are intragastrically challenged with soluble ovalbumin (1 mg) in PBS (200 μl) or control PBS on days 12 and 15. Mice are euthanized and parmeteres are measured 72 hours after the last antigen challenge. The gastrointestinal tract tissue is examined for eosinophilic inflammation. Complete protocol details for another model can be found in Forbes et al. (Gastroenterology 127:105-118, 2004). Briefly, mice are sensitized by an intraperitoneal injection of 50 μg of ovalbumin/1 mg of alum in 200 μL of 0.9% sterile saline on day 0. On days 12, 14, and 16, mice are orally administered 20 mg of either encapsulated ovalbumin enteric coated beads or placebo beads, followed by 200 uL of acidified water 9, pH 2.0). 72 hours after the last antigen challenge, mice are euthanized and disease parameters are measured in various ways. In some experiments, mice are intraperitoneally injected on days 0, 1, and 3 with either rat IgG2b-depleting anti-D4 onoclonal antibody or rat IgG control antibody. Methacholine-induced bronchial hyperresponsiveness is determined on day 4.

### Experimental Oral Allergen-Induced Diarrhea

Complete protocol details can be found in, for example, Brandt et al. (J. Clin. Invest. 112(11):1666-1677, 2003). Briefly, mice are sensitized twice, 2 weeks apart, with 50 μg of ovalbumin/1 mg of aluminum potassium sulfate adjuvant by intraperitoneal injection. Two weeks later, mice are held in the supine position 3 times a week and orally administered 250 μL of sterile saline that contains up to 50 mg of ovalbumin. Before each intragastric challenge, mice are deprived of food for 3-4 hours with the aim of limiting antigen degraduation in the stomach. Diarrhea is assessed by visually monitoring mice for up to 1 hour following intragastric challenge.

### Animal Models of Hypertension

Various animal models of hypertenstion can be used to screen guanylin potentiating agents. In general, hypertension can be induced in rats in at least four ways, including: genetically-induced, environmentally-induced, pharmacologically-induced, and renal-induced. A variety of rodent hypertension models are described in Pinto et al. (1998 Cardiovascular Research 39:77-88) and the references cited therein. One of the most widely used rodent models of hypertension is the Spontaneously Hypertensive Rat (SHR). Other models include: (1) the two-kidney one-clip, (2) transgenic rats overexpressing the murine Ren2 gene, (3) the Dahl salt sensitive rat, and (4) DOCA (deoxycorticosterone acetate)-salt model.

### Administration of Guanylin Potentiating Agents

For treatment of gastrointestinal disorders, hypertension and other disorders, guanylin potentiating agents and other agents (and compounds co-administered with the guanylin potentiating agent) can be administered orally, e.g., as a tablet or cachet containing a predetermined amount of the active ingredient, pellet, gel, paste, syrup, bolus, electuary, slurry, capsule; sachet; “flash dosage”; powder; lyophilized powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agent can also be administered rectally, e.g., by suppository.

Guanylin potentiating agents can be co-administered with other therapeutic agents, including but not limited to those described herein. The guanylin potentiating agent can be administered together with guanylin or a guanylin variant or analogue. For treatment of gastrointestinal disorders, the agent can be administered orally. The agents can also be administered by rectal suppository. For the treatment of disorders outside the gastrointestinal tract such as hypertension, congestive heart failure and benign prostatic hypertrophy, the agents are preferably administered parenterally or orally. In some cases, the therapeutic agents preferably reach the small and/or large intestine in order to effectively reduce the activity of chymotrypsin that proteolytically digests guanylin. If the agent is to be administered orally, it is preferably formulated with an enteric coating. For example, the formulation can be provided with a non-porous, gastric acid-resistant polymer coating, e.g., a coating that is insoluble or only slightly soluble at pH 1.5 to pH 5, but is soluble above pH 5 or pH 5.5 up to or above pH 9. The polymer can include, for example, hydroxypropyl methyl cellulose phthalate, cellulose acetate phthalate, diethyl phthalate, dibutyl phthalate, and acrylic based polymers. The formulation can also be buffered by inclusion of a buffering agent, for example, sodium bicarbonate, potassium carbonate, potassium bicarbonate, ammonium carbonate, tromethamine, di(tris)hydroxymethylaminomethane) carbonate, tris-glycine, di-arginine, tri-arginine, poly-arginine, di-lysine, tri-lysine, poly-lysine, diethylamine and triethanolamine. It can be desirable for the buffering agent to provide a pH of from about 7 to about 9 in the small intestine or large intestine of a human patient. The formulation can also include a disintegrant, e.g., ursodiol, starch, modified starches, microcrystalline cellulose and propylene glycol alginate.

The guanylin potentiating agents can be used alone or in combination with other agents. For example, they can be administered together with an agent for treating a gastrointestinal disorder. They can be administered in a combination therapy with guanylin or a biologically active variant or fragment thereof. The guanylin potentiating agents can be administered together with an analgesic peptide or compound. The analgesic peptide or compound can be covalently attached to a guanylin potentiating agent described herein or it can be a separate agent that is administered together with or sequentially with a guanylin potentiating agent in a combination therapy.

Combination therapy can be achieved by administering two or more agents, e.g., a guanylin potentiating agent and an agent for treating a gastrointestinal disorder or an analgesic peptide or compound, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

Compositions may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof,
FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,
DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,
LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,
ANTI-CAKING AGENTS: calcium silicate, magnesium silicate, silicon dioxide, colloidal silicon dioxide, talc, or mixtures thereof,
ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and
COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(M-caprolactone) and poly(alkylene oxide) (U.S. 20030068384) to create a sustained release formulation. Such formulations can be used to implants that release a peptide or another agent over a period of a few days, a few weeks or several months depending on the polymer, the particle size of the polymer, and the size of the implant (see, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles of peptide are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), via intracavernosal injection, by transurethral application or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115-124)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranassaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

Compositions for oral administration can be in the form a of a “flash dosage”, i.e., a solid dosage form that is administered orally, which rapidly disperses in the mouth, and hence does not require great effort in swallowing and allows the compound to be rapidly ingested or absorbed through the oral mucosal membranes. In some embodiments, suitable rapidly dispersing dosage forms are also used in other applications, including the treatment of wounds and other bodily insults and diseased states in which release of the medicament by externally supplied moisture is not possible.

“Flash dose” forms are known in the art; see for example, effervescent dosage forms and quick release coatings of insoluble microparticles in U.S. Pat. Nos. 5,578,322 and 4,607,697; freeze dried foams and liquids in U.S. Pat. Nos. 4,642,903 and 5,631,023; melt spinning of dosage forms in U.S. Pat. Nos. 4,855,326; 5,380,326; and 5,518,730; solid, freeform fabrication in U.S. Pat. No. 6,471,992; saccharide-based carrier matrix and a liquid binder in U.S. Pat. Nos. 5,587,172; 5,616,344; 6,277,406; and 5,622,719; and other forms known to the art.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccaride are advantageous in that some of them also reportedly enhance absorption of the peptide in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means. The agent can be fused to immunoglobulins or albumin, or incorporated into a lipsome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. Peptides can also be modified with alkyl groups (e.g., C1-C20 straight or branched alkyl groups); fatty acid radicals; and combinations of PEG, alkyl groups and fatty acid radicals (see U.S. Pat. No. 6,309,633; Soltero et al., 2001 Innovations in Pharmaceutical Technology 106-110). The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

Suitable pharmaceutical compositions will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences (18th Edition, Mack Publishing Company, 1995).

The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Methods to increase chemical and/or physical stability of the agents the described herein are found in U.S. Pat. No. 6,541,606, U.S. Pat. No. 6,068,850, U.S. Pat. No. 6,124,261, U.S. Pat. No. 5,904,935, and WO 00/15224, U.S. 20030069182 (via the addition of nicotinamide), U.S. 20030175230A1, U.S. 20030175230A1, U.S. 20030175239A1, U.S. 20020045582, U.S. 20010031726, WO 02/26248, WO 03/014304, WO 98/00152A1, WO 98/00157A1, WO 90/12029, WO 00/04880, and WO 91/04743, WO 97/04796 and the references cited therein.

Methods to increase bioavailability of the agents described herein are found in U.S. Pat. No. 6,008,187, U.S. Pat. No. 5,424,289, U.S. 20030198619, WO 90/01329, WO 01/49268, WO 00/32172, and WO 02/064166. Glycyrrhizinate can also be used as an absorption enhancer (see, e.g., EP397447). WO 03/004062 discusses Ulex europaeus I (UEA1) and UEAI mimetics which may be used to target the agents of the invention to the GI tract.

The agents described herein can be fused to a modified version of the blood serum protein transferrin. U.S. 20030221201, U.S. 20040023334, U.S. 20030226155, WO 04/020454, and WO 04/019872 discuss the manufacture and use of transferrin fusion proteins. Transferrin fusion proteins may improve circulatory half life and efficacy, decrease undesirable side effects and allow reduced dosage.

### Combitherapy

### Analgesic Agents in Combitherapy

The guanylin potentiating agents described herein can be used in combination therapy with an analgesic agent, e.g., an analgesic compound or an analgesic peptide. These peptides and compounds can be administered simultaneously or sequentially with the agents described herein. They can also be optionally covalently linked or attached to an agent described herein to create therapeutic conjugates. Among the useful analgesic agents are: Ca channel blockers, 5HT receptor antagonists (for example 5HT3, 5HT4 and 5HT1 receptor antagonists), opioid receptor agonists (loperamide, fedotozine, and fentanyl), NK1 receptor antagonists, CCK receptor agonists (e.g., loxiglumide), NK1 receptor antagonists, NK3 receptor antagonists, norepinephrine-serotonin reuptake inhibitors (NSRI), vanilloid and cannabanoid receptor agonists, and sialorphin. Analgesics agents in the various classes are described in the literature.

Among the useful analgesic peptides are sialorphin-related peptides, including those comprising the amino acid sequence QHNPR (SEQ ID NO: 11), including: VQHNPR (SEQ ID NO: 12); VRQHNPR (SEQ ID NO: 13); VRGQHNPR (SEQ ID NO: 14); VRGPQHNPR (SEQ ID NO: 15); VRGPRQHNPR (SEQ ID NO: 16); VRGPRRQHNPR (SEQ ID NO: 17); and RQHNPR (SEQ ID NO: 18). Sialorphin-related peptides bind to neprilysin and inhibit neprilysin-mediated breakdown of substance P and Met-enkephalin. Thus, compounds or peptides that are inhibitors of neprilysin are useful analgesic agents which can be administered with the peptides of the invention in a co-therapy or linked to the peptides of the invention, e.g., by a covalent bond. Sialophin and related peptides are described in U.S. Pat. No. 6,589,750; U.S. 20030078200 A1; and WO 02/051435 A2.

Opioid receptor antagonists and agonists can be administered with the guanylin potentiating agents described herein in co-therapy or linked to the agent of the invention, e.g., by a covalent bond. For example, opioid receptor antagonists such as naloxone, naltrexone, methyl nalozone, nalmefene, cypridime, beta funaltrexamine, naloxonazine, naltrindole, and nor-binaltorphimine are thought to be useful in the treatment of IBS. It can be useful to formulate opioid antagonists of this type is a delayed and sustained release formulation such that initial release of the antagonist is in the mid to distal small intestine and/or ascending colon. Such antagonists are described in WO 01/32180 A2. Enkephalin pentapeptide (HOE825; Tyr-D-Lys-Gly-Phe-L-homoserine) is an agonist of the mu and delta opioid receptors and is thought to be useful for increasing intestinal motility (Eur. J. Pharm. 219:445, 1992), and this peptide can be used in conjunction with the peptides of the invention. Also useful is trimebutine which is thought to bind to mu/delta/kappa opioid receptors and activate release of motilin and modulate the release of gastrin, vasoactive intestinal peptide, gastrin and glucagons. Kappa opioid receptor agonists such as fedotozine, asimadoline, and ketocyclazocine, and compounds described in WO 03/097051 A2 can be used with or linked to the peptides of the invention. In addition, mu opioid receptor agonists such as morphine, diphenyloxylate, frakefamide (H-Tyr-D-Ala-Phe(F)-Phe-NH2; WO 01/019849 A1) and loperamide can be used.

Tyr-Arg (kyotorphin) is a dipeptide that acts by stimulating the release of met-enkephalins to elicit an analgesic effect (J. Biol. Chem. 262:8165, 1987). Kyotorphin can be used with or linked to the guanylin potentiating agents described herein.

Chromogranin-derived peptide (CgA 47-66; see, e.g., Ghia et al. 2004 Regulatory Peptides 119:199) can be used with or linked to the guanylin potentiating agents described herein.

CCK receptor agonists such as caerulein from amphibians and other species are useful analgesic agents that can be used with or linked to the guanylin potentiating agents described herein.

Conotoxin peptides represent a large class of analgesic peptides that act at voltage gated Ca channels, NMDA receptors or nicotinic receptors. These peptides can be used with or linked to the guanylin potentiating agents described herein.

Peptide analogs of thymulin (FR Application 2830451) can have analgesic activity and can be used with or linked to the guanylin potentiating agents described herein.

CCK (CCKa or CCKb) receptor antagonists, including loxiglumide and dexloxiglumide (the R-isomer of loxiglumide) (WO 88/05774) can have analgesic activity and can be used with or linked to the guanylin potentiating agents described herein.

Other useful analgesic agents include 5-HT4 agonists such as tegaserod (ZelnormÒ), mosapride, metoclopramide, zacopride, cisapride, renzapride, benzimidazolone derivatives such as BIMU 1 and BIMU 8, and lirexapride. Such agonists are described in: EP1321142 A1, WO 03/053432A1, EP 505322 A1, EP 505322 B1, U.S. Pat. No. 5,510,353, EP 507672 A1, EP 507672 B1, and U.S. Pat. No. 5,273,983.

Calcium channel blockers such as ziconotide and related compounds described in, for example, EP625162B1, U.S. Pat. No. 5,364,842, U.S. Pat. No. 5,587,454, U.S. Pat. No. 5,824,645, U.S. Pat. No. 5,859,186, U.S. Pat. No. 5,994,305, U.S. Pat. No. 6,087,091, U.S. Pat. No. 6,136,786, WO 93/13128 A1, EP 1336409 A1, EP 835126 A1, EP 835126 B1, U.S. Pat. No. 5,795,864, U.S. Pat. No. 5,891,849, U.S. Pat. No. 6,054,429, WO 97/01351 A1, can be used with or linked to a guanylin potentiating agent described herein.

Various antagonists of the NK-1, NK-2, and NK-3 receptors (for a review see Giardina et al. 2003 Drugs 6:758) can be can be used with or linked to the guanylin potentiating agents described herein.

NK1 receptor antagonists such as: aprepitant (Merck & Co Inc), vofopitant, ezlopitant (Pfizer, Inc.), R-673 (Hoffmann-La Roche Ltd), SR-48968 (Sanofi Synthelabo), CP-122,721 (Pfizer, Inc.), GW679769 (Glaxo Smith Kline), TAK-637 (Takeda/Abbot), SR-14033, and related compounds described in, for example, EP 873753 A1, US 20010006972 A1, US 20030109417 A1, WO 01/52844 A1, can be used with or linked to the guanylin potentiating agents described herein.

NK-2 receptor antagonists such as nepadutant (Menarini Ricerche SpA), saredutant (Sanofi-Synthelabo), GW597599 (Glaxo Smith Kline), SR-144190 (Sanofi-Synthelabo) and UK-290795 (Pfizer Inc) can be used with or linked to the prodrugs of chymotrypsin inhibitors described herein.

NK3 receptor antagonists such as osanetant (SR-142801; Sanofi-Synthelabo), SSR-241586, talnetant and related compounds described in, for example, WO 02/094187 A2, EP 876347 A1, WO 97/21680 A1, U.S. Pat. No. 6,277,862, WO 98/11090, WO 95/28418, WO 97/19927, and Boden et al. (J Med Chem. 39:1664-75, 1996) can be used with or linked to the guanylin potentiating agents described herein.

Norepinephrine-serotonin reuptake inhibitors (NSRI) such as milnacipran and related compounds described in WO 03/077897 A1 can be used with or linked to the guanylin potentiating agents described herein.

Vanilloid receptor antagonists such as arvanil and related compounds described in WO 01/64212 A1 can be used with or linked to the guanylin potentiating agents described herein.

The analgesic peptides and compounds can be administered with guanylin potentiating agents (simultaneously or sequentially). The analgesic agents can also be covalently linked to the guanylin potentiating agents described herein to create therapeutic conjugates. Where the analgesic is a peptide and is covalently linked to a guanylin potentiating agent the resulting peptide may also include at least one trypsin cleavage site. When present within the peptide, the analgesic peptide may be preceded by (if it is at the carboxy terminus) or followed by (if it is at the amino terminus) a trypsin cleavage site that allows release of the analgesic peptide.

In addition to sialorphin-related peptides, analgesic peptides include: AspPhe, endomorphin-1, endomorphin-2, nocistatin, dalargin, lupron, ziconotide, and substance P.

### Diabetes, Obesity and Other Disorders.

Pharmaceutical compositions comprising at least two of: 1) an agent that stimulates the production of cAMP (e.g., glucagon-like peptide 1 (GLP-1)); 2) an agent that inhibits the degradation of a cyclic nucleotide (e.g., a phosphodiesterase inhibitor); and 3) a guanlyin potentiating agent can be useful for treating diabetes and obesity. Such compositions may also be useful for treating secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions. In certain embodiments, the compositions further comprise guanylin or a biologically active variant or fragment thereof. In certain embodiments, the methods further comprise administering guanylin or a biologically active variant or fragment thereof.

The phosphodiesterase inhibitor can be specific for a particular phosphodiesterase (e.g., Group III or Group IV) or a non-specific phosphodiesterase inhibitor, such as papaverine, theophylline, enprofyllines and/or IBMX. Specific phosphodiesterase inhibitors which inhibit group III phosphodiesterases (cGMP-inhibited phosphodiesterases), including indolidane (LY195115), cilostamide (OPC 3689), lixazinone (RS 82856), Y-590, imazodane (CI914), SKF 94120, quazinone, ICI 153,110, cilostazole, bemorandane (RWJ 22867), siguazodane (SK&F 94-836), adibendane (BM 14,478), milrinone (WIN 47203), enoximone (MDL 17043), pimobendane (UD-CG 115), MCI-154, saterinone (BDF 8634), sulmazole (ARL 115), UD-CG 212, motapizone, piroximone, and ICI 118233 can be useful. In addition, phosphodiesterase inhibitors which inhibit group IV phosphodiesterases (cAMP-specific phosphodiesterases), such as rolipram ZK 62711; pyrrolidone), imidazolidinone (RO 20-1724), etazolate (SQ 65442), denbufylline (BRL 30892), ICI63197, and RP73401 can be used.

### Other Agents for Use in Combitherapy

Pharmaceutical compositions comprising a guanylin potentiating agent and a second therapeutic agent are useful, The second therapeutic agent can be administered to treat any condition for which it is useful, including conditions that are not considered to be the primary indication for treatment with the second therapeutic agent.

Examples of additional therapeutic agents to treat gastrointestinal and other disorders include:

agents to treat constipation (e.g., a chloride channel activator such as the bicylic fatty acid, Lubiprostone (formerly known as SPI-0211; Sucampo Pharmaceuticals, Inc.; Bethesda, Md.), a laxative (eg. a bulk-forming laxative (e.g. nonstarch polysaccharides, Colonel Tablet (polycarbophil calcium), Plantago Ovata®, Equalactin® (Calcium Polycarbophil)), fiber (e.g. FIBERCON® (Calcium Polycarbophil), an osmotic laxative, a stimulant laxative (such as diphenylmethanes (e.g. bisacodyl), anthraquinones (e.g. cascara, senna), and surfactant laxatives (e.g. castor oil, docusates), an emollient/lubricating agent (such as mineral oil, glycerine, and docusates), MiraLax (Braintree Laboratories, Braintree Mass.), dexloxiglumide (Forest Laboratories, also known as CR 2017 Rottapharm (Rotta Research Laboratorium SpA)), saline laxatives, enemas, suppositories, and CR 3700 (Rottapharm (Rotta Research Laboratorium SpA);
acid reducing agents such as proton pump inhibitors (e.g., omeprazole (Prilosec®), esomeprazole (Nexium®), lansoprazole (Prevacid®), pantoprazole (Protonix®) and rabeprazole (Aciphex®)) and Histamine H2-receptor antagonist (also known as H2 receptor blockers including cimetidine, ranitidine, famotidine and nizatidine);
prokinetic agents including itopride, octreotide, bethanechol, metoclopramide (Reglan®), domperidone (Motilium®), erythromycin (and derivatives thereof) or cisapride (Propulsid®);

pro-motility agents such as the vasostatin-derived peptide, chromogranin A (4-16) (see, e.g., Ghia et al. 2004 Regulatory Peptides 121:31) or motilin agonists (e.g., GM-611 or mitemcinal fumarate) or nociceptin/Orphanin FQ receptor modulators (US20050169917);

complete or partial 5HT (e.g. 5HT1, 5HT2, 5HT3, 5HT4) receptor agonists or antagonists (including 5HT1A antagonists (e.g. AGI-001 (AGI therapeutics), 5HT2B antagonists (e.g. PGN1091 and PGN1164 (Pharmagene Laboratories Limited), and 5HT4 receptor agonists (such as tegaserod (ZELNORM®), prucalopride, mosapride, metoclopramide, zacopride, cisapride, renzapride, benzimidazolone derivatives such as BIMU 1 and BIMU 8, and lirexapride). Such agonists/modulatos are described in: EP1321142 A1, WO 03/053432A1, EP 505322 A1, EP 505322 B1, U.S. Pat. No. 5,510,353, EP 507672 A1, EP 507672 B1, U.S. Pat. No. 5,273,983, and U.S. Pat. No. 6,951,867); 5HT3 receptor agonists such as MKC-733; and 5HT3 receptor antagonists such as DDP-225 (MCI-225; Dynogen Pharmaceuticals, Inc.), cilansetron (Calmactin®), alosetron (Lotronex®), Ondansetron HCl (Zofran®), Dolasetron (ANZEMET®), palonosetron (Aloxi®), Granisetron (Kytril®), YM060(ramosetron; Astellas Pharma Inc.; ramosetron may be given as a daily dose of 0.002 to 0.02 mg as described in EP01588707) and ATI-7000 (Aryx Therapeutics, Santa Clara Calif.);
muscarinic receptor agonists;
anti-inflammatory agents;
antispasmodics including but not limited to anticholinergic drugs (like dicyclomine (e.g. Colimex®, Formulex®, Lomine®, Protylol®, Viscerol®, Spasmoban®, Bentyl®, Bentylol®, hyoscyamine (e.g. IB-Stat®, Nulev®, Levsin®, Levbid®, Levsinex Timecaps®, Levsin/SL®, Anaspaz®, A-Spas S/L®, Cystospaz®, Cystospaz-M®, Donnamar®, Colidrops Liquid Pediatric®, Gastrosed®, Hyco Elixir®, Hyosol®, Hyospaz®, Hyosyne®, Losamine®, Medispaz®, Neosol®, Spacol®, Spasdel®, Symax®, Symax SL®), Donnatal (e.g. Donnatal Extentabs®), clidinium (e.g. Quarzan, in combination with Librium=Librax), methantheline (e.g. Banthine), Mepenzolate (e.g. Cantil), homatropine (e.g. hycodan, Homapin), Propantheline bromide (e.g. Pro-Banthine), Glycopyrrolate (e.g. Robinul®, Robinul Forte®), scopolamine (e.g. Transderm-Scop®, Transderm-V®), hyosine-N-butylbromide (e.g. Buscopan®), Pirenzepine (e.g. Gastrozepin®) Propantheline Bromide (e.g. Propanthel®), dicycloverine (e.g. Merbentyl®), glycopyrronium bromide (e.g. Glycopyrrolate®), hyoscine hydrobromide, hyoscine methobromide, methanthelinium, and octatropine); peppermint oil; and direct smooth muscle relaxants like cimetropium bromide, mebeverine (DUSPATAL®, DUSPATALIN®, COLOFAC MR®, COLOTAL®), otilonium bromide (octilonium), pinaverium (e.g. Dicetel® (pinaverium bromide; Solvay S.A.)), Spasfon® (hydrated phloroglucinol and trimethylphloroglucinol) and trimebutine (including trimebutine maleate (Modulon®);
antidepressants, including but not limited to those listed herein, as well as tricyclic antidepressants like amitriptyline (Elavil®), desipramine (Norpramin®), imipramine (Tofranil®), amoxapine (Asendin®), nortriptyline; the selective serotonin reuptake inhibitors (SSRI's) like paroxetine (Paxil®), fluoxetine (Prozac®), sertraline (Zoloft®), and citralopram (Celexa®); and others like doxepin (Sinequan®) and trazodone (Desyrel®);
centrally-acting analgesic agents such as opioid receptor agonists, opioid receptor antagonists (e.g., naltrexone);
agents for the treatment of Inflammatory bowel disease;
agents for the treatment of Crohn's disease and/or ulcerative colitis (e.g., alequel (Enzo Biochem, Inc.; Farmingsale, N.Y.), the anti-inflammatory peptide RDP58 (Genzyme, Inc.; Cambridge, Mass.), and TRAFICET-EN™ (ChemoCentryx, Inc.; San Carlos, Calif.);
agents that treat gastrointestinal or visceral pain;
agents that increase cGMP levels (as described in US20040121994) like adrenergic receptor antagonists, dopamine receptor agonists and PDE (phosphodiesterase) inhibitors including but not limited to those disclosed herein;
purgatives that draw fluids to the intestine (e.g., VISICOL®, a combination of sodium phosphate monobasic monohydrate and sodium phosphate dibasic anhydrate);
Corticotropin Releasing Factor (CRF) receptor antagonists (including NBI-34041 (Neurocrine Biosciences, San Diego, Calif.), CRH9-41, astressin, R121919 (Janssen Pharmaceutica), CP154,526, NBI-27914, Antalarmin, DMP696 (Bristol-Myers Squibb) CP-316,311 (Pfizer, Inc.), SB723620 (GSK), GW876008 (Neurocrine/Glaxo Smith Kline), ONO-2333Ms (Ono Pharmaceuticals), TS-041 (Janssen), AAG561 (Novartis) and those disclosed in U.S. Pat. No. 5,063,245, U.S. Pat. No. 5,861,398, US20040224964, US20040198726, US20040176400, US20040171607, US20040110815, US20040006066, and US20050209253);
glucagon-like peptides (glp-1) and analogues thereof (including exendin-4 and GTP-010 (Gastrotech Pharma A)) and inhibitors of DPP-IV (DPP-IV mediates the inactivation of glp-1);
tofisopam, enantiomerically-pure R-tofisopam, and pharmaceutically-acceptable salts thereof (US 20040229867);
tricyclic anti-depressants of the dibenzothiazepine type including but not limited to Dextofisopam® (Vela Pharmaceuticals), tianeptine (Stablon®) and other agents described in U.S. Pat. No. 6,683,072;
(E)-4 (1,3bis(cyclohexylmethyl)-1,2,34,-tetrahydro-2,6-diono-9H-purin-8-yl)cinnamic acid nonaethylene glycol methyl ether ester and related compounds described in WO 02/067942;
the probiotic PROBACTRIX® (The BioBalance Corporation; New York, N.Y.) which contains microorganisms useful in the treatment of gastrointestinal disorders;
antidiarrheal drugs including but not limited to loperamide (Imodium, Pepto Diarrhea), diphenoxylate with atropine (Lomotil, Lomocot), cholestyramine (Questran, Cholybar), atropine (Co-Phenotrope, Diarsed, Diphenoxylate, Lofene, Logen, Lonox, Vi-Atro, atropine sulfate injection) and Xifaxan® (rifaximin; Salix Pharmaceuticals Ltd), TZP-201 (Tranzyme Pharma Inc.), the neuronal acetylcholine receptor (nAChR) blocker AGI-004 (AGI therapeutics), and bismuth subsalicylate (Pepto-bismol);
anxiolytic drugs including but not limited to Ativan (lorazepam), alprazolam (Xanax®), chlordiazepoxide/clidinium (Librium®, Librax®), clonazepam (Klonopin®), clorazepate (Tranxene®), diazepam (Valium®), estazolam (ProSom®), flurazepam (Dalmane®), oxazepam (Serax®), prazepam (Centrax®), temazepam (Restoril®), triazolam (Halcion®;
Bedelix® (Montmorillonite beidellitic; Ipsen Ltd), Solvay SLV332 (ArQule Inc), YKP (SK Pharma), Asimadoline (Tioga Pharmaceuticals/Merck), AGI-003 (AGI Therapeutics);
the serotonin modulator AZD7371 (AstraZeneca Plc);
M3 muscarinic receptor antagonists such as darifenacin (Enablex; Novartis AG and zamifenacin (Pfizer);
herbal and natural therapies including but not limited to acidophilus, chamomile tea, evening primrose oil, fennel seeds, wormwood, comfrey, and compounds of Bao-Ji-Wan (magnolol, honokiol, imperatorin, and isoimperatorin) as in U.S. Pat. No. 6,923,992; and
compositions comprising lysine and an anti-stress agent for the treatment of irritable bowel syndrome as described in EP01550443.

The guanylin potentiating agents described herein can be used in combination therapy with insulin and related compounds including primate, rodent, or rabbit insulin including biologically active variants thereof including allelic variants, more preferably human insulin available in recombinant form. Sources of human insulin include pharmaceutically acceptable and sterile formulations such as those available from Eli Lilly (Indianapolis, Ind. 46285) as Humulin™ (human insulin rDNA origin). See the THE PHYSICIAN'S DESK REFERENCE, 55.sup.th Ed. (2001) Medical Economics, Thomson Healthcare (disclosing other suitable human insulins). The guanylin potentiating agents described herein can also be used in combination therapy with agents that can boost insulin effects or levels of a subject upon administration, e.g. glipizide and/or rosiglitazone. The guanylin potentiating agents herein can be used in combitherapy with SYMLIN® (pramlintide acetate) and Exenatide® (synthetic exendin-4; a 39 aa peptide).

The guanylin potentiating agents described herein can also be used in combination therapy with agents (e.g., Entereg™ (alvimopan; formerly called adolor/ADL 8-2698), conivaptan and related agents describe in U.S. Pat. No. 6,645,959) for the treatment of postoperative ileus.

The guanylin potentiating agents described herein can be used in combination therapy with an anti-hypertensive agent including but not limited to:

(1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like;
(2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like;
(3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like;
(4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like;
(5) neutral endopeptidase inhibitors such as omapatrilat, cadoxatril and ecadotril, fosidotril, sampatrilat, AVE7688, ER4030, and the like;
(6) endothelin antagonists such as tezosentan, A308165, and YM62899, and the like;
(7) vasodilators such as hydralazine, clonidine, minoxidil, and nicotinyl alcohol, and the like;
(8) angiotensin II receptor antagonists such as aprosartan, candesartan, eprosartan, irbesartan, losartan, olmesartan, pratosartan, tasosartan, telmisartan, valsartan, and EXP-3137, FT6828K, and RNH6270, and the like;
(9) α/β adrenergic blockers such as nipradilol, arotinolol and amosulalol, and the like;
(10) alpha 1 blockers, such as terazosin, urapidil, prazosin, tamsulosin, bunazosin, trimazosin, doxazosin, naftopidil, indoramin, WHP 164, and XEN010, and the like;
(11) alpha 2 agonists such as lofexidine, tiamenidine, moxonidine, rilmenidine and guanobenz, and the like;
(12) aldosterone inhibitors, and the like; and
(13) angiopoietin-2-binding agents such as those disclosed in WO03/030833.

Specific anti-hypertensive agents that can be used in combination with guanylin potentiating agents described herein include, but not limited to:

diuretics, such as thiazides (e.g., chlorthalidone, cyclothiazide (CAS RN 2259-96-3), chlorothiazide (CAS RN 72956-09-3, which may be prepared as disclosed in US2809194), dichlorophenamide, hydroflumethiazide, indapamide, polythiazide, bendroflumethazide, methyclothazide, polythiazide, trichlormethazide, chlorthalidone, indapamide, metolazone, quinethazone, althiazide (CAS RN 5588-16-9, which may be prepared as disclosed in British Patent No. 902,658), benzthiazide (CAS RN 91-33-8, which may be prepared as disclosed in U.S. Pat. No. 3,108,097), buthiazide (which may be prepared as disclosed in British Patent Nos. 861,367), and hydrochlorothiazide), loop diuretics (e.g. bumetanide, ethacrynic acid, furosemide, and torasemide), potassium sparing agents (e.g. amiloride, and triamterene (CAS Number 396-01-0)), and aldosterone antagonists (e.g. spironolactone (CAS Number 52-01-7), epirenone, and the like); β-adrenergic blockers such as Amiodarone (Cordarone, Pacerone), bunolol hydrochloride (CAS RN 31969-05-8, Parke-Davis), acebutolol (±N-[3-Acetyl-4-[2-hydroxy-3-[(1 methylethyl)amino]propoxy]phenyl]-butanamide, or (±)-3′-Acetyl-4′-[2-hydroxy-3-(isopropylamino) propoxy]butyranilide), acebutolol hydrochloride (e.g. Sectral®, Wyeth-Ayerst), alprenolol hydrochloride (CAS RN 13707-88-5 see Netherlands Patent Application No. 6,605,692), atenolol (e.g. Tenormin®, AstraZeneca), carteolol hydrochloride (e.g. Cartrol® Filmtab®, Abbott), Celiprolol hydrochloride (CAS RN 57470-78-7, also see in U.S. Pat. No. 4,034,009), cetamolol hydrochloride (CAS RN 77590-95-5, see also U.S. Pat. No. 4,059,622), labetalol hydrochloride (e.g. Normodyne®, Schering), esmolol hydrochloride (e.g. Brevibloc®, Baxter), levobetaxolol hydrochloride (e.g. Betaxon™ Ophthalmic Suspension, Alcon), levobunolol hydrochloride (e.g. Betagan® Liquifilm® with C CAP® Compliance Cap, Allergan), nadolol (e.g. Nadolol, Mylan), practolol (CAS RN 6673-35-4, see also U.S. Pat. No. 3,408,387), propranolol hydrochloride (CAS RN 318-98-9), sotalol hydrochloride (e.g. Betapace AF™, Berlex), timolol (2-Propanol,1-[(1,1-dimethylethyl)amino]-3-[[4-4(4-morpholinyl)-1,2,5-thiadiazol-3-yl]oxy]-, hemihydrate, (S)—, CAS RN 91524-16-2), timolol maleate (S)-1-[(1,1-dimethylethyl) amino]-3-[[4-(4-morpholinyl)-1,2,5-thiadiazol-3-yl]oxy]-2-propanol (Z)-2-butenedioate (1:1) salt, CAS RN 26921-17-5), bisoprolol (2-Propanol, 1-[4-[[2-(1-methylethoxy)ethoxy]-methyl]phenoxyl]-3-[(1-meth-ylethyl)amino]-, (+), CAS RN 66722-44-9), bisoprolol fumarate (such as (±)-1-[4-[[2-(1-Methylethoxy) ethoxy]methyl]phenoxy]-3-[(1-methylethyl)amino]-2-propanol (E)-2-butenedioate (2:1) (salt), e.g., Zebeta™, Lederle Consumer), nebivalol (2H-1-Benzopyran-2-methanol, αα′-[iminobis(methylene)]bis[6-fluoro-3,4-dihydro-, CAS RN 99200-09-6 see also U.S. Pat. No. 4,654,362), cicloprolol hydrochloride, such 2-Propanol, 1-[4-[2-(cyclopropylmethoxy)ethoxy]phenoxy]-3-[1-methylethyl)amino]-, hydrochloride, A.A.S. RN 63686-79-3), dexpropranolol hydrochloride (2-Propanol,1-[1-methylethy)-amino]-3-(1-naphthalenyloxy)-hydrochloride (CAS RN 13071-11-9), diacetolol hydrochloride (Acetamide, N-[3-acetyl-4-[2-hydroxy-3-[(1-methyl-ethyl)amino]propoxy][phenyl]-, monohydrochloride CAS RN 69796-04-9), dilevalol hydrochloride (Benzamide, 2-hydroxy-5-[1-hydroxy-2-[1-methyl-3-phenylpropyl)amino]ethyl]-, monohydrochloride, CAS RN 75659-08-4), exaprolol hydrochloride (2-Propanol, 1-(2-cyclohexylphenoxy)-3-[(1-methylethyl)amino]-, hydrochloride CAS RN 59333-90-3), flestolol sulfate (Benzoic acid, 2-fluoro-,3-[[2-[aminocarbonyl)amino]-dimethylethyl]amino]-2-hydroxypropyl ester, (±)-sulfate (1:1) (salt), CAS RN 88844-73-9; metalol hydrochloride (Methanesulfonamide, N-[4-[1-hydroxy-2-(methylamino)propyl]phenyl]-, monohydrochloride CAS RN 7701-65-7), metoprolol 2-Propanol, 1-[4-(2-methoxyethyl)phenoxy]-3-[1-methylethyl)amino]-; CAS RN 37350-58-6), metoprolol tartrate (such as 2-Propanol, 1-[4-(2-methoxyethyl)phenoxy]-3-[(1-methylethyl)amino]-, e.g., Lopressor®, Novartis), pamatolol sulfate (Carbamic acid, [2-[4-[2-hydroxy-3-[(1-methylethyl)amino]propoxyl]phenyl]-ethyl]-, methyl ester, (±) sulfate (salt) (2:1), CAS RN 59954-01-7), penbutolol sulfate (2-Propanol, 1-(2-cyclopentylphenoxy)-3-[1,1-dimethyle-thyl)amino]1, (S)—, sulfate (2:1) (salt), CAS RN 38363-32-5), practolol (Acetamide, N-[4-[2-hydroxy-3-[(1-methylethyl)amino]-propoxy]phenyl]-, CAS RN 6673-35-4;) tiprenolol hydrochloride (Propanol, 1-[(1-methylethyl)amino]-3-[2-(methylthio)-phenoxy]-, hydrochloride, (±), CAS RN 39832-43-4), tolamolol (Benzamide, 4-[2-[[2-hydroxy-3-(2-methylphenoxy)-propyl]amino]ethoxyl]-, CAS RN 38103-61-6), bopindolol, indenolol, pindolol, propanolol, tertatolol, and tilisolol, and the like; calcium channel blockers such as besylate salt of amlodipine (such as 3-ethyl-5-methyl-2-(2-aminoethoxymethyl)-4-(2-chlorophenyl)-1,4-dihydro-6-methyl-3,5-pyridinedicarboxylate benzenesulphonate, e.g., Norvasc®, Pfizer), clentiazem maleate (1,5-Benzothiazepin-4(5H)-one, 3-(acetyloxy)-8-chloro-5-[2-(dimethylamino)ethyl]-2,3-dihydro-2-(4-methoxyphenyl)-(2S-cis)-, (Z)-2-butenedioate (1:1), see also U.S. Pat. No. 4,567,195), isradipine (3,5-Pyridinedicarboxylic acid, 4-(4-benzofurazanyl)-1,4-dihydro-2,6-dimethyl-, methyl 1-methylethyl ester, (±)-4(4-benzofurazanyl)-1,4-dihydro-2,6-dimethyl-3,5-pyridinedicarboxylate, see also U.S. Pat. No. 4,466,972); nimodipine (such as is isopropyl (2-methoxyethyl) 1,4-dihydro-2,6-dimethyl-4-(3-nitrophenyl)-3,5-pyridine-dicarboxylate, e.g. Nimotop®, Bayer), felodipine (such as ethyl methyl 4-(2,3-dichlorophenyl)-1,4-dihydro-2,6-dimethyl-3,5-pyridinedicarboxylate-, e.g. Plendil® Extended-Release, AstraZeneca LP), nilvadipine (3,5-Pyridinedicarboxylic acid, 2-cyano-1,4-dihydro-6-methyl-4-(3-nitrophenyl)-,3-methyl 5-(1-methylethyl) ester, also see U.S. Pat. No. 3,799,934), nifedipine (such as 3,5-pyridinedicarboxylic acid, 1,4-dihydro-2,6-dimethyl-4-(2-nitrophenyl)-, dimethyl ester, e.g., Procardia XL® Extended Release Tablets, Pfizer), diltiazem hydrochloride (such as 1,5-Benzothiazepin-4(5H)-one, 3-(acetyloxy)-5[2-(dimethylamino)ethyl]-2,-3-dihydro-2(4-methoxyphenyl)-, monohydrochloride, (+)-cis., e.g., Tiazac®, Forest), verapamil hydrochloride (such as benzeneacetronitrile, (alpha)-[[3-[[2-(3,4-dimethoxyphenyl)ethyl]methylamino]propyl]-3,4-dimethoxy-(alpha)-(1-methylethyl)hydrochloride, e.g., Isoptin® SR, Knoll Labs), teludipine hydrochloride (3,5-Pyridinedicarboxylic acid, 2-[(dimethylamino)methyl]4-[2-[(1E)-3-(1,1-dimethylethoxy)-3-oxo-1-propenyl]phenyl]-1,4-dihydro-6-methyl-, diethyl ester, monohydrochloride) CAS RN 108700-03-4), belfosdil (Phosphonic acid, [2-(2-phenoxyethyl)-1,3-propane-diyl]bis-, tetrabutyl ester CAS RN 103486-79-9), fostedil (Phosphonic acid, [[4-(2-benzothiazolyl)phenyl]methyl]-, diethyl ester CAS RN 75889-62-2), aranidipine, azelnidipine, barnidipine, benidipine, bepridil, cinaldipine, clevidipine, efonidipine, gallopamil, lacidipine, lemildipine, lercanidipine, monatepil maleate (1-piperazinebutanamide, N-(6,11-dihydrodibenzo(b,e)thiepin-11-yl)₄-(4-fluorophenyl)-, (±)-, (Z)-2-butenedioate (1:1)(±)—N-(6,11-Dihydrodibenzo(b,e)thiep-in-11-yl)-4-(p-fluorophenyl)-1-piperazinebutyramide maleate (1:1) CAS RN 132046-06-1), nicardipine, nisoldipine, nitrendipine, manidipine, pranidipine, and the like;
T-channel calcium antagonists such as mibefradil; angiotensin converting enzyme (ACE) inhibitors such as benazepril, benazepril hydrochloride (such as 3-[[1-(ethoxycarbonyl)-3-phenyl-(1 S)-propyl]amino]-2,3,4,5-tetrahydro-2-oxo-1H-1-(3S)-benzazepine-1-acetic acid monohydrochloride, e.g., Lotrel®, Novartis), captopril (such as i-[(2S)-3-mercapto-2-methylpropionyl]-L-proline, e.g., Captopril, Mylan, CAS RN 62571-86-2 and others disclosed in U.S. Pat. No. 4,046,889), ceranapril (and others disclosed in U.S. Pat. No. 4,452,790), cetapril (alacepril, Dainippon disclosed in Eur. Therap. Res. 39:671 (1986); 40:543 (1986)), cilazapril (Hoffman-LaRoche) disclosed in J. Cardiovasc. Pharmacol. 9:39 (1987), indalapril (delapril hydrochloride (2H-1,2,4-Benzothiadiazine-7-sulfonamide, 3-bicyclo[2.2.1]hept-5-en-2-yl-6-chloro-3,4-dihydro-, 1,1-dioxide CAS RN 2259-96-3); disclosed in U.S. Pat. No. 4,385,051), enalapril (and others disclosed in U.S. Pat. No. 4,374,829), enalopril, enaloprilat, fosinopril, ((such as L-proline, 4-cyclohexyl-1-[[[2-methyl-1-(1-oxopropoxy) propoxy](4-phenylbutyl) phosphinyl]acetyl]-, sodium salt, trans-, e.g., Monopril, Bristol-Myers Squibb and others disclosed in U.S. Pat. No. 4,168,267), fosinopril sodium (L-Proline, 4-cyclohexyl-1-[[(R)-[(1S)-2-methyl-1-(1-ox-opropoxy)propox), imidapril, indolapril (Schering, disclosed in J. Cardiovasc. Pharmacol. 5:643, 655 (1983)), lisinopril (Merck), losinopril, moexipril, moexipril hydrochloride (3-Isoquinolinecarboxylic acid, 2-[(2S)-2-[[(1S)-1-(ethoxycarbonyl)-3-phenylpropyl]amino]-1-oxopropyl]-1,-2,3,4-tetrahydro-6,7-dimethoxy-, monohydrochloride, (3S)-CAS RN 82586-52-5), quinapril, quinaprilat, ramipril (Hoechsst) disclosed in EP 79022 and Curr. Ther. Res. 40:74 (1986), perindopril erbumine (such as 2S,3aS,7aS-1-[(S)—N—[(S)-1-Carboxybutyl]alanyl]hexahydro-2-indolinecarboxylic acid, 1-ethyl ester, compound with tert-butylamine (1:1), e.g., Aceon®, Solvay), perindopril (Servier, disclosed in Eur. J. clin. Pharmacol. 31:519 (1987)), quanipril (disclosed in U.S. Pat. No. 4,344,949), spirapril (Schering, disclosed in Acta. Pharmacol. Toxicol. 59 (Supp. 5):173 (1986)), tenocapril, trandolapril, zofenopril (and others disclosed in U.S. Pat. No. 4,316,906), rentiapril (fentiapril, disclosed in Clin. Exp. Pharmacol. Physiol. 10:131 (1983)), pivopril, YS980, teprotide (Bradykinin potentiator BPP9a CAS RN 35115-60-7), BRL 36,378 (Smith Kline Beecham, see EP80822 and EP60668), MC-838 (Chugai, see C.A. 102:72588v and Jap. J. Pharmacol. 40:373 (1986), CGS 14824 (Ciba-Geigy, 3-([1-ethoxycarbonyl-3-phenyl-(1S)-propyl]amino)-2,3,4,5-tetrahydro-2-ox-o-1-(3S)-benzazepine-1 acetic acid HCl, see U.K. Patent No. 2103614), CGS 16,617 (Ciba-Geigy, 3(S)-[[(1S)-5-amino-1-carboxypentyl]amino]-2,3,4,-5-tetrahydro-2-oxo-1H-1-benzazepine-1-ethanoic acid, see U.S. Pat. No. 4,473,575), Ru 44570 (Hoechst, see Arzneimittelforschung 34:1254 (1985)), R 31-2201 (Hoffman-LaRoche see FEBS Lett. 165:201 (1984)), CI925 (Pharmacologist 26:243, 266 (1984)), WY-44221 (Wyeth, see J. Med. Chem. 26:394 (1983)), and those disclosed in US2003006922 (paragraph 28), U.S. Pat. No. 4,337,201, U.S. Pat. No. 4,432,971 (phosphonamidates); neutral endopeptidase inhibitors such as omapatrilat (Vanlev®), CGS 30440, cadoxatril and ecadotril, fasidotril (also known as aladotril or alatriopril), sampatrilat, mixanpril, and gemopatrilat, AVE7688, ER4030, and those disclosed in U.S. Pat. No. 5,362,727, U.S. Pat. No. 5,366,973, U.S. Pat. No. 5,225,401, U.S. Pat. No. 4,722,810, U.S. Pat. No. 5,223,516, U.S. Pat. No. 4,749,688, U.S. Pat. No. 5,552,397, U.S. Pat. No. 5,504,080, U.S. Pat. No. 5,612,359, U.S. Pat. No. 5,525,723, EP0599444, EP0481522, EP0599444, EP0595610, EP0534363, EP534396, EP534492, EP0629627; endothelin antagonists such as tezosentan, A308165, and YM62899, and the like; vasodilators such as hydralazine (apresoline), clonidine (clonidine hydrochloride (1H-Imidazol-2-amine, N-(2,6-dichlorophenyl)4,5-dihydro-, monohydrochloride CAS RN 4205-91-8), catapres, minoxidil (loniten), nicotinyl alcohol (roniacol), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis, e.g., Tiazac®, Forest), isosorbide dinitrate (such as 1,4:3,6-dianhydro-D-glucitol 2,5-dinitrate e.g., Isordil® Titradose®, Wyeth-Ayerst), sosorbide mononitrate (such as 1,4:3,6-dianhydro-D-glucito-1,5-nitrate, an organic nitrate, e.g., Ismo®, Wyeth-Ayerst), nitroglycerin (such as 2,3 propanetriol trinitrate, e.g., Nitrostat® Parke-Davis), verapamil hydrochloride (such as benzeneacetonitrile, (±)-(alpha)[3-[[2-(3,4 dimethoxyphenyl)ethyl]methylamino]propyl]-3,4-dimethoxy-(alpha)-(1-methylethyl) hydrochloride, e.g., Covera HS® Extended-Release, Searle), chromonar (which may be prepared as disclosed in U.S. Pat. No. 3,282,938), clonitate (Annalen 1870 155), droprenilamine (which may be prepared as disclosed in DE2521113), lidoflazine (which may be prepared as disclosed in U.S. Pat. No. 3,267,104); prenylamine (which may be prepared as disclosed in U.S. Pat. No. 3,152,173), propatyl nitrate (which may be prepared as disclosed in French Patent No. 1,103,113), mioflazine hydrochloride (1-piperazineacetamide, 3-(aminocarbonyl)-4-[4,4-bis(4-fluorophenyl)butyl]-N-(2,6-dichlorophenyl)-, dihydrochloride CAS RN 83898-67-3), mixidine (Benzeneethanamine, 3,4-dimethoxy-N-(1-methyl-2-pyrrolidinylidene)-Pyrrolidine, 2-[(3,4-dimethoxyphenethyl)imino]-[1-methyl-1-Methyl-2-[(3,4-dimethoxyphenethyl)imino]pyrrolidine CAS RN 27737-38-8), molsidomine (1,2,3-Oxadiazolium, 5-[(ethoxycarbonyl)amino]-3-(4-morpholinyl)-, inner salt CAS RN 25717-80-0), isosorbide mononitrate (D-Glucitol, 1,4:3,6-dianhydro-, 5-nitrate CAS RN 16051-77-7), erythrityl tetranitrate (1,2,3,4-Butanetetrol, tetranitrate, (2R,3S)-rel-CAS RN 7297-25-8), clonitrate(1,2-Propanediol, 3-chloro-, dinitrate (7CI, 8CI, 9CI) CAS RN 2612-33-1), dipyridamole Ethanol, 2,2′,2″,2′″-[(4,8-di-1-piperidinylpyrimido[5,4-d]pyrimidine-2,6-diyl)dinitrilo]tetrakis-CAS RN 58-32-2), nicorandil (CAS RN 65141-46-0 3-), pyridinecarboxamide (N-[2-(nitrooxy)ethyl]-Nisoldipine3,5-Pyridinedicarboxylic acid, 1,4-dihydro-2,6-dimethyl-4-(2-nitrophenyl)-, methyl 2-methylpropyl ester CAS RN 63675-72-9), nifedipine3,5-Pyridinedicarboxylic acid, 1,4-dihydro-2,6-dimethyl-4-(2-nitrophenyl)-, dimethyl ester CAS RN 21829-25-4), perhexyline maleate (Piperidine, 2-(2,2-dicyclohexylethyl)-, (2Z)-2-butenedioate (1:1) CAS RN 6724-53-4), oxprenolol hydrochloride (2-Propanol, 1-[(1-methylethyl)amino]-3-[2-(2-propenyloxy)phenoxy]-, hydrochloride CAS RN 6452-73-9), pentrinitrol (1,3-Propanediol, 2,2-bis[(nitrooxy)methyl]-mononitrate (ester) CAS RN 1607-17-6), verapamil (Benzeneacetonitrile, α-[3-[[2-(3,4-dimethoxyphenyl)ethyl]-methylamino]propyl]-3,4-dimethoxy-α-(1-methylethyl)-CAS RN 52-53-9) and the like; angiotensin II receptor antagonists such as, aprosartan, zolasartan, olmesartan, pratosartan, FI6828K, RNH6270, candesartan (1H-Benzimidazole-7-carboxylic acid, 2-ethoxy-1-[[2′-(1H-tetrazol-5-yl)[1,1′-biphenyl]4-yl]methyl]-CAS RN 139481-59-7), candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole carboxylate, CAS RN 145040-37-5, U.S. Pat. No. 5,703,110 and U.S. Pat. No. 5,196,444), eprosartan (3-[1-4-carboxyphenylmethyl)-2-n-butyl-imidazol-5-yl]-(2-thienylmethyl) propenoic acid, U.S. Pat. No. 5,185,351 and U.S. Pat. No. 5,650,650), irbesartan (2-n-butyl-3-[[2′-(1 h-tetrazol-5-yl)biphenyl-4-yl]methyl]1,3-diazazspiro[4,4]non-1-en-4-one, U.S. Pat. No. 5,270,317 and U.S. Pat. No. 5,352,788), losartan (2-N-butyl-4-chloro-5-hydroxymethyl-1-[(2′-(1H-tetrazol-5-yl)biphenyl-4-yl)-methyl]imidazole, potassium salt, U.S. Pat. No. 5,138,069, U.S. Pat. No. 5,153,197 and U.S. Pat. No. 5,128,355), tasosartan (5,8-dihydro-2,4-dimethyl-8-[(2′-(1H-tetrazol-5-yl)[1,1′-biphenyl]4-yl)methyl]-pyrido[2,3-d]pyrimidin-7(6H)-one, U.S. Pat. No. 5,149,699), telmisartan (4′-[(1,4-dimethyl-2′-propyl-(2,6′-bi-1H-benzimidazol)-1′-yl)]-[1,1′-biphenyl]-2-carboxylic acid, CAS RN 144701-48-4, U.S. Pat. No. 5,591,762), milfasartan, abitesartan, valsartan (Diovan® (Novartis), (S)—N-valeryl-N-[[2′-(1H-tetrazol-5-yl)biphenyl-4-yl)methyl]valine, U.S. Pat. No. 5,399,578), EXP-3137 (2-N-butyl-4-chloro-1-[(2′-(1H-tetrazol-5-yl)biphenyl-4-yl)-methyl]imidazole-5-carboxylic acid, U.S. Pat. No. 5,138,069, U.S. Pat. No. 5,153,197 and U.S. Pat. No. 5,128,355), 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-)IH-tetrazol-5-yl)biphenyl-4-ylmethyl]quinazolin-4(3H)-one, 3-[2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5[[2′-(1H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo[1,5-c]pyrimidin-2(3H)-one, 2,7-diethyl-5-[[2′-(5-tetrazoly)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol[4,5-c]pyridine-5-ylmethyl]benzoic acid, ethyl ester, potassium salt, 3-methoxy-2,6-dimethyl-4-[[2′(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′ 1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinylisodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidzole-5-carboxylic acid 1-ethoxycarbonyloxyethyl ester, those disclosed in patent publications EP475206, EP497150, EP539086, EP539713, EP535463, EP535465, EP542059, EP497121, EP535420, EP407342, EP415886, EP424317, EP435827, EP433983, EP475898, EP490820, EP528762, EP324377, EP323841, EP420237, EP500297, EP426021, EP480204, EP429257, EP430709, EP434249, EP446062, EP505954, EP524217, EP514197, EP514198, EP514193, EP514192, EP450566, EP468372, EP485929, EP503162, EP533058, EP467207 EP399731, EP399732, EP412848, EP453210, EP456442, EP470794, EP470795, EP495626, EP495627, EP499414, EP499416, EP499415, EP511791, EP516392, EP520723, EP520724, EP539066, EP438869, EP505893, EP530702, EP400835, EP400974, EP401030, EP407102, EP411766, EP409332, EP412594, EP419048, EP480659, EP481614, EP490587, EP467715, EP479479, EP502725, EP503838, EP505098, EP505111 EP513,979 EP507594, EP510812, EP511767, EP512675, EP512676, EP512870, EP517357, EP537937, EP534706, EP527534, EP540356, EP461040, EP540039, EP465368, EP498723, EP498722, EP498721, EP515265, EP503785, EP501892, EP519831, EP532410, EP498361, EP432737, EP504888, EP508393, EP508445, EP403159, EP403158, EP425211, EP427463, EP437103, EP481448, EP488532, EP501269, EP500409, EP540400, EP005528, EP028834, EP028833, EP411507, EP425921, EP430300, EP434038, EP442473, EP443568, EP445811, EP459136, EP483683, EP518033, EP520423, EP531876, EP531874, EP392317, EP468470, EP470543, EP502314, EP529253, EP543263, EP540209, EP449699, EP465323, EP521768, EP415594, WO92/14468, WO93/08171, WO93/08169, WO91/00277, WO91/00281, WO91/14367, WO92/00067, WO92/00977, WO92/20342, WO93/04045, WO93/04046, WO91/15206, WO92/14714, WO92/09600, WO92/16552, WO93/05025, WO93/03018, WO91/07404, WO92/02508, WO92/13853, WO91/19697, WO91/11909, WO91/12001, WO91/11999, WO91/15209, WO91/15479, WO92/20687, WO92/20662, WO92/20661, WO93/01177, WO91/14679, WO91/13063, WO92/13564, WO91/17148, WO91/18888, WO91/19715, WO92/02257, WO92/04335, WO92/05161, WO92/07852, WO92/15577, WO93/03033, WO91/16313, WO92/00068, WO92/02510, WO92/09278, WO9210179, WO92/10180, WO92/10186, WO92/10181, WO92/10097, WO92/10183, WO92/10182, WO92/10187, WO92/10184, WO92/10188, WO92/10180, WO92/10185, WO92/20651, WO93/03722, WO93/06828, WO93/03040, WO92/19211, WO92/22533, WO92/06081, WO92/05784, WO93/00341, WO92/04343, WO92/04059, U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138,069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,048, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, U.S. Pat. No. 5,041,152, and U.S. Pat. No. 5,210,204, and pharmaceutically acceptable salts and esters thereof, α/β adrenergic blockers such as nipradilol, arotinolol, amosulalol, bretylium tosylate (CAS RN: 61-75-6), dihydroergtamine mesylate (such as ergotaman-3′, 6′,18-trione,9,-10-dihydro-12′-hydroxy-2′-methyl-5′-(phenylmethyl)-, (5′(a))-, monomethanesulfonate, e.g., DHE 45® Injection, Novartis), carvedilol (such as (±)-1-(Carbazol-4-yloxy)-3-[[2-(o-methoxyphenoxy)ethyl]amino]-2-propanol, e.g., Coreg®, SmithKline Beecham), labetalol (such as 5-[1-hydroxy-2-[(1-methyl-3-phenylpropyl) amino]ethyl]salicylamide monohydrochloride, e.g., Normodyne®, Schering), bretylium tosylate (Benzenemethanaminium, 2-bromo-N-ethyl-N,N-dimethyl-, salt with 4-methylbenzenesulfonic acid (1:1) CAS RN 61-75-6), phentolamine mesylate (Phenol, 3-[[(4,5-dihydro-1H-imidazol-2-yl)methyl](4-methylphenyl)amino]-, monomethanesulfonate (salt) CAS RN 65-28-1), solypertine tartrate (5H-1,3-Dioxolo[4,5-f]indole, 7-[2-[4-(2-methoxyphenyl)-1-piperazinyl]ethyl]-, (2R,3R)-2,3-dihydroxybutanedioate (1:1) CAS RN 5591-43-5), zolertine hydrochloride (piperazine, 1-phenyl4-[2-(1H-tetrazol-5-yl)ethyl]-, monohydrochloride (8Cl, 9Cl) CAS RN 7241-94-3) and the like;
α adrenergic receptor blockers, such as alfuzosin (CAS RN: 81403-68-1), terazosin, urapidil, prazosin (Minipress®), tamsulosin, bunazosin, trimazosin, doxazosin, naftopidil, indoramin, WHP 164, XEN010, fenspiride hydrochloride (which may be prepared as disclosed in U.S. Pat. No. 3,399,192), proroxan (CAS RN 33743-96-3), and labetalol hydrochloride and combinations thereof, a 2 agonists such as methyldopa, methyldopa HCL, lofexidine, tiamenidine, moxonidine, rilmenidine, guanobenz, and the like;
aldosterone inhibitors, and the like; renin inhibitors including Aliskiren (SPP 100; Novartis/Speedel); angiopoietin-2-binding agents such as those disclosed in WO03/030833;
anti-angina agents such as ranolazine (hydrochloridel-piperazineacetamide, N-(2,6-dimethylphenyl)-4-[2-hydroxy-3-(2-methoxyphenoxy)propyl]-, dihydrochloride CAS RN 95635-56-6), betaxolol hydrochloride (2-Propanol, 1-[4-[2 (cyclopropylmethoxy)ethyl]phenoxy]-3-[(1-methylethyl)amino]-, hydrochloride CAS RN 63659-19-8), butoprozine hydrochloride (Methanone, [4-[3(dibutylamino)propoxy]phenyl](2-ethyl-3-indolizinyl)-, monohydrochloride CAS RN 62134-34-3), cinepazet maleatel-piperazineacetic acid, 4-[1-oxo-3-(3,4,5-trimethoxyphenyl)-2-propenyl]-, ethyl ester, (2Z)-2-butenedioate (1:1) CAS RN 50679-07-7), tosifen (Benzenesulfonamide, 4-methyl-N-[[[(1S)-1-methyl-2-phenylethyl]amino]carbonyl]-CAS RN 32295-184), verapamilhydrochloride (Benzeneacetonitrile, α-[3-[[2-(3,4-dimethoxyphenyl)ethyl]methylamino]propyl]-3,4-dimethoxy-α-(1-methylethyl)-, monohydrochloride CAS RN 152-114), molsidomine (1,2,3-Oxadiazolium, 5-[(ethoxycarbonyl)amino]-3-(4-morpholinyl)-, inner salt CAS RN 25717-80-0), and ranolazine hydrochloride (1-Piperazineacetamide, N-(2,6-dimethylphenyl)₄-[2-hydroxy-3-(2-meth-oxyphenoxy)propyl]-, dihydrochloride CAS RN 95635-56-6); tosifen (Benzenesulfonamide, 4-methyl-N-[[[(1S)-1-methyl-2-phenylethyl]amino]carbonyl]-CAS RN 32295-184); adrenergic stimulants such as guanfacine hydrochloride (such as N-amidino-2-(2,6-dichlorophenyl) acetamide hydrochloride, e.g., Tenex® Tablets available from Robins); methyldopa-hydrochlorothiazide (such as levo-3-(3,4-dihydroxyphenyl)-2-methylalanine) combined with Hydrochlorothiazide (such as 6-chloro-3,4-dihydro-2H-1,2,4-benzothiadiazine-7-sulfonamide 1,1-dioxide, e.g., the combination as, e.g., Aldoril® Tablets available from Merck), methyldopa-chlorothiazide (such as 6-chloro-2H-1,2,4-benzothiadiazine-7-sulfonamide 1,1-dioxide and methyldopa as described above, e.g., Aldoclor®, Merck), clonidine hydrochloride (such as 2-(2,6-dichlorophenylamino)-2-imidazoline hydrochloride and chlorthalidone (such as 2-chloro-5-(1-hydroxy-3-oxo-1-isoindolinyl) benzenesulfonamide), e.g., Combipres®, Boehringer Ingelheim), clonidine hydrochloride (such as 2-(2,6-dichlorophenylamino)-2-imidazoline hydrochloride, e.g., Catapres®, Boehringer Ingelheim), clonidine (1H-Imidazol-2-amine, N-(2,6-dichlorophenyl)4,5-dihydro-CAS RN 4205-90-7), Hyzaar (Merck; a combination of losartan and hydrochlorothiazide), Co-Diovan (Novartis; a combination of valsartan and hydrochlorothiazide, Lotrel (Novartis; a combination of benazepril and amlodipine) and Caduet (Pfizer; a combination of amlodipine and atorvastatin), and those agents disclosed in US20030069221.

The guanylin potentiating agents described herein can be used in combination therapy with one or more of the following agents useful in the treatment of respiratory and other disorders including but not limited to:

(1) β-agonists including but not limited to: albuterol (PROVENTIL®, SALBUTAMOI®, VENTOLIN®), bambuterol, bitoterol, clenbuterol, fenoterol, formoterol, isoetharine (BRONKOSOL®, BRONKOMETER®), metaproterenol (ALUPENT®, METAPREL®), pirbuterol (MAXAIR®), reproterol, rimiterol, salmeterol, terbutaline (BRETHAIRE®, BRETHINE®, BRICANYL®), adrenalin, isoproterenol (ISUPREL®), epinephrine bitartrate (PRIMATENE®), ephedrine, orciprenline, fenoterol and isoetharine;
(2) steroids, including but not limited to beclomethasone, beclomethasone dipropionate, betamethasone, budesonide, bunedoside, butixocort, dexamethasone, flunisolide, fluocortin, fluticasone, hydrocortisone, methyl prednisone, mometasone, predonisolone, predonisone, tipredane, tixocortal, triamcinolone, and triamcinolone acetonide;
(3) β-agonist-corticosteroid combinations [e.g., salmeterol-fluticasone (ADVAIR®), formoterol-budesonid (SYMBICORT®)];
(4) leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (SINGULAIR®), pranlukast, iralukast, pobilukast, SKB-106,203 and compounds described as having LTD4 antagonizing activity described in U.S. Pat. No. 5,565,473;
(5) 5-lipoxygenase inhibitors and/or leukotriene biosynthesis inhibitors [e.g., zileuton and BAY1005 (CA registry 128253-31-6)];
(6) histamine H1 receptor antagonists/antihistamines (i.e., any compound that is capable of blocking, inhibiting, reducing or otherwise interrupting the interaction between histamine and its receptor) including but not limited to: astemizole, acrivastine, antazoline, azatadine, azelastine, astamizole, bromopheniramine, bromopheniramine maleate, carbinoxamine, carebastine, cetirizine, chlorpheniramine, chloropheniramine maleate, cimetidine, clemastine, cyclizine, cyproheptadine, descarboethoxyloratadine, dexchlorpheniramine, dimethindene, diphenhydramine, diphenylpyraline, doxylamine succinate, doxylamine, ebastine, efletirizine, epinastine, farnotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norasternizole, noraztemizole, phenindamine, pheniramine, picumast, promethazine, pynlamine, pyrilamine, ranitidine, temelastine, terfenadine, trimeprazine, tripelenamine, and triprolidine;
(7) an anticholinergic including but not limited to: atropine, benztropine, biperiden, flutropium, hyoscyamine, ilutropium, ipratropium, ipratropium bromide, methscopolamine, oxybutinin, rispenzepine, scopolamine, and tiotropium;
(8) an anti-tussive including but not limited to: dextromethorphan, codeine, and hydromorphone;
(9) a decongestant including but not limited to: pseudoephedrine and phenylpropanolamine;
(10) an expectorant including but not limited to: guafenesin, guaicolsulfate, terpin, ammonium chloride, glycerol guaicolate, and iodinated glycerol;
(11) a bronchodilator including but not limited to: theophylline and aminophylline;
(12) an anti-inflammatory including but not limited to: fluribiprofen, diclophenac, indomethacin, ketoprofen, S-ketroprophen, tenoxicam;
(13) a PDE (phosphodiesterase) inhibitor including but not limited to those disclosed herein;
(14) a recombinant humanized monoclonal antibody [e.g. xolair (also called omalizumab), rhuMab, and talizumab];
(15) a humanized lung surfactant including recombinant forms of surfactant proteins SP-B, SP—C or SP-D [e.g. SURFAXIN®, formerly known as dsc-104 (Discovery Laboratories)],
(16) agents that inhibit epithelial sodium channels (ENaC) such as amiloride and related compounds;
(17) antimicrobial agents used to treat pulmonary infections such as acyclovir, amikacin, amoxicillin, doxycycline, trimethoprin sulfamethoxazole, amphotericin B, azithromycin, clarithromycin, roxithromycin, clarithromycin, cephalosporins(ceffoxitin, cefmetazole etc), ciprofloxacin, ethambutol, gentimycin, ganciclovir, imipenem, isoniazid, itraconazole, penicillin, ribavirin, rifampin, rifabutin, amantadine, rimantidine, streptomycin, tobramycin, and vancomycin;
(18) agents that activate chloride secretion through Ca++ dependent chloride channels (such as purinergic receptor (P2Y(2) agonists);
(19) agents that decrease sputum viscosity, such as human recombinant DNase 1, (Pulmozyme®);
(20) nonsteroidal anti-inflammatory agents (acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam, tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac); and
(21) aerosolized antioxidant therapeutics such as S-Nitrosoglutathione.

The guanylin potentiating agents described herein can be used in combination therapy with an anti-obesity agent. Suitable such agents include, but are not limited to:

11β HSD-1 (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO01/90091, WO01/90090, WO01/90092 and WO02/072084;
5HT antagonists such as those in WO03/037871, WO03/037887, and the like;
5HT1a modulators such as carbidopa, benserazide and those disclosed in U.S. Pat. No. 6,207,699, WO03/031439, and the like;
5HT2c (serotonin receptor 2c) agonists, such as BVT933, DPCA37215, IK264, PNU 22394, WAY161503, R-1065, SB 243213 (Glaxo Smith Kline) and YM 348 and those disclosed in U.S. Pat. No. 3,914,250, WO00/77010, WO02/36596, WO02/48124, WO02/10169, WO01/66548, WO02/44152, WO02/51844, WO02/40456, and WO02/40457;
5HT6 receptor modulators, such as those in WO03/030901, WO03/035061, WO03/039547, and the like;
acyl-estrogens, such as oleoyl-estrone, disclosed in del Mar-Grasa, M. et al., Obesity Research, 9:202-9 (2001) and Japanese Patent Application No. JP 2000256190;
anorectic bicyclic compounds such as 1426 (Aventis) and 1954 (Aventis), and the compounds disclosed in WO00/18749, WO01/32638, WO01/62746, WO01/62747, and WO03/015769;
CB 1 (cannabinoid-1 receptor) antagonist/inverse agonists such as rimonabant (Acomplia; Sanofi), SR-147778 (Sanofi), SR-141716 (Sanofi), BAY 65-2520 (Bayer), and SLV 319 (Solvay), and those disclosed in patent publications U.S. Pat. No. 4,973,587, U.S. Pat. No. 5,013,837, U.S. Pat. No. 5,081,122, U.S. Pat. No. 5,112,820, U.S. Pat. No. 5,292,736, U.S. Pat. No. 5,532,237, U.S. Pat. No. 5,624,941, U.S. Pat. No. 6,028,084, U.S. Pat. No. 6,509,367, U.S. Pat. No. 6,509,367, WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and EP658546;
CCK-A (cholecystokinin-A) agonists, such as AR-R 15849, GI 181771 (GSK), JMV-180, A-71378, A-71623 and SR146131 (Sanofi), and those described in U.S. Pat. No. 5,739,106;
CNTF (Ciliary neurotrophic factors), such as GI-181771 (Glaxo-SmithKline), SR146131 (Sanofi Synthelabo), butabindide, PD170,292, and PD 149164 (Pfizer);
CNTF derivatives, such as Axokine® (Regeneron), and those disclosed in WO94/09134, WO98/22128, and WO99/43813;
dipeptidyl peptidase IV (DP-IV) inhibitors, such as isoleucine thiazolidide, valine pyrrolidide, NVP-DPP728, LAF237, P93/01, P 3298, TSL 225 (tryptophyl-1,2,3,4-tetrahydroisoquinoline-3-carboxylic acid; disclosed by Yamada et al, Bioorg. & Med. Chem. Lett. 8 (1998) 1537-1540), TMC-2A/2B/2C, CD26 inhibtors, FE 999011, P9310/K364, VIP 0177, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) and the compounds disclosed patent publications. WO99/38501, WO99/46272, WO99/67279 (Probiodrug), WO99/67278 (Probiodrug), WO99/61431 (Probiodrug), WO02/083128, WO02/062764, WO03/000180, WO03/000181, WO03/000250, WO03/002530, WO03/002531, WO03/002553, WO03/002593, WO03/004498, WO03/004496, WO03/017936, WO03/024942, WO03/024965, WO03/033524, WO03/037327 and EP1258476; growth hormone secretagogue receptor agonists/antagonists, such as NN703, hexarelin, MK-0677 (Merck), SM-130686, CP-424391 (Pfizer), LY 444,711 (Eli Lilly), L-692,429 and L-163,255, and such as those disclosed in U.S. Ser. No. 09/662,448, U.S. provisional application 60/203,335, U.S. Pat. No. 6,358,951, US2002049196, US2002/022637, WO01/56592 and WO02/32888;
H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO02/15905, WO03/024928 and WO03/024929;
leptin derivatives, such as those disclosed in U.S. Pat. No. 5,552,524, U.S. Pat. No. 5,552,523, U.S. Pat. No. 5,552,522, U.S. Pat. No. 5,521,283, WO96/23513, WO96/23514, WO96/23515, WO96/23516, WO96/23517, WO96/23518, WO96/23519, and WO96/23520;
leptin, including recombinant human leptin (PEG-OB, Hoffman La Roche) and recombinant methionyl human leptin (Amgen);
lipase inhibitors, such as tetrahydrolipstatin (orlistat/Xenical®), Triton WR1339, RHC80267, lipstatin, teasaponin, diethylumbelliferyl phosphate, FL-386, WAY-121898, Bay-N-3176, valilactone, esteracin, ebelactone A, ebelactone B, and RHC 80267, and those disclosed in patent publications WO01/77094, U.S. Pat. No. 4,598,089, U.S. Pat. No. 4,452,813, U.S. Pat. No. 5,512,565, U.S. Pat. No. 5,391,571, U.S. Pat. No. 5,602,151, U.S. Pat. No. 4,405,644, U.S. Pat. No. 4,189,438, and U.S. Pat. No. 4,242,453;
lipid metabolism modulators such as maslinic acid, erythrodiol, ursolic acid uvaol, betulinic acid, betulin, and the like and compounds disclosed in WO03/011267; Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410;
Mc5r (melanocortin 5 receptor) modulators, such as those disclosed in WO97/19952, WO00/15826, WO00/15790, US20030092041;
melanin-concentrating hormone 1 receptor (MCHR) antagonists, such as T-226296 (Takeda), SB 568849, SNP-7941 (Synaptic), and those disclosed in patent publications WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480, JP13226269, and JP1437059;
mGluR5 modulators such as those disclosed in WO03/029210, WO03/047581, WO03/048137, WO03/051315, WO03/051833, WO03/053922, WO03/059904, and the like;
serotoninergic agents, such as fenfluramine (such as Pondimin® (Benzeneethanamine, N-ethyl-alpha-methyl-3-(trifluoromethyl)-, hydrochloride), Robbins), dexfenfluramine (such as Redux® (Benzeneethanamine, N-ethyl-alpha-methyl-3-(trifluoromethyl)-, hydrochloride), Interneuron) and sibutramine ((Meridia®, Knoll/Reductil™) including racemic mixtures, as optically pure isomers (+) and (−), and pharmaceutically acceptable salts, solvents, hydrates, clathrates and prodrugs thereof including sibutramine hydrochloride monohydrate salts thereof, and those compounds disclosed in U.S. Pat. No. 4,746,680, U.S. Pat. No. 4,806,570, and U.S. Pat. No. 5,436,272, US20020006964, WO01/27068, and WO01/62341;
NE (norepinephrine) transport inhibitors, such as GW 320659, despiramine, talsupram, and nomifensine;
NPY 1 antagonists, such as BIBP3226, J-115814, BIBO 3304, LY-357897, CP-671906, GI-264879A, and those disclosed in U.S. Pat. No. 6,001,836, WO96/14307, WO01/23387, WO99/51600, WO01/85690, WO01/85098, WO01/85173, and WO01/89528;

NPY5 (neuropeptide Y Y5) antagonists, such as 152,804, GW-569180A, GW-594884A, GW-587081×, GW-548118×, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in patent publications U.S. Pat. No. 6,140,354, U.S. Pat. No. 6,191,160, U.S. Pat. No. 6,218,408, U.S. Pat. No. 6,258,837, U.S. Pat. No. 6,313,298, U.S. Pat. No. 6,326,375, U.S. Pat. No. 6,329,395, U.S. Pat. No. 6,335,345, U.S. Pat. No. 6,337,332, U.S. Pat. No. 6,329,395, U.S. Pat. No. 6,340,683, EP01010691, EP-01044970, WO97/19682, WO97/20820, WO97/20821, WO97/20822, WO97/20823, WO98/27063, WO00/107409, WO00/185714, WO00/185730, WO00/64880, WO00/68197, WO00/69849, WO/0113917, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al., J. Med. Chem. 43:4288-4312 (2000);

opioid antagonists, such as nalmefene (REVEX®), 3-methoxynaltrexone, naloxone, and naltrexone and those disclosed in WO00/21509;

orexin antagonists, such as SB-334867-A and those disclosed in patent publications WO01/96302, WO01/68609, WO02/44172, WO02/51232, WO02/51838, WO02/089800, WO02/090355, WO03/023561, WO03/032991, and WO03/037847;
PDE inhibitors (e.g. compounds which slow the degradation of cyclic AMP (cAMP) and/or cyclic GMP (cGMP) by inhibition of the phosphodiesterases, which can lead to a relative increase in the intracellular concentration of cAMP and cGMP; possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors) such as those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as Rx-RA-69, SCH-51866, KT-734, vesnarinone, zaprinast, SKF-96231, ER-21355, BF/GP-385, NM-702 and sildenafil (Viagra™)), PDE4 inhibitors (such as etazolate, ICI63197, RP73401, imazolidinone (RO-20-1724), MEM 1414 (R1533/R1500; Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, aminone, pimobendan, cilostazol, quazinone and N-(3,5-dichloropyrid-4-yl)-3-cyclopropylmethoxy-4-difluoromethoxybenzamide, PDE3 inhibitors (such as ICI153, 100, bemorandane (RWJ 22867), MCI-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, tadalafil (Clalis®), theophylline, and vardenafil (Levitra®);
Neuropeptide Y2 (NPY2) agonists include but are not limited to: peptide YY and fragments and variants thereof (e.g. YY3-36 (PYY3-36)(N. Engl. J. Med. 349:941, 2003; IKPEAPGE DASPEELNRY YASLRHYLNL VTRQRY (SEQ ID NO:19) and PYY agonists such as those disclosed in WO03/026591, WO03/057235, and WO03/027637;
serotonin reuptake inhibitors, such as, paroxetine, fluoxetine (Prozac™), fluvoxamine, sertraline, citalopram, and imipramine, and those disclosed in U.S. Pat. No. 6,162,805, U.S. Pat. No. 6,365,633, WO03/00663, WO01/27060, and WO01/162341;
thyroid hormone β agonists, such as KB-2611 (KaroBioBMS), and those disclosed in WO02/15845, WO97/21993, WO99/00353, GB98/284425, U.S. Provisional Application No. 60/183,223, and Japanese Patent Application No. JP 2000256190; UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in WO99/00123;
β3 (beta adrenergic receptor 3) agonists, such as AJ9677/TAK677 (Dainippon/Takeda), L750355 (Merck), CP331648 (Pfizer), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), SR 59119A, and those disclosed in U.S. Pat. No. 5,541,204, U.S. Pat. No. 5,770,615, U.S. Pat. No. 5,491,134, U.S. Pat. No. 5,776,983, U.S. Pat. No. 4,880,64, U.S. Pat. No. 5,705,515, U.S. Pat. No. 5,451,677, WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881; noradrenergic agents including, but not limited to, diethylpropion (such as Tenuate® (1-propanone, 2-(diethylamino)-1-phenyl-, hydrochloride), Merrell), dextroamphetamine (also known as dextroamphetamine sulfate, dexamphetamine, dexedrine, Dexampex, Ferndex, Oxydess II, Robese, Spancap #1), mazindol ((or 5-(p-chlorophenyl)-2,5-dihydro-3H-imidazo[2,1-a]isoindol-5-ol) such as Sanorex®, Novartis or Mazanor®, Wyeth Ayerst), phenylpropanolamine (or Benzenemethanol, alpha-(1-aminoethyl)-, hydrochloride), phentermine ((or Phenol, 3-[[4,5-duhydro-1H-imidazol-2-yl)ethyl](4-methylpheny-1)amino], monohydrochloride) such as Adipex-P®, Lemmon, FASTIN®, Smith-Kline Beecham and Ionamin®, Medeva), phendimetrazine ((or (2S,3S)-3,4-Dimethyl-2phenylmorpholine L-(+)-tartrate (1:1)) such as Metra®(Forest), Plegine® (Wyeth-Ayerst), Prelu-2® (Boehringer Ingelheim), and Statobex® (Lemmon), phendamine tartrate (such as Thephorin® (2,3,4,9-Tetrahydro-2-methyl-9-phenyl-1H-indenol[2,1-c]pyridine L-(+)-tartrate (1:1)), Hoffmann-LaRoche), methamphetamine (such as Desoxyn®, Abbot ((S)—N, (alpha)-dimethylbenzeneethanamine hydrochloride)), and phendimetrazine tartrate (such as Bontril® Slow-Release Capsules, Amarin (−3,4-Dimethyl-2-phenylmorpholine Tartrate);
fatty acid oxidation upregulator/inducers such as Famoxin® (Genset);
monamine oxidase inhibitors including but not limited to befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO01/12176; and
other anti-obesity agents such as 5HT-2 agonists, ACC (acetyl-CoA carboxylase) inhibitors such as those described in WO03/072197, alpha-lipoic acid (alpha-LA), AOD9604, appetite suppressants such as those in WO03/40107, ATL-962 (Alizyme PLC), benzocaine, benzphetamine hydrochloride (Didrex), bladderwrack (focus vesiculosus), BRS3 (bombesin receptor subtype 3) agonists, bupropion, caffeine, CCK agonists, chitosan, chromium, conjugated linoleic acid, corticotropin-releasing hormone agonists, dehydroepiandrosterone, DGAT1 (diacylglycerol acyltransferase 1) inhibitors, DGAT2 (diacylglycerol acyltransferase 2) inhibitors, dicarboxylate transporter inhibitors, ephedra, exendin-4 (an inhibitor of glp-1) FAS (fatty acid synthase) inhibitors (such as Cerulenin and C75), fat resorption inhibitors (such as those in WO03/053451, and the like), fatty acid transporter inhibitors, natural water soluble fibers (such as psyllium, plantago, guar, oat, pectin), galanin antagonists, galega (Goat's Rue, French Lilac), garcinia cambogia, germander (teucrium chamaedrys), ghrelin antibodies and ghrelin antagonists (such as those disclosed in WO01/87335, and WO02/08250), peptide hormones and variants thereof which affect the islet cell secretion, such as the hormones of the secretin/gastric inhibitory peptide (GIP)/vasoactive intestinal peptide (VIP)/pituitary adenylate cyclase activating peptide (PACAP)/glucagon-like peptide II (GLP-II)/glicentin/glucagon gene family and/or those of the adrenomedullin/amylin/calcitonin gene related peptide (CGRP) gene family including GLP-1 (glucagon-like peptide 1) agonists (e.g. (1) exendin-4, (2) those GLP-1 molecules described in US20050130891 including GLP-1(7-34), GLP-1(7-35), GLP-1(7-36) or GLP-1(7-37) in its C-terminally carboxylated or amidated form or as modified GLP-1 peptides and modifications thereof including those described in paragraphs 17-44 of US20050130891, and derivatives derived from GLP-1-(7-34)COOH and the corresponding acid amide are employed which have the following general formula:

R-NH-HAEGTFTSDVSYLEGQAAKEFIAWLVK-CONH₂ (SEQ ID NO:4853)

wherein R═H or an organic compound having from 1 to 10 carbon atoms. Preferably, R is the residue of a carboxylic acid. Particularly preferred are the following carboxylic acid residues: formyl, acetyl, propionyl, isopropionyl, methyl, ethyl, propyl, isopropyl, n-butyl, sec-butyl, tert-butyl.) and glp-1 (glucagon-like peptide-1), glucocorticoid antagonists, glucose transporter inhibitors, growth hormone secretagogues (such as those disclosed and specifically described in U.S. Pat. No. 5,536,716), interleukin-6 (IL-6) and modulators thereof (as in WO03/057237, and the like), L-carnitine, Mc3r (melanocortin 3 receptor) agonists, MCH2R (melanin concentrating hormone 2R) agonist/antagonists, melanin concentrating hormone antagonists, melanocortin agonists (such as Melanotan II or those described in WO 99/64002 and WO 00/74679), nomame herba, phosphate transporter inhibitors, phytopharm compound 57 (CP 644,673), pyruvate, SCD-1 (stearoyl-CoA desaturase-1) inhibitors, T71 (Tularik, Inc., Boulder Colo.), Topiramate (Topimax®, indicated as an anti-convulsant which has been shown to increase weight loss), transcription factor modulators (such as those disclosed in WO03/026576), β-hydroxy steroid dehydrogenase-1 inhibitors (β-HSD-1), β-hydroxy-β-methylbutyrate, p57 (Pfizer), Zonisamide (Zonegran™, indicated as an anti-epileptic which has been shown to lead to weight loss), and the agents disclosed in US20030119428 paragraphs 20-26.
The guanylin potentiating agents described herein can be used in therapeutic combination with one or more anti-diabetic agents, including but not limited to:
PPARγ agonists such as glitazones (e.g., WAY-120,744, AD 5075, balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), rivoglitazone (CS-011, Sankyo), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), NN-2344 (Dr. Reddy/NN), YM-440 (Yamanouchi), LY-300512, LY-519818, R483 (Roche), T131 (Tularik), and the like and compounds disclosed in U.S. Pat. No. 4,687,777, U.S. Pat. No. 5,002,953, U.S. Pat. No. 5,741,803, U.S. Pat. No. 5,965,584, U.S. Pat. No. 6,150,383, U.S. Pat. No. 6,150,384, U.S. Pat. No. 6,166,042, U.S. Pat. No. 6,166,043, U.S. Pat. No. 6,172,090, U.S. Pat. No. 6,211,205, U.S. Pat. No. 6,271,243, U.S. Pat. No. 6,288,095, U.S. Pat. No. 6,303,640, U.S. Pat. No. 6,329,404, U.S. Pat. No. 5,994,554, WO97/10813, WO97/27857, WO97/28115, WO97/28137, WO97/27847, WO00/76488, WO03/000685, WO03/027112, WO03/035602, WO03/048130, WO03/055867, and pharmaceutically acceptable salts thereof;
biguanides such as metformin hydrochloride (N,N-dimethylimidodicarbonimidic diamide hydrochloride, such as Glucophage™, Bristol-Myers Squibb); metformin hydrochloride with glyburide, such as Glucovance™, Bristol-Myers Squibb); buformin (Imidodicarbonimidic diamide, N-butyl-); etoformine (1-Butyl-2-ethylbiguanide, Schering A. G.); other metformin salt forms (including where the salt is chosen from the group of, acetate, benzoate, citrate, ftimarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate and phosphate), and phenformin;
protein tyrosine phosphatase-1B (PTP-1B) inhibitors, such as A-401,674, KR 61639, OC-060062, OC-83839, OC-297962, MC52445, MC52453, ISIS 113715, and those disclosed in WO99/585521, WO99/58518, WO99/58522, WO99/61435, WO03/032916, WO03/032982, WO03/041729, WO03/055883, WO02/26707, WO02/26743, JP2002114768, and pharmaceutically acceptable salts and esters thereof;
sulfonylureas such as acetohexamide (e.g. Dymelor, Eli Lilly), carbutamide, chlorpropamide (e.g. Diabinese®, Pfizer), gliamilide (Pfizer), gliclazide (e.g. Diamcron, Servier Canada Inc), glimepiride (e.g. disclosed in U.S. Pat. No. 4,379,785, such as Amaryl™, Aventis), glipentide, glipizide (e.g. Glucotrol or Glucotrol XL Extended Release, Pfizer), gliquidone, glisolamide, glyburide/glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof;
meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof;
α glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-alpha-D-glucopyranoside, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, U.S. Pat. No. 5,109,1 and WO01/47528 (polyamines);
α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765;
SGLT2 inhibitors including those disclosed in U.S. Pat. No. 6,414,126 and U.S. Pat. No. 6,515,117;
an aP2 inhibitor such as disclosed in U.S. Pat. No. 6,548,529;
insulin secreatagogues such as linogliride, A-4166, forskilin, dibutyrl cAMP, isobutylmethylxanthine (IBMX), and pharmaceutically acceptable salts and esters thereof;
fatty acid oxidation inhibitors, such as clomoxir, and etomoxir, and pharmaceutically acceptable salts and esters thereof;
A2 antagonists, such as midaglizole, isaglidole, deriglidole, idazoxan, earoxan, and fluparoxan, and pharmaceutically acceptable salts and esters thereof,
insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-1 (1-36) amide, GLP-1 (73-7) (insulintropin, disclosed in U.S. Pat. No. 5,614,492), LY-315902 (Lilly), GLP-1 (7-36)-NH2), AL-401 (AutoImmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN'S DESK REFERENCE, 55.sup.th Ed. (2001) Medical Economics, Thomson Healthcare (disclosing other suitable human insulins);
non-thiazolidinediones such as JT-501 and farglitazar (GW-2570/GI-262579), and pharmaceutically acceptable salts and esters thereof;
PPARα/γ dual agonists such as AR-HO39242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methyl]benzamide), L-796449, LR-90, MK-0767 (Merck/Kyorin/Banyu), SB 219994, muraglitazar (BMS), tesaglitzar (Astrazeneca), reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976, U.S. application Ser. No. 09/664,598, filed Sep. 18, 2000, Murakami et al. Diabetes 47, 1841-1847 (1998), and pharmaceutically acceptable salts and esters thereof;
other insulin sensitizing drugs;
VPAC2 receptor agonists;
GLK modulators, such as those disclosed in WO03/015774;
retinoid modulators such as those disclosed in WO03/000249;
GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like; glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof;
ATP consumption promotors such as those disclosed in WO03/007990;
TRB3 inhibitors;
vanilloid receptor ligands such as those disclosed in WO03/049702;
hypoglycemic agents such as those disclosed in WO03/015781 and WO03/040114;
glycogen synthase kinase 3 inhibitors such as those disclosed in WO03/035663 agents such as those disclosed in WO99/51225, US20030134890, WO01/24786, and WO03/059870;
insulin-responsive DNA binding protein-1 (IRDBP-1) as disclosed in WO03/057827, and the like;
adenosine A2 antagonists such as those disclosed in WO03/035639, WO03/035640, and the like;
PPARδ agonists such as GW 501516, GW 590735, and compounds disclosed in JP10237049 and WO02/14291;
dipeptidyl peptidase IV (DP-IV) inhibitors, such as isoleucine thiazolidide, NVP-DPP728A (1-[[[2-[(5-cyanopyridin-2-yl)amino]ethyl]amino]acetyl]-2-cyano-(S)-pyrrolidine, disclosed by Hughes et al, Biochemistry, 38(36), 11597-11603, 1999), P32/98, NVP-LAF-237, P3298, TSL225 (tryptophyl-1,2,3,4-tetrahydro-isoquinoline-3-carboxylic acid, disclosed by Yamada et al, Bioorg. & Med. Chem. Lett. 8 (1998) 1537-1540), valine pyrrolidide, TMC-2A/2B/2C, CD-26 inhibitors, FE999011, P9310/K364, VIP 0177, DPP4, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996), and the compounds disclosed in U.S. Pat. No. 6,395,767, U.S. Pat. No. 6,573,287, U.S. Pat. No. 6,395,767 (compounds disclosed include BMS-477118, BMS-471211 and BMS 538,305), WO99/38501, WO99/46272, WO99/67279, WO99/67278, WO99/61431 WO03/004498, WO03/004496, EP 1258476, WO02/083128, WO02/062764, WO03/000250, WO03/002530, WO03/002531, WO03/002553, WO03/002593, WO03/000180, and WO03/000181;
GLP-1 agonists such as exendin-3 and exendin-4 (including the 39 aa peptide synthetic exendin-4 called Exenatide®), and compounds disclosed in US2003087821 and NZ 504256, and pharmaceutically acceptable salts and esters thereof;
peptides including amlintide and Symlin® (pramlintide acetate); and glycokinase activators such as those disclosed in US2002103199 (fused heteroaromatic compounds) and WO02/48106 (isoindolin-1-one-substituted propionamide compounds).

The guanylin potentiating agents described herein useful in the treatment of obesity can be administered as a cotherapy with electrostimulation (US20040015201).

The guanylin potentiating agents described herein can be used in combination therapy with agents that activate soluble guanylate cyclase, for example those described in US20040192680.

The guanylin potentiating agents described herein can be used in combination therapy with a phosphodiesterase inhibitor. PDE inhibitors are those compounds which slow the degradation of cyclic AMP (cAMP) and/or cyclic GMP (cGMP) by inhibition of the phosphodiesterases, which can lead to a relative increase in the intracellular concentration of cAMP and/or cGMP. Possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors. By way of example, those PDE inhibitors may be mentioned such as are described and/or claimed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577) and WO9307124, EP0163965, EP0393500, EP0510562, EP0553174, WO9501338 and WO9603399. PDE5 inhibitors which may be mentioned by way of example are RX-RA-69, SCH-51866, KT-734, vesnarinone, zaprinast, SKF-96231, ER-21355, BF/GP-385, NM-702 and sildenafil (Viagra®). PDE4 inhibitors which may be mentioned by way of example are RO-20-1724, MEM 1414 (R1533/R1500; Pharmacia Roche), DENBUFYLLINE, ROLIPRAM, OXAGRELATE, NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840, SKF-107806, PICLAMILAST, RS-17597, RS-25344-000, SB-207499, TIBENELAST, SB-210667, SB-211572, SB-211600, SB-212066, SB-212179, GW-3600, CDP-840, MOPIDAMOL, ANAGRELIDE, IBUDILAST, AMRINONE, PIMOBENDAN, CILOSTAZOL, QUAZINONE and N-(3,5-dichloropyrid-4-yl)-3-cyclopropylmethoxy4-difluoromethoxybenzamide. PDE3 inhibitors which may be mentioned by way of example are SULMAZOLE, AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, ENOXIMONE and MILRINONE. PDE3/4 inhibitors which may be mentioned by way of example are BENAFENTRINE, TREQUINSIN, ORG-30029, ZARDAVERINE, L-686398, SDZ-ISQ-844, ORG-20241, EMD-54622, and TOLAFENTRINE. Other PDE inhibitors include: cilomilast, pentoxifylline, roflumilast, tadalafil (Clalis®), theophylline, and vardenafil (Levitra®), zaprinast (PDE5 specific).

The guanylin potentiating agents described herein can be used in combination therapy (for example, in order to decrease or inhibit uterine contractions) with a tocolytic agent including but not limited to beta-adrenergic agents, magnesium sulfate, prostaglandin inhibitors, and calcium channel blockers.
The guanylin potentiating agents of the invention can be used in combination therapy with an anti-neoplastic agents including but not limited to alkylating agents, epipodophyllotoxins, nitrosoureas, antimetabolites, vinca alkaloids, anthracycline antibiotics, nitrogen mustard agents, and the like. Particular anti-neoplastic agents may include tamoxifen, taxol, etoposide and 5-fluorouracil. The peptides and agonists of the invention can be used in combination therapy (for example as in a chemotherapeutic composition) with an antiviral and monoclonal antibody therapies.
The guanylin potentiating agents of the invention can be used in combination therapy (for example, in prevention/treatment of congestive heart failure or another method described herein) with the partial agonist of the nociceptin receptor ORL1 described by Dooley et al. (The Journal of Pharmacology and Experimental Therapeutics, 283 (2): 735-741, 1997). The agonist is a hexapeptide having the amino acid sequence Ac-RYY (RK) (WI) (RK)-NH2 (SEQ ID NO:4835) (“the Dooley peptide”), where the brackets show allowable variation of amino acid residue. Thus Dooley peptide can include but are not limited to KYYRWR (SEQ ID NO:4836), RYYRWR (SEQ ID NO:4837), KWRYYR (SEQ ID NO:4838), RYYRWK (SEQ ID NO:4839), RYYRWK (all-D amin acids), RYYRIK (SEQ ID NO:4840), RYYRIR (SEQ ID NO:4831), RYYKIK (SEQ ID NO:4842), RYYKIR (SEQ ID NO:4843), RYYKWR (SEQ ID NO:4844), RYYKWK (SEQ ID NO:4845), RYYRWR (SEQ ID NO:4846), RYYRWK (SEQ ID NO:4847), RYYRIK (SEQ ID NO:4848), RYYKWR (SEQ ID NO:4849), RYYKWK (SEQ ID NO:4850), RYYRWK (SEQ ID NO:4851) and KYYRWK (SEQ ID NO:4852), wherein the amino acid residues are in the L-form unless otherwise specified. The peptides and agonists of the invention can *also be used in combination therapy with peptide conjugate modifications of the Dooley peptide described in WO0198324.

### Methods of Treatment

Guanylin potentiating agents can be used to treat conditions in which it would be useful to potentiate the action of guanylin whether the guanylin is endogenous or is administered exogenously.

The guanylin potentiating agents described herein can be used alone or in combination therapy for the treatment or prevention of congestive heart failure. Such agents can be used in combination with natriuretic peptides (e.g., atrial natriuretic peptide, brain natriuretic peptide or C-type natriuretic peptide), a diuretic, or an inhibitor of angiotensin converting enzyme.

The guanylin potentiating agents described herein can be used alone or in combination therapy for the treatment or prevention of benign prostatic hyperplasia (BPH). Such agents can be used in combination with one or more agents for treatment of BPH, for example, a 5-alpha reductase inhibitor (e.g., finasteride) or an alpha adrenergic inhibitor (e.g., doxazosine).

The guanylin potentiating agents described herein can be used alone or in combination therapy for the treatment, prevention or reduction of visceral pain associated with a gastrointestinal disorder or pain associated with another disorder.

The guanylin potentiating agents described herein can be used alone or in combination therapy for the treatment or prevention of obesity-related disorders (e.g. disorders that are associated with, caused by, or result from obesity). Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrhythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. The agents may be used to reduce or control body weight (or fat) or to prevent and/or treat obesity or other appetite related disorders related to the excess consumption of food, ethanol and other appetizing substances. The agents may be used to modulate lipid metabolism, reduce body fat (e.g. via increasing fat utilization) or reduce (or suppress) appetite (e.g. via inducing satiety). Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastroesophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The agents are also useful for reducing the risk of secondary outcomes of obesity, such as reducing the risk of left ventricular hypertrophy.

The guanylin potentiating agents described herein can be used alone or in combination therapy for the treatment or prevention of gastrointestinal related disorders including: chronic intestinal pseudo-obstruction (Ogilvie's syndrome), colonic pseudoobstruction, Crohn's disease, dyspepsia (including functional dyspepsia or nonulcer dyspepsia), duodenogastric reflux, functional bowel disorder, functional gastrointestinal disorders, functional heartburn, gastroesophageal reflux disease (GERD), gastrointestinal motility disorders, gastroparesis (e.g. idopathic gastroparesis), hypertrophic pyloric stenosis, Inflammatory bowel disease, irritable bowel syndrome (IBS), post-operative ileus, and ulcerative colitis. The guanylin potentiating agents described herein can be used alone or in combination therapy to patient suffering from or susceptible to GI disorders relating to damage to the GI tract stemming from impact or surgical intervention. The guanylin potentiating agents described herein can be used alone or in combination therapy to patients at risk for or having particular diseases associated with hypomotility or stasis in the GI tract. For example, diabetic neuropathy, anorexia nervosa, and achlorhydria are frequently accompanied by gastric hypomotility. Damage to the GI tract following surgical intervention, for instance, can result in substantial gastric stasis. The guanylin potentiating agents described herein can be administered alone or in combination therapy to patients susceptible to or having a GI disorder associated with diabetes (e.g. diabetic gastropathy). The guanylin potentiating agents described herein can be used alone or in combination therapy to prevent and/or treat GI disorders characterized by at least one of nausea, vomiting, heartburn, postprandial discomfort, diarrhea, constipation, indigestion or related symptoms. guanylin potentiating agents described herein can be used alone or in combination therapy to prevent and/or treat GI disorders associated with at least one of diabetes, anorexia nervosa, bulimia, achlorhydria, achalasia, anal fissure, irritable bowel syndrome, intestinal pseudoobstruction, scleroderma and gastrointestinal damage.

The guanylin potentiating agents described herein can be used to prevent and/or treat constipation. Constipation can be used to describe bowel patterns which include one or more of hard, small, infrequent stools; the sensation of difficulty in passing stool, specifically excessive or ineffectual straining; the sensation of incomplete evacuation. Constipation has also been described as the passage of stool less than a certain number (e.g. 3) of times per week. A number of conditions can be associated with constipation. Constipation can be associated with numerous disorders and conditions. For example, constipation can be (1) associated with the use of a therapeutic agent (e.g. antihypertensives, anticonvulsants, antispasmodics, analgesics, anticholinergics, antidepressants, antipsychotics, cation-containing agents, anticonvulsants, ganglion blockers, vinca alkaloids); (2) associated with a muscular, neuropathic, metabolic or endocrine disorder (including but not limited to myotonic dystrophy, dermamyositis, systemic sclerosis, sclerodoma, amyloidosis (neurologic or muscular), ischemia, tumor of the central nervous system, autonomic neuropathy, Chagas disease, cystic fibrosis, diabetes mellitus, Hirschsprung disease, hyperthyroidism, hypocalcaemia, hypothyroidism, Multiple Sclerosis, neurofibromatosis, Parkinson's disease, and spinal cord lesions (for example, related to sacral nerve damage related to trauma or a tumor or the enteric nervous system)); (3) post-surgical constipation (postoperative ileus); (4) associated with a structural colon alteration (for example that associated with Neoplasm, stricture, volvulus, anorectal, inflammation, prolapse, rectocele, or fissure); (5) associated with the a gastrointestinal disorder; (6) associated with a systemic illness or disorder (for example, electrolyte abnormalities, thyroid disease, diabetes mellitus, panhypopituitarism, Addison's disease, pheochromocytoma, uremia, porphyria); (7) chronic constipation; (8) associated with the use of analgesic drugs (e.g. opioid induced constipation); (9) associated with megacolon; and (10) idiopathic constipation (functional constipation). Functional constipation can be associated with normal transit, slow transit (e.g. one or fewer bowel movements per week) and pelvic floor dyssynergia. Pelvic floor dyssynergia is considered a disorder of the rectum and anus although these patients also have abnormal contractions throughout the colon. Patients with pelvic floor dyssynergia have abnormal colonic pressure waves prior to defecation and present with symptoms that may include a sensation of incomplete evacuation, excessive straining, a need for digital disimpaction, perianal heaviness, and tenesmus. Constipation can be associated with bloating and abdominal pain. The guanylin potentiating agents of the invention can be used to prevent and/or treat low stool frequency or poor stool consistency.

The guanylin potentiating agents described herein can be used to treat decreased intestinal motility, slow digestion or slow stomach emptying. The guanylin potentiating agents described herein can be used to relieve one or more symptoms of IBS (bloating, pain, constipation), GERD (acid reflux into the esophagus), duodenogastric reflux, functional dyspepsia, or gastroparesis (nausea, vomiting, bloating, delayed gastric emptying) and other disorders described herein. The guanylin potentiating agents of the invention can be used to treat flatulence.

The guanylin potentiating agents described herein can be used to increase intestinal motility and to prevent and/or treat gastrointestinal immotility and other conditions calling for laxative or stool softener therapy. Gastrointestinal immotility can include constipation, and also includes delayed oral cecal transit time, irregular taxation, and other related gastrointestinal motility disfunction including impaction. Impaction is a condition where a large mass of dry, hard stool develops in the rectum, often due to chronic constipation. This mass may be so hard that it cannot be excreted. The subjects affected by constipation or gastrointestinal immotility can be refractory to laxative therapy and/or stool softener therapy.

The guanylin potentiating agents described herein can be used for the treatment or prevention of cancer, pre-cancerous growths, or metastatic growths. For example, they can be used for the prevention or treatment of: colorectal/local metastasized colorectal cancer, intestinal polyps, gastrointestinal tract cancer, lung cancer, cancer or pre-cancerous growths or metastatic growths of epithelial cells, polyps, breast, colorectal, lung, ovarian, pancreatic, prostatic, renal, stomach, bladder, liver, esophageal and testicular carcinoma, carcinoma (e.g., basal cell, basosquamous, Brown-Pearce, ductal carcinoma, Ehrlich tumor, Krebs, Merkel cell, small or non-small cell lung, oat cell, papillary, bronchiolar, squamous cell, transitional cell, (Walker), leukemia (e.g., B-cell, T-cell, HTLV, acute or chronic lymphocytic, mast cell, myeloid), histiocytonia, histiocytosis, Hodgkin's disease, non-Hodgkin's lymphoma, plasmacytoma, reticuloendotheliosis, adenoma, adeno-carcinoma, adenofibroma, adenolymphoma, ameloblastoma, angiokeratoma, angiolymphoid hyperplasia with eosinophilia, sclerosing angioma, angiomatosis, apudoma, branchionia, malignant carcinoid syndrome, carcinoid heart disease, carcinosarcoma, cementoma, cholangioma, cholesteatoma, chondrosarcoma, chondroblastoma, chondrosarcoma, chordoma, choristoma, craniopharyngioma, chrondrorna, cylindroma, cystadenocarcinoma, cystadenoma, cystosarconia phyllodes, dysgenninoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendothelioma, hemangioma, hemangio-pericytoma, hemangiosarcoma, hepatoma, islet cell tumor, Kaposi sarcoma, leiomyoma, leiomyosarcoma, leukosarcoma, Leydig cell tumor, lipoma, liposarcoma, lymphaugioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuroblastoma, neuroepithelioma, neurofibroma, neurofibromatosis, odontoma, osteoma, osteosarcoma, papilloma, paraganglioma, paraganglionia. nonchroinaffin, pinealoma, rhabdomyoma, rhabdomyosarcoma, Sertoli cell tumor, teratoma, theca cell tumor, and other diseases in which cells have become dysplastic, immortalized, or transformed.

The guanylin potentiating agents described herein can be used for the treatment or prevention of: Familial Adenomatous Polyposis (FAP) (autosomal dominant syndrome) that precedes colon cancer, hereditary nonpolyposis colorectal cancer (HNPCC), and inherited autosomal dominant syndrome.

For treatment or prevention of cancer, pre-cancerous growths and metastatic growths, the guanylin potentiating agents described herein can be used in combination therapy with radiation or chemotherapeutic agents, an inhibitor of a cGMP-dependent phosphodiesterase or a selective cyclooxygenase-2 inhibitor. A number of selective cyclooxygenase-2 inhibitors are described in US20010024664, U.S. Pat. No. 5,380,738, U.S. Pat. No. 5,344,991, U.S. Pat. No. 5,393,790, U.S. Pat. No. 5,434,178, U.S. Pat. No. 5,474,995, U.S. Pat. No. 5,510,368, WO02/062369, WO 96/06840, WO 96/03388, WO 96/03387, WO 96/19469, WO 96/25405, WO 95/15316, WO 94/15932, WO 94/27980, WO 95/00501, WO 94/13635, WO 94/20480, and WO 94/26731, the disclosures of which are herein incorporated by reference. [Pyrazol-1-yl]benzenesulfonamides have also been described as inhibitors of cyclooxygenase-2.

The guanylin potentiating agents described herein can be used in the treatment or prevention of inflammation. Thus, they can be used alone or in combination with an inhibitor of cGMP-dependent phosphodiesterase or a selective cyclooxygenase-2 inhibitor for treatment of: organ inflammation, IBD (e.g, Crohn's disease, ulcerative colitis), asthma, nephritis, hepatitis, pancreatitis, bronchitis, cystic fibrosis, ischemic bowel diseases, intestinal inflammations/allergies, coeliac disease, proctitis, eosnophilic gastroenteritis, mastocytosis, and other inflammatory disorders. The guanylin potentiating agents described herein can be used alone or in combination therapy in the treatment or prevention of gastrointestinal tract inflammation (e.g. inflammation associated with a gastrointestinal disorder, gastrointestinal tract infection, or another disorder). They can be used alone or in combination therapy with phenoxyalkycarboxylic acid derivatives for the treatment of interstitial cystitis, irritable bowel syndrome, ulcerative colitis, and other inflammatory conditions, as mentioned in US20050239902A1.

The guanylin potentiating agents described herein can also be used to treat or prevent insulin-related disorders, for example: II diabetes mellitus, hyperglycemia, obesity, disorders associated with disturbances in glucose or electrolyte transport and insulin secretion in cells, or endocrine disorders. They can be also used in insulin resistance treatment and post-surgical and non-post surgery decrease in insulin responsiveness.

The guanylin potentiating agents described herein can be used to prevent and/or treat pulmonary and respiratory related disorders, including, inhalation, ventilation and mucus secretion disorders, pulmonary hypertension, chronic obstruction of vessels and airways, and irreversible obstructions of vessels and bronchi. One may administer an agent for treating bronchospasm, for inducing bronchodilation, for treating chronic obstructive pulmonary disease (including chronic bronchitis with normal airflow), for treating asthma (including bronchial asthma, intrinsic asthma, extrinsic asthma, chronic or inveterate asthma (e.g. late asthma and airways hyper-responsiveness), dust-induced asthma, allergen-induced asthma, viral-induced asthma, cold-induced asthma, pollution-induced asthma and exercise-induced asthma) and for treating rhinitis (including acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis). The guanylin potentiating agents described herein may also be useful in the treatment of dry eye disease and chronic sinusitis. The guanylin potentiating agents described herein may also be used to prevent and/or treat disorders characterized by acute pulmonary vasoconstriction such as may result from pneumonia, traumatic injury, aspiration or inhalation injury, fat embolism in the lung, acidosis inflammation of the lung, adult respiratory distress syndrome, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, herapin-protamine reactions, sepsis, status asthmaticus or hypoxia (including iatrogenic hypoxia) and other forms of reversible pulmonary vasoconstriction. Such pulmonary disorders are also characterized by inflammation of the lung including those associated with the migration into the lung of nonresident cell types including the various leucocyte subclasses. Also included in the respiratory disorders contemplated are: bullous disease, cough, chronic cough associated with inflammation or iatrogenic induced, airway constriction, pigeon fancier's disease, eosinophilic bronchitis, asthmatic bronchitis, chronic bronchitis with airway obstruction (chronic obstructive bronchitis), eosinophilic lung disease, emphysema, farmer's lung, allergic eye diseases (including allergic conjunctivitis, vernal conjunctivitis, vernal keratoconjunctivitis, and giant papillary conjunctivitis), idiopathic pulmonary fibrosis, cystic fibrosis, diffuse pan bronchiolitis and other diseases which are characterized by inflammation of the lung and/or excess mucosal secretion. Other physiological events which are contemplated to be prevented, treated or controlled include platelet activation in the lung, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), chronic obstructive pulmonary disease (COPD)(such as irreversible COPD), chronic sinusitis, fibroid lung, hypersensitivity lung diseases, hypersensitivity pneumonitis, idiopathic interstitial pneumonia, nasal congestion, nasal polyposis, and otitis media.

The guanylin potentiating agents described herein can be used alone or in combitherapy to prevent or treat: retinopathy, nephropathy, diabetic angiopathy, and edema formation.

The guanylin potentiating agents described herein can be used alone or in combitherapy to prevent or treat neurological disorders, for example, headache, migraines, anxiety, stress, cognitive disorders, cerebral ischemia, brain trauma, movement disorders, aggression, psychosis, seizures, panic attacks, hysteria, sleep disorders, depression, schizoaffective disorders, sleep apnea, attention deficit syndromes, memory loss, dementia, memory and learning disorders as discussed in Moncada and Higgs 1995 FASEB J. 9:1319-1330; Severina 1998 Biochemistry 63:794; Lee et al. 2000 PNAS 97: 10763-10768; Hobbs 1997 TIPS 18:484-491; Murad 1994 Adv. Pharmacol. 26:1-335; and Denninger et al. 1999 Biochim. Biophys. Acta 1411:334-350 and narcolepsy. They may also be used as a sedative.

The guanylin potentiating agents described herein can be used in combination with GC-C agonists including detectably labeled GC-C agonists as markers to identify, detect, stage, or diagnosis diseases and conditions of small intestine, including, without limitation: Crohn's disease, colitis, inflammatory bowel disease, tumors, benign tumors, such as benign stromal tumors, adenoma, angioma, adenomatous (pedunculated and sessile) polyps, malignant, carcinoid tumors, endocrine cell tumors, lymphoma, adenocarcinoma, foregut, midgut, and hindgut carcinoma, gastroinstestinal stromal tumor (GIST), such as leiomyorna, cellular leiomyoma, leiomyoblastoma, and leiomyosarcoma, gastrointestinal autonomic nerve tumor, malabsorption syndromes, celiac diseases, diverticulosis, Meckel's diverticulum, colonic diverticula, megacolon, Hirschsprung's disease, irritable bowel syndrome, mesenteric ischemia, ischemic colitis, colorectal cancer, colonic polyposis, polyp syndrome, intestinal adenocarcinoma, Liddle syndrome, Brody myopathy, infantile convulsions, and choreoathetosis.

The guanylin potentiating agents described herein can used in combination with GC-C agonists conjugated to another molecule (e.g., a diagnostic or therapeutic molecule) to target cells bearing the GC-C receptor, e.g., cystic fibrosis lesions and specific cells lining the intestinal tract. Thus, they can be used to target radioactive moieties or therapeutic moieties to the intestine to aid in imaging and diagnosing or treating colorectal/metastasized or local colorectal cancer and to deliver normal copies of the p53 tumor suppressor gene to the intestinal tract.

The guanylin potentiating agents described herein can also be used to increase the number of GC-C molecules on the surface of a cell. In some embodiments the cell is a metastasized colorectal cancer cell. In one embodiment the GC-C agonist is therapeutically conjugated to a second agent. In certain embodiments, the second agent can be radioactive or radiostable. In certain embodiments the second agent can be selected from the group consisting of a compound that causes cell death, a compound that inhibits cell division, a compound that induces cell differentiation, a chemotherapeutic, a toxin and a radiosensitizing agent. In certain embodiments the second agent can be selected from the group consisting of: methotrexate, doxorubicin, daunorubicin, cytosinarabinoside, etoposide, 5-4 fluorouracil, melphalan, chlorambucil, cis-platin, vindesine, mitomycin, bleomycin, purothionin, macromomycin, 1,4-benzoquinone derivatives, trenimon, ricin, ricin A chain, Pseudomonas exotoxin, diphtheria toxin, Clostridium perfringens phospholipase C, bovine pancreatic ribonuclease, pokeweed antiviral protein, abrin, abrin A chain, cobra venom factor, gelonin, saporin, modeccin, viscumin, volkensin, nitroimidazole, metronidazole and misonidazole. In certain embodiments the second agent can be a cytoxic agent selected from the group consisting of cemadotin, a derivative of cemadotin, a derivative of hemiasterlin, esperamicin C, neocarzinostatin, maytansinoid DM1, 7-chloromethyl-10,11 methylenedioxy-camptothecin, rhizoxin, and the halichondrin B analog, ER-086526.

The guanylin potentiating agents described herein can be used alone or in combination therapy to prevent and/or treat inner ear disorders, e.g., to prevent and/or treat Meniere's disease (including symptoms thereof such as vertigo, hearing loss, tinnitus, sensation of fullness in the ear), Mal de debarquement syndrome, otitis extema, otitis media, otorrhea, acute mastoiditis, otosclerosis, otic pain, otic bleeding, otic inflammation, Lermoyez's syndrome, vestibular neuronitis, benign paroxysmal positional vertigo (BPPV), herpes zoster oticus, Ramsay Hunt's syndrome, herpes, labyrinthitis, purulent labyrinthitis, perilymph fistulas, presbycusis, ototoxicity (including drug-induced ototoxicity), neuromias (including acoustic neuromas), aerotitis media, infectious myringitis, bullous myringitis, squamous cell carcinoma, basal cell carcinoma, pre-cancerous otic conditions, nonchromaffin paragangliomas, chemodectomas, glomus jugulare tumors, glomus tympanicum tumors, perichondritis, aural eczematoid dermatitis, malignant external otitis, subperichondrial hematoma, ceruminomas, impacted cerumen, sebaceous cysts, osteomas, keloids, otalgia, tinnitus, tympanic membrane infection, tympanitis, otic furuncles, petrositis, conductive and sensorineural hearing loss, epidural abscess, lateral sinus thrombosis, subdural empyema, otitic hydrocephalus, Dandy's syndrome, bullous myringitis, diffuse external otitis, foreign bodies, keratosis obturans, otic neoplasm, otomycosis, trauma, acute barotitis media, acute eustachian tube obstruction, postsurgical otalgia, cholesteatoma, infections related to an otic surgical procedure, and complications associated with any of said disorders. The agents described herein can be used alone or in combination therapy to maintain fluid homeostasis in the inner ear and to prevent or treat neuronitis (including viral neuronitis), ganglionitis, and geniculate.

The guanylin potentiating agents described herein can be used alone or in combination therapy to prevent and/or treat disorders associated with fluid and sodium retention, e.g., diseases of the electrolyte-water/electrolyte transport system within the kidney, gut and urogenital system, congestive heart failure, hypertension, hypotension, salt dependent forms of high blood pressure, hepatic edema, and liver cirrhosis. In addition they can be used to facilitate diuresis or control intestinal fluid. Guanylin potentiating agents described herein can also be used to treat disorders where there is abnormal proliferation of epithelial cells within the kidney (e.g. as in the case of renal cancer). The guanylin potentiating agents described herein can be used alone or in combination therapy to prevent and/or treat kidney disease. “Kidney disease” includes renal failure (including acute renal failure), renal insufficiency, nephrotic edema, glomerulonephritis, pyelonephritis, kidney failure, chronic renal failure, nephritis, nephrosis, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome, along with any disease or disorder that relates to the renal system and related disorders, as well as symptoms indicative of, or related to, renal or kidney disease and related disorders.

The guanylin potentiating agents described herein can be used alone or in combination therapy to prevent or treat polycystic kidney disease. Polycystic kidney disease “PKD” (also called “polycystic renal disease”) refers to a group of disorders characterized by a large number of cysts distributed throughout dramatically enlarged kidneys. The resultant cyst development leads to impairment of kidney function and can eventually cause kidney failure. “PKD” specifically includes autosomal dominant polycystic kidney disease (ADPKD) and recessive autosomal recessive polycystic kidney disease (ARPKD), in all stages of development, regardless of the underlying cause.

The guanylin potentiating agents described herein can be used alone or in combination therapy to prevent and/or treat disorders associated with bicarbonate secretion, e.g., Cystic Fibrosis.

The guanylin potentiating agents described herein can be used alone or in combination therapy to prevent and/or treat disorders associated with bile secretion. In addition, they can be used to facilitate or control chloride and bile fluid secretion in the gall bladder.

The guanylin potentiating agents described herein can be used alone or in combination therapy to prevent and/or treat disorders associated with liver cell regeneration. This may include administration of the peptides and agonists to liver transplant recipients and to patients with drug or alcohol induced-liver damage. Furthermore, agents may be useful to treat liver damage as in the case of viral mediated hepatitis. The guanylin potentiating agents described herein may be used alone or in combination to prevent and/or treat liver abscess, liver cancer (either primary or metastatic), cirrhosis (such as cirrhosis caused by the alcohol consumption or primary biliary cirrhosis), amebic liver abscess, autoimmune hepatitis, biliary atresia, coccidioidomycosis disseminated, δ agent (hepatitis δ), hemochromatosis, hepatitis a, hepatitis b, hepatitis c, or any other acute, subacute, fulminant or chronic hepatitis of viral, metabolic or toxic etiology, hepatocellular carcinoma, pyogenic liver abscess, Reye's syndrome, sclerosing cholangitis, Wilson's disease, drug induced hepatotoxicity, or fulminant or acute liver failure. The agents described herein may be used in stimulating hepatic regeneration after surgical hepatectomy.

The guanylin potentiating agents described herein can be used alone or in combination therapy to prevent and/or treat myocardial infraction, diastolic dysfunction, angina pectoris, stable, unstable and variant (Prinzmetal) angina, atherosclerosis, thrombosis, endothelial dysfunction, cardiac edema, stroke, conditions of reduced blood vessel patency, e.g., postpercutaneous transluminal coronary angioplasty (post-PTCA) and peripheral vascular disease.

The guanylin potentiating agents described herein can be used alone or in combination therapy to prevent and/or treat glaucoma. The guanylin potentiating agents described herein can be used alone or in combination therapy to prevent and/or treat immunodeficiency. The guanylin potentiating agents described herein can be used alone or in combination therapy to prevent and/or treat bladder outlet obstruction and incontinence.

The guanylin potentiating agents described herein can be used alone or in combination therapy to prevent and/or treat male (e.g. erectile dysfunction) or female sexual dysfunction, premature labor, dysmenorrhoea, endometriosis, polycystic ovary syndrome, vaginal dryness, uterine pain, or pelvic pain. These guanylin potentiating agents of the invention can be utilized as tocolytic agents that decrease or arrest uterine contractions. The guanylin potentiating agents of the invention can be used to prevent/treat premature/preterm labor. Premature or preterm labor can be associated with, for example, an illness/disorder/condition of the mother (such as pre-eclampsia, high blood pressure or diabetes, abnormal shape or size of the uterus, weak or short cervix, hormone imbalance, vaginal infection that spreads to the uterus, abnormalities of the placenta, such as placenta previa, and excessive amniotic fluid), premature rupture of the amniotic membranes (“water breaks”), large fetus, and more than one fetus. The guanylin potentiating agents of the invention can be used to prevent uterine rupture. The guanylin potentiating agents of the invention can be used treat rapid uterine contractions (for example, associated with placental abruption wherein the placental abruption is associated with hypertension, diabetes, a multiply pregnancy, an unusually large amount of amniotic fluid, numerous previous deliveries, or advanced maternal age (e.g. >40 years old). In certain embodiments they can be used in combination with a phosphodiesterase inhibitor. The guanylin potentiating agents of the invention can be used alone or in combination therapy to prevent and/or treat infertility, for example, male infertility due to poor sperm quality, decreased sperm motility or low sperm count.

The guanylin potentiating agents described herein can be used alone or in combination therapy to prevent and/or treat osteopenia disorders (bone loss disorders). “Bone loss disorders” include conditions and diseases wherein the inhibition of bone loss and/or the promotion of bone formation is desirable. Among such conditions and diseases are osteoporosis, osteomyelitis, Paget's disease (osteitis deformans), periodontitis, hypercalcemia, osteonecrosis, osteosarcoma, osteolyic metastases, familial expansile osteolysis, prosthetic loosening, periprostetic osteolysis, bone loss attendant rheumatoid arthritis, and cleiodocranial dysplasia (CCD). Osteoporosis includes primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Rile-Day syndrome) and osteoporosis due to immobilization of extremitiesosteomyelitis, or an infectious lesion in bone leading to bone loss. The agents can be used alone or in combination therapy to stimulating bone regeneration. The bone regeneration may be following reconstruction of bone defects in cranio-maxillofacial surgery, or following an implant into bone, for example a dental implant, bone supporting implant, or prosthesis. The bone regeneration may also be following a bone fracture.

The guanylin potentiating agents of the invention may be used alone or in combination therapy (for example, with other agents that increase cGMP) to prevent or treat disorders related to an alteration in cGMP including, but not limited to Alzheimer's disease, psoriasis, skin necrosis, scarring, fibrosis, baldness, Kawasaki's Disease, nutcracker oesophagus (US20050245544), septic shock, NSAID-induced gastric disease or disorder, ischemic renal disease or disorder, peptic ulcer, sickle cell anemia, epilepsy, and a neuroinflammatory disease or disorder (for example as described in WO05105765).

1-47. (canceled)

48. A method for increasing GC-C receptor activity, the method comprising administering a composition comprising a guanylin potentiating agent

49. A method for treating a disorder ameliorated by increasing cGMP levels, the method comprising administering a composition comprising a guanylin potentiating agent.

50. The method of claim 49 wherein the disorder is selected from: congestive heart failure, hypertension, a gastrointestinal disorder, and obesity.

51. The method of claim 50 wherein the gastrointestinal disorder is selected from the group consisting of: a gastrointestinal motility disorder, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, and constipation.

52. The method of claim 49 further comprising administering guanylin or a biologically active variant or fragment thereof.

53. The method of claim 49 further comprising administering a GC-C receptor agonist.

54. The method of claim 49 wherein the guanylin potentiating agent is a chymotrypsin inhibitor.

55. A pharmaceutical composition comprising a guanylin potentiating agent inhibitor and a pharmaceutically acceptable carrier.

56. The pharmaceutical composition of claim 55 further comprising guanylin or a biologically active variant or fragment thereof.

57. A pharmaceutical composition comprising at least two of:
(a) an agent that stimulates the production of cAMP
(b) an agent that inhibits the degradation of a cyclic nucleotide; and
(c) a guanylin potentiating agent.

58. The pharmaceutical composition of claim 57 wherein (a) is glucagon-like peptide 1.

59. The pharmaceutical composition of claim 57 wherein (b) is a phosphodiesterase inhibitor.

60. The method of claim 52 wherein the guanylin or biologically active variant or fragment thereof is a polypeptide comprising the amino acid sequence:
A′-B′-C′ wherein:
A′ is an amino acid sequence comprising a pre sequence depicted in FIG. 6 or is missing;
B′ is an amino acid sequence comprising a pro sequence depicted in FIG. 6 or is missing;
C′ is an amino acid sequence comprising a GC-C receptor agonist polypeptide amino acid sequence.

61. The method of claim 60 wherein C′ comprises an amino acid sequence selected from:

| | |
|---|---|
| PGTCEICASAACTGC | (SEQ ID NO: 4690) |
| PGTCEICATAACTGC | (SEQ ID NO: 4691) |
| PGTCEICANAACTGC | (SEQ ID NO: 4692) |
| PGTCEICAQAACTGC | (SEQ ID NO: 4693) |
| PGTCEICARAACTGC | (SEQ ID NO: 4694) |
| PGTCEICAEAACTGC | (SEQ ID NO: 4695) |
| PGTCEICADAACTGC | (SEQ ID NO: 4696) |
| PGTCEICAGAACTGC | (SEQ ID NO: 4697) |
| PGTCEICAAAACTGC | (SEQ ID NO: 4698) |
| PGTCEICAMAACTGC | (SEQ ID NO: 4699) |
| PGTCEICAIAACTGC | (SEQ ID NO: 4700) |
| PGTCEICALAACTGG | (SEQ ID NO: 4701) |
| PGTCEICAVAACTGC | (SEQ ID NO: 4702) |
| PGTCEICAHAACTGC | (SEQ ID NO: 4703) |
| PGTGEGICAYAACTGC | (SEQ ID NO: 4704) |
| PGTCEIGCAYAACTGC | (SEQ ID NO: 4705) |
| PGTCEICGAYAACTGC | (SEQ ID NO: 4706) |
| PGTGEICAGYAACTGC | (SEQ ID NO: 4707) |
| PGTCEICAYGAACTGC | (SEQ ID NO: 4708) |
| PGTCEICAYAGACTGC | (SEQ ID NO: 4709) |
| PGTCEICAYAAGCTGC | (SEQ ID NO: 4710) |
| PGTCEICAYAACGTGC | (SEQ ID NO: 4711) |
| PGTCEICAYAACTGGC | (SEQ ID NO: 4712) |
| PGTCAEICAYAACTGC | (SEQ ID NO: 4713) |
| PGTCEAICAYAACTGC | (SEQ ID NO: 4714) |
| PGTCEIACAYAACTGC | (SEQ ID NO: 4715) |
| PGTCEICAAYAACTGC | (SEQ ID NO: 4716) |
| PGTCEICAYAAACTGC | (SEQ ID NO: 4717) |
| PGTCEICAYAACATGC | (SEQ ID NO: 4718) |
| PGTCEICAYAACTAGC | (SEQ ID NO: 4719) |
| PGTCEICAYAACTGAC | (SEQ ID NO: 4720) |
| PGTCAEICAAYAACTGC | (SEQ ID NO: 4721) |
| PGTCEAICAAYAACTGC | (SEQ ID NO: 4722) |
| and | |
| PGTCEIACAAYAACTGC. | (SEQ ID NO: 4723) |

62. The method of claim 60 wherein C′ comprises an amino acid sequence selected from the processed active peptide sequences shown in FIG. 6.

63. The method of claim 60 wherein A′ is missing and B′ is an amino acid sequence comprising a pro sequence depicted in FIG. 6.

64. The pharmaceutical composition of claim 56 wherein the guanylin or biologically active variant or fragment thereof comprises the amino acid sequence:
A′-B′-C′ wherein:
A′ is an amino acid sequence comprising a pre sequence depicted in FIG. 6 or is missing;
B′ is an amino acid sequence comprising a pro sequence depicted in FIG. 6 or is missing;
C′ is an amino acid sequence comprising a GC-C receptor agonist polypeptide amino acid sequence.

65. The composition of claim 64 wherein C′ comprises an amino acid sequence selected from:

| | |
|---|---|
| PGTCEICASAACTGC | (SEQ ID NO: 4690) |
| PGTCEICATAACTGC | (SEQ ID NO: 4691) |
| PGTCEICANAACTGC | (SEQ ID NO: 4692) |
| PGTCEICAQAACTGC | (SEQ ID NO: 4693) |
| PGTCEICARAACTGC | (SEQ ID NO: 4694) |
| PGTCEICAEAACTGC | (SEQ ID NO: 4695) |
| PGTCEICADAACTGC | (SEQ ID NO: 4696) |
| PGTCEICAGAACTGC | (SEQ ID NO: 4697) |
| PGTCEICAAAACTGC | (SEQ ID NO: 4698) |
| PGTCEICAMAACTGC | (SEQ ID NO: 4699) |
| PGTCEICAIAACTGC | (SEQ ID NO: 4700) |
| PGTCEICALAACTGC | (SEQ ID NO: 4701) |
| PGTCEICAVAACTGC | (SEQ ID NO: 4702) |
| PGTCEICAHAACTGC | (SEQ ID NO: 4703) |
| PGTCEGICAYAACTGC | (SEQ ID NO: 4704) |
| PGTCEIGCAYAACTGC | (SEQ ID NO: 4705) |
| PGTCEICGAYAACTGC | (SEQ ID NO: 4706) |
| PGTCEICAGYAACTGC | (SEQ ID NO: 4707) |
| PGTCEICAYGAACTGC | (SEQ ID NO: 4708) |
| PGTCEICAYAGACTGC | (SEQ ID NO: 4709) |
| PGTCEICAYAAGCTGC | (SEQ ID NO: 4710) |
| PGTCEICAYAACGTGC | (SEQ ID NO: 4711) |
| PGTCEICAYAACTGGC | (SEQ ID NO: 4712) |
| PGTCAEICAYAACTGC | (SEQ ID NO: 4713) |
| PGTCEAICAYAACTGC | (SEQ ID NO: 4714) |
| PGTCEIACAYAACTGC | (SEQ ID NO: 4715) |
| PGTCEICAAYAACTGC | (SEQ ID NO: 4716) |
| PGTCEICAYAAACTGC | (SEQ ID NO: 4717) |
| PGTCEICAYAACATGC | (SEQ ID NO: 4718) |
| PGTCEICAYAACTAGC | (SEQ ID NO: 4719) |
| PGTCEICAYAACTGAC | (SEQ ID NO: 4720) |
| PGTCAEICAAYAACTGC | (SEQ ID NO: 4721) |
| PGTCEAICAAYAACTGC | (SEQ ID NO: 4722) |
| and | |
| PGTCEIACAAYAACTGC. | (SEQ ID NO: 4723) |

66. The composition of claim 64 wherein C′ comprises an amino acid sequence selected from the processed active peptide sequences shown in FIG. 6.

67. The composition of claim 64 wherein A′ is missing and B′ is an amino acid sequence comprising a pro sequence depicted in FIG. 6.
